SEQUENCE LISTING (79 sequences)

SEQ ID NO: 1 - protein, 65 aa, synthetic construct
  REGION 1..65: note = partial oligomerization domain of C4BP
SAGAHAGWETPEGCEQVLTGKRLMQCLPNPEDVKMALEVYKLSLEIEQLELQRDSARQSTLDKEL

SEQ ID NO: 2 - protein, 55 aa, synthetic construct
  REGION 1..55: note = partial oligomerization domain of C4BP
WVIPEGCGHVLAGRKVMQCLPNPEDVKMALEVYKLSLEIELLEIQRDKARDPAMD

SEQ ID NO: 3 - protein, 52 aa, synthetic construct
  REGION 1..52: note = partial oligomerization domain of C4BP
WEYAEGCEQVVKGKKLMQCLPTPEEVRLALEVYKLYLEIQKLELQKDEAKQA

SEQ ID NO: 4 - protein, 58 aa, synthetic construct
  REGION 1..58: note = partial oligomerization domain of C4BP
WVVPAGCEQVIAGRELTQCLPSVEDVKMALELYKLSLEIELLELQKDKAKKSTLESPL

SEQ ID NO: 5 - protein, 135 aa, synthetic construct
  REGION 1..135: note = Anti-tcdB VHH 5D
QVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSEPKTPKPQ

SEQ ID NO: 6 - protein, 119 aa, synthetic construct
  REGION 1..119: note = Anti-tcdB VHH E3
QVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSSEPKTPKPQ

SEQ ID NO: 7 - protein, 123 aa, synthetic construct
  REGION 1..123: note = Anti-tcdB VHH 5E
VQLVESGGGLVQAGGSLRLSCAASGLMFGAMTMGWYRQAPGKEREMVAYITAGGTESYSESVKGRFTISRINANNMVYLQMTNLKVEDTAVYYCNAHNFWRTSRNWGQGTQVTVSSEPKTPKP

SEQ ID NO: 8 - protein, 131 aa, synthetic construct
  REGION 1..131: note = Anti-tcdB VHH B12
VQLVESGGGLVQAGDSLTLSCAASESTFNTFSMAWFRQAPGKEREYVAAFSRSGGTTNYADSVKGRATISTDNAKNTVYLHMNSLKPEDTAVYFCAADRPAGRAYFQSRSYNYWGQGTQVTVSSAHHSEDP

SEQ ID NO: 9 - protein, 125 aa, synthetic construct
  REGION 1..125: note = Anti-tcdB VHH A11
VQLVESGGGSVQIGGSLRLSCVASGFTFSKNIMSWARQAPGKGLEWVSTISIGGAATSYADSVKGRFTISRDNANDTLYLQMNNLKPEDTAVYYCSRGPRTYINTASRGQGTQVTVSSEPKTPKP

SEQ ID NO: 10 - protein, 129 aa, synthetic construct
  REGION 1..129: note = Anti-tcdB VHH A1
VQLVESGGGLVQAGGSLRLSCAAPGLTFTSYRMGWFRQAPGKEREYVAAITGAGATNYADSAKGRFTISKNNTASTVHLQMNSLKPEDTAVYYCAASNRAGGYWRASQYDYWGQGTQVTVSSAHHSEDP

SEQ ID NO: 11 - protein, 135 aa, synthetic construct
  REGION 1..135: note = Anti-tcdB VHH 2D
QVQLVESGGGLVQPGGSLRLSCAASGFSLDYYGIGWFRQAPGKERQEVSYISASAKTKLYSDSVKGRFTISRDNAKNAVYLEMNSLKREDTAVYYCARRRFDASASNRWLAADYDYWGQGTQVTVSSEPKTPKPQ

SEQ ID NO: 12 - protein, 123 aa, synthetic construct
  REGION 1..123: note = Anti-tcdB VHH 2Ds
QVQLVESGGGLVQAGGSLRLSCVSSERNPGINAMGWYRQAPGSQRELVAIWQTGGSLNYADSVKGRFTISRDNLKNTVYLQMNSLKPEDTAVYYCYLKKWRDQYWGQGTQVTVSSEPKTPKPQ

SEQ ID NO: 13 - protein, 124 aa, synthetic construct
  REGION 1..124: note = Anti-tcdB VHH 7F
QVQLVESGGGLVEAGGSLRLSCVVTGSSFSTSTMAWYRQPPGKQREWVASFTSGGAIKYTDSVKGRFTMSRDNAKKMTYLQMENLKPEDTAVYYCALHNAVSGSSWGRGTQVTVSSEPKTPKPQ

SEQ ID NO: 14 - protein, 131 aa, synthetic construct; identical to SEQ ID NO: 8
  REGION 1..131: note = Anti-tcdB VHH B12

SEQ ID NO: 15 - protein, 121 aa, synthetic construct
  REGION 1..121: note = Anti-tcdB VHH AB8
VQLVESGGGLVQAGGSLRLSCVGSGRNPGINAMGWYRQAPGSQRELVAVWQTGGSTNYADSVKGRFTISRDNLKNTVYLQMNSLKPEDTAVYYCYLKKWRDEYWGQGTQVTVSSAHHSEDP

SEQ ID NO: 16 - protein, 124 aa, synthetic construct
  REGION 1..124: note = Anti-tcdB VHH C6
VQLVESGGGLVQAGESLRLSCVVSESIFRINTMGWYRQTPGKQREVVARITLRNSTTYADSVKGRFTISRDDAKNTLYLKMDSLKPEDTAVYYCHRYPLIFRNSPYWGQGTQVTVSSEPKTPKP

SEQ ID NO: 17 - protein, 122 aa, synthetic construct
  REGION 1..122: note = Anti-tcdB VHH C12
VQLVESGGGLVQAGESLRLSCVVSESIFRINTMGWYRQTPGKQREVVARITLRNSTTYADSVKGRFTISRDDAKNTLYLKMDSLKPEDTAVYYCHRYPLIFRNSPYWGQGTQVTVSSEPKTP

SEQ ID NO: 18 - protein, 126 aa, synthetic construct
  REGION 1..126: note = K922 VHH
QVQLQESGGGLVQPGGSLRLSCALVSGGTFSWYAMGWFRQAPGKEREFVATVSRGGGSSYYADSVKGRFTISRDNAKNTVYLQMNSLKPEDTAVYYCAAGRGAPSDTGRPDEYDYWGQGTQVTVSS

SEQ ID NO: 19 - protein, 8 aa, synthetic construct
  REGION 1..8: note = VHH 5D helix 1
AEAAAKAS

SEQ ID NO: 20 - protein, 13 aa, synthetic construct
  REGION 1..13: note = VHH 5D helix 2
AEAAAKEAAAKAS

SEQ ID NO: 21 - protein, 23 aa, synthetic construct
  REGION 1..23: note = VHH 5D helix 4
AEAAAKEAAAKEAAAKEAAAKAS

SEQ ID NO: 22 - protein, 12 aa, synthetic construct
  REGION 1..12: note = VHH 5D PA5
APAPSPAPSPAS

SEQ ID NO: 23 - protein, 22 aa, synthetic construct
  REGION 1..22: note = VHH 5D PA10
APAPSPAPAPSPAPAPSPAPAS

SEQ ID NO: 24 - protein, 34 aa, synthetic construct
  REGION 1..34: note = VHH 5D PA15
APAPSPAPAPSPAPAPSPAPAPSPAPAPSPAPAS

SEQ ID NO: 25 - protein, 25 aa, synthetic construct
  REGION 1..25: note = VHH 5D IgA linker
APPPVPSTPPTPSPSTPPTPSPPPS

SEQ ID NO: 26 - DNA, 729 bp, synthetic construct
  misc_feature 1..729: note = Malaria vaccine; P. yoelii CSP B cell epitope in monomer WHcAg
  source 1..729: mol_type = other DNA
atggacatagatccctataaagaatttggttcatcttatcagttgttgaattttcttcctttggacttctttcctgaccttaatgctttggtggacactgctactgccttgtatgaagaagagctaacaggtagggaacattgctctccgcaccatacagctattagacaagctttagtatgctgggatgaattaactaaattgatagcttggatgagctctaacataacttctcagggtccaggtgctccacaaggacctggagcacctcagggccctggcgctccccaagggcctggagcccctcagggacccggtgcaccgcaaggtccgggcgctccccaagaaccacctcaacagcctccacagcaacctcctcagcagccaccacaacagccccctcagcaagaacaagtaagaacaatcatagtaaatcatgtcaatgatacctggggacttaaggtgagacaaagtttatggtttcatttgtcatgtctcacttttggacaacatacagttcaagaatttttagtaagttttggagtatggatcagaactccagctccatatagacctcctaatgcacccattctctcgactcttccggaacatacagtcattaatgaagatagttatgttccttctgctgaacaaattttagaatttgagcaaaaattaattagcgaggaagacctagaacaaaaactgatctctgaagaggatctgtaa

SEQ ID NO: 27 - protein, 242 aa, synthetic construct
  REGION 1..242: note = Malaria vaccine; P. yoelii CSP B cell epitope in monomer WHcAg
MDIDPYKEFGSSYQLLNFLPLDFFPDLNALVDTATALYEEELTGREHCSPHHTAIRQALVCWDELTKLIAWMSSNITSQGPGAPQGPGAPQGPGAPQGPGAPQGPGAPQGPGAPQEPPQQPPQQPPQQPPQQPPQQEQVRTIIVNHVNDTWGLKVRQSLWFHLSCLTFGQHTVQEFLVSFGVWIRTPAPYRPPNAPILSTLPEHTVINEDSYVPSAEQILEFEQKLISEEDLEQKLISEEDL

SEQ ID NO: 28 - DNA, 1155 bp, synthetic construct
  misc_feature 1..1155: note = Malaria vaccine; P. falciparum CSP B cell epitope in tandem WHcAg dimer
  source 1..1155: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattggtggaagtggagggtctggtgggtccgggggtagtggtgggtctgatatcgatccctacaaagaattcggcagttcttatcagttactaaatttcctgccgctggatttttttcccgatctgaacgccttggtcgatactgccaccgccttgtacgaggaagagctaaccgggcgagagcattgtagtccacatcatactgctatccgccaggctctggtctgctgggacgaattgaccaagttaattgcatggatgagctccaatattactagtgaagagggtaatgcaaaccctaatgcgaacccgaacgctaaccctaatgccaatcctaacgctaatcccaatgccaacccgaatgcaaacccaaatgcgaatccgaatgctaacccgaacgctaacccgaatgcgaatccaaacgcgaaccccaacgcaaatccgaatgcaaaccctaatgcaaatccaggtgaagaggagcaggtccgcacgatcattgttaaccacgtcaacgatacctggggcctaaaggttcgccaatctttgtggttccatctgtcgtgcctgacctttgggcaacacaccgtccaggagttcctggtgagcttcggcgtttggatccgcaccccagcaccctaccgcccgccaaatgctcccattttaagtaccttgcccgaacacaccgtgattcaccaccatcatcaccactaa

SEQ ID NO: 29 - protein, 384 aa, synthetic construct
  REGION 1..384: note = Malaria vaccine; P. falciparum CSP B cell epitope in tandem WHcAg dimer
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIGGSGGSGGSGGSGGSDIDPYKEFGSSYQLLNFLPLDFFPDLNALVDTATALYEEELTGREHCSPHHTAIRQALVCWDELTKLIAWMSSNITSEEGNANPNANPNANPNANPNANPNANPNANPNANPNANPNANPNANPNANPNANPNANPNANPGEEEQVRTIIVNHVNDTWGLKVRQSLWFHLSCLTFGQHTVQEFLVSFGVWIRTPAPYRPPNAPILSTLPEHTVIHHHHHH

SEQ ID NO: 30 - DNA, 1971 bp, synthetic construct
  misc_feature 1..1971: note = Malaria vaccine; P. falciparum CSP B cell epitope + Salmonella fliC sequences in tandem WHcAg dimer
  source 1..1971: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattggtggaagtggagggtctggtgggtccgggggtagtggtgggtctgatatcgatccctacaaagaattcggcagttcttatcagttactaaatttcctgccgctggatttttttcccgatctgaacgccttggtcgatactgccaccgccttgtacgaggaagagctaaccgggcgagagcattgtagtccacatcatactgctatccgccaggctctggtctgctgggacgaattgaccaagttaattgcatggatgagctccaatattactagtgaagagggtgcgcaggtcattaacactaattcgctgagtttactaacacagaataatctaaacaagagccaatccgcccttggcacggcgatcgagcggctgagttcggggctgcgtatcaatagtgccaaagatgacgcggccggccaggcgattgcaaatcgttttacggccaatataaaggggcttacacaggcttctcgtaatgccaacgacggtatttccattgcccaaacaacggaaggcgcgctgaatgaaatcaataataatctgcagcgggtccgggagttagcggtgcagtctgccaactcaacaaattctcaatcagatctggattctatccaggcagaaataactcagaggcttaatgaaatcgatcgtgtttctggacaaacccagtttaatggtgtcaaggtccttgctcaggacaacaccctgaccatccaggtaggcgcgaacgatggagaaaccattgatattgatctgaaacagattaattctcagactctaggtcttgacaccttgaatgtgcagggttctaatgcaaaccctaatgcgaacccgaacgctaaccctaatgccaatcctaacgctaatcccaatgccaacccgaatgcaaacccaaatgcgaatccgaatgctaacccgaacgctaacccgaatgcgaatccaaacgcgaaccccaacgcaaatccgaatgcaaaccctaatgcaaatccaggttctaccacaaccgagaatcctctgcagaaaatcgatgctgctctcgcgcaagtggacactttgcgttcagatttgggagctgtgcaaaatcgtttcaacagcgcgattacaaacctgggtaacaccgtaaacaatctgactagtgcccggagtcggattgaagatagcgattatgcgaccgaagtgtctaacatgagccgggcccaaatcttgcagcaagccggcactagtgttctggcgcaagcaaatcaggtcccccaaaacgttctcagccttctgcggggtgaagaggagcaggtccgcacgatcattgttaaccacgtcaacgatacctggggcctaaaggttcgccaatctttgtggttccatctgtcgtgcctgacctttgggcaacacaccgtccaggagttcctggtgagcttcggcgtttggatccgcaccccagcaccctaccgcccgccaaatgctcccattttaagtaccttgcccgaacacaccgtgattcaccaccatcatcaccactaa

SEQ ID NO: 31 - protein, 656 aa, synthetic construct
  REGION 1..656: note = Malaria vaccine; P. falciparum CSP B cell epitope + Salmonella fliC sequences in tandem WHcAg dimer
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIGGSGGSGGSGGSGGSDIDPYKEFGSSYQLLNFLPLDFFPDLNALVDTATALYEEELTGREHCSPHHTAIRQALVCWDELTKLIAWMSSNITSEEGAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQGSNANPNANPNANPNANPNANPNANPNANPNANPNANPNANPNANPNANPNANPNANPNANPGSTTTENPLQKIDAALAQVDTLRSDLGAVQNRFNSAITNLGNTVNNLTSARSRIEDSDYATEVSNMSRAQILQQAGTSVLAQANQVPQNVLSLLRGEEEQVRTIIVNHVNDTWGLKVRQSLWFHLSCLTFGQHTVQEFLVSFGVWIRTPAPYRPPNAPILSTLPEHTVIHHHHHH

SEQ ID NO: 32 - DNA, 621 bp, synthetic construct
  misc_feature 1..621: note = Canine parvovirus vaccine; 2L21 peptide epitope in 2x configuration; monomer WhcAg
  source 1..621: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaagagggttctgacggtgctgtgcagcccgatggcggtcaacccgctgttcgtaatgaacgtgctactggtggtggctctagtgatggtgctgttcagcctgacggtggtcaacctgctgtgcgcaacgagcgtgcaacaggaggtgaagaggaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattcaccaccatcatcaccactaa

SEQ ID NO: 33 - protein, 206 aa, synthetic construct
  REGION 1..206: note = Canine parvovirus vaccine; 2L21 peptide epitope in 2x configuration; monomer WhcAg
MDIDPYKEFGSSYQLLNFLPLDFFPDLNALVDTATALYEEELTGREHCSPHHTAIRQALVCWDELTKLIAWMSSNITSEEGSDGAVQPDGGQPAVRNERATGGGSSDGAVQPDGGQPAVRNERATGGEEEQVRTIIVNHVNDTWGLKVRQSLWFHLSCLTFGQHTVQEFLVSFGVWIRTPAPYRPPNAPILSTLPEHTVIHHHHHH

SEQ ID NO: 34 - DNA, 717 bp, synthetic construct
  misc_feature 1..717: note = Canine parvovirus vaccine; 3L17 peptide epitope in 4x configuration; monomer WhcAg
  source 1..717: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaagagggtgacggtgctgtgcagcccgatggcggtcaacccgctgttcgtaatgaacgtggtggctctgatggtgctgttcagcctgacggtggtcaacctgctgtgcgcaacgagcgtggtggttccgacggtgccgttcaacccgacggtggccaacccgccgtgcgtaatgagcgcggtggttctgacggcgctgtgcaacctgacggcggtcagcccgccgttcgtaacgagcgtggtgaagaggaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattcaccaccatcatcaccactaa

SEQ ID NO: 35 - protein, 238 aa, synthetic construct
  REGION 1..238: note = Canine parvovirus vaccine; 3L17 peptide epitope in 4x configuration; monomer WhcAg
MDIDPYKEFGSSYQLLNFLPLDFFPDLNALVDTATALYEEELTGREHCSPHHTAIRQALVCWDELTKLIAWMSSNITSEEGDGAVQPDGGQPAVRNERGGSDGAVQPDGGQPAVRNERGGSDGAVQPDGGQPAVRNERGGSDGAVQPDGGQPAVRNERGEEEQVRTIIVNHVNDTWGLKVRQSLWFHLSCLTFGQHTVQEFLVSFGVWIRTPAPYRPPNAPILSTLPEHTVIHHHHHH

SEQ ID NO: 36 - DNA, 1152 bp, synthetic construct
  misc_feature 1..1152: note = IHNV vaccine; E1+E2 epitopes in tandem WHcAg dimer
  source 1..1152: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattggtggaagtggagggtctggtgggtccgggggtagtggtgggtctgatatcgatccctacaaagaattcggcagttcttatcagttactaaatttcctgccgctggatttttttcccgatctgaacgccttggtcgatactgccaccgccttgtacgaggaagagctaaccgggcgagagcattgtagtccacatcatactgctatccgccaggctctggtctgctgggacgaattgaccaagttaattgcatggatgagctccaatattactagtcctggtggaagtggagacgatgaaaatcgtggcttgatcgcttatcctaccagtatccgttccttgagtgtcggcggaagtggagggtctgatctgattagcgtggtttacaacagtggaagcgagatcctgtcgtttcctggtggatcaggggagcaggtccgcacgatcattgttaaccacgtcaacgatacctggggcctaaaggttcgccaatctttgtggttccatctgtcgtgcctgacctttgggcaacacaccgtccaggagttcctggtgagcttcggcgtttggatccgcaccccagcaccctaccgcccgccaaatgctcccattttaagtaccttgcccgaacacaccgtgattgagcaaaaattaattagcgaggaagacctagaacaaaaactgatctctgaagaggatctgtaa

SEQ ID NO: 37 - protein, 383 aa, synthetic construct
  REGION 1..383: note = IHNV vaccine; E1+E2 epitopes in tandem WHcAg dimer
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIGGSGGSGGSGGSGGSDIDPYKEFGSSYQLLNFLPLDFFPDLNALVDTATALYEEELTGREHCSPHHTAIRQALVCWDELTKLIAWMSSNITSPGGSGDDENRGLIAYPTSIRSLSVGGSGGSDLISVVYNSGSEILSFPGGSGEQVRTIIVNHVNDTWGLKVRQSLWFHLSCLTFGQHTVQEFLVSFGVWIRTPAPYRPPNAPILSTLPEHTVIEQKLISEEDLEQKLISEEDL

SEQ ID NO: 38 - DNA, 1356 bp, synthetic construct
  misc_feature 1..1356: note = IHNV vaccine; DIII epitope in tandem WHcAg dimer
  source 1..1356: mol_type = other DNA
atggacatagatccctataaagaatttggttcatcttatcagttgttgaattttctacctttggacttctttcctgacctaaatgctttggtggacactgctactgccttgtatgaagaagagctaacaggtcgagaacattgctctccgcaccatacagctattagacaagctttagtatgctgggatgaattaactaaattgatagcttggatgagctctaacataacttctgaacaagtaagaacaatcatagtaaatcatgtcaatgatacctggggattaaaggtgagacaaagtttatggtttcatttgtcatgtttgacttttggacaacatacagttcaagaatttttagtaagttttggagtatggatcagaactccagctccatatagacctcctaatgcacccattctctccactcttcccgaacatacagtcattggtggaagtggagggtctggtgggtccgggggtagtggtgggtctgatatcgatccctacaaagaattcggcagttcttatcagttactaaatttcctgccgctggatttttttcccgatctgaacgccttggtcgatactgccaccgccttgtacgaggaagagctaaccgggcgagagcattgtagtccacatcatactgctatccgccaggctctggtctgctgggacgaattgaccaagttaattgcatggatgagctccaatattactagtcctggtggaagtggagacgatgagaacagggggctaattgcctatcccacatccatccggtccctgtcagtcggaaacgacggtggcagtggagggtctagccaagagataaaagctcacctctttgttgataaaatctccaatcgagtcgtgaaggcaacgagctacggacaccacccctggggactgcatcaggcctgtatgattgaattctgtgggcaacagtggatacggacagatctcggtgacctaatatctgtcgtatacaattctggatcagaaatcctctcgttcccgaagtgtgaagacaagaccgtgggaccagcagagggtggcggtccagcaggtggatcaggggagcaggtccgcacgatcattgttaaccacgtcaacgatacctggggcctaaaggttcgccaatctttgtggttccatctgtcgtgcctgacctttgggcaacacaccgtccaggagttcctggtgagcttcggcgtttggatccgcaccccagcaccctaccgcccgccaaatgctcccattttaagtaccttgcccgaacacaccgtgattgagcaaaaattaattagcgaggaagacctagaacaaaaactgatctctgaagaggatctgtaa

SEQ ID NO: 39 - protein, 451 aa, synthetic construct
  REGION 1..451: note = IHNV vaccine; DIII epitope in tandem WHcAg dimer
MDIDPYKEFGSSYQLLNFLPLDFFPDLNALVDTATALYEEELTGREHCSPHHTAIRQALVCWDELTKLIAWMSSNITSEQVRTIIVNHVNDTWGLKVRQSLWFHLSCLTFGQHTVQEFLVSFGVWIRTPAPYRPPNAPILSTLPEHTVIGGSGGSGGSGGSGGSDIDPYKEFGSSYQLLNFLPLDFFPDLNALVDTATALYEEELTGREHCSPHHTAIRQALVCWDELTKLIAWMSSNITSPGGSGDDENRGLIAYPTSIRSLSVGNDGGSGGSSQEIKAHLFVDKISNRVVKATSYGHHPWGLHQACMIEFCGQQWIRTDLGDLISVVYNSGSEILSFPKCEDKTVGPAEGGGPAGGSGEQVRTIIVNHVNDTWGLKVRQSLWFHLSCLTFGQHTVQEFLVSFGVWIRTPAPYRPPNAPILSTLPEHTVIEQKLISEEDLEQKLISEEDL

SEQ ID NO: 40 - protein, 131 aa, Lama glama
QVQLQESGGGLVQPGGSLRLSCAASESTPSINTMGWYRQAPGKERELVATITSGGMTNYADSVKGRFTISRDNGKNTVYLQMNSLEPGDTAVYYCNLKRRDLQARFGGYWGQGTQVTVSSEPKTQTTTSGR

SEQ ID NO: 41 - protein, 132 aa, Lama glama
QVKLQQSGGGLVQPGGSLRLSCAASESTISINTLGWYRQAPGNQRELVATITTGGTTNYADSVKGRFTISRDNAKNTVYLQMNNLEPGDTAVYYCNLKRRDLQSRFGGYWGQGTQVTVSSEPQDTKTTTSGR

SEQ ID NO: 42 - protein, 131 aa, Lama glama
QVQLQQSGGGLVQPGGSLRLSCVASESTVSINIMGWYRQAPGKQRELVATITTGGTTNYADSVKGRFTISRDNAKNTVYLQMNSLEPEDTAVYYCNLKRRDLQARFGGYWGQGTQVTVSSEPKTPKPTSGR

SEQ ID NO: 43 - protein, 131 aa, Lama glama
QVKLQQSGGGLVQPGGSLRLSCAASESTPSINTMGWYRQAPGKERELVATITSGGMTNYADSVKGRFTISRDNGKNTVYLQMNSLEPGDTAVYYCNLKRRDLQARFGGYWGQGTQVTVSSEPKTPKPQSGR

SEQ ID NO: 44 - protein, 132 aa, Lama glama
QVKLQQSGGGLVQPGGSLRLSCAASGFTFRNYAMSWVRQAPGKGLEWVSAIAAGGAVTKYADSVKGRFTISRDNARDTLYLQMNSLKPEDTAVYYCAKPRDFWYSPEFDFRGQGTQVTVSSEPKNQNQTSGR

SEQ ID NO: 45 - protein, 133 aa, Lama glama
QVQLQQSGGGLVQAGDSLTISCASSLFTFSTSTMGWFRQAPGKEREFVAAIKSSGSSMYYADSVQGRFTISRDNAKKTVTLQMNSLKPEDTAVYYCAKGVYGSRRSADFGSWGQGTQVTVSSEPKTPKPQSGR

SEQ ID NO: 46 - protein, 133 aa, Lama glama
QVQLQQSGGGLVQAGDSLRISCAASLFTFSTSTMGWFRQAPGKEREFVAAIRSTGDSMYYADSVQGRFTISRDNAKKMVYLQMNNLKPEDTAVYYCAKGVYGSRRSADFGSWGQGTQVTVSSEPKTPKPQSGR

SEQ ID NO: 47 - protein, 133 aa, Lama glama
DVQLQASGGGLVQAGGSLRISCTADGYTFSTSTMGWFRQAPGKEREFVAAIKSDGSIMYYADSVAGRFIISRDNAKKMVFLQMDRLKPEDTAVYYCAKGVYGSRRSADFGWWGQGTQVTVSSEPKTPKPQSGR

SEQ ID NO: 48 - protein, 134 aa, Lama glama
QVKLQESGGGLVQAGESLRLSCAASGSNFSINGVGWYRQAPGKQRELVAGITNGGYTSYADSVKGRFTISTDNAKNTVYLQMNSLRPEDTAVYYCNANFQIHRSGADYVRNYWGQGTQVTVSAEPRTKTTTSGR

SEQ ID NO: 49 - protein, 134 aa, Lama glama
QVKLQQSGGGLVQAGGSLRLSCAASGNFFTLNGVAWYRQAPGKQRELVAGITSGGWTNYADSVKGRFTISADNAKNTVYLQMNSLRPEDTAVYYCNANLQIHRDSSGDVRNVWGQGTQVTVSSEPKTPKPQSGR

SEQ ID NO: 50 - protein, 136 aa, Lama glama
DVQLQASGGGLVQAGGSLRLSCAASGSFFSINGVGWYRQAPGKQRELVAGITNGGFTNYADSVKGRFTISRDNAKNTVYLQMNSLKPEDTAVYYCNANLQISRSEDGAYVVRNYWGQGTQITVSSEPKTPKPQSGR

SEQ ID NO: 51 - protein, 134 aa, Lama glama
DVQLQASGGGLVQAGGSLRLSCAASGSGFSINGVGWYRQTPGRQRELVAGITIGGYTNYADSVKGRFTISSDNAKNTVYLQMNSLKPEDTAVYYCNANLQFYRGGGSDVKNYWGQGTQVTVSSEPKTPKPQSGR

SEQ ID NO: 52 - protein, 135 aa, Lama glama
QVQLQESGGGLVQAGGSLRLSCAASGLTFSSYAMGWFHQAPGKEREFVAAINWSGRDTYYADSVKGRFTISRDNRKNTVYLQMNSLKPEDTAVYYCAAAEFFSSGDPLPGMDYWGKGTLVTVSSEPKTQNHNSGR

SEQ ID NO: 53 - protein, 135 aa, Lama glama
QVKLQQSGGGLVQAGGSLRLSCAASGLTFSSYAMGWFRQAPGKEREFVAAINWSGRDTYYADSVKGRFTISRDNAKNTVYLQTNSLKPEDTAVYYCAAAEFLPTQRSPREYDYWGLGTQVTVSSEPKTPKPQSGR

SEQ ID NO: 54 - protein, 136 aa, Lama glama
QVKLQQSGGGLVQAGGSLRLSCAASGYAFNTYTMAWFRQAPGKEREFVALVGMKVDGKIYADSVKGRFTISRDNEQKTVLLEMNHLEPEDTAIYYCAASRRFWTAALNGADYPYWGQGTQVTVSSEPKTPKPQSGR

SEQ ID NO: 55 - protein, 129 aa, Lama glama
QVKLQQSGGGLVQAGGSLRLSCAASGIVFSFNAMGWYRVPPGKQRELVADILKSGGTNVVDSVKGRFAISRDSAQNTLYLQMNRLKPEDTAVYYCNARDWSDGFDEYWGQGTQVTVSSEPKTPKPQSGR

SEQ ID NO: 56 - protein, 135 aa, Lama glama
QVQLQQSGGGLVQAGGSLRLSCSASGRTFSNYVMGWFRQAPGKEREFVATISASGGSTYCADSVEGRFTISRDNAKNTAYLQMNNLEPEDTAVYYCASGPRANASIRRSGYNYWGQGTQVTVSSEPKTPKPQSGR

SEQ ID NO: 57 - protein, 136 aa, Lama glama
QVQLQQSGGGLVQAGGSLRLSCAASGLAFSSYAITWLRQAPGTEREFVALISGSGSSTYYADSVKGRFTISRNNAKNTVYLQMNSLKPEDTAVYYCTASEFLLHPPPPNQKYDYWGQGTQVTVSSEPKTPKHTSGR

SEQ ID NO: 58 - protein, 135 aa, Lama glama
QVQLQQSGGGLVRAGGSLRLSCSASGRTFGNEVMGWFRQAPGKEREFVAAINWSSGNTYYRDSVKGRFTISRDNAKNTVYLQMNSLEPEDTAVYYCAARSRPAISTRRPDYFAWGQGTQVTVSSEPKTPKPQSGR

SEQ ID NO: 59 - protein, 133 aa, Lama glama
QVQLQQSGGGLVQAGGSLRLSCTASGRIDRTYTVSWFRQGPGKEREFVATISWDGSIYYDNAVEGRFSISGDNAKTTVALQMNSLKPEDTAVYYCAARRRVFSRAAAAYNYWGQGTQVTVSSEPKTPKPQSGR

SEQ ID NO: 60 - protein, 124 aa, Lama glama
QVKLQQSGGGLVQPGGSLRLTCAASGFPFSTYAIRWVRRPPGKGLEWVSTIHPDFTTNYADSVSRRFTISRDNAKNTVYLQMNSLKPEDTAVYYCSRGVSGERGQGTQVTVSSEPKTPKPHSGR

SEQ ID NO: 61 - protein, 132 aa, Lama glama
QVQLQQSGGGLVQPGGSLRLSCAASGSIFSIHTMGWYRQAPGKQRELVTTITTGGTTNYADSVKGRFTISRDDAKNTVYLQMNNLKPEDTAVYYCYALIQTASTTWYRQYWGQGTQVTVSSEPKTQNHNSGR

SEQ ID NO: 62 - protein, 126 aa, Lama glama
QVKLQESGGGLVQPGGSLRLSCTASRSIFTRAMAWYRQAPGKQRELVAAIDSGDRTHYADSVKGRFTISRNNAKDTLYLQMNSLKSEDTAVYYCNANLGALLDYWGQGTQVTVSSEPKTQTTTSGR

SEQ ID NO: 63 - protein, 129 aa, Lama glama
QVKLQESGGGLVQAGGSLRLSCAASGFTFATYAMAWYRQAPGKQRELVASISNFGSTAYGDSVKGRFTISRDNAKNTVYLEMNSLKSEDTAVYYCKRVRDVIGRPELWGQGTQVTVSSEPKTPKPHSGR

SEQ ID NO: 64 - protein, 132 aa, Lama glama
QVKLQESGGGLVQPGGSLRLSCLPSINIFSLAAMGWYRQAPGKQRELVASISSGGTANYADSYADSVKGRFTISRDIAKNTVDLQMNSLKPEDTAVYYCKVDSYTYGTDIWGKGVLVTVSSEPQDTKTTSGR

SEQ ID NO: 65 - protein, 140 aa, Lama glama
QVKLQQSGGGLVQAGGSLRLSCAASGTFSRYAMGWFRQAPGKEREFVAAINWTGGSTYYADSVKGRFTISGDNAKNTVYLQMNSLKPGDTAVYYCAAEVHPGDYGLTYMQSQYEYDYWGQGTQVTVSSEPKTPKPTTSGR

SEQ ID NO: 66 - protein, 131 aa, Lama glama
DVQLQASGGGLVQAGGSLRLSCAASTSRFSSYAMGWSRQAPGKQRELVASISSSGLTTNYADSVKGRFTISRDNAKNTVYLQMNSLKPEDTAVYYCKADGRRYSLNEYWGQGTQVTVSSEPKTPKPQPSGR

SEQ ID NO: 67 - protein, 133 aa, Lama glama
DVQLQASGGGLVQAGGSLRLSCAASGSTISSYAMAWYRQAPGKRRELVAHISSGGSTNYADSVKGRFTISRDNAKNTVYLQMNSLKPEDTAVYYCNIYYGGDYYYTGVKPNPWGQGTQVTVSSAHHSEDPRGR

SEQ ID NO: 68 - protein, 137 aa, Lama glama
QVKLQESGGGWVQAGGSLRLSCAASALTASITTMGWFRQTPEKEREFLAAINWTGDYKYYADSVKGRFTISRDNAKNTVDLQMNQLKPEDTAVYYCAASKIRNDIYLNDYTWYQYWGQGTQVTVSSEPKTPKPQSGR

SEQ ID NO: 69 - protein, 143 aa, Lama glama
DVQLQASGGGLVQAGGSLRLSCVASARTFSSYAMGWFRQAPGKEREFVAAISWSGASTDYADSVKGRFTISRDNAKKTVYLQMNTLKPEDTAVYYCAAHHITPTGSYYYSEPLPVDMVYDYWGQGTQVTVSSEPKTKTTTSGR

SEQ ID NO: 70 - DNA, 360 bp, synthetic construct
  misc_feature 1..360: note = Nano85 (Targets capsids of Norovirus strains with GII genotypes)
  source 1..360: mol_type = other DNA
caggtccagcttcaggaaagcggcgggggtttagttcagccaggcggatctcttcgtctttcctgtgcggcgagtggctcaattttttcgatttatgctatgggatggtatcgtcaggccccaggcaagcagcgtgagttggtcgcaagtatcagcagtgggggaggtacgaactatgcggacagtgtgaaggggcgcttcactattagcggagataacgcgaaaaataccgtatatttgcaaatgaatagtttgaaaccagaagacaccgccgtatattattgtaaacgcgaagattactcggcttacgcaccgcctagtggtagtcgtggtcgcggtactcaggtaactgtttcctca

SEQ ID NO: 71 - protein, 120 aa, synthetic construct
  REGION 1..120: note = Nano85 (Targets capsids of Norovirus strains with GII genotypes)
QVQLQESGGGLVQPGGSLRLSCAASGSIFSIYAMGWYRQAPGKQRELVASISSGGGTNYADSVKGRFTISGDNAKNTVYLQMNSLKPEDTAVYYCKREDYSAYAPPSGSRGRGTQVTVSS

SEQ ID NO: 72 - DNA, 345 bp, synthetic construct
  misc_feature 1..345: note = Nano26 (Targets capsids of Norovirus strains with GII genotypes)
  source 1..345: mol_type = other DNA
caagtgcaattgcaagagagtgggggcggtttagtgcaacctggtgggagcctgcgcctgtcttgcaccgccccacgtattatcttttttatgtatgatgtaggatggtaccgtcaggctcctgaaaagcagcgtgaacttgtagctcagatcaatagcgatgtatctaccaaatatgctgacagtgtcaaagggcgcttcactatcagccgtgataacgccaagcgcacggtctatttacaaatgaacgacctgaaaccggaagatgctgcggtctactattgcaatgtacgtcgtgcttcagcggattactgggggcaggggactcaagtgacggtgtcctcg

SEQ ID NO: 73 - protein, 115 aa, synthetic construct
  REGION 1..115: note = Nano26 (Targets capsids of Norovirus strains with GII genotypes)
QVQLQESGGGLVQPGGSLRLSCTAPRIIFFMYDVGWYRQAPEKQRELVAQINSDVSTKYADSVKGRFTISRDNAKRTVYLQMNDLKPEDAAVYYCNVRRASADYWGQGTQVTVSS

SEQ ID NO: 74 - DNA, 363 bp, synthetic construct
  misc_feature 1..363: note = Nano94 (Targets capsids of Norovirus strains with GI genotypes)
  source 1..363: mol_type = other DNA
caagtacagttacaggaaagtggcggaggattggtacaggcgggagggtcattacgtctttcgtgcgcggcctccggtcgtatgttcagcatcaattcgatggggtggtaccgtcaagccccagggaaggagcgtgagttagtagcgacaatttctgaagcgggaacaactacctatgcggattcggtgcgtgggcgtttcacgattgctcgtgacaacgccaaaaacacggtttacttacaaatgaacagcttgaatcccgaagacaccgcggtatattactgcaatgcttatatccaacttgactccaccatctggtttcgtgcttattggggtcaggggacgcaggtaacagtaagctct

SEQ ID NO: 75 - protein, 121 aa, synthetic construct
  REGION 1..121: note = Nano94 (Targets capsids of Norovirus strains with GI genotypes)
QVQLQESGGGLVQAGGSLRLSCAASGRMFSINSMGWYRQAPGKERELVATISEAGTTTYADSVRGRFTIARDNAKNTVYLQMNSLNPEDTAVYYCNAYIQLDSTIWFRAYWGQGTQVTVSS

SEQ ID NO: 76 - DNA, 357 bp, synthetic construct
  misc_feature 1..357: note = Nano14 (Targets capsids of Norovirus strains with GII.10 genotypes, not cross-reactive)
  source 1..357: mol_type = other DNA
caggtccagttacaagaaagtgggggaggtttggttcaatctggaggttcattgcgtttgtcctgtgcggcatcacgcaacattaactccatgcatgtggtaggatggtatcgtcaggcaccaggaaaccagcgcgagttggttgcctccattactgacgatggctctactgactatgtagattcagtcaaggggcgttttactatctcgcgcgacattgccgaaaacacggtctatcttcaaatgaattcattaaatccggaggacaccgcagtttactactgtaaagggactatcgtagtgttcacgaccccaatgcattactgggggaaagggactcaggtgacggtttcctcg

SEQ ID NO: 77 - protein, 119 aa, synthetic construct
  REGION 1..119: note = Nano14 (Targets capsids of Norovirus strains with GII.10 genotypes, not cross-reactive)
QVQLQESGGGLVQSGGSLRLSCAASRNINSMHVVGWYRQAPGNQRELVASITDDGSTDYVDSVKGRFTISRDIAENTVYLQMNSLNPEDTAVYYCKGTIVVFTTPMHYWGKGTQVTVSS

SEQ ID NO: 78 - DNA, 363 bp, synthetic construct; identical to SEQ ID NO: 74
  misc_feature 1..363: note = Nano32 (Targets capsids of Norovirus strains with GII genotypes, not cross-reactive)
  source 1..363: mol_type = other DNA

SEQ ID NO: 79 - protein, 135 aa, synthetic construct
  REGION 1..135: note = Nano32 (Targets capsids of Norovirus strains with GII genotypes, not cross-reactive)
QVQLQESGGGLVQPGGSLRLSCAASGFTLGYYPIGWFRQAPGKGLEGVSCISGSGGSANYAASVKGRFTISRDNAKNTVYLQMNSLKPEDTAIYYCAADLSSLTTVQAMCVIPRPGFSAKAYDYWGLGTQVTVSS

(19) United States
(12) Patent Application Publication
(10) Pub. No.: US 2026/0109941 A1
(43) Pub. Date: Apr. 23, 2026

(54) ARTHROSPIRA PLATENSIS NON-PARENTERAL THERAPEUTIC DELIVERY PLATFORM

(71) Applicant: Lumen Bioscience, Inc., Seattle, WA (US)

(72) Inventors: James ROBERTS, Seattle, WA (US); Michael TASCH, Seattle, WA (US); Mesfin GEWE, Bothell, WA (US); Benjamin JESTER, Seattle, WA (US); Tracy SAVERIA, Seattle, WA (US)

(21) Appl. No.: 19/305,605

(22) Filed: Aug. 20, 2025

Related U.S. Application Data
(63) Continuation of application No. 17/246,837, filed on May 3, 2021, now Pat. No. 12,503,682.
Application No. 17/246,837 is a continuation of application No. PCT/US2020/040794, filed on Jul. 2, 2020.
(60) Provisional application No. 62/870,478, filed on Jul. 3, 2019.
(60) Provisional application No. 62/937,995, filed on Nov. 20, 2019.
(60) Provisional application No. 62/943,075, filed on Dec. 3, 2019.

(51) Int. Cl. C12N 1/20; A61K 35/742; A61K 35/748; C07K 16/104; C12N 1/125; C12N 1/205

(52) U.S. Cl. CPC C12N 1/20; A61K 35/742; A61K 35/748; C07K 16/104; C12N 1/125; C12N 1/205

(57) ABSTRACT

The present disclosure provides non-parenteral compositions comprising a recombinant Spirulina comprising at least one exogenous therapeutic. Compositions of the present disclosure can be used as vaccines and/or therapeutic drugs. The present disclosure also provides methods of making recombinant Spirulina comprising at least one exogenous therapeutic, and methods of treatment.

### CROSS REFERENCE TO RELATED APPLICATIONS

This Application is a continuation of U.S. patent application Ser. No. 17/246,837, filed May 3, 2021, which is a continuation of International Patent Application No. PCT/US2020/040794 filed Jul. 2, 2020 which claims priority to U.S. Provisional Patent Application No. 62/870,478 filed Jul. 3, 2019, U.S. Provisional Patent Application No. 62/937,995 filed Nov. 20, 2019, and U.S. Provisional Patent Application No. 62/943,075 filed Dec. 3, 2019, the entire contents of each which are incorporated by reference herein.

### INCORPORATION BY REFERENCE OF THE SEQUENCE LISTING

The contents of the electronic sequence listing (LUBI_029_04US_SeqList_ST26.xml; Size: 107,313 bytes; and Date of Creation: Aug. 14, 2025) are herein incorporated by reference in its entirety.

### FIELD

The disclosure is directed to non-parenteral therapeutic compositions. In particular, the disclosure provides oral, nasal, and respiratory (inhalation) compositions comprising recombinant Spirulina, wherein the recombinant Spirulina comprises one or more exogenous therapeutics.

### BACKGROUND

Non-parenteral administration of therapeutics is a convenient, portable, and inexpensive mode of administration. Nasal and oral administration of therapeutics is commonly practices, however, oral therapeutics are exposed to harsh conditions in the digestive tract and may be degraded before they can exert their effect. Further, these therapeutics are expensive to make and require purification of the therapeutic along with the development of compositions that will protect the oral therapeutics from the digestive enzymes and low pH the therapeutic is subjected to after administration. More cost-effective and stable compositions are required for non-parenteral administration.

### SUMMARY OF THE INVENTION

The present application solves the problem of cost and exposure of the therapeutic to degradation in the digestive, nasal and respiratory tract by administering the therapeutic to the subject in Spirulina. Spirulina is a cyanobacterium that can last in the digestive, nasal and respiratory tract, thus protecting the encapsulated therapeutic until the Spirulina reaches its destination (e.g. in the gastrointestinal tract). Moreover, Spirulina are easily cultivated and harvested, grows rapidly, can be dried to avoid spoilage, and can be consumed raw. Indeed, Spirulina is approved for human consumption and is commonly consumed as a supplement.

Provided herein are non-parenteral compositions comprising a recombinant Spirulina, wherein the recombinant Spirulina comprises at least one exogenous therapeutic, prophylactic molecule or combinations of two or more exogenous therapeutics or prophylactic molecules. The exogenous therapeutic may be a compound produced by microorganisms or plants. In particular, the exogenous therapeutic may be anti-microbial compound or a polypeptide. In some embodiments, the exogenous therapeutic or prophylactic molecule is a VHH and/or a lysin.

In some embodiments, the present disclosure provides non-parenterally delivered compositions comprising a recombinant Spirulina, wherein the recombinant Spirulina comprises at least one therapeutic or prophylactic molecule, or a combination of two or more therapeutics or prophylactic molecules. In some embodiments, the therapeutic or prophylactic molecule is delivered to the gastrointestinal tract. In some embodiments, the therapeutic or prophylactic molecule is delivered nasally. In some embodiments, the therapeutic or prophylactic molecule is delivered by respiration (inhalation). In some embodiments, the therapeutic or prophylactic molecule is delivered systemically. In some embodiments, the therapeutic or prophylactic molecule is delivered locally.

In some embodiments, the therapeutic or prophylactic molecule is or combination of two or more therapeutics or prophylactic molecules are endogenous Spirulina molecule. In some embodiments, the endogenous Spirulina molecule is found in higher concentrations than found in naturally-occurring Spirulina.

In some embodiments, the therapeutic or prophylactic molecule or combination of two or more therapeutics or prophylactic molecules are exogenous to Spriulina. In some embodiments, the exogenous molecule is produced by a different bacteria, parasite, protozoa, virus, phage, algae, animal, or plant.

In some embodiments, the combination contains two or more therapeutic or prophylactic molecules that are endogenous to Spirulina. In some embodiments, the combination contains two or more therapeutic or prophylactic molecules that are exogenous to Spirulina. In some embodiments, the combination contains two or more therapeutic or prophylactic molecules that are a mixture of endogenous and exogenous to Spriulina. In some embodiments, the combination contains two or more therapeutic or prophylactic molecules, where at least one of the therapeutic or prophylactic molecules is present in greater copy numbers (e.g. two times, three times, four times, five times, or more) than another therapeutic or prophylactic molecule present in the combination.

In some embodiments, the exogenous molecule is a polypeptide or a fragment thereof. In some embodiments, the exogenous polypeptide is an antibody or fragment thereof. In some embodiments, the antibody or fragment thereof is selected from the group consisting: of full length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, nanobody, IgNAR, V-NAR, HcIgG, or a combination thereof.

In some embodiments, the exogenous polypeptide is selected from the group consisting of: insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, and chlorotoxin.

In some embodiments, the exogenous polypeptide is an antigen or epitope. In some embodiments, the antigen or epitope is derived from an infectious microorganism, a tumor antigen or a self-antigen associated with an autoimmune disease.

In some embodiments, the exogenous polypeptide is a catalytic enzyme or fragment thereof, such as a lysin, that cleaves the cell wall.

In some embodiments, the recombinant Spirulina contains a combination of one or more different antibodies or antibody fragments. In some embodiments, the recombinant Spirulina contains a combination of one or more different VHHs. In some embodiments, the recombinant Spirulina contains a combination of one or more different antibodies or antibody fragments and one or more polypeptides. In some embodiments, the recombinant Spirulina contains a combination of one or more different VHHs and one or more polypeptides. In some embodiments, the recombinant Spirulina contains a combination of one or more different VHHs and one or more lysin polypeptides.

In some embodiments, administration of the recombinant Spirulina to a subject prevents, treats or ameliorates a disease or disorder. In some embodiments, the disease or disorder is selected from the group consisting of: Celiac Disease, Type 1 diabetes, Type 2 diabetes, cancer, an inflammatory disorder, a gastrointestinal disease, an autoimmune disease or disorder, an endocrine disorder, gastroesophageal reflux disease (GERD), ulcers, high cholesterol, inflammatory bowel disorder, irritable bowel syndrome, crohn's disease, ulcerative colitis, constipation, vitamin deficiency, iron deficiency, and diarrhea.

In some embodiments, administration of the recombinant Spirulina to a subject treats, prevents, or ameliorates an infection. In some embodiments, the infection results in disorders such as acute respiratory distress syndrome (ARDS), pneumonia, pericarditis, stroke, and COVID-19.

In some embodiments, the infection is bacterial, viral, fungal, or parasitical. In some embodiments, the bacteria causing the infection is selected from the group consisting of: E. coli, Enterotoxigenic E. coli (ETEC), Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella, Helicobacter, Anthrax, Enterohemmorrhagic E. coli (EHEC), Enteroaggregative E. coli (EAEC), and Legionella.

In some embodiments, the virus causing the infection is selected from the group consisting of: bacteriophage, RNA bacteriophage (e.g. MS2, AP205, PP7 and Qβ), Coronavirus, Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, White Spot Syndrome Virus, a coronavirus, MERS, SARS, and SARS-CoV-2.

In some embodiments, the fungus causing the infection is selected from the group consisting of: Aspergillus, Candida, Blastomyces, Coccidioides, Cryptococcus, and Histoplasma.

In some embodiments, the parasite causing the infection is selected from the group consisting of: Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. and Wuchereria spp., Taenia spp., Echinococcus spp., and Diphyllobothrium spp., Fasciola spp., and Schistosoma spp..

In some embodiments, the exogenous polypeptide or a fragment thereof is in a fusion protein.

In some embodiments, the recombinant Spirulina comprises a nucleic acid encoding the exogenous polypeptide or fragment thereof. In some embodiments, at least 2, at least 3, at least 4, or at least 5 copies of a nucleic acid sequence encoding the at least one exogenous polypeptide or fragment thereof are present in the recombinant Spirulina. In some embodiments, 2, 3, 4, 5, 6, 8, 10, 15, 20, 25, 30, 40, or 50 copies of a nucleic acid sequence encoding the at least one exogenous polypeptide or fragment thereof are present in the recombinant Spirulina. In some embodiments, at least 2, at least 3, at least 4, or at least 5 copies of the at least one exogenous polypeptide or fragment thereof are present in a single molecule of the exogenous polypeptide expressed in the recombinant Spirulina.

In some embodiments, 2, 3, 4, 5, 6, 8, 10, 15, 20, 25, 30, 40, or 50 copies of the at least one exogenous polypeptide or fragment thereof are present in a single molecule of the exogenous polypeptide expressed in the recombinant Spirulina.

In some embodiments, within the molecule of the exogenous polypeptide or fragment thereof, the copies of the exogenous polypeptide are linked in tandem.

In some embodiments, within the molecule of exogenous polypeptide or fragment thereof, the copies of the exogenous polypeptide or fragment thereof are separated by a spacer sequence.

In some embodiments, within the molecule of exogenous polypeptide or fragment thereof, some of the copies of the exogenous polypeptide or fragment thereof are linked in tandem and the remaining copies of the exogenous polypeptide or fragment thereof are separated by a spacer sequence. In some embodiments, the spacer sequence is between about 1 and 50 amino acids long. In some embodiments, more than one spacer sequence is present within the molecule of the exogenous polypeptide or fragment thereof. In some embodiments, the recombinant Spirulina comprises at least 2, at least 3, at least 4, or at least 5 different exogenous polypeptides or fragments thereof.

In some embodiments, the fusion protein comprises a carrier or a chaperone protein. In some embodiments, the carrier protein is selected from the group consisting of: maltose binding protein, hedgehog hepatitis virus-like particle, thioredoxin, and phycocyanin. In some embodiments, the fusion protein comprises a scaffold protein.

In some embodiments, the at least one exogenous polypeptide is linked to a scaffold protein at the N-terminus or the C-terminus, or in the body of the scaffold protein. In some embodiments, the scaffold protein is selected from the oligomerization domain of C4b-binding protein (C4BP), cholera toxin b subunit, or oligomerization domains of extracellular matrix proteins. In some embodiments, the at least one exogenous polypeptide and the scaffold protein are separated by about 1 to about 50 amino acids.

In some embodiments, the fusion protein comprises multiple copies of the at least one exogenous polypeptide or fragment thereof, wherein the at least one exogenous polypeptide or fragment thereof and the scaffold protein are arranged in any one of the following patterns: (E)n-(SP), (SP)-(E)n, (SP)-(E)n-(SP), (E)n1-(SP)-(E)n2, (SP)-(E)n1-(SP)-(E)n2, and (SP)-(E)n1-(SP)-(E)n2-(SP), wherein E is the at least one exogenous polypeptide or fragment thereof, SP is the scaffold protein, n, n1, and n2 represent the number of copies of the at least one exogenous polypeptide or fragment thereof.

In some embodiments, the recombinant Spirulina comprises an anti-Campylobacter VHH. In some embodiments, the campylobacter is a C. jejuni. In some embodiments, the VHH binds to a campylobacter component. In some embodiments, the VHH binds flagellin. In some embodiments, administration increases Campylobacter shedding. In some embodiments, administration reduces the levels of biomarkers. In some embodiments, the biomarker is an inflammation biomarker.

In some embodiments, the recombinant Spirulina comprises a VHH that binds to an anti-Clostridium toxin. In some embodiments, the Clostridium is C. difficile. In some embodiments, the VHH binds to a Clostridium component, toxin A, or toxin B or both. In some embodiments, the VHH comprises the amino acid sequence of any of SEQ ID NO:s 5-17 or fragment thereof.

In some embodiments, the recombinant Spirulina comprises a VHH that binds to a norovirus P domain. In some embodiments, the VHH comprises the amino acid sequence of any of SEQ ID NOs: 40-79 or a fragment thereof.

In some embodiments, the recombinant Spirulina comprises a VHH that binds to a malaria polypeptide. In some embodiments, the recombinant Spirulina comprises a malaria antigen. In some embodiments, the malaria antigen is Circumsporozoite protein (CSP). In some embodiments, the malaria antigen comprises at least one NANP repeat. In some embodiments, the recombinant Spirulina comprises a nucleotide sequence encoding a malaria antigen. In some embodiments, the recombinant Spirulina comprises an amino acid sequence comprising a malaria antigen. In some embodiments, the recombinant Spirulina comprises the molecules of any of SEQ ID NOs: 26-31. In some embodiments, the recombinant Spirulina comprising a malaria antigen or VHH is administered intranasally. In some embodiments, the extract of a recombinant Spirulina comprising a malaria antigen or VHH is administered intranasally.

In some embodiments, the therapeutic or prophylactic molecule is monomeric.

In some embodiments, the therapeutic or prophylactic molecule is multimeric.

In some embodiments, the therapeutic or prophylactic molecule is trimeric. In some embodiments, the therapeutic or prophylactic molecule is pentameric. In some embodiments, the therapeutic or prophylactic molecule is heptameric. In some embodiments, the multimer is heteromeric. In some embodiments, the multimer is homomeric. In some embodiments, the multimer is arranged in a nanoparticle. In some embodiments, the multimer binds to a target or target molecule at a high affinity. In some embodiments, the multimer binding affinity is greater than that of a monomer or a dimer.

In some embodiments, the multimer has an EC₅₀ of over 5 μg/mL. In some embodiments, the multimer has an EC₅₀ of over 10 μg/mL. In some embodiments, the multimer has an EC₅₀ of about 5 μg/mL to about 40 μg/mL. In some embodiments, the multimer has an EC₅₀ of between about 0.10 to about 100 nM. In some embodiments, the multimer has an EC₅₀ of between about 0.2 nM to about 55 nM. In some embodiments, the multimer binding affinity is greater than that of a multimer comprising fewer copies of the exogenous therapeutic or fewer copies of combinations of exogenous therapeutics. In some embodiments, administration of Spirulina comprising multimeric exogenous therapeutics results in a smaller dose of Spirulina for efficacy than administration of a Spirulina comprising a monomer of the same exogenous therapeutic.

In some embodiments, the recombinant Spirulina is selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In some embodiments, the recombinant Spirulina is non-living. In some embodiments, the recombinant Spirulina is dried, spray dried, freeze-dried, or lyophilized.

In some embodiments, the non-parenteral compositions comprise a pharmaceutically acceptable excipient.

In some embodiments, the composition survives in the gastrointestinal tract or a simulated stomach environment. In some embodiments, the composition survives in the gastrointestinal tract or a simulated stomach environment for at least 5 minutes. In some embodiments, the composition survives in the gastrointestinal tract or a simulated stomach environment overnight.

In some embodiments, the composition survives in the nasal cavity. In some embodiments, the composition survives in the upper respiratory tract. In some embodiments, the composition survives in the airway. In some embodiments, the composition survives in the nasal cavity, upper respiratory tract and/or the airway for at least 5 minutes. In some embodiments, the composition survives in the nasal cavity, upper respiratory tract and/or the airway overnight.

In some embodiments, the present disclosure provides method of treating or preventing a disease or disorder in a subject in need thereof, comprising administering to the subject the non-parenterally delivered composition of the disclosure.

In some embodiments, administration of the non-parenterally delivered composition decreases or prevents development of campylobacter symptoms.

In some embodiments, administration of the delivered composition decreases or prevents the development of inflammation in the subject.

In some embodiments, the present disclosure provides methods of treating or preventing a C. difficile infection comprising administering to a subject the non-parenterally delivered composition of the instant disclosure. In some embodiments, administration of the non-parenterally delivered composition decreases or prevents development of C. difficile symptoms. In some embodiments, the present disclosure provides methods of treating or preventing a malaria infection comprising administering the compositions of the instant disclosure via inhalation or intranasally. In some embodiments, inhaled or instranasal administration of the composition decreases or prevents development of malaria symptoms.

In some embodiments, the present disclosure provides methods of treating or preventing a coronavirus infection comprising administering the compositions of the instant disclosure via inhalation or intranasally. In some embodiments, inhaled or instranasal administration of the composition decreases or prevents development of coronavirus symptoms.

In some embodiments, the present disclosure provides methods of treating or preventing a malaria infection comprising administering to a subject the non-parenterally delivered composition of the instant disclosure. In some embodiments, administration of the non-parenterally delivered composition decreases or prevents development of malaria symptoms.

In some embodiments, the present disclosure provides methods of treating or preventing a coronavirus (e.g. SARS, SARS-CoV-2) infection comprising administering to a subject the non-parenterally delivered composition of the instant disclosure. In some embodiments, administration of the non-parenterally delivered composition decreases or prevents development of coronavirus infection symptoms (e.g. ARDS, inflammation).

In some embodiments, provided herein are methods of making the non-parenteral compositions described herein, the method comprising introducing at least one exogenous therapeutic into a Spirulina.

In some embodiments, provided herein are methods of making the non-parenteral compositions described herein, the method comprising introducing a nucleic acid sequence encoding the at least one exogenous therapeutic into a Spirulina.

In some embodiments, provided herein are non-parenteral antigenic compositions comprising a recombinant Spirulina, wherein the recombinant Spirulina comprises at least one exogenous antigenic epitope, wherein a nucleic acid sequence encoding the at least one exogenous antigenic epitope is integrated into the Spirulina via homologous recombination.

In some embodiments, provided herein are non-parenteral antigenic compositions prepared by a method comprising: introducing a nucleic acid sequence encoding at least one exogenous antigenic epitope into a Spirulina and integrating the nucleic acid sequence into the Spirulina via homologous recombination.

### BRIEF DESCRIPTION OF THE DRAWINGS

FIG. 1A-B shows oral Spirulina monomeric anti-campylobacter VHH provides complete protection against campylobacter infection in mice. Administration of an oral gavage containing 10% Spirulina biomass (425 μg of the monomeric VHH per dose) daily for five days stops incidence of diarrhea in Campylobacter-infected mice (Panel A) and decreases Campylobacter shedding (Panel B) compared to controls.

FIG. 2A-B demonstrates Spirulina expressing trimeric anti-campylobacter VHH have an anti-inflammatory effect in Campylobacter-infected mice. Administration of an oral gavage containing 0.5% Spirulina biomass (19 μg of the trimeric VHH per dose) daily for three days decreases markers of inflammation, stool lipocalin (Panel A) and myeloid cell infiltration of gut lamina propria (Panel B).

FIG. 3A-B: Weight changes and histology scores for mice pretreated with spirulina and infected with C. jejuni. Mice were pretreated with one dose (left) or three doses (right) of spirulina. FIG. 3A. Mice were infected with 10⁸ CFU C. jejuni at time 0, and treated with PBS (infected), spirulina strain SP651 (anti-C. jejuni), or SP257 (irrelevant VHH). Body weight variation represents weight change 72 hours post-infection. FIG. 3 B. Caeca from animals were examined and scored for histopathology at 72 hours post-infection.

FIG. 4A-C: Weight changes and pathogen shedding in mice pretreated with a single dose of spirulina and infected with C. jejuni. Mice were pretreated with 1.33 mg of spirulina, inoculated with 10⁸ CFU C. jejuni at time 0, and treated with PBS (infected), spirulina SP651 (anti-C. jejuni VHH), or SP257 (irrelevant VHH). FIG. 4A. body weight variation at 72 hours post-infection. FIG. 4B. pathogen shedding at 24 and 72 hours post infection. C. stool lipocalin-2 (LCN2) levels and percentage of myeloid cells infiltrating the lamina propria (% PMNs) at 72 hours post-infection. LCN2 was measured by ELISA. Gr1+, CD11b+ myeloid cells infiltrating lamina propria were identified by FACS.

FIG. 5A-B: Weight changes and pathogen shedding in mice pretreated with a protease-resistant VHH variant in spirulina and infected with C. jejuni. Mice were pretreated with a single dose of varying concentration of spirulina-VHH, and infected with 10⁸ CFU of C. jejuni. Each row of data represents a different treatment strain (SP526, SP806, or SP651). FIG. 5A. body weight changes at 72 hours post-infection. FIG. 5B. pathogen shedding at 24 and 72 hours post infection. White circles represent uninfected control mice. Mice treated with SP526 and SP806 were treated concurrently and therefore used the same uninfected and infected control groups.

FIG. 6A-B: Inflammatory markers and lamina propria leukocyte infiltration in mice pretreated with spirulina and infected with C. jejuni. Mice were pretreated with a single dose of varying concentrations of spirulina-VHH and infected with 10⁸ CFU of C. jejuni. Each row of data represents a different treatment strain (SP526, SP806, or SP651). A), stool lipocalin-2 (LCN2) levels 72 hours post-infection. B), Gr1+, CD11b+ myeloid cells infiltrating lamina propria (% PMNs) were identified by FACS. White circles represent uninfected control mice. Mice treated with SP526 and SP806 were treated concurrently and therefore used the same uninfected and infected control groups.

FIG. 7: SP1182 construct both as schematic and ribbon structure.

FIG. 8: Sequence of SP1182 construct. The VHH binds to the flagellin protein flaA from C. jejuni. The CDR1, CDR2, and CDR3 are noted above the corresponding segment of the VHH sequence. Mass spectrometry data of the intact protein indicates that the N-terminal methionine is removed. The maltose binding protein serves to increase expression levels and solubility of the fused VHH, while the hexahistidine tae serves as an affinity tag for detection reagents. Two short flexible linkers, a G-G and a G-S-G bridge the VHH and MBP and the MBP and hexahistidine tag respectively.

FIG. 9: Bacterial shedding (CFU/g feces) measured in stool at 40 and 72 hours after infection. C. jejuni-only mice received no treatment. Two-dose (24 and 48 hours after infection) and three-dose (24, 36, and 48 hours after infection) mice received 1.33 mg of the indicated spirulina-VHH per dose.

FIG. 10: Lipocalin (LCN2) levels measured in stool at 72 hours after infection. Uninfected and C. jejuni-only mice received no treatment. Two-dose (24 and 48 hours post infection) and three-dose (24, 36, and 48 hours after infection) mice received 1.33 mg of the indicated spirulina-VHH per dose.

FIG. 11A-B demonstrates that encapsulation of anti-campylobacter VHH in Spirulina protects the polypeptide in a simulated stomach environment. The anti-campylobacter VHH in Spirulina can still be detected after overnight exposure (Panel A), and the Spirulina cells themselves remain intact (Panel B).

FIG. 12 demonstrates that anti-campylobacter expressed in Spirulina are stable long-term at elevated temperatures in dried biomass. Each curve represents serial 1:5 dilutions of biomass resuspended in PBS, incubated in ELISA plate wells coated with flagellin antigen, and detected with an anti-His-tag antibody. Results were normalized to the binding activity of the purified VHH assayed at the same.

FIG. 13 demonstrates post-C. jejuni infection mouse weights. On day 0, mice were weighed, infected with C. jejuni, and treated with the indicated spirulina strain (SP257, SP526, SP742, or SP806). Mice were then weighed every 2 days post-infection, and % weight change was calculated based on initial weight.

FIG. 14 demonstrates C. jejuni shedding. Groups of mice were challenged with C. jejuni on day 0 and treated with the indicated spirulina strain (SP257, SP526, SP742, SP806). Every 2 days post-infection, stool samples were collected from each mouse, and the mean C. jejuni colony counts (cfu) per 10 mg stool for was measured.

FIG. 15 demonstrates Inflammatory biomarkers in C. jejuni-infected mice treated with spirulina. On day 11 post-infection and treatment with the indicated spirulina strain (SP257, SP526, SP742, or SP806), the levels of two inflammatory biomarkers, lipocalin-2 (LCN2) (left) and myeloperoxidase (MPO) (right), were measured in stool samples. Group numbers refer to spirulina strains used for treatment.

FIG. 16 demonstrates weight changes in mice pretreated with spirulina and infected with C. jejuni. Mice were pretreated with one dose (left) or three doses (right) of spirulina. Mice were infected with 10⁸ CFU C. jejuni at time 0, and treated with PBS (infected), spirulina strain SP651 (anti-C. jejuni), or SP257 (irrelevant VHH). Body weight variation represents weight change 72 hours post-infection.

FIG. 17A-C demonstrates weight changes and pathogen shedding in mice pretreated with a single dose of spirulina and infected with C. jejuni. Mice were pretreated with 1.2 mg of spirulina, inoculated with 108 CFU C. jejuni at time 0, and treated with PBS (infected), spirulina SP651 (anti-C. jejuni VHH), or SP257 (irrelevant VHH). A, body weight variation at 72 hours post-infection. B, pathogen shedding at 24 and 72 hours post infection. C, stool lipocalin-2 (LCN2) levels at 72 hours post-infection. D, Gr1+, CD11b+ myeloid cells infiltrating lamina propria were identified by FACS.

FIG. 18A-B demonstrates weight changes and pathogen shedding in mice pretreated with a protease-resistant VHH variant in spirulina and infected with C. jejuni. Mice were pretreated with a single dose of varying concentration of Spirulina-VHH, and infected with 10⁸ CFU of C. jejuni. Each row of data represents a different treatment strain (SP526, SP806, or SP651). A, body weight changes at 72 hours post-infection. B) pathogen shedding at 24 and 72 hours post infection. White circles represent uninfected control mice. Mice treated with SP526 and SP806 were treated concurrently and therefore used the same uninfected and infected control groups.

FIG. 19A-B demonstrates inflammatory markers and lamina propria leukocyte infiltration in mice pretreated with spirulina and infected with C. jejuni. Mice were pretreated with a single dose of varying concentrations of Spirulina-VHH and infected with 10⁸ CFU of C. jejuni. Each row of data represents a different treatment strain (SP526, SP806, or SP651). A, stool lipocalin-2 (LCN2) levels 72 hours post-infection. B, Gr1+, CD11b+ myeloid cells infiltrating lamina propria were identified by FACS. White circles represent uninfected control mice. Mice treated with SP526 and SP806 were treated concurrently and therefore used the same uninfected and infected control groups

FIG. 20 demonstrates chick body weight following inoculation with C. jejuni. Birds were treated with therapeutic (SP526, SP651), irrelevant (SP257), or no Spirulina (Campy) prior to inoculation with C. jejuni 81-176, and weights measured at intervals.

FIG. 21 demonstrates quantitative Campylobacter colonization moderated by Spirulina-expressed VHH. Birds were treated as in FIG. 12. At 72 hours post inoculation with 10⁸ CFU Campylobacter birds were euthanized and cecal contents were collected for quantitative bacterial load determination.

FIG. 22A-C shows Spirulina Expression Constructs. A) Expression constructs designed for Spirulina expression. VHH orientation, chaperone fusion partner used and oligomeric state of the final product indicated. Selected Spirulina strains expressing the anti-CfaE VHHs reported with a number preceded by the designation “SP.” B) The complement binding protein C4B heptamerization domain molecular structure (PDB ID 4B0F). Intermolecular disulfide bonds link monomers to form heptamer. C) The dimerization domain of cAMP-dependent protein kinase type I-alpha regulatory subunit used to express homo dimeric VHHs. Intermolecular disulfide bonds link monomers to form dimer.

FIG. 23A-C shows VHH Expression in Spirulina. A) Example Spirulina expression of monomeric and dimeric VHHs as analyzed by Western Blotting. B) Intermolecular disulfide bond formation in the dimerization domain is confirmed by SDS-PAGE gel under reducing (R) and non-reducing (NR) conditions. Corresponding fragments and molecular sizes are indicated. C) Expression of a hetero-heptameric VHH that target the adhesion domains on F4+ and F18+ pig ETEC.

FIG. 24A-B shows A) ELISA based VHH activity of Spirulina strains binding to the F4+ adhesin tip domain, FaeG. Antibody titration was measured as a dilutions of total protein extracts from starting concentration of 1000 μg/ml. The homo-dimeric and hetero-heptameric constructs bind antigen well. B) ELISA based VHH activity of Spirulina strains binding to the F18+ adhesin tip domain, FedF. Antibody titration was measured as a dilutions of total protein extracts from starting concentration of 1000 μg/ml. The hetero-heptameric constructs bind antigen the antigen well while VHHs that are raised against F4+ adhesin show no binding to F18+ adhesin.

FIG. 25A-C: A) shows Western Blot demonstrating the protein expresseion in dried Spirulina biomass. B) shows that VHH in a Spirulina slurry from spray-dried (SD) and freeze-dried (FD) powder show comparable ELISA based binding. C) shows the antigen binding efficiency of Spirulina expressing VHHs assessed using BLI-based kinetics measurement; biotin-tagged FaeG was loaded on Strptavidin biosensors, and binding to Spirulina extract was measured.

FIG. 26A-C. Gnobiotic bacterial challenge study. A0 shows an overview of oral gavage protocol using the gnobiotic piglet model. B) shows the effect of administration of SP795 on gut bacterial load. C) shows the effect of SP795 and SP-1156 on bacterial shedding in K88-resistant piglets.

FIG. 27A-C shows anti-Norovirus Spirulina Expression Constructs. A) Expression constructs designed for Spirulina expression. VHH orientation, and chaperone fusion partner used with selected Spirulina strains expressing the anti-CfaE VHHs reported with a number preceded by the designation “SP.” B) Protein expression in Spirulina strains is assessed by Western Blotting. C) NI-NTA purified protein from strains expressing VHHs are assayed by SDS-PAGE gel and Coomassie staining. Expected full length fragments are indicated with red boxes.

FIG. 28A-C shows anti-Norovirus VHH Binding Activity. A) ELISA based VHH activity of Spirulina strains binding to GII.4 HuNoV genotype capsid protrusion protein (P1). Spirulina expressed, Ni-NTA purified VHHs were tittered in dilution series from starting concentration of 20 μg/m. SP834 (Nano-26-MBP) show good binding to the GII.4 P1 domain. B) ELISA based VHH activity of Spirulina strains binding to GII.10 HuNoV genotype capsid protrusion protein (P1). Spirulina expressed, Ni-NTA purified VHHs were tittered in dilution series from starting concentration of 20 μg/m. SP834 (Nano-26-MBP) show good binding to the GII. 10 P1 domain. C) ELISA based VHH activity of Spirulina strains binding to GI.1 HuNoV genotype capsid protrusion protein (P1). Spirulina expressed, Ni-NTA purified VHHs were tittered in dilution series from starting concentration of 20 μg/m. SP835 (Nano-94-TxnA), SP836 (Nano-94-MBP), SP864 (Nano-94) show good binding to the GI.1 P1 domain.

FIG. 29A-B shows a Surrogate Neutralization Assay. Plates were coated with Pig Gastric Mucin (PGM) and blocked with skim milk. GII.10 (2 μg/ml) or GI.1 VLPs (1ug/ml) were pre-incubated with serially diluted samples for 1 h at RT and added to the plates. Bound VLPs were detected with GI. 1 specific biotinylated nanobody NB60 or GII. 10 polyclonal sera. Antibodies were detected with corresponding secondary antibodies (strep-HRP or anti-rabbit-HRP). (A) Spirulina expressed and Ni-NTA purified VHHs show HBGA blocking properties a similar range to the controls. (B) Spirulina expressed Ni-NTA purified VHHs show comparable HBGA blocking properties with controls.

FIG. 30A-B: Sequence alignment of Nano85 and K922, anti-human norovirus (HuNoV) protrusion (P) domain antibody. Antibody CDRs are highlighted in blue. Amino acid positions that affect antigen binding are boxed. B) shows structural analysis of framework region amino acid differences between Nano85 and K922 based on the HuNoV GII. 10 P domain bound Nano85 structure (PDB ID 4X7E). The boxed amino acid sidechains indicate mutations incorporated in loop grafted Nano85. Nano85 CDR3 that dominate interactions in antigen binding are circled.

FIG. 31A-C: A) shows Western Blot analysis of Spirulina strains transformed with C-terminal MBP fused original Nano 85 (SP1371) and loop grafted nano85 (SP1372) show protein expression. B & C) show bacterial expressed original Nano85 (B), and loop grafted Nano85 (C) show binding to recombinant P domains derived from various HuNoV Gii strains (GII.2, GII.4, and GII.17).

FIG. 32A-B: Binding kinetics and cross-reactivity of bacterial expressed recombinant anti-Human Norovirus (HuNoV) P domain targeting VHHs. A shows ELISA based binding and cross-reactivity of various VHHs raised against HuNoV Genotype GII. 10 Protrusion (P) domain (Nano85 loop grafted and Nano26) or GII.4 P domain (VHH3.2, VHH4.1, and VHH5.4) expressed recombinantly in a bacterial expression system. Nano26 and Nano85 show broad cross-reactivity while VHH3.2, VHH4.1, and VHH5.4 show no binding against the recombinant GII.17 P domain. B shows BLI based binding kinetics of various VHHs raised against HuNoV Genotype GII.10 P domain (Nano85 loop grafted and Nano26) or GII.4 P domain (VHH3.2, VHH4.1, and VHH5.4). Biotin tagged recombinant GII.2 P domain at 100 nM was used as an antigen. VHH concentrations used to generate binding kinetics are indicated for each VHH.

FIG. 33A-B: ELISA based binding and cross-reactivity of anti-Human Norovirus (HuNoV) P domain targeting VHHs. A) shows ELISA based binding of VHHs raised against HuNoV Genotype GI.1 Protrusion (P) domain, Nano94, VHH10.4, VHH6.3, VHH7.3. The VHHs tested exhibit binding EC50 ranging from 0.21 nM to 50.07 nM, where spirulina expressed recombinant nano94-TxnA shows the weakest binding. B) shows cross-reactivity of VHHs against the recombinant HuNoV GI.3 P domain. The VHH7.3 was cross-reactive binding against the GI.3 P domain.

FIG. 34A-B: Spirulina expressed and Ni-NTA purified proteins were stable following freeze-drying by lyophilization. A) shows binding activity of recombinant anti-Norovirus VHH expressed in Spirulina exhibit no loss in binding activity against recombinant HuNoV GII.10 P domain following freeze-drying (SP833_lyo, SP834_Lyo, and SP1241_Lyo) when compared to purified protein stored at 4° C. after purification (SP833, SP834, and SP1241 respectively). Observed ELISA based binding as measured by EC50 is given in the accompanying table. B) shows binding activity of recombinant anti-Norovirus VHH expressed in Spirulina exhibit no loss in binding activity against recombinant HuNoV GI.1 P domain following freeze-drying (SP835_lyo, and SP864_Lyo) when compared to purified protein stored at 4° C. after purification (SP835, and SP864 respectively). Observed ELISA based binding as measured by EC50 is given in the accompanying table.

FIG. 35: Anti-Norovirus capsid protrusion domain (P) targeting VHHs show varying degrees of protease sensitivity, with the GII genogroup targeting loop grafted Nano85 exhibiting the best resistance against Chymotrypsin and Trypsin. Bacterial expressed recombinant VHHs (1 μg total protein) were incubated with 20 μL of chymotrypsin (0.1 mg/mL or 0.01 mg/mL) or Trypsin (0.01 mg/mL or 0.001 mg/mL) in digestion buffer (1 mM Tris pH 8.0, 20 mM CaCl2). Samples were incubated for 1 hour, 2 hours, or 4 hours. Protease sensitivity was assayed using ELISA based binding. High binding ELISA plates were coated with the recombinant GII.2 P domain. The level of active VHH post-protease digestion was determined by assessing VHH binding to antigen. The percentage of active VHH after digestion was calculated as a ratio of activity from VHH incubated with PBS.

FIG. 36A-C shows anti-TNFα Spirulina Expression Constructs design, expression and activity. A) Anti-TNF-α VHH, ID34F designed as monomer (SP865) and dimer (SP1030) for Spirulina expression. Expression is confirmed by western blotting. B) Spirulina expressed VHHs exhibit binding activity against recombinant human TNF-α on ELISA plates where high affinity plates are coated with human TNF-α and VHHs in the form of Spirulina crude lysates were titrated in dilution series starting from 20,000 μg/ml. C) Binding efficiency was calculated as EC50. Both monomeric and dimeric forms of the VHH bind comparably.

FIG. 37 shows an overview of the development and testing of anti-toxinB (C. difficile) VHHs.

FIGS. 38A-D: Western blot expression analysis of spriulina strains expressing anti-TcdB VHHs 5D and E3 in various hybridization contexts. “ssPsbU” and “ssPsbP2” indicate the presence of putative thylakoid targeting signal sequences on the N-terminus of designated proteins, derived from the cyanobacterial photosystem proteins PsbU and PsbP2, respectively. pAP205, pMS2, pQb and PP7 are enforced single peptide dimers derived from the capsid protein of RNA phages AP205, MS2, Qb and PP7, respectively. CCMk2 denotes the spirulina carboxysome shell protein CCMk2, which was circularly permuted to position N- and C-termini to face outward to allow genetic fusion with denoted VHH. Trx denotes thioredoxin. “Tri” and “pent” denote the synthetically designed non-covalent multimers 1na0C3 and DHR5C5_G2, respectively. Single and double VHHs are appended to said multimers in the orientations designated on the blots. SP 744, 745, 746, and 747 are thiredoxin fusions with 5D and E3 in both the N- and C-terminal orientations.

FIG. 39 and Table 1 demonstrates the potency of various anti-tcdB VHH constructs.

FIG. 40 demonstrates colorimetric assays testing the anti-tcdB VHH constructs.

FIGS. 41A-O demonstrate morphology and cytotoxicity assays testing the anti-tcdB VHH constructs. FIGS. 41I-K and FIGS. 41M-41O: Characterization of anti-TcdB neutralizing potency of high-performing spirulina strains in the Vero cell rounding assay, using both the 027 and 10463 forms of TcdB. Spirulina lysates were normalized to transgene mass and compared in high and low concentration against a titration of toxin. FIG. 41I: SP744: VHH 5D-Trx neutralizing curves; FIG. 41J: SP985: VHH 5D-d.PP7 VLP neutralizing curves: FIG. 41K: SP1087: Trx-Trimer-VHH.5D neutralizing curves: FIG. 41M: SP1095: VHH.E3-Trx-Trimer-VHH.5D neutralizing curves; FIG. 41N: SP977: VHH.5D-dMS2 VLP neutralizing curves; FIG. 41O: SP1091: Trx-Pentamer-VHH.5D. FIG. 41L: Characterization of anti-TcdB neutralizing potency of select spirulina strains in the Vero cell rounding assay, using the 027 form of TcdB. Spirulina lysates were normalized to transgene mass and compared in high and low concentration against a titration of toxin. Best performers are denoted with red ovals.

FIG. 42 demonstrates the binding strength of various VHH sequences to C. difficile TcdB toxin.

FIG. 43 demonstrates the binding strength of combinations of different VHH sequences to C. difficile TcdB toxin.

FIG. 44A-B demonstrates the binding strength of the combination of the 5D, E3, and 7F VHHs to C. difficile TcdB toxin both alone and in combination.

FIGS. 45A-B demonstrate the effect of VHH concentration on binding to C. difficile TcdB toxin. While concentration increases of any of the single VHH sequences had little effect on efficacy, surprisingly, concentration increases of the combination of VHH sequences showed a large increase in efficacy.

FIG. 46 shows putative synergistic action of different VHHs on C. difficile infection and signaling.

FIG. 47A-B: Two-way synergy among anti-TcdB VHHs. Scoring denotes cell rounding index: 7=normal, 1=100% rounding, 4=50% rounding, as determined by visual inspection. Scores of 5 and 6 are on a gradient from 50% round to 100% normal, and scores of 3 and 2 are on a similar gradient from 50% round to 100% round.

FIG. 48: Individual and 2-way synergy combinations of anti-TcdB VHHs, measured in Vero cell rounding assay using TcdB 027.

FIG. 49 describes a putative cocktail for preventing and/or treating a C. difficile infection.

FIG. 50: Schema of VHH hybridization with candidate scaffold partners. VHHs were selected based on our evaluation, and published structure/function studies with TcdB.

FIG. 51 shows constructs for assessment of rigid inter-domain linkers.

FIG. 52A-B shows the crystal structure of VHH E3 co-crystallized with TcdB.

FIG. 53 shows exemplary sequences engineering VHH.E3-like activity onto other frameworks.

FIG. 54 shows adherence values for individual VHHs produced in Spirulina.

FIG. 55 demonstrates that a mixture of three Spirulina-expressed VHHs (5D+E3+7F) is substantially more potent than individual constituents.

FIG. 56 shows adherence values for mixtures of Spirulina-produced VHHs.

FIG. 57 shows that mixtures of Spirulina-produced VHHs neutralizes high-doses of TcdB.

FIG. 58 shows how the present disclosure can be employed to rapidly discover antibodies tailor-made for oral delivery.

FIG. 59 shows maximized strain cross-reactivity. A comparison of the domain in FlaA targeted by LMN-101 from the Navy (NCBIC) C. jejuni database (>10,000 sequences) shows that 79% of the sequences share at least 75% homology suggesting that cross-reactivity of this one lead VHH may extend to 79% of campylobacter strains.

FIG. 60 shows a proposed model for prevention of C. difficile in mice.

FIG. 61 shows an exemplary double-blind, placebo-controlled study to evaluate the safety and tolerability of LMN-101.

FIG. 62 shows an exemplary double-blind, placebo-controlled study to evaluate the safety and prophylactic activity of LMN-101 against C. jejuni CG8421 (human challenge strain.

FIG. 63 shows cell lysis assay results for both E. coli-expressed and Spirulina-expressed proteins. Log-phase cultures, O.D.600=1, of C. difficile were treated with the indicated concentrations of lysin. Cell lysis was measured by reduction in optical density over time. Spirulina-expressed lysins are biologically active.

FIG. 64: the effect of rigid linkers on VHH 5D neutralizing activity. The assay has a numeric read out from 1 (totally detached and dying) to 7 (normal).

FIG. 65: overview of Spirulina stability assay

FIG. 66: aqueous stability study of SP1308, MBP-5HVZ-VHH 5D. The lysates were incubated in media for four hours.

FIG. 67: aqueous stability study of SP1312, MBP-5HVZ-VHH E3. The lysates were incubated in media for four hours.

FIG. 68: aqueous stability study of SP1308+SP1312+SP1313. The lysates were incubated in media for four hours.

FIG. 69A-B: VHH aqueous stability. The aqueous stability of VHHs was measured at 12 hours using a VERO cell, cell rounding assay. FiA) shows neutralizing activity. B) shows the cell rounding assay.

FIG. 70: Overview of gnotobiotic pig model to assess the effect of anti-TcdB VHH on C. difficile infection.

FIG. 71A-B: Clinical Data: Piglet III treated with 3-VHH combination+/−lysin. A) shows diarrhea burden among animals experimentally infected with 027-strain Clostridium difficile. B) shows diarrhea burden among individual animals. Animals were treated from day −1 until end of study with either PBS (negative control), wildtype Spirulina (negative control), or spirulina containing three different anti-TcdB VHHs (Mix 1), or containing the same three VHHs and an anti-clostridium lysin (Mix 2).

FIG. 72: overview of Monash mouse CDI model study of anti-TcdB VHHs

FIG. 73A-C: Prophylactic activity of anti-TcdB VHHs, with and without a C. difficile specific lysin, in a mouse model of CDI. Mice were treated daily, beginning on day −1 and continuing to day 4, with the indicated spirulina biomass, or with vancomycin as a positive control. Mice were inoculated with pandemic 027 C. difficile on day 0. A) shows the effect on weight loss associated with CDI. B) shows the effect on survival. C) shows the effect on C. difficile spore shedding. (Dashed line is the limit of detection).

FIG. 74: ELISA titration curves of SP1182 extracts prepared in various pH buffers. Each binding curve represents 4-fold serial dilutions of protein extracts from spirulina biomass (μg/mL) resuspended in a different pH. Each curve was internally normalized to 1, and results are the mean of two replicates.

FIG. 75: Western blot gel analysis of SP1182 extracts prepared in various pH buffers. Lanes represent a 600-fold dilution of clarified spirulina extracts from spirulina biomass resuspended at 50 mg/mL and extracted in different pH buffers for 60 min.

FIG. 76: Western blot of intestinal-phase digestion of dried spirulina-VHH biomass. Spray-dried spirulina-VHH of SP806 was incubated in SIF for the indicated times. All incubation times are shown in minutes or overnight (ON). The experiment was performed twice with different time points (left and right panels). Intact biomass (Pellet) was analyzed alongside released sample (Supernatant). Samples were run on a western blot and detected with an anti-VHH antibody. The arrows indicate expected band size for full-length VHH protein.

FIG. 77: Western blot of in vitro intestinal-phase digestion of SP1182 Drug Substance. Dried spirulina biomass was incubated in SIF for the indicated times. Intact biomass (Pellet) was analyzed alongside released sample (Supernatant). Samples were run on a western blot and detected with an anti-VHH antibody. Red box indicates bands of VHH (aa682).

FIG. 78: Western blot of in vitro intestinal-phase digestion of aa682. Purified aa682 was incubated in simulated intestinal fluid for the indicated times. Samples were run on a western blot and detected with an anti-VHH antibody.

FIG. 79: SDS-PAGE analysis of gastric-phase digestion of dried spirulina biomass. Spray-dried spirulina biomass of (SP806, containing a trimeric VHH) was incubated in SGF for the indicated time periods or overnight (O/N). Intact biomass (Pellet) was analyzed alongside released sample (Supernatant). Samples were run on SDS-PAGE gels and analyzed by Coomassie stain (upper gel) and western blot (lower gel). Proteins were detected on the western blot with an anti-VHH antibody. The black box highlights bands corresponding to the VHH.

FIG. 80: Western blot of gastric-phase digestion of SP1182 drug substance. Dried spirulina was incubated in SGF for the indicated times or overnight (O/N). Intact biomass (Pellet) was analyzed alongside released sample (Supernatant). Samples were run on a western blot and detected with an anti-VHH antibody. Red box indicates bands of VHH (aa682).

FIG. 81: Serum IgG response to maltose binding protein (MBP) on day 14. Sera was diluted as indicated. PO=oral administration; IN=intranasal administration. Pos. cont=the positive control of hyperimmune sera beginning at 1/200 with 3× serial dilution.

FIG. 82: Serum IgG response to NANP on day 14. Sera was diluted as indicated. PO=oral administration; IN=intranasal administration. Pos. cont=the positive control of hyperimmune sera beginning at 1/200 with 3× serial dilution. Groups 2 and 3 show production of IgG antibodies by day 14.

FIG. 83: Serum IgG response to NANP on day 27. Sera was diluted as indicated. PO=oral administration; IN=intranasal administration. Pos. cont=the positive control of hyperimmune sera beginning at 1/200 with 3× serial dilution. Groups 2 and 3 show production of IgG antibodies by day 27.

FIG. 84: Serum IgG response to NANP on day 41. Sera was diluted as indicated. PO=oral administration; IN=intranasal administration. Pos. cont=the positive control of hyperimmune sera beginning at 1/200 with 3× serial dilution. Groups 2 and 3 show production of IgG antibodies by day 41.

FIG. 85: Serum IgG response to NANP on day 56. Sera was diluted as indicated. PO=oral administration; IN=intranasal administration. Pos. cont=the positive control of hyperimmune sera beginning at 1/200 with 3× serial dilution. Groups 2 and 3 show production of IgG antibodies by day 56.

FIG. 86: Serum IgG response to NANP on day 69. Sera was diluted as indicated. PO=oral administration; IN=intranasal administration. Pos. cont=the positive control of hyperimmune sera beginning at 1/200 with 3× serial dilution. Groups 2 and 3 show production of IgG antibodies by day 69.

FIG. 87: Survival of vaccinated mice after challenge with P. falciparum.

### DETAILED DESCRIPTION

The present disclosure teaches exogenous therapeutics or prophylactic molecules are packaged in prokaryotic algae and then administered by non-parenteral means to a subject. In some embodiments, the recombinant prokaryotic algae are edible and can serve as an edible composition for the delivery of the payload expressed in the algae. In the case of polypeptide therapeutics or prophylactic molecules (e.g. antibodies, antigens, etc.), the expression levels of the exogenous polypeptides in the Spirulina delivery systems of the present disclosure are 10 to 100-fold higher compared to other systems.

Provided herein are non-parenteral compositions comprising a recombinant Spirulina comprising at least one exogenous therapeutic or prophylactic molecule, methods of making, and use thereof.

Before describing certain embodiments in detail, it is to be understood that this disclosure is not limited to particular compositions or biological systems, which can vary. It is also to be understood that the terminology used herein is for the purpose of describing particular illustrative embodiments only, and is not intended to be limiting. The terms used in this specification generally have their ordinary meaning in the art, within the context of this disclosure and in the specific context where each term is used. Certain terms are discussed below or elsewhere in the specification, to provide additional guidance to the practitioner in describing the compositions and methods of the disclosure and how to make and use them. The scope and meaning of any use of a term will be apparent from the specific context in which the term is used. As such, the definitions set forth herein are intended to provide illustrative guidance in ascertaining particular embodiments of the disclosure, without limitation to particular compositions or biological systems.

Following long-standing patent law convention, the terms “a”, “an”, and “the” refer to “one or more” when used in this application, including the claims, unless clearly indicated otherwise. By way of example, “an antigenic epitope” means one epitope or more than one epitope.

As use herein, the term “antigenic composition” refers to a preparation which, when administered to a subject will induce a protective immune response that provides immunity to a disease or disorder, or can be used to treat a disease or disorder as described herein.

The term “antigen” as used herein refers to a protein or a peptide that binds to a receptor of an immune cell and induces an immune response in a human or an animal. The antigen can be from infectious microorganisms including viruses, bacteria, parasite, or fungi or the antigen can be a tumor antigen or a self-antigen associated with an autoimmune disease.

The term “antigenic epitope” as used herein refers to a short amino acid sequence, for example, of about 4 to 1000 amino acids, of an antigen that is recognized by, and binds to, a receptor of an immune cell and induces an immune response in a human or an animal. The antigenic epitopes of the present disclosure are from the antigens described above.

The term “subject” as used herein refers to a vertebrate or an invertebrate, and includes mammals, birds, fish, reptiles, and amphibians. Subjects include humans and other primates, including non-human primates such as chimpanzees and other apes and monkey species. Subjects include farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like; and aquatic animals such as fish, shrimp, and crustaceans.

### Non-Parenteral Therapeutic Compositions

Provided herein are non-parenteral compositions comprising a recombinant Spriulina, wherein the Spirulina is engineered to contain at least one exogenous therapeutic or fragment thereof. As used herein, the term “therapeutic” refers to any molecule that may be used to treat a disease or disorder and/or have a therapeutic effect in a subject. As used herein, the term “prophylactic” refers to any molecule that may be used to prevent the development of a disease or disorder in a subject.

There are several advantages to non-parenterally delivering therapeutics or prophylactic molecules encapsulated in Spirulina. One of these advantages is the increased resistance of the encapsulated therapeutic or prophylactic molecule to proteolysis. For example, when delivered orally, encapsulation in Spirulina protects the therapeutic or prophylactic molecule from the enzymes and conditions of the digestive tract, thereby allowing the therapeutic to be delivered to the portion of the digestive tract that digests the Spirulina cells and releases the therapeutic or prophylactic molecule. In some embodiments, the orally delivered compositions of the present disclosure survive (e.g. remain substantially intact) at a pH of about 1.3 to about 8.0. In some embodiments, the orally delivered compositions of the present disclosure survive in the oral cavity. In some embodiments, the orally delivered compositions of the present disclosure survive in the stomach. In some embodiments, the orally delivered compositions survive in the small and/or large intestine. In some embodiments, the orally delivered compositions survive in the colon. In some embodiments, the orally delivered compositions survive in a simulated stomach environment. In some embodiments, the simulated stomach environment has an acidic pH and contains pepsin. In some embodiments, the simulated stomach environment has a pH of about 3.0 and about 2000 U/mL of pepsin. In some embodiments, the orally delivered compositions can survive in the gastrointestinal conditions or simulated stomach environment for about 5 minutes to about 1 day. In some embodiments, the orally delivered compositions survive in the gastrointestinal conditions or simulated stomach environment for about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 12 hours, about 24 hours. In some embodiments, the orally delivered compositions survive in the gastrointestinal conditions or simulated stomach environment overnight.

In some embodiments, the non-parenterally delivered compositions of the present disclosure survive (e.g. remain substantially intact) at a pH of about 5.0 to about 8.0. In some embodiments, the non-parenterally delivered compositions of the present disclosure survive (e.g. remain substantially intact) at a pH of about 5.5 to about 6.5. In some embodiments, the non-parenterally delivered compositions of the present disclosure survive in the oral cavity. In some embodiments, the non-parenterally delivered compositions of the present disclosure survive in the nose. In some embodiments, the non-parenterally delivered compositions survive in the pharynx. In some embodiments, the non-parenterally delivered compositions survive in the trachea. In some embodiments, the non-parenterally delivered compositions survive in the bronchi. In some embodiments, the non-parenterally delivered compositions survive in the lungs. In some embodiments, the non-parenterally delivered compositions survive in the alveoli. In some embodiments, the non-parenterally delivered compositions survive in the airway. In some embodiments, the non-parenterally delivered compositions survive in a simulated nasal cavity and/or respiratory tract environment. In some embodiments, the simulated nasal cavity environment has a pH of about 5 to about 7. In some embodiments, the simulated nasal cavity environment has a pH of about 5.5 to about 6.5. In some embodiments, the simulated respiratory tract environment has a pH of about 7 to about 8. In some embodiments, the simulated respiratory tract environment has a pH of about 7.3 to about 7.5. In some embodiments, the non-parenterally delivered compositions can survive in nasal cavity conditions, respiratory tract conditions, or a simulated respiratory tract environment for about 5 minutes to about 1 day. In some embodiments, the non-parenterally delivered compositions can survive in nasal cavity conditions, respiratory tract conditions, or a simulated respiratory tract environment for about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 12 hours, about 24 hours. In some embodiments, the non-parenterally delivered compositions can survive in nasal cavity conditions, respiratory tract conditions, or a simulated respiratory tract environment overnight. In some embodiments, the non-parenterally delivered composition is an extract of a recombinant Spriulina biomass.

Another advantage of the non-parenterally delivered compositions of the present disclosure is their stability in storage. In some aspects, the non-parenterally delivered compositions of the present disclosure are stable at elevated temperatures (e.g. greater than room temperature). In some embodiments, the non-parenterally delivered compositions of the present disclosure are stable at 42° C. In some embodiments, the non-parenterally delivered compositions of the present disclosure are stable at 42° C. for about one day to 5 years. In some embodiments, the non-parenterally delivered compositions of the present disclosure are stable at 42° C. for about one day, two days, three days, four days, five days, six days, seven days, one week, two weeks, three weeks, four weeks, one month, two months, three months, four months, five months, six months, or one year. In some embodiments, the non-parenterally delivered compositions of the present disclosure are stable at 42° C. for one month or three months. In some embodiments, the non-parenterally delivered compositions of the present disclosure are stable at room temperature (e.g. about 20° to about 29° C.). In some embodiments, the non-parenterally delivered compositions of the present disclosure are stable at 27° C. In some embodiments, the non-parenterally delivered compositions of the present disclosure are stable at 27° C. for about one day to 5 years. In some embodiments, the non-parenterally delivered compositions of the present disclosure are stable at 27° C. for about one day, two days, three days, four days, five days, six days, seven days, one week, two weeks, three weeks, four weeks, one month, two months, three months, four months, five months, six months, or one year. In some embodiments, the non-parenterally delivered compositions of the present disclosure are stable at 27° C. for one month or three months.

### Therapeutics

Any exogenous (i.e. non-Spriulina) therapeutic or prophylactic molecule appropriate for non-parenteral administration may be used in the compositions and methods of the present disclosure. In some embodiments, the therapeutic or prophylactic molecule is a small-molecule. In some embodiments, the therapeutic or prophylactic molecule is a polypeptide or a fragment thereof. In some embodiments, the recombinant Spriulina comprises a mixture of therapeutics, including a mixture of polypeptides or fragments thereof, a mixture of small molecules, or prophylactic molecules and/or a mixture of polypeptides or fragments thereof and small molecules.

In some embodiments, the therapeutic or prophylactic molecule is a small molecule is produced by a cell. In some embodiments, the small molecule is produced by a microorganism such as a bacteria, virus, fungus, or parasite. In some embodiments, the small molecule is produced by a plant.

In some embodiments, the small molecule has an anti-microbial effect. In some embodiments, the small molecule has an anti-fungal effect. In some embodiments, the small molecule has an anti-viral effect. In some embodiments, the small molecule has an anti-parasite effect. In some embodiments, the small molecule is selected from the group consisting of, but not limited to, antibiotics, malacidins, penicillin, streptomycin, polymyxin, colistin, circulin, bacillomycin, mycobacillin, fungistatin. tannins, terpenoids, saponins, alkaloids, flavonoids, polyphenols, saponins, chloroquine, quinine, amodiaquine, hydroxychloroquine, Metronidazole, tinidazole, iodoquinol, paromomycin, metronidazole and tinidazole, or a combination thereof.

In some embodiments, the exogenous therapeutic or prophylactic molecule is a polypeptide or a fragment thereof. In some embodiments, the polypeptide or prophylactic molecule is selected from the group consisting of, but not limited to, a receptor, an agonist, a hormone, a neurotransmitter, a secreted polypeptide, an anchored polypeptide, a transcription factor, an antimicrobial peptide, a chemokine, a cytokine, a pro-protein, a pre-pro-protein, an interferon, an antibody, a neuropeptide, an antigen, an epitope from an antigen, a self-antigen, a secretin, a G-protein coupled receptor, an opioid peptide, cell-surface protein, a cytoplasmic protein, a mitochondrial protein, a cell-signalling protein, insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, and chlorotoxin, or a combination thereof.

In some aspects, the present disclosure does not comprise compositions or methods of Spirulina comprising an antigen, antigenic epitope, or fragment thereof. In some embodiments, the present disclosure does not comprise the subject matter of PCT/US2019/032998 filed May 17, 2019. In some embodiments, the present disclosure does not comprise compositions or methods to elicit or increase an immune response in a subject. In some embodiments, the present disclosure does not comprise compositions or methods to elicit or increase the production of antibodies or fragments thereof against the exogenous polypeptide contained in the Spirulina.

In some embodiments, the polypeptide is an antibody or fragment thereof. In some embodiments, the antibody or fragment thereof is selected from the group consisting of, but not limited to, full length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, nanobody, IgNAR, V-NAR, HcIgG, or a combination thereof.

In some embodiments, the therapeutic peptide is associated with, or derived from, or treats or prevents infection by any microorganism, including, but not limited to, E. coli, Enterotoxigenic E. coli (ETEC), anthrax, EHEC, EAEC, Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella, Legionella, bacteriophage, RNA bacteriophage (e.g. MS2, AP205, PP7 and Qβ), Helicobacter pylori, Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, White Spot Syndrome Virus, a coronavirus, SARS, MERS, SARS-CoV-2, Aspergillus, Candida, Blastomyces, Coccidioides, Cryptococcus, Histoplasma, Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. and Wuchereria spp., Taenia spp., Echinococcus spp., and Diphyllobothrium spp., Fasciola spp., and Schistosoma spp. or a combination thereof.

In some embodiments, the exogenous polypeptide is an antigen or a self-antigen. In some embodiments, the self-antigen is associated with an autoimmune disease or disorder. In some embodiments, the self-antigen is a tumor antigen. In some embodiments, the exogenous polypeptide binds to an antigen or self-antigen.

In various embodiments, the compositions of the present disclosure comprise a recombinant Spirulina comprising at least one exogenous polypeptide derived from (e.g. a portion or fragment thereof, or antigenic variant thereof) an infectious microorganism, a tumor antigen or a self-antigen associated with an autoimmune disease.

In some embodiments, the compositions comprise a recombinant Spirulina comprising at least one exogenous antigenic epitope derived from an infectious microorganism such as a virus, bacterium, parasite, or fungus. The infectious microorganism can be a microorganism that causes infections in a human or an animal such as a species of livestock, poultry, and fish.

In some embodiments, the compositions of the present disclosure comprise a recombinant Spirulina comprising at least one polypeptide, antigen, or antigenic epitope from a virus including but not limited to, bacteriophage, RNA bacteriophage (e.g. MS2, AP205, PP7 and Qβ), Helicobacter pylori, infectious haematopoietic necrosis virus (IHNV), parvovirus, Herpes Simplex Virus, Hepatitis A virus, Hepatitis B virus, Hepatitis C virus, Measles virus, Mumps virus, Rubella virus, Human Immunodeficiency Virus (HIV), Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, White Spot Syndrome Virus, a coronavirus, MERS, SARS, and SARS-CoV-2. In some embodiments, the compositions of the present disclosure comprise a recombinant Spirulina comprising at least one polypeptide, antigen, or antigenic epitope from IHNV. In some embodiments, the compositions of the present disclosure comprise the recombinant Spirulina SP105 or SP113. In some embodiments, the compositions of the present disclosure comprise a recombinant Spirulina comprising at least one polypeptide, antigen, or antigenic epitope from a coronavirus. In some embodiments, the compositions of the present disclosure comprise a recombinant Spirulina comprising at least one polypeptide, antigen, or antigenic epitope from SARS-CoV-2. In some embodiments, oral compositions of the present disclosure comprise a recombinant Spirulina comprising at least one polypeptide, antigen, or antigenic epitope from a parvovirus, e.g., canine parvovirus. In some embodiments, the compositions of the present disclosure comprise the recombinant Spirulina SP673 or SP678.

In some embodiments, the compositions of the present disclosure comprise a recombinant Spirulina comprising at least one polypeptide or fragment thereof that binds to a virus or portion thereof, including but not limited to, bacteriophage, RNA bacteriophage (e.g. MS2, AP205, PP7 and Qβ), Helicobacter pylori, infectious haematopoietic necrosis virus (IHNV), parvovirus, Herpes Simplex Virus, Hepatitis A virus, Hepatitis B virus, Hepatitis C virus, Measles virus, Mumps virus, Rubella virus, Human Immunodeficiency Virus (HIV), Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, White Spot Syndrome Virus, a coronavirus, MERS, SARS, and SARS-CoV-2.

In some embodiments, the recombinant Spirulina comprises a polypeptide or fragment thereof that binds to a Norovirus polypeptide or antigen. In some embodiments, the recombinant Spirulina comprises a polypeptide or fragment thereof that binds to a norovirus P domain. In some embodiments, the polypeptide or fragment thereof is a VHH. In some embodiments, the recombinant Spirulina comprises a VHH that binds to a Norovirus polypeptide. In some embodiments, the recombinant Spirulina comprises a VHH that binds to a norovirus P domain. In some embodiments, the recombinant Spirulina comprises a polypeptide or fragment thereof that binds to a GII genotype, a G1 genotype, a G11.10 genotype. In some embodiments, the recombinant Spirulina comprises a polypeptide or fragment thereof that binds to a polypeptide from two or more norovirus genotypes. In some embodiments, the recombinant Spirulina comprises a VHH comprising a Nano85 nanobody, a Nano26 nanobody, a Nano94 nanobody, a K922 antibody or a modified sequence or fragment thereof. In some embodiments, the recombinant Spirulina comprises a VHH comprising Nano85 and/or a loop grafted modification thereof. In some embodiments, the VHH comprises the amino acid sequence of any of SEQ ID NOs: 40-79 or a fragment thereof. In some embodiments, the recombinant Spirulina comprises a polypeptide or fragment thereof that binds a Norovirus polypeptide or antigen, or fragment thereof in fusion with a chaperone polypeptide. In some embodiments, the recombinant Spirulina comprises multiple copies of a polypeptide or fragment thereof that binds a Norovirus polypeptide or antigen, or fragment thereof in fusion with a chaperone polypeptide. In some embodiments, the chaperone polypeptide is maltose binding protein (MBP) or Thioredoxin A (TxnA). In some embodiments, the recombinant Spirulina comprises a monomer, dimer, or heptamer of a polypeptide or fragment thereof that binds an anti-Clostridium toxin or fragment thereof. I In some embodiments, the recombinant Spirulina comprises a VHH comprising a Nano85 and/or a loop grafted modification thereof in fusion with a chaperone polypeptide. In some embodiments, the chaperone polypeptide is maltose binding protein (MBP) or Thioredoxin A (TxnA). In some embodiments, the recombinant Spirulina comprises multiple copies of VHH comprising a Nano85 and/or a loop grafted modification thereof in fusion with a chaperone polypeptide. In some embodiments, the recombinant Spirulina is SP833, SP834, SP835, SP864, SP1241, SP1371 or SP1372.

In some embodiments, the compositions comprise a recombinant Spirulina comprising at least one antigenic epitope from a bacterium including but not limited to, Mycobacterium, Streptococcus, Staphylococcus, Shigella, Campylobacter, Salmonella, Clostridium, Corynebacterium, Pseudomonas, Neisseria, Listeria, Vibrio, Bordetella, E. coli (including pathogenic E. coli), and Legionella.

In some embodiments, the recombinant Spirulina comprises a polypeptide that binds to a ETEC polypeptide or antigen or fragment thereof. In some embodiments, the recombinant Spirulina comprises a polypeptide that binds to a fimbriae polypeptide or fragment thereof. In some embodiments, the recombinant Spirulina comprises a VHH that binds to a ETEC polypeptide. In some embodiments, the recombinant Spirulina comprises a VHH that binds to a fimbriae polypeptide or fragment thereof. In some embodiments, the recombinant Spirulina comprises a VHH that binds to an adhesion or fragment thereof. In some embodiments, the recombinant Spirulina comprises a VHH that binds to a polypeptide from two or more adhesions. In some embodiments, the recombinant Spirulina comprises a polypeptide or fragment thereof that binds to the F4+ adhesin domain FaeG or the F18+ adhesin domain FedF. In some embodiments, the recombinant Spirulina comprises a polypeptide or fragment thereof that binds one or more of the adhesins K88 (also called F4), K99 (F5), 987P (F6), F41, and F18 or a modification or a fragment thereof. In some embodiments, the recombinant Spirulina comprises a polypeptide or fragment thereof that binds K88. In some embodiments, the recombinant Spirulina comprises a polypeptide or fragment thereof that binds an ETEC polypeptide or antigen or fragment thereof in fusion with a chaperone polypeptide. In some embodiments, the chaperone polypeptide is maltose binding protein (MBP) or Thioredoxin A (TxnA). In some embodiments, the recombinant Spirulina comprises multiple copies of a polypeptide or fragment thereof that binds an ETEC polypeptide or antigen or fragment thereof in fusion with a chaperone polypeptide. In some embodiments, the recombinant Spirulina comprises a monomer, dimer, or heptamer of a polypeptide or fragment thereof that binds an ETEC polypeptide or fragment thereof or of an ETEC polypeptide or antigen or fragment thereof. In some embodiments, the dimer or heptamer is homodimer or homoheptamer. In some embodiments, the dimer or heptamer is a heterodimer or heteroheptamer. In some embodiments, the recombinant Spirulina is SP795 or SP1156.

In some embodiments, the recombinant Spirulina comprises a polypeptide that binds to an anti-Clostridium toxin. In some embodiments, the Clostridium is C. difficile. In some embodiments, the recombinant Spirulina comprises a VHH that binds to an anti-Clostridium toxin. In some embodiments, the polypeptide or fragment thereof binds to a Clostridium component, toxin A, or toxin B, or both. In some embodiments, the polypeptide is a VHH comprising the amino acid sequence of any of SEQ ID NO:s 5-17 or fragment thereof. In some embodiments, the recombinant Spirulina comprises a Clostridium antigen or fragment thereof or a polypeptide or fragment thereof that binds an anti-Clostridium toxin or fragment thereof in fusion with a chaperone polypeptide. In some embodiments, the recombinant Spirulina comprises multiple copies of or a Clostridium antigen or fragment thereof or a polypeptide or fragment thereof that binds an anti-Clostridium toxin or fragment thereof in fusion with a chaperone polypeptide. In some embodiments, the chaperone polypeptide is maltose binding protein (MBP) or Thioredoxin A (TxnA). In some embodiments, the recombinant Spirulina comprises a monomer, dimer, or heptamer of or a Clostridium antigen or fragment thereof or a polypeptide or fragment thereof that binds an anti-Clostridium toxin or fragment thereof. In some embodiments, the dimer or heptamer is homodimer or homoheptamer. In some embodiments, the dimer or heptamer is a heterodimer or heteroheptamer. In some embodiments, the recombinant Spirulina is SP744, SP977, SP985, SP1087, SP1091, or SP1095.

In some embodiments, the recombinant Spirulina comprises a polypeptide that binds to a Campylobacter polypeptide or antigen or fragment thereof. In some embodiments, the Campylobacter is C. jejuni. In some embodiments, the recombinant Spirulina comprises a polypeptide that binds to a flagellin component. In some embodiments, the recombinant Spirulina comprises a polypeptide or fragment thereof that binds to a flagellin polypeptide or fragment thereof. In some embodiments, the recombinant Spirulina comprises a polypeptide or fragment thereof that binds to flaA or a fragment thereof. In some embodiments, the recombinant Spirulina comprises a VHH that binds to a Campylobacter polypeptide or antigen or fragment thereof. In some embodiments, the recombinant Spirulina comprises a VHH that binds to a flagellin polypeptide. In some embodiments, the recombinant Spirulina comprises a VHH that binds flaA or fragment thereof. In some embodiments, the recombinant Spirulina comprises a polypeptide or fragment thereof that binds a Campylobacter or antigen or fragment thereof in fusion with a chaperone polypeptide. In some embodiments, the chaperone polypeptide is maltose binding protein (MBP) or Thioredoxin A (TxnA). In some embodiments, the recombinant Spirulina comprises multiple copies of a polypeptide or fragment thereof that binds a Campylobacter polypeptide or antigen or fragment thereof in fusion with a chaperone polypeptide. In some embodiments, the recombinant Spirulina comprises a monomer, dimer, trimer, pentamer, or heptamer of a polypeptide or fragment thereof that binds a Campylobacter polypeptide, antigen, or fragment thereof. In some embodiments, the dimer, trimer, pentamer, or heptamer is homodimer, homotrimer, homopentamber, or homoheptamer. In some embodiments, the dimer, trimer, pentamer, or heptamer is a heterodimer, heterotrimer, heteropentamer, or heteroheptamer. In some embodiments, the recombinant Spirulina is SP526, SP651, SP742, or SP806.

In some embodiments, the recombinant Spirulina comprises a polypeptide that binds to a malaria polypeptide or antigen or fragment thereof. In some embodiments, the malaria is P. falciparum. In some embodiments, the recombinant Spirulina comprises a polypeptide that binds to a Circumsporozoite protein (CSP) or fragment thereof. In some embodiments, the recombinant Spirulina comprises a polypeptide or fragment thereof that binds to a polypeptide comprising one or more NANP repeats. In some embodiments, the recombinant Spirulina comprises a VHH that binds to a malaria polypeptide or antigen or fragment thereof. In some embodiments, the recombinant Spirulina comprises a VHH that binds to a CSP polypeptide. In some embodiments, the recombinant Spirulina comprises a VHH that binds a polypeptide comprising one or more NANP repeats. In some embodiments, the recombinant Spirulina comprises malaria antigen. In some embodiments, the malaria is P. falciparum. In some embodiments, the recombinant Spirulina comprises a Circumsporozoite protein (CSP) or fragment thereof. In some embodiments, the recombinant Spirulina comprises a polypeptide comprising one or more NANP repeats. In some embodiments, the polypeptide or fragment thereof is a VHH comprising the amino acid sequence of any of SEQ ID NOs: 26-31 or a fragment thereof. In some embodiments, the recombinant Spirulina comprises a polypeptide or fragment thereof that binds a malaria or antigen or fragment thereof in fusion with a chaperone polypeptide. In some embodiments, the chaperone polypeptide is maltose binding protein (MBP) or Thioredoxin A (TxnA). In some embodiments, the recombinant Spirulina comprises multiple copies of a polypeptide or fragment thereof that binds a malarai polypeptide or antigen or fragment thereof in fusion with a chaperone polypeptide. In some embodiments, the recombinant Spirulina comprises a monomer, dimer, trimer, pentamer, or heptamer of a polypeptide or fragment thereof that binds a malaria polypeptide, antigen, or fragment thereof. In some embodiments, the dimer, trimer, pentamer, or heptamer is homodimer, homotrimer, homopentamber, or homoheptamer. In some embodiments, the dimer, trimer, pentamer, or heptamer is a heterodimer, heterotrimer, heteropentamer, or heteroheptamer. In some embodiments, the recombinant Spirulina is SP648, SP803, or SP856.

In some embodiments, the at least one exogenous polypeptide is expressed in Spirulina by itself, i.e., the polypeptide is not fused to another protein.

In some embodiments, the at least one exogenous polypeptide expressed in Spirulina is comprised in an exogenous antigen. In some embodiments, the exogenous antigen is a natural antigen. For example, a recombinant Spirulina may express the entire circumsporozoite protein containing one or more antigenic epitopes or a portion or a domain of the circumsporozoite protein that contains one or more antigenic epitopes. In this case, the exogenous antigen is considered a natural antigen. Other examples of natural antigens that can be expressed in Spirulina to prepare oral antigenic compositions include hemagglutinin (HA), neuraminidase (NA), and matrix (M1) proteins of an influenza virus.

In addition to immunogenic epitopes, the present disclosure provides structures and/or ligands to stimulate the innate immune system (e.g. by engineering the epitopes into VLP structures). The innate immune system can be activated by adjuvant-like properties inherent in the VLP and/or adjuvants added to vaccine compositions. In some embodiments, these structures and/or ligands that stimulate the innate immune system include, but are not limited to, fragments of Salmonella flagellin, fliC, human and mouse TNF-alpha, and human and mouse CD40-Ligand. In some embodiments, the exogenous polypeptide is a fusion protein. For example, in some embodiments, a recombinant Spirulina may express a fusion protein comprising at least one exogenous polypeptide and a portion of another protein such as a viral protein or a scaffold protein. In some embodiments, the exogenous polypeptide or fragment thereof is in a fusion protein. In some embodiments, the fusion protein is a fusion of two or more polypeptides or fragments thereof. In some embodiments, the fusion protein is one or more polypeptides or fragments thereof attached to one or more scaffolding polypeptides. In some embodiments, the fusion protein is one or more polypeptides or fragments thereof attached to one or more chaperone polypeptides. In some embodiments, the fusion protein comprises a tag for separation and/or purification (e.g. a 6× His tag). In some embodiments, the fusion protein comprises one or more targeting signals or polypeptides. In some embodiments, the fusion protein comprises one or more VHH sequences in fusion with one or more chaperone polypeptides. In some embodiments, the fusion protein comprises one or more VHH sequences in fusion with one or more chaperone polypeptides and one or more scaffolding polypeptides.

In some embodiments, the exogenous antigenic epitopes can be from different antigens that activate different types of immunity (e.g. innate, cellular, or humoral). In some embodiments, the one or more exogenous antigenic epitopes from different antigens are from at least one B-cell antigen and at least one T-cell antigen. In some embodiments, the one or more exogenous antigenic epitopes are in a fusion protein with a viral protein (e.g. a coronavirus spike protein). In some embodiments, the one or more exogenous antigenic epitopes are in a fusion protein with a viral protein (e.g. a coronavirus spike protein) with one epitope at either terminus. In some embodiments, the one or more exogenous antigenic epitopes are a B-cell epitope fused to one terminus of a virus protein and a T-cell epitope fused to the other terminus of the virus protein.

In some embodiments, the compositions of the present disclosure comprise a recombinant Spirulina comprising multiple copies of one or more therapeutic and/or prophylactic molecules. In some embodiments, the compositions of the present disclosure comprise a recombinant Spirulina comprising a combination of therapeutic and/or prophylactic molecules. In some embodiments, oral compositions of the present disclosure comprise a recombinant Spirulina comprising multiple copies of one therapeutic or prophylactic and at least one other therapeutic or prophylactic molecule. In some embodiments, the compositions of the present disclosure comprise a recombinant Spirulina comprising at least one antibody and at least one other therapeutic or prophylactic molecule. In some embodiments, the compositions of the present disclosure comprise at least one VHH and at least one other therapeutic or prophylactic molecule. In some embodiments, the compositions of the present disclosure comprise at least one VHH and a polypeptide. In some embodiments, the compositions of the present disclosure comprise at least one VHH and a lysin polypeptide.

In some embodiments, one or more therapeutic and/or prophylactic molecule is an enzyme. In some embodiments, the enzyme is a hydrolytic enzyme. In some embodiments, the hydrolytic enzyme cleaves the wall of a cell. In some embodiments, the hydrolytic enzyme targets the bonds in peptidoglycan. In some embodiments, the hydrolytic enzyme includes, but is not limited to, lysin, a phage lysin, a cytolysin, an egg lysin, hemolysin, NK-lysin, streptolysin, an autolysin, a LytC amidase, a LytD glucosaminidase, a N-acetylmuramolyl-L-alanine amidase, a polypeptide comprising or consisting of one or more catalytic domains from a lysin or autolysin, or a combination and/or fragment thereof.

### Fusion Proteins

In some aspects of the disclosure, the therapeutic or prophylactic molecule may reside in the Spirulina as part of a complex. In some embodiments, the Spirulina comprise multiple copies of the one or more therapeutic and/or prophylactic molecule in a complex. In some embodiments, the Spirulina comprise a combination of one or more therapeutic and/or prophylactic molecules in a complex. In some embodiments, the Spirulina comprise one or more therapeutic and/or prophylactic molecules in a fusion protein.

In some embodiments, the Spirulina comprise one or more therapeutic and/or prophylactic molecules in a complex containing a linker. In some embodiments, the construct inserted into the recombinant Spirulina comprises a linker. In some embodiments, the polypeptide expressed from the recombinant Spirulina comprises a linker. In some embodiments, the linker is a rigid linker. In some embodiments, the linker is a flexible linker. In some embodiments, the linker attaches two or more VHH sequences. In some embodiments, the linker attaches one or more VHH sequences with another polypeptide. In some embodiments, the other polypeptide is selected from, but not limited to, a chaperone protein, a targeting protein, a scaffold, an oligomerization domain, an enzyme, or a lysin. In some embodiments, the linker is a helix 1 linker (SEQ ID NO: 19), helix 2 linker (SEQ ID NO: 20), helix 4 linker (SEQ ID NO: 21), a PA5 linker (SEQ ID NO: 22), a PA10 lin25).

In some embodiments, at least one exogenous polypeptide is expressed in Spirulina as a fusion protein, wherein the fusion protein forms a three-dimensional structure (sometimes referred to herein as “particles”). In some embodiments, the fusion protein that forms a three-dimensional structure may comprise multiple functional domains and one or more exogenous polypeptide. Such fusion proteins can be engineered in a number of ways. In some embodiments, a fusion protein is a single polypeptide with multiple modular domains. An example of this is the woodchuck hepadnavirus core antigen (WHcAg) engineered with a B cell antigen at the Major Insertion Region/spike position, and a T cell epitope at the C-terminus. Another example is an RNA bacteriophage (ie, MS2, PP7, AP205 or Q_{β}), engineered to be a tandem dimer, with an antigen at the N-terminus, and a fragment of Salmonella flagellin at the C-terminus, thus combining an immunogenic epitope with an innate immune system stimulant to act as an intrinsic adjuvant, which self-organizes into a three-dimensional structure with two functional domains displayed on its surface. In some embodiments, recombinant Spirulina may express two heterologous polypeptides. For example, a recombinant Spirulina may express one gene that encodes a tandem RNA bacteriophage capsid protein dimer with an N-terminal antigenic structure, and a second gene that encodes an identical capsid dimer but with an adjuvant like Salmonella flagellin at its C-terminus. These two nearly identical polypeptides expressed in Spirulina can cooperatively form a three-dimensional mosaic particle in which the two polypeptides contribute to the “tiling” that forms a VLP capsid. Another example of this is to express a gene encoding a viral capsid protein like WHcAg or one of the RNA phage particles with a polypeptide genetically linked, and a second gene with the native viral protein. This allows for the avoidance of stearic conflicts that might arise if every particle had a bulky hybrid partner attached. The particles formed in this example can self-organize forming further higher-order structures.

In some embodiments, the recombinant Spirulina comprises a fusion protein comprising at least one exogenous polypeptide and a trimerization domain of certain proteins that naturally exist as trimers. Exemplary proteins that comprise trimerization domains are described below. For example, the HA protein from influenza virus (either the whole ectodomain or the minimal stem region)naturally forms trimers, and interfaces between monomeric subunits are considered to be important immunodominant epitopes. The fusion protein (F protein) from respiratory syncytial virus (RSV) is an obligate trimer. Similarly, Tumor Necrosis Factor alpha (TNFα) and the ligand for CD40 (CD40L) are obligate trimers. A recombinant Spirulina comprising a fusion protein comprising at least one exogenous antigenic epitope and a trimerization domain of any of these proteins is encompassed by the present disclosure. In exemplary embodiments, to facilitate trimerization, the inventors have genetically linked the WHcAg monomer to a number of coiled-coil domains that both facilitate trimer formation and situate bulky domains like influenza HA away from the potential stearic interference by the spike domains of the WHcAg. The inventors have used a trimerization derivative of the Saccharomyces cerevisiae transcription factor GCN4, a parallel trimeric-coiled coil, and a related structure based on CGN4 with the addition of mutations informed by the HIV GP41 trimer structure. The inventors have genetically linked these two trimers, with varying length linker sequences, to WHcAg, as well as to a number of RNA bacteriophages.

In some embodiments, the recombinant Spirulina comprises a fusion protein comprising at least one exogenous polypeptide and a viral protein capable of forming a virus-like particle (VLP). In these embodiments, the exogenous polypeptide is expressed in Spirulina as a protein macromolecular particle, such as virus-like particles (VLPs). VLPs mimic the overall structure of a virus particle by retaining the three-dimensional structure of a virus without containing infectious material. VLPs have the ability to stimulate B-cell and T-cell mediated responses. When viral proteins are expressed in a heterologous system, such as Spirulina, they can spontaneously form VLPs. Accordingly, in some embodiments, the at least one exogenous antigenic epitope is fused to a VLP-forming viral protein. When this fusion protein is expressed in Spirulina, it forms a VLP.

In some embodiments, tethering the exogenous polypeptide to a VLP-forming viral protein (or other protein that forms tertiary structures) allows the expression of hundreds of monomer proteins per VLP (e.g. 180-240 monomer proteins per VLP when using the hepatitis VLP). This allows the expression of thousands of millions of VLPs per cell. In some embodiments, the exogenous polypeptide is tethered to a VLP-forming viral protein. In some embodiments, the exogenous antigenic epitope is tethered to a VLP-forming viral protein at the C-terminus or the N-terminus of the viral protein. That is, the amino acid sequence for the polypeptide is preceded by (attachment of the viral protein at the N-terminus of the antigen or the epitope), or followed by (attachment of the viral protein at the N-terminus of the antigen or the epitope), the amino acid sequence of the viral protein. In some other embodiments, the exogenous antigenic epitope is inserted into a VLP-forming viral protein. For example, the at least one exogenous polypeptide can be inserted between two adjacent amino acid residues of the viral protein. Alternatively, a region of the viral protein that is not required for the formation of a VLP can be replaced by inserting the at least one exogenous polypeptide in that region. Throughout this disclosure, when it is said that the at least one exogenous polypeptide is comprised in a VLP or is present in a VLP, it refers to the fusion protein comprising at least one exogenous polypeptide and a VLP-forming viral protein described herein.

Viral proteins that can be used to form polypeptide-containing VLPs of the present disclosure include capsid proteins of various viruses. Exemplary capsid proteins that can be used in the VLPs of the present disclosure include capsid proteins of viruses from the Hepadnaviridae family, papillomaviruses, picornaviruses, caliciviruses, rotaviruses, and reoviruses. In some embodiments, viral proteins that can be used to form polypeptide, antigen- or antigenic epitope-expressing VLPs of the present disclosure include the Hepadnaviridae core antigen (HBcAg). An exemplary HBcAg that can be used in the present disclosure is Woodchuck Hepadnaviral core antigen (WHcAg) from the Woodchuck Hepadnavirus (also referred to herein as Woodchuck Hepatitis Virus).

In some embodiments, the recombinant Spirulina comprises a fusion protein comprising at least one exogenous therapeutic and a protein that forms a trimer. In some embodiments, the trimer-forming protein is from an RNA bacteriophage or Helicobacter pylori. In some embodiments, the trimer-forming protein is the Helicobacter pylori ferritin protein. The at least one exogenous polypeptide, antigen, or antigenic epitope can be attached at the C-terminus or the N-terminus, or within the body of the protein that forms a trimer. In some embodiments, these proteins that form a trimer include but are not limited to, GCN4 polypeptides from S. cerevisiae and/or HIV or fragments, mutants or variants thereof.

In some embodiments, the recombinant Spirulina comprises a fusion protein comprising at least one exogenous polypeptide, antigen, or antigenic epitope and a scaffold protein. The term “scaffold protein” as used herein refers to a protein that acts as a docking protein and facilitates the interaction between two or more proteins. For example, a fusion protein comprising at least one exogenous polypeptide and a scaffold protein can facilitate the binding of the exogenous polypeptide with a receptor on a cell. In some embodiments, the exogenous polypeptide is tethered to a scaffold protein at the C-terminus or the N-terminus of the scaffold protein. In some other embodiments, the exogenous polypeptide is inserted into a scaffold protein (e.g. in the body of the scaffold protein). For example, the at least one exogenous polypeptide can be inserted between two adjacent amino acid residues of the scaffold protein. Alternatively, a region of the scaffold protein that is not required for the scaffolding function can be replaced by inserting the at least one polypeptide in that region. For example, in a recombinant Spirulina comprising multiple copies of the exogenous polypeptide and a scaffold protein, the exogenous antigenic epitope and the scaffold protein can be arranged in any one of the following patterns: (E)n-(SP), (SP)-(E)n, (SP)-(E)n-(SP), (E)n1-(SP)-(E)n2, (SP)-(E)n1-(SP)-(E)n2, and (SP)-(E)n1-(SP)-(E)n2-(SP), wherein E is the exogenous polypeptide, SP is the scaffold protein, and n, n1, and n2 represent the number of copies of the exogenous polypeptide. It is understood that the recombinant Spirulina may comprise more than one exogenous polypeptide and one or more scaffold proteins, where the multiple exogenous polypeptide and the scaffold proteins can be arranged in various patterns as described above.

In some embodiments, recombinant Spirulina may comprise a fusion protein comprising at least one exogenous polypeptide, a scaffold protein, a VLP-forming viral protein, and/or a trimer-forming protein. In these embodiments, the at least one exogenous polypeptide can be tethered to or inserted into one or more scaffold proteins as described above and the fusion protein comprising the scaffold proteins and the at least one exogenous polypeptide is tethered to or inserted into a VLP-forming viral protein and/or the trimer-forming protein.

Exemplary scaffold proteins include the oligomerization domain of C4b-binding protein (C4BP), a cholera toxin b subunit, or oligomerization domains of extracellular matrix proteins. In some embodiments, a scaffold protein used in the oral antigenic compositions of the present disclosure comprises a sequence from the oligomerization domain of C4BP selected from the group consisting of:

(SEQ ID NO: 1)
SAGAHAGWETPEGCEQVLTGKRLMQCLPNPEDVKMALEVYKLSLEIEQL
ELQRDSARQSTLDKEL,
(SEQ ID NO: 2)
WVIPEGCGHVLAGRKVMQCLPNPEDVKMALEVYKLSLEIELLEIQRDKA
RDPAMD,
(SEQ ID NO: 3)
WEYAEGCEQVVKGKKLMQCLPTPEEVRLALEVYKLYLEIQKLELQKDEA
KQA,
and
(SEQ ID NO: 4)
WVVPAGCEQVIAGRELTQCLPSVEDVKMALELYKLSLEIELLELQKDKA
KKSTLESPL.

In some embodiments, the exogenous polypeptide binds to a target or target molecule. In some embodiments, multimers of the exogenous polypeptide bind to the target or target molecule with a higher affinity than monomers or smaller multimers. For example, heptameric VHH may bind with higher affinity to a target than a dimer of the same exogenous polypeptide. In some embodiments, multimers are heteromeric. In some embodiments, the different components of the heteromer bind to different targets or target molecules.

The recombinant Spirulina present in the non-parenteral compositions of the present disclosure can comprise multiple copies of the at least one exogenous polypeptide. In some embodiments, the recombinant Spirulina expresses an exogenous polypeptide, or a fusion protein as described above, wherein the exogenous polypeptide comprises at least 1, 2, 3, 4, 5, 6, 7, 8, 9, or 10 copies of the at least one exogenous polypeptide per single molecule of the exogenous antigen. In some embodiments, the recombinant Spirulina expresses an exogenous polypeptide, wherein the exogenous polypeptide comprises 1-5, 2-5, 2-4, 3-6, 3-8, or 4-5 copies of the at least one exogenous polypeptide per single molecule of the exogenous antigen. In some embodiments, the recombinant Spirulina comprises at least 1, 2, 3, 4, 5, 6, 7, 8, 9, 10, 15, 20, 25, 30, 35, 40, 45, 50, 55, or 60 copies of the at least one exogenous polypeptide per single molecule of the exogenous antigen. In some embodiments, the recombinant Spirulina expresses an exogenous polypeptide, wherein the exogenous polypeptide comprises 1-10, 1-15, 1-20, 1-25, 1-30, 1-40, 1-50, 5-10, 5-15, 5-20, 5-25, 5-30, 5-40, 5-50, 10-25, 10-50, 10-60, 15-30, 15-45, 15-60, 20-50, 20-60, 20-70, 25-50, 25-60, 30-60, or 2-100 copies of the at least one exogenous polypeptide epitope per single molecule of the exogenous polypeptide. In some embodiments, the recombinant Spirulina cell can comprise thousands of copies of the at least one exogenous polypeptide (e.g. by expressing the corresponding nucleic acid sequences via one or more vectors in the cell or via integration into the Spirulina genome).

The recombinant Spirulina present in the non-parenteral compositions of the present disclosure can comprise multiple copies of a nucleic acid sequence encoding the at least one exogenous polypeptide. The multiple copies of the nucleic acid sequence encoding the at least one exogenous polypeptide can be integrated into the genome of the Spirulina or can be present on one or more vectors introduced into the Spirulina. In some embodiments, the recombinant Spirulina comprises between 2 and 100 copies of the nucleic acid sequence encoding the at least one exogenous polypeptide. In some embodiments, the recombinant Spirulina comprises at least 1, 2, 3, 4, 5, 6, 7, 8, 9, or 10 copies of a nucleic acid sequence encoding the at least one exogenous polypeptide integrated into its genome or present on one or more vectors. In some embodiments, the recombinant Spirulina comprises 1-5, 2-5, 2-4, 3-6, 3-8, or 4-5 copies of a nucleic acid sequence encoding the at least one exogenous polypeptide integrated into its genome or present on one or more vectors. In some embodiments, the recombinant Spirulina comprises at least 1, 2, 3, 4, 5, 6, 7, 8, 9, 10, 15, 20, 25, 30, 35, 40, 45, 50, 55, or 60 copies of a nucleic acid sequence encoding the at least one exogenous polypeptide integrated into its genome or present on one or more vectors. In some embodiments, the recombinant Spirulina comprises 1-10, 1-15, 1-20, 1-25, 1-30, 1-40, 1-50, 5-10, 5-15, 5-20, 5-25, 5-30, 5-40, 5-50, 10-25, 10-50, 10-60, 15-30, 15-45, 15-60, 20-50, 20-60, 20-70, 25-50, 25-60, or 30-60 copies of a nucleic acid sequence encoding the at least one exogenous polypeptide integrated into its genome or present on one or more vectors.

In some embodiments, multiple copies of the at least one exogenous polypeptide are linked in tandem, i.e., the first copy is immediately followed by the second copy without being separated by any amino acids, the second copy is immediately followed by the third copy, and so on. In some embodiments, where the recombinant Spirulina comprises more than one exogenous polypeptide, the individual polypeptide can be similarly linked in tandem to the other antigenic epitope. For example, in a recombinant Spirulina comprising E1 and E2 as exogenous polypeptide, these two polypeptides can be linked in tandem in the following ways: (E1E2)x, (E2E1)x, (E1)x(E2)y, (E1)x(E2)y(E1)z, (E2)x(E1)y(E2)z, where x, y, and z represent the number of copies of the polypeptides. Similar arrangement patterns for more than two exogenous polypeptides are contemplated.

In some embodiments, multiple copies of the at least one exogenous polypeptide present in a protein can be separated by spacer sequences. In some embodiments, multiple copies of the exogenous polypeptide can be separated by about 1 to about 50 amino acid space sequences. For example, in some embodiments, multiple copies of the exogenous polypeptide can be separated by about 1, 2, 3, 4, 5, 6, 7, 8, 9, 10, 15, 20, 30, 40, or 50 amino acid spacer sequences. It is understood that in these embodiments, when more than 2 copies of the exogenous polypeptide are present, some copies can be linked in tandem and some copies can be separated by spacer sequences. For example, in a recombinant Spirulina comprising multiple copies of E1 as the at least one exogenous polypeptide, the multiple copies of this epitope can be separated in the following ways: (E1)x-S-(E1)y, (E1)(E1)x-S-(E1)y, (E1)x-S-(E1)y-S-(E1)z, where S represents the spacer sequence and x, y, and z represent the number of copies of the exogenous polypeptide. When multiple spacer sequences are present, these sequences can be identical or different in length and/or the amino acid sequence.

In embodiments, where the recombinant Spirulina comprises a protein comprising more than one exogenous polypeptide, the first exogenous polypeptide can be separated from the other polypeptide epitope by spacer sequences of about 1, 2, 3, 4, 5, 6, 7, 8, 9, 10, 15, 20, 30, 40, or 50 amino acids. If multiple copies of each of the exogenous polypeptide are present, some of the copies can be linked in tandem with the other polypeptide while some copies can be separated by spacer sequences; alternatively, all copies of one polypeptide can be linked in tandem followed by a spacer sequence followed by all copies of the second polypeptide, and the like. For example, in a recombinant Spirulina comprising E1 and E2 as exogenous polypeptide, the two polypeptides can be arranged in the following ways: (E1)x-S-(E2)y, (E2)x-S-(E1)y, (E1)x-S-(E2)y-S(E1)z-S-(E2)v, (E1)x-S-(E2)y(E1)z, (E1)x-S-(E2)y-S-(E1)z, (E2)x-S-(E1)y (E2)z, and the like, where v, x, y, and z represent the number of copies of the polypeptide.

In some embodiments, a recombinant Spirulina may comprise one or more exogenous polypeptide and multiple copies thereof in the arrangement patterns described above directly, i.e., without being part of or fused to another protein.

In some embodiments, recombinant Spirulina comprises a fusion protein comprising a VLP-forming viral protein or a trimer-forming protein and one or more exogenous polypeptide, antigen, and/or antigenic epitopes, where the exogenous polypeptide, antigen, and/or antigenic epitopes and multiple copies thereof, if present, can be arranged within the fusion protein in various patterns as described above. In some other embodiments, recombinant Spirulina may comprise a fusion protein comprising a scaffold protein and one or more exogenous polypeptides, antigens, and/or antigenic epitopes, where the exogenous antigenic epitopes and multiple copies thereof, if present, can be arranged within the fusion protein in various patterns as described above. In some other embodiments, recombinant Spirulina may comprise a fusion protein comprising a VLP-forming viral protein, a trimer-forming protein, and/or a scaffold protein, and one or more exogenous polypeptides, antigens, and/or antigenic epitopes, where the exogenous polypeptide, antigens, and/or antigenic epitopes and multiple copies thereof, if present, can be arranged within the fusion protein in various patterns as described above.

The non-parenteral compositions provided by the present disclosure comprise a recombinant Spirulina, wherein the recombinant Spirulina comprises at least one exogenous polypeptide, small molecule, antigen or epitope in any of the ways described above.

### Spirulina

Non-parenteral compositions of the present disclosure comprise recombinant Spirulina in a non-living form. These non-living Spirulina containing an expressed exogenous polypeptide, small molecule, antigen or epitope are then administered to a subject to elicit an immune response in the subject. In some embodiments, non-living recombinant Spirulina comprising at least one exogenous polypeptide, antigen, or at least one exogenous antigenic epitope is prepared by drying the live culture of the recombinant Spirulina. Methods of drying include heat drying, e.g., drying in an oven; air-drying, spray drying, lyophilizing, or freeze-drying. Accordingly, in some embodiments, non-parenteral compositions of the present disclosure comprise a dried biomass of a recombinant Spirulina comprising at least one exogenous polypeptide, antigen, or at least one exogenous antigenic epitope as described herein.

As used herein “Spirulina” is synonymous with “Arthrospira.” Non-parenteral compositions of the present disclosure can comprise any one of the following species of Spirulina: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jennerif. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

### Pharmaceutical Compositions and Dosing

As used herein, the terms “oral composition” or “orally delivered composition” comprise compositions administered or delivered to the gastrointestinal tract (e.g. orally, compositions administered to the stomach via a feeding tube, etc.). Any appropriate area of the gastrointestinal tract may be targeted by the compositions of the present disclosure.

In some aspects, the compositions of the present disclosure are administered via the airway. In some embodiments, the compositions of the present disclosure are administered by inhalation. In some embodiments, the compositions of the present disclosure are administered intranasaly. In some embodiments, the compositions of the present disclosure are administered by a nebulizer, an inhaler, or a mist. In some embodiments, the compositions of the present disclosure are lyophilized and delivered as a powder or a powder resuspended in a liquid.

In some embodiments, the compositions of the present disclosure are formulated for administration via the airway. In some embodiments, the compositions of the present disclosure are formulated for administration by inhalation. In some embodiments, the compositions of the present disclosure are formulated for intranasal administration. In some embodiments, the compositions of the present disclosure are formulated for administration by a nebulizer, an inhaler, or a mist.

In some embodiments, compositions of the present disclosure can comprise one or more pharmaceutically acceptable excipients. Pharmaceutically acceptable carriers include but are not limited to saline, buffered saline, dextrose, water, glycerol, sterile isotonic aqueous buffer, and combinations thereof. In some embodiments, a pharmaceutically acceptable excipient is sodium bicarbonate.

In some embodiments, compositions of the present disclosure may comprise an adjuvant. As known in the art, the immunogenicity of a particular composition can be enhanced by the use of non-specific stimulators of the immune response, known as adjuvants. Exemplary adjuvants include a water-in-oil (W/O) emulsion composed of a mineral oil and a surfactant from the mannide monooleate family (e.g. MONTANIDE™ class of adjuvants) and flagellin adjuvants.

In some embodiments, compositions of the present disclosure comprise about 0.1% to about 5% of the total Spirulina biomass. In some embodiments, compositions of the present disclosure comprise about 1 mg to about 50 mg of the exogenous antigenic epitope per gram of dried Spirulina biomass. In some embodiments, compositions of the present disclosure comprise at least about 1 mg, 5 mg, 10 mg, 25 mg, 50 mg, 100 mg, 200 mg, 300 mg, 500 mg, 750 mg, 1 mg, 5 mg, 10 mg, or 50 of the exogenous antigenic epitope per gram of dried Spirulina biomass.

### Uses of Compositions

In some embodiments, compositions of the present disclosure can be used to reduce the severity of a disease or disorder in a subject in need thereof. In some embodiments, compositions can be used to prevent a disease or disorder in a subject. In some embodiments, compositions can be used to prevent initiation of a disease or disorder in a subject. In some embodiments, compositions can be used to reduce the severity of a disease or disorder in a subject. In some embodiments, compositions can be used to prevent or delay recurrence of a disease in a subject. In some embodiments, compositions can be used to treat, prevent, or delay recurrence of a cancer in a subject.

Compositions of the present disclosure can be used as a vaccine. In some embodiments, compositions can be used to induce an immune response in a subject. For example, compositions can be used to induce an immune response directed to an infectious microorganism, a tumor antigen, or a self-antigen.

In some embodiments, provided herein are methods of inducing an immune response in a subject in need thereof comprising administering to the subject any of the compositions described herein. Without wishing to be bound to a theory, it is expected that when the composition of the present disclosure is administered to a subject, the at least one exogenous antigenic epitope is recognized by immune cells of the subject, such as T cells or B cells, thereby activating an immune response against the exogenous antigenic epitope. In some embodiments, administration of compositions described herein can induce a humoral immune response and/or a cellular immune response.

The compositions of the present disclosure may be administered daily, weekly, biweekly, every other week, monthly, etc. In some embodiments, the compositions of the present disclosure are administered to a subject for about 1 day to about 1 year. In some embodiments, the compositions of the present disclosure are administered to a subject for about 1 day, 2 days, 3 days, 4 days, 5 days, 6 days, 7 days, 8 days, 9 days, 10 days, one week, two weeks, three weeks, four weeks, five weeks, six weeks, one month, two months, three months, four months, five months or more. In some embodiments, the compositions of the present disclosure are administered on consecutive days. In some embodiments, the compositions of the present disclosure are administered on non-consecutive days. In some embodiments, the compositions of the present disclosure are administered once a day. In some embodiments, the compositions of the present disclosure are administered multiple times a day. In some embodiments, the compositions of the present disclosure are administered twice a day, three times a day, four times a day, or more. In some embodiments, the compositions of the present disclosure are administered continuously (e.g. via a feeding tube). In some embodiments, the compositions of the present disclosure are administered with meals. In some embodiments, the compositions of the present disclosure are administered when the subject is in a fasting state.

Compositions of the present disclosure can be administered according to a schedule, for example, administering a priming dose of an antigenic composition and subsequently administering one or more booster doses of the antigenic composition. In some embodiments, a first booster dose of the antigenic composition can be administered anywhere from about two weeks to about 10 years after the priming dose. In some embodiments, a first booster dose of the antigenic composition can be administered anywhere from about two weeks, 1 month, 2 months, 3 months, 4 months, 6 months, 9 months, 1 year, 2 years, 3 years, or 5 years after the priming dose. A second booster dose of the antigenic composition can be administered after the first booster dose and anywhere from about 3 months to about 10 years after the priming dose. In some embodiments, a second booster dose of the antigenic composition can be administered after the first booster dose and from about 3 months, 4 months, 6 months, 9 months, 1 year, 2 years, 3 years, or 5 years after the priming dose. The third booster dose may be optionally administered when no or low levels of specific immunoglobulins are detected in the serum and/or other bodily fluids of the subject after the second booster dose.

In some embodiments, compositions other than the compositions of the present disclosure can be administered prior to the administration of the present compositions to prime the subject's immune response. In these embodiments, methods of the present disclosure comprise administering an composition other than the present antigenic composition as a priming dose and subsequently administering one or more booster doses of the present composition.

Compositions of the present disclosure can be used to treat and/or prevent or reduce the severity of a disease or disorder. In some embodiments, the disease or disorder is selected from the group including, but not limited to, Type 1 diabetes, Type 2 diabetes, cancer, an inflammatory disorder, a gastrointestinal disease, an autoimmune disease or disorder, an endocrine disorder, gastroesophageal reflux disease (GERD), ulcers, high cholesterol, inflammatory bowel disorder, irritable bowel syndrome, crohn's disease, ulcerative colitis, constipation, and diarrhea.

Compositions of the present disclosure can be used as a vaccine or to treat and/or prevent or reduce the severity of a disease or an infection caused by a virus, bacterium, parasite, or fungus.

In some embodiments, compositions can be used as a vaccine or to treat, and/or reduce the severity of an infection such as tetanus, diphtheria, pertussis, pneumonia, meningitis, campylobacteriosis, mumps, measles, rubella, polio, flu, hepatitis, chickenpox, malaria, toxoplasmosis, giardiasis, or leishmaniasis.

In some embodiments, compositions described herein can be used to induce an immune response to, to treat and/or reduce the severity of an infection caused by a virus including, but not limited to, bacteriophage, RNA bacteriophage (e.g. MS2, AP205, PP7 and Qβ), Helicobacter pylori, infectious haematopoietic necrosis virus (IHNV), parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, White Spot Syndrome Virus, a coronavirus, SARS, MERS, and SARS-CoV-2.

In some embodiments, the compositions described herein can be used to induce an immune response to, to treat, and/or reduce the severity of an infection caused by IHNV.

In some embodiments, thecompositions described herein can be used to induce an immune response to and/or reduce the severity of an infection caused by a parvovirus, e.g., canine parvovirus.

In some embodiments, the compositions described herein can be used to induce an immune response to and/or reduce the severity of an infection caused by a coronavirus, e.g., ARDS, COVID-19.

In some embodiments, compositions described herein can be used to induce an immune response to, to treat and/or reduce the severity of an infection caused by a bacterium including, but not limited to, Mycobacterium, Streptococcus, Staphylococcus, Shigella, Campylobacter, Salmonella, Clostridium, Corynebacterium, Pseudomonas, Neisseria, Listeria, Vibrio, Bordetella, and Legionella.

In some embodiments, compositions described herein can be used to induce an immune response to and/or reduce the severity of an infection caused by a parasite including, but not limited to, Plasmodium, Trypanosoma, Toxoplasma, Giardia, and Leishmania, Cryptosporidium, helminthic parasites: Trichuris spp. (whipworms), Enterobius spp. (pinworms), Ascaris spp. (roundworms), Ancylostoma spp. and Necatro spp. (hookworms), Strongyloides spp. (threadworms), Dracunculus spp. (Guinea worms), Onchocerca spp. and Wuchereria spp. (filarial worms), Taenia spp., Echinococcus spp., and Diphyllobothrium spp. (human and animal cestodes), Fasciola spp. (liver flukes) and Schistosoma spp. (blood flukes).

In some embodiments, compositions described herein can be used to induce an immune response to and/or reduce the severity of an infection caused by Plasmodium. In some embodiments, compositions of the present disclosure can be used to induce an immune response to and/or reduce the severity of an infection caused by a Plasmodium selected from the group consisting of: P. falciparum, P. malariae, P. ovale and P. vivax.

In some embodiments, compositions described herein can be used to induce an immune response to and/or reduce the severity of an infection caused by a fungus including but not limited to Aspergillus, Candida, Blastomyces, Coccidioides, Cryptococcus, and Histoplasma. In some embodiments, compositions can be used to induce an immune response to and/or reduce the severity of a Candida albicans or a Candida auris infection.

In some embodiments, compositions described herein can be used to induce an immune response to a tumor antigen. In some embodiments, the compositions can be used to induce an immune response to a tumor antigen expressed on a cancer cell including but not limited to breast cancer cell, colon cancer cell, brain cancer cell, pancreatic cancer cell, lung cancer cell, cervical cancer cell, uterine cancer cell, prostate cancer cell, ovarian cancer cell, melanoma cancer cell, lymphoma cancer cell, myeloma cancer cell, and leukemic cancer cell.

In some embodiments, compositions described herein can be used to induce an immune response to a self-antigen. In some embodiments, the compositions can be used to induce an immune response to a self-antigen associated with an autoimmune disease including but not limited to ulcerative colitis, rheumatoid arthritis, systemic lupus erythematosus (SLE), celiac disease, inflammatory bowel disease, Hashimoto's disease, Addison's disease, Grave's disease, type I diabetes, autoimmune thrombocytopeniarpura (ATP), idiopathic pulmonary fibrosis, idiopathic thrombocytopenia purpura (ITP), Crohn's disease, multiple sclerosis, and myasthenia gravis.

In some embodiments, compositions of the present disclosure are administered orally. In some embodiments, compositions of the present disclosure are administered via the respiratory tract (e.g. intranasally or via inhalation). In some embodiments, compositions of the present disclosure are administered as Spirulina biomass. In some embodiments, compositions of the present disclosure are administered as lyophilized Spirulina biomass. In some embodiments, compositions of the present disclosure are administered as extracts of Spirulina biomass.

The dosage of the composition can be determined readily by the skilled artisan, for example, by first identifying doses effective to elicit a prophylactic or therapeutic effect. Said dosages can be determined from animal studies. A non-limiting list of animals used to study the efficacy of vaccines include the guinea pig, hamster, ferrets, chinchilla, mouse and cotton rat. Study animals may not be the natural hosts to infectious agents but can still serve in studies of various aspects of the disease. For example, any of the above animals can be dosed with an composition of the present disclosure, e.g. a recombinant Spirulina comprising a VLP comprising a polypeptide.

In some embodiments, administration of the compositions of the present disclosure decreases infectious agent burden. In some embodiments, administration of the compositions of the present disclosure decreases colonization of the infection agent. In some embodiments, administration of the compositions of the present disclosure decrease shedding of the infectious agent (e.g. viral shedding). In some embodiments, administration of the compositions of the present disclosure decrease shedding of the infectious agent. In some embodiments, administration of the compositions of the present disclosure increase shedding for one period (e.g. 24 hours) and then decrease shedding afterward (e.g. at 72 hours). In some embodiments, administration of the compositions decreases expression of a biomarker. In some embodiments, the biomarker is a marker of inflammation.

In some embodiments, administration of the compositions of the present disclosure neutralizes or blocks the activity of a target. In some embodiments, administration of the present disclosure neutralizes or blocks the activity of the target by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 98%, about 99%, or about 100%.

In addition, human clinical studies can be performed to determine the preferred effective dose for humans by a skilled artisan. Such clinical studies are routine and well known in the art. Effective doses may be extrapolated from dose-response curves derived from in vitro studies, animal studies, and/or clinical studies.

### Methods of Making Non-Parenteral Compositions

Provided are methods of making non-parenteral compositions described herein. Methods of making non-parenteral compositions comprise introducing into a Spirulina a nucleic acid sequence encoding the at least one exogenous polypeptide, antigen, and/or antigenic epitope. In some embodiments, the methods of making non-parenteral compositions comprise introducing into a Spirulina a polypeptide, antigen, and/or antigenic epitope. In some embodiments, methods of making non-parenteral compositions comprise introducing into a Spirulina a small molecule.

Any appropriate means for transforming Spirulina may be used in the present disclosure. Exemplary methods for transforming Spirulina to express a heterologous protein are described in U.S. Pat. No. 10,131,870, which is incorporated by reference herein in its entirety.

In some embodiments, methods of making an non-parenteral composition comprising introducing an expression vector having a nucleic acid sequence encoding the at least one exogenous polypeptide, antigen, and/or antigenic epitope into a Spirulina cell. In some embodiments, the vector is not integrated into the Spirulina genome. In some embodiments, the vector is a high copy or a high expression vector. In some embodiments the nucleic acid sequence encoding the at least one exogenous polypeptide, antigen, and/or antigenic epitope is under the control of a strong promoter. In some embodiments the nucleic acid sequence encoding the at least one exogenous polypeptide, antigen, and/or antigenic epitope is under the control of a constitutive promoter. In some embodiments the nucleic acid sequence encoding the at least one exogenous polypeptide, antigen, and/or antigenic epitope is under the control of an inducible promoter.

In some embodiments, methods of making a composition comprise introducing a vector (e.g. via homologous recombination) having homology arms and a nucleic acid sequence encoding the at least one exogenous polypeptide antigen, and/or antigenic epitope into a Spirulina cell.

In some embodiments, a vector having homology arms and a nucleic acid sequence encoding the at least one exogenous polypeptide, antigen, and/or antigenic epitope can be introduced into Spirulina using electroporation. The electroporation is preferably carried out in the presence of an appropriate osmotic stabilizer.

Prior to introduction of the vector into Spirulina, Spirulina may be cultured in any suitable media for growth of cyanobacteria such as SOT medium. SOT medium includes NaHCO₃1.68 g, K₂HPO₄ 50 mg, NaNO₃ 250 mg, K₂50₄ 100 mg, NaCl 100 mg, MgSO₄·7H₂O, 20 mg, CaCl₂·2H₂O 4 mg, FeSO₄·7H₂O 1 mg, Na₂EDTA·2H₂O 8 mg, A₅ solution 0.1 mL, and distilled water 99.9 mL. As solution includes H₃BO₃ 286 mg, MnSO₄·5H₂O) 217 mg, ZnSO₄·7H₂O 22.2 mg, CuSO₄·5H₂O 7.9 mg, Na₂MoO₄·2H₂O 2.1 mg, and distilled water 100 mL. Cultivation may occur with shaking (e.g., 100-300 rpm) at a temperature higher than room temperature (e.g. 25-37° C.) and under continuous illumination (e.g. 20-2,000, 50-500, or 100-200 μmol photon m⁻² s⁻¹). The growing cells may be harvested when the optical density at 750 nm reaches a predetermined threshold (e.g., OD₇₅₀ of 0.3-2.0, 0.5-1.0, or 0.6-0.8). A volume of the harvested cells may be concentrated by centrifugation then resuspended in a solution of pH balancer and salt. The pH balancer may be any suitable buffer that maintains viability of Spirulina while keeping pH of the media between 6 and 9 pH, between 6.5 and 8.5 pH, or between 7 and 8 pH. Suitable pH balancers include HEPES, HEPES-NaOH, sodium or potassium phosphate buffer, and TES. The salt solution may be NaCl at a concentration of between 50 mM and 500 mM, between 100 mM and 400 mM, or between 200 mM and 300 mM. In an embodiment between 1-50 mL of 1-100 mM pH balance may be used to neutralize the pH.

Cells collected by centrifugation may be washed with an osmotic stabilizer and optionally a salt solution (e.g. 1-50 mL of 0.1-100 mM NaCl). Any amount of the culture may be concentrated by centrifugation. In an embodiment between 5-500 mL of the culture may be centrifuged. The osmotic stabilizer may be any type of osmotic balancer that stabilizes cell integrity of Spriulina during electroporation. In an embodiment, the osmotic stabilizer may be a sugar (e.g. w/v 0.1-25%) such as glucose or sucrose. In an embodiment the osmotic stabilizer may be a simple polyol (e.g. v/v 1-25%) including glycerine, glycerin, or glycerol. In an embodiment the osmotic stabilizer may be a polyether including (e.g. w/v 0.1-20%) polyethylene glycol (PEG), poly(oxyethylene), or poly(ethylene oxide) (PEO). The PEG or PEO may have any molecular weight from 200 to 10,000, from 1000 to 6000, or from 2000 to 4000. In an embodiment the pH balancer or buffer may be used instead of or in addition to the osmotic stabilizer.

A vector having homology arms and a nucleic acid sequence encoding the at least one exogenous polypeptide, antigen, and/or antigenic epitope can be introduced into Spirulina cells that are cultured and washed with an osmotic stabilizer as described above. Electroporation can be used to introduce the vector.

Electroporation may be performed in a 0.1-, 0.2- or 0.4-cm electroporation cuvette at between 0.6 and 10 kV/cm, between 2.5 and 6.5 kV/cm, or between 4.0 and 5.0 kV/cm; between 1 and 100 μF, between 30 and 70 μF, or between 45 and 55 μF; and between 10 and 500 mΩ, between 50 and 250 mΩ, or between 90 and 110 mΩ. In some embodiments, electroporation may be performed at 4.5 kV/cm, 50 μf, and 100 mΩ.

Following electroporation the cells may be grown in the presence of one or more antibiotics selected based on resistance conferred through successful transformation with the plasmid. Post-electroporation culturing may be performed at reduced illumination levels (e.g. 5-500, 10-100, or 30-60 μmol photon m⁻² s⁻¹). The culturing may also be performed with shaking (e.g. 100-300 rpm). The level of antibiotics in the media may be between 5 and 100 μg/mL. Post-electroporation culturing may be continued for 1-5 days or longer. Successful transformants identified by antibiotic resistance may be selected over a time course of 1 week to 1 month on plates or in 5-100 mL of SOT medium supplemented with 0.1-2.0 μg of appropriate antibiotics.

A vector used in the methods can be a plasmid, bacteriophage, or a viral vector into which a nucleic acid sequence encoding the at least one exogenous polypeptide, antigen, and/or antigen can be inserted or cloned. A vector may comprise one or more specific sequences that allow recombination into a particular, desired site of the Spirulina's chromosome. These specific sequences may be homologous to sequences present in the wild-type Spirulina. A vector system can comprise a single vector or plasmid, two or more vectors or plasmids, some of which increase the efficiency of targeted mutagenesis, or a transposition. The choice of the vector will typically depend on the compatibility of the vector with the Spirulina cell into which the vector is to be introduced. The vector can include a reporter gene, such as a green fluorescent protein (GFP), which can be either fused in frame to one or more of the encoded antigenic epitopes, or expressed separately. The vector can also include a positive selection marker such as an antibiotic resistance gene that can be used for selection of suitable transformants. The vector can also include a negative selection marker such as the type II thioesterase (tesA) gene or the Bacillus subtilis structural gene (sacB). Use of a reporter or marker allows for identification of those cells that have been successfully transformed with the vector.

In some embodiments, the vector includes one or two homology arms that are homologous to DNA sequences of the Spirulina genome that are adjacent to the targeted locus. The sequence of the homology arms can be partially or fully complementary to the regions of Spirulina genome adjacent to the targeted locus.

The homology arms can be of any length that allows for site-specific homologous recombination. A homology arm may be any length between about 2000 bp and 500 bp. For example, a homology arm may be about 2000 bp, about 1500 bp, about 1000 bp, or about 500 bp. In some embodiments having two homology arms, the homology arms may be the same or different length. Thus, each of the two homology arms may be any length between about 2000 bp and 500 bp. For example, each of the two homology arms may be about 2000 bp, about 1500 bp, about 1000 bp, or about 500 bp.

A portion of the vector adjacent to one homology arm or flanked by two homology arms modifies the targeted locus in the Spirulina genome by homologous recombination. The modification may change a length of the targeted locus including a deletion of nucleotides or addition of nucleotides. The addition or deletion may be of any length. The modification may also change a sequence of the nucleotides in the targeted locus without changing the length. The targeted locus may be any portion of the Spirulina genome including coding regions, non-coding regions, and regulatory sequences.

### EXAMPLES

### Example 1: Oral Spirulina-VHH Provides Complete Protection Against Campylobacter

### Spirulina Expressing a Monomeric VHH

Mice were inoculated with 107 Campylobacter jejuni. Spirulina were transfected with vectors expressing monomeric VHH antibodies targeting campylobacter. After growth of the Spirulina to allow expression of the monomeric VHH antibodies, the Spirulina were dried, and campylobacter infected mice were administered a daily gavage of 200 μl of PBS and 10% Spirulina biomass (13 mg) for five days. The 13 mg of Spirulina contains 425 μg of monomeric VHH per dose. As controls, mice were administered with daily gavage of either 1) PBS; 2) wild type Spirulina, or 3) Spirulina expressing an irrelevant VHH

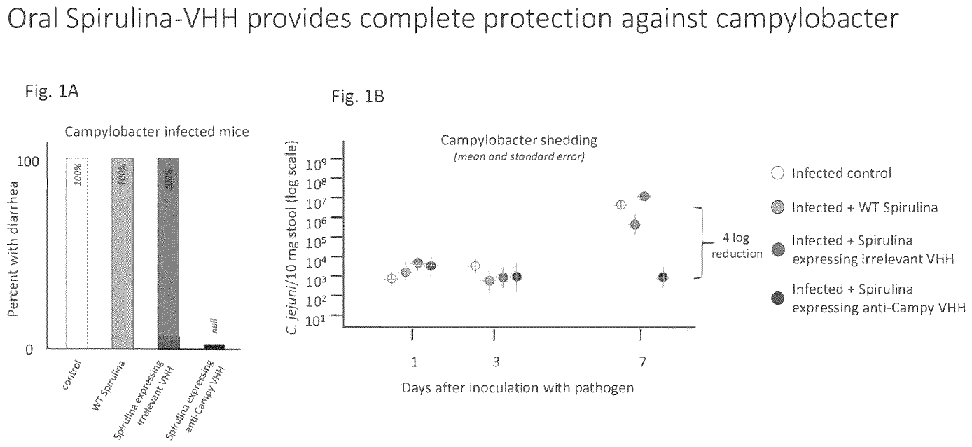

As shown in FIG. 1A, 100% of campylobacter-infected mice treated with any one of the control treatments presented with diarrhea. In contrast, no mice administered the Spirulina expressing the monomeric anti-campylobacter VHH presented with diarrhea. Further, at 7 days after inoculation, mice treated with the Spirulina expressing the monomeric anti-campylobacter VHH demonstrated a four-log reduction in Campylobacter shedding (FIG. 1B).

### Example 2: Spirulina Expressing Trimeric VHH

Oral Spirulina-VHH has anti-inflammatory activity in Campylobacter infection. Mice were inoculated with 10⁸ Campylobacter jejuni. Spirulina were transfected with vectors expressing trimeric VHH antibodies targeting Campylobacter. After growth of the Spirulina to allow expression the trimeric VHH antibodies, the Spirulina were dried, and Campylobacter-infected mice were administered a daily gavage of 400 μl of PBS+0.5% Spirulina biomass (1.3 mg) for three days. The 1.3 mg of Spirulina contains 19 μg of trimeric VHH per dose. As controls, mice were administered with a daily gavage of Spirulina expressing an irrelevant VHH.

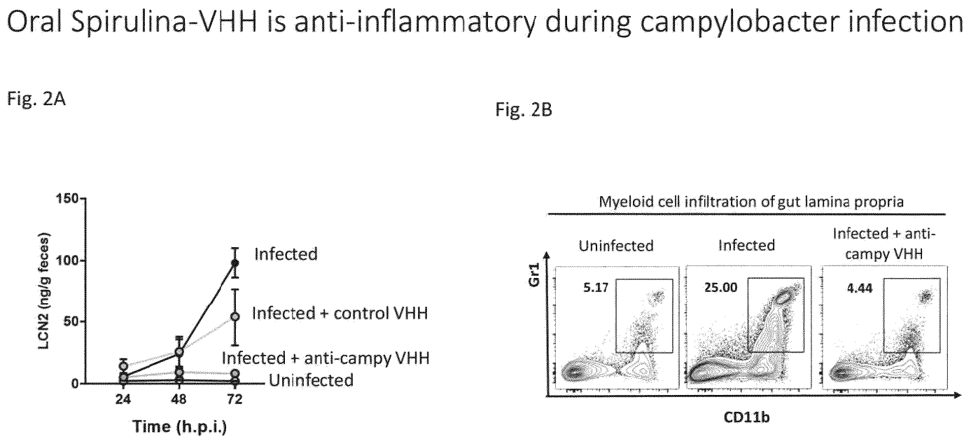

As shown in FIG. 2A, the expression of stool lipocalin, a marker of inflammation, is reduced in mice treated with the Spirulina expressing trimeric anti-campylobacter VHH compared to controls, and indeed stool lipocalin in these treated mice mirror that of uninfected mice. Further, FIG. 2B demonstrates that treatment of the infected mice with Spirulina expressing trimeric anti-campylobacter VHH prevents myeloid cell infiltration of gut lamina propria.

### Example 3: Prophylactic Effect of Spirulina-VHH in Mice Challenged with C. jejuni Strain 81-176

### Test Article:

Spirulina strain SP257 (irrelevant VHH)
Spirulina strain SP526 (anti-C. jejuni VHH FlagV6)
Spirulina strain SP651 (anti-C. jejuni VHH FlagV6)

A mouse model of C. jejuni infection was used to assess the prophylactic efficacy of an anti-C. jejuni VHH expressed in spirulina [Giallourou et al]. Spirulina strains expressing either the VHH FlagV6 (SP526), a protease-resistant form of FlagV6 (FlagV6-F23) (SP806), or an irrelevant VHH (SP257) were tested. Biomass was prepared by spray drying a 4% spirulina-VHH biomass resuspension in a solution containing 2% trehalose.

To prepare for C. jejuni infection, 21-day-old C57BL/6 female mice were treated with vancomycin 48, 24, and 12 hours prior to treatment. On day 0, mice were given an inoculum of 10⁸ C. jejuni strain 81-176 resuspended in PBS. Food and water were provided ad libitum throughout the trial.

To determine how well the mice tolerated being administered spirulina-VHH by gavage, a high, three-dose regimen was tested. Spirulina-VHH was resuspended in PBS, and 400 μL of the slurry was delivered by oral gavage at 90 min before, and 24 and 48 hours after inoculation with C. jejuni. Mice were separated into four different groups:
13.3 mg spirulina-VHH (670 mg/kg) containing an irrelevant VHH;
13.3 mg of an anti-campylobacter VHH on a trimeric scaffold (SP651);
13.3 mg of an anti-campylobacter VHH on a pentameric scaffold (SP737)
Control group treated with PBS.
   Relative to the infected control group treated with PBS, all mice treated with a spirulina-VHH demonstrated non-specific flushing of C. jejuni in stool at 24 hours, followed by reduced bacterial burden at 48 and 72 hours (data not shown). No adverse events were observed in any mice at this dose.

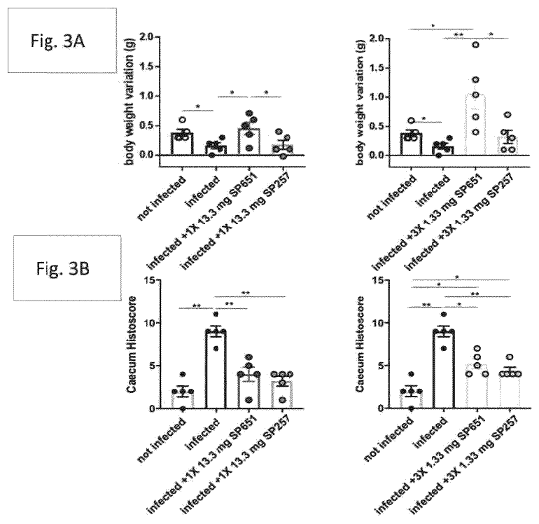

To identify a dose regimen of spirulina-VHH conferring a specific anti-campylobacter effect, mice were tested as below:
Administration of a single 400-μL dose of SP561 5% spirulina-VHH powder w/v resuspended in PBS, gavaged 1.5 hours before inoculation (equivalent to 13.3 mg spirulina-VHH per dose);
Administration of three 400-μL doses of SP561 0.5% spirulina-VHH powder w/v resuspended in PBS, gavaged at 1.5 hours before and 24 and 48 hours after inoculation (equivalent to 1.33 mg spirulina-VHH per dose);
Administration of one 400-μL dose of SP257, an irrelevant VHH 0.5% spirulina-VHH powder w/v resuspended in PBS, gavaged at 1.5 hours before and 24 and 48 hours after inoculation (equivalent to 1.33 mg spirulina-VHH per dose);
Administration of three 400-μL doses of SP257, an irrelevant VHH 0.5% spirulina-VHH powder w/v resuspended in PBS, gavaged at 1.5 hours before and 24 and 48 hours after inoculation (equivalent to 1.33 mg spirulina-VHH per dose);
Control mice administered PBS gavage.
   There were five mice in each experimental group. Three days after campylobacter inoculation, control infected mice (PBS gavage) showed a significant weight deficit compared to uninfected mice (FIG. 3A). Infected mice treated with SP257 showed a similar weight deficit. In contrast, infected mice treated under both dosing regimens with SP651, which expresses the anti-campylobacter-binding protein on a trimeric scaffold, showed a weight gain comparable to or significantly better than uninfected mice (FIG. 3A).

Caeca from all animals were examined at necropsy 72 hours after infection. Tissue sections were processed and scored in a blinded manner by a histopathologist on a scale of 0-24. Each section was assessed for submucosal edema, crypt hyperplasia, goblet cell depletion, epithelial integrity, mucosal mononuclear cell infiltration, and submucosal PMN and mononuclear cell infiltration. Animals that were treated with either SP651 or SP257 scored significantly lower than the infected control and closer to uninfected control (FIG. 3B). These results suggested that spirulina itself had a positive impact on reducing the histopathology of the C. jejuni-infected animals. Without wishing to be bound by theory, this effect could be due to spirulina's inherent health benefits (i.e. it is considered a superfood).

Spirulina-VHH was well tolerated, and no adverse effects were observed in mice treated with the highest dose of 13.3 mg spirulina-VHH.

In a second experiment, a single 1.33-mg dose of spirulina-VHH (SP651) was used to determine the efficacy of an anti-C. jejuni VHH strain compared to spirulina expressing an irrelevant VHH (SP257).

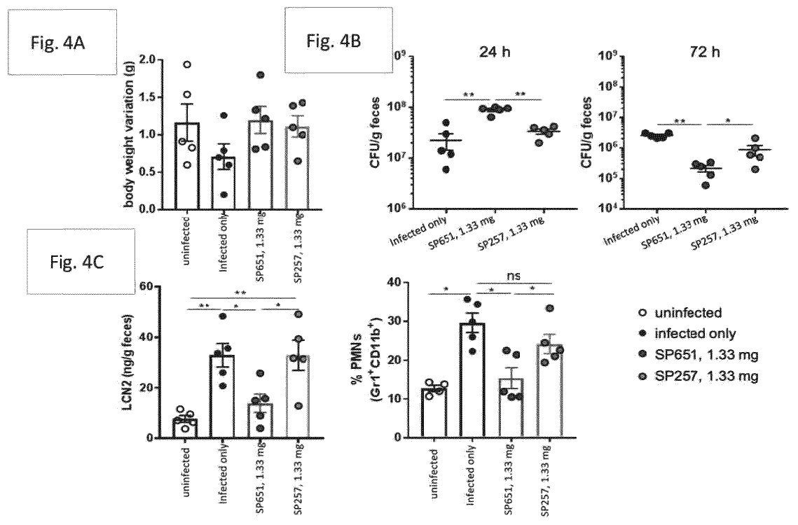

1.5 hours before infection with C. jejuni, mice were given a single 400-μL dose containing 1.33 mg of spirulina-VHH in PBS. Four cohorts, each containing 5 mice, were treated as below:
uninfected,
infected and treated with PBS gavage,
infected and treated with SP257 gavage,
infected and treated with SP651 gavage.
   Treatment with this single prophylactic dose of spirulina containing an anti-campylobacter VHH was sufficient to significantly accelerate campylobacter flushing at 24 hours post infection and reduce campylobacter shedding at 72 hours post infection as measured by fecal campylobacter CFUs. (FIG. 4B). Inflammation following infection was measured by stool lipocalin amounts and by flow cytometric quantitation of myeloid cell infiltration in the cecal lamina propria. Campylobacter infection caused a significant increase in both inflammation biomarkers (FIG. 4C). This increase was prevented by the single prophylactic dose of SP651 (expressing the anti-campylobacter binding VHH), whereas a prophylactic dose of SP257 (expressing the irrelevant VHH) was without effect (FIG. 4). Also, as in the previous experiment, the weight deficit caused by campylobacter infection was prevented by the prophylactic dose of SP651. (FIG. 4A) However, in this experiment, spirulina expressing the irrelevant VHH also suppressed the infection-associated weight deficit, again suggesting a possible nutritional benefit of spirulina per se. Importantly, the spirulina expressing the irrelevant VHH did not exert an effect on the biomarkers of inflammation or myeloid cell infiltration in the cecal lamina propria.

In a third experiment, increasingly dilute single doses of spirulina-VHH were tested to determine the minimal effective dose (MED) of spirulina-VHH required to observe a positive result. Two spirulina-VHH strains were compared: SP526 and SP806. SP526 exhibits a high expression level of the anti-C. jejuni FlagV6 VHH, and SP806 expresses a protease-resistant form of FlagV6 (FlagV6-F23) which contains two mutations in the VHH reported to confer resistance to chymotrypsin (Hussack et al. 2014). The results were also compared retrospectively to the efficacy of SP651 in the previous experiment.

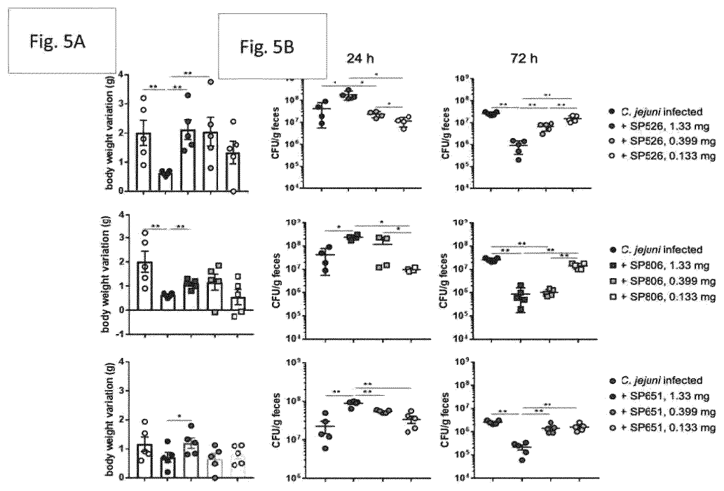

1.5 hours prior to infection with C. jejuni, mice were given a single 400-μL dose containing 1.33 mg, 0.399 mg, or 0.133 mg of spirulina-VHH (SP526, SP806, or SP651) in PBS. Measurements of body weight variation showed, as in previous experiments, that campylobacter caused a weight gain deficit at 72 hours post infection. Treatment with each of the three spirulina-VHH strains suppressed this loss at the 1.33-mg dose (FIG. 5A). In this assay the minimal effective dose (MED) for SP526 was 0.133 mg (6.7 mg/kg), for SP806 it was 0.399 mg (20 mg/kg), and for SP651 it was 1.33 mg (67 mg/kg).

Measurements of fecal campylobacter CFUs showed, as in previous experiments, that all three spirulina-VHH strains accelerated campylobacter flushing at 24 hours post treatment and reduced long-term shedding at 72 hours after treatment (FIG. 5B). Again, VHH expression level and protease resistance independently increased efficacy, with SP526 and SP806 both showing a MED, in this assay, of 0.399 mg (20 mg/kg).

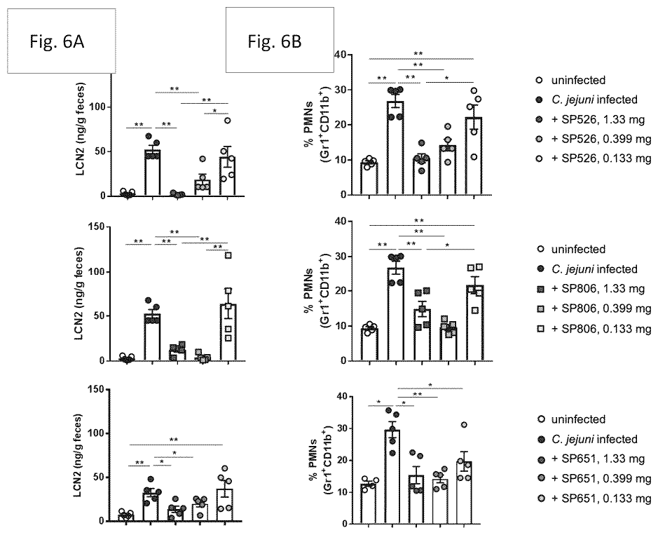

Biomarkers of inflammation—stool lipocalin and myeloid cell infiltration of the cecal lamina propria—showed, as in previous experiments, that all three strains suppressed intestine inflammation following campylobacter infection (FIG. 6). The protease-resistant strain (SP806) conferred the greatest reduction in both lipocalin-2 levels and lamina propria infiltrating myeloid cells. The MED for all three strains was 0.399 mg (20 mg/kg), with partial efficacy at 0.133 mg (6.7 mg/kg).

Caeca from all animals were examined at necropsy 72 hours after infection. Tissue sections were processed and scored in a blinded manner by a histopathologist as before. The only group to demonstrate a significant reduction in histopathology relative to the infected control was treatment with 1.33 mg (67 mg/kg) of SP526. Below this dose or in groups treated with different spirulina-VHH (SP806 or SP651), the positive effect of treatment was determined with other metrics of efficacy (i.e., bacterial shedding, inflammatory biomarkers, etc.).

Conclusion: Administration of all spirulina-VHH strains expressing the anti-C. jejuni VHH FlagV6 gave favorable results for treating C. jejuni-infected mice with a single dose of 1.33 or 0.399 mg spirulina-VHH. Compared to mice receiving no treatment, these mice had better weight gain and reduced levels of inflammatory markers.

No adverse events were observed in these experiments, up to the highest biomass dose administered. The drug material was well tolerated and no signs of toxicity were observed.

The most significant new observation made using the Grassi model is that the minimally effective dose was 0.399 mg dried spirulina-VHH. A single oral dose administered by gavage 90 minutes prior to campylobacter inoculation was sufficient to prevent infection-associated weight loss, reduce fecal shedding of campylobacter at day three, and maintain control (baseline) levels of both molecular and cellular metrics of infection-associated intestine inflammation (stool lipocalin and myeloid cell infiltration of the intestinal lamina propria).

### Example 4—Effect of Post-Challenge Treatment with Anti-Campylobacter Spirulina-VHH in Mice Challenged with C. jejuni Strain CG8421

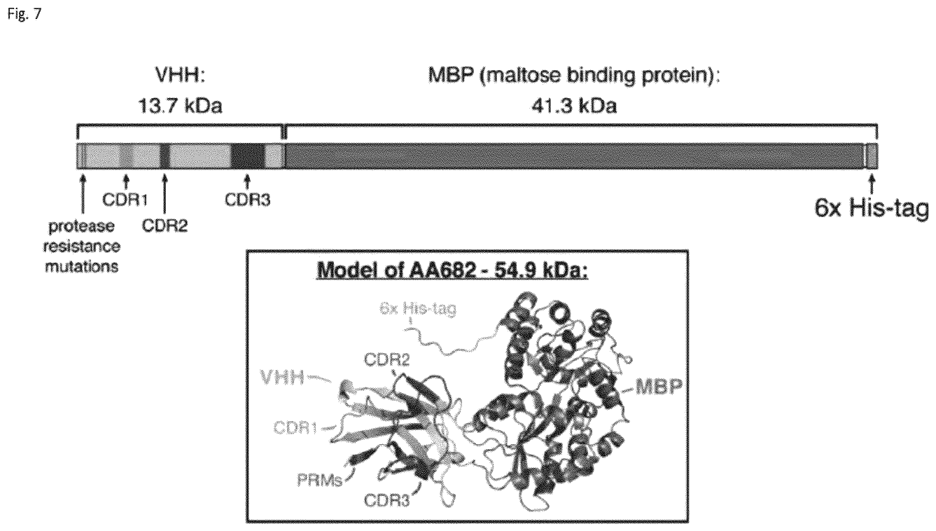

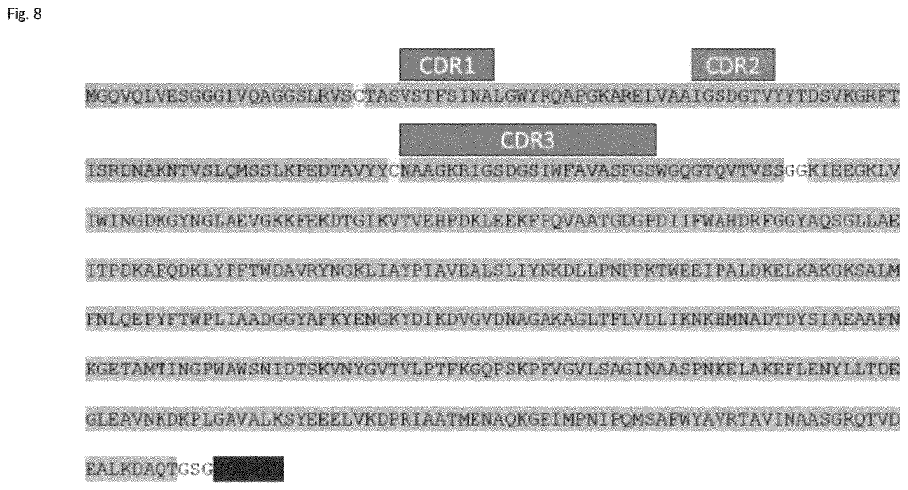

The SP1182 construct is described in FIGS. 7 and 8. This fusion protein comprises a camelin VHH FLAGV6-F23 that binds the flagellin protein flaA from C. jejuni. As the SP1182 fusion protein does not contain a targeting protein, it remains in the cytoplasm of the Spirulina cell.

A mouse campylobacter challenge experiment was performed to test the efficacy of orally delivered SP1182 administered in a treatment modality.

Twenty-one-day-old C57BL/6 mice were given a 48-hour vancomycin conditioning regimen and then challenged with 10⁸ CFU of C. jejuni CG8421 (in PBS). Food and water were provided ad libitum throughout the trial. Three cohorts containing 5 mice each were treated as below beginning 24 hours after the campylobacter challenge:
Two treatment doses at 24 hours and 48 hours post challenge of 67 mg/kg SP1182;
Three treatment doses at 24, 36, and 48 hours post-challenge of 67 mg/kg SP1182;
Two doses at 24 hours and 48 hours of 67 mg/kg wild-type Spirulina (SP3);
Three doses at 24, 36, and 48 hours of 67 mg/kg wild-type Spirulina (SP3).

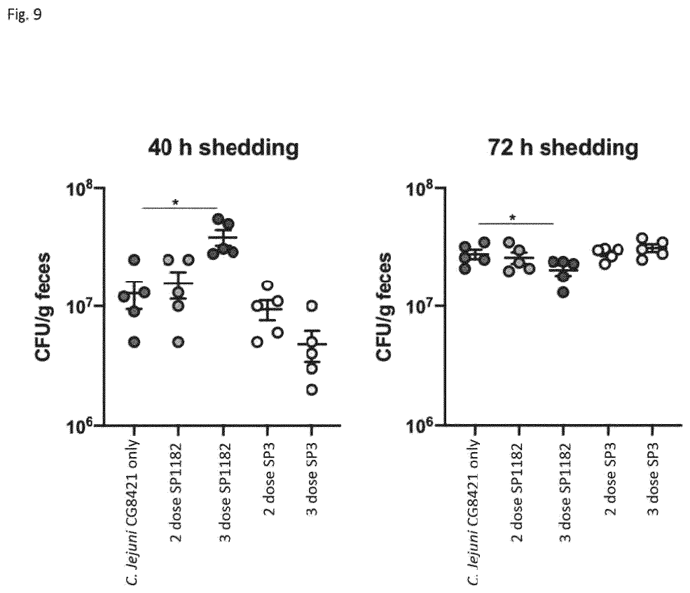

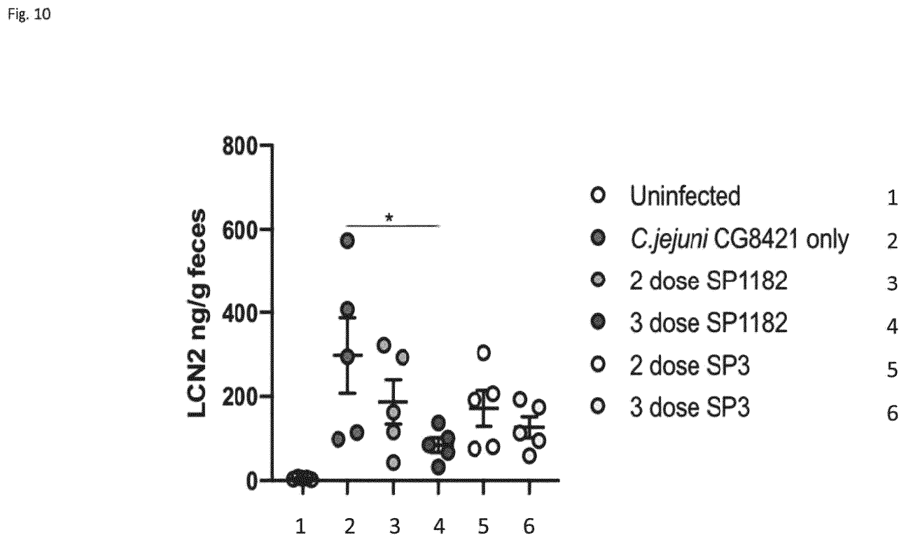

Fecal campylobacter shedding was measured at 40 and 72 hours after infection. At the 40-hour time point there was a significant (p<0.05) burst of campylobacter expulsion only in the 3-dose cohort that received SP1182 at 36 hours (FIG. 9). At 72 hours after infection there was a significant (p<0.05) reduction in fecal campylobacter shedding. Further, there was a significant (p<0.05) reduction in stool lipocalin (a metric for inflammation) only in the cohort of mice that received 3 doses of SP1182 (FIG. 10). Overall these results were very similar to the effect of a single pre-inoculation (prophylactic) dose of SP1182.

Caeca from all animals were examined at necropsy 72 hours after infection. Tissue sections were processed and scored in a blinded manner by a histopathologist on a scale of 0-24. Each section was assessed for submucosal edema, crypt hyperplasia, goblet cell depletion, epithelial integrity, mucosal mononuclear cell infiltration, and submucosal PMN and mononuclear cell infiltration. No treatment group exhibited a reduction in histopathology, with all treatment groups scoring similarly to C. jejuni-infected control.

### Example 5: Encapsulation by Spirulina Protects Polypeptides in the Stomach

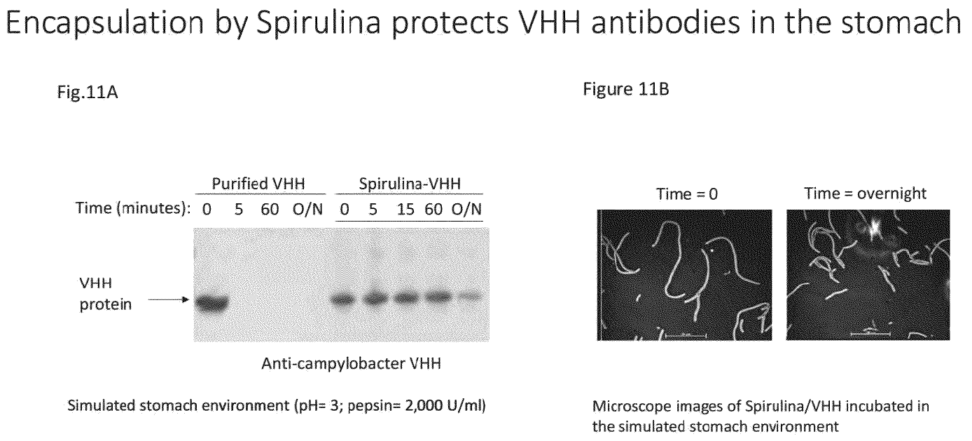

To demonstrate the protective effect of Spirulina on polypeptides, Spirulina were transfected to express an anti-campylobacter VHH. These Spirulina along with the purified anti-campylobacter VHH were subjected to a simulated stomach environment (pH of 3; pepsin at 2,000 U/ml) overnight. Samples were collected at 0 minutes, 5 minutes, 60 minutes, and overnight. As shown in FIG. 11A, the VHH protein encapsulated in Spirulina could be detected after overnight treatment, while those of purified VHH could not be detected after 5 minutes of exposure to the simulated stomach environment. FIG. 11B shows microscopic images of the anti-campylobacter VHH expressing Spirulina at times 0 and overnight; the Spirulina maintained their integrity in the simulated stomach environment.

### Example 6: Polypeptides Expressed in Spirulina are Stable Long-Term in Dried Biomass

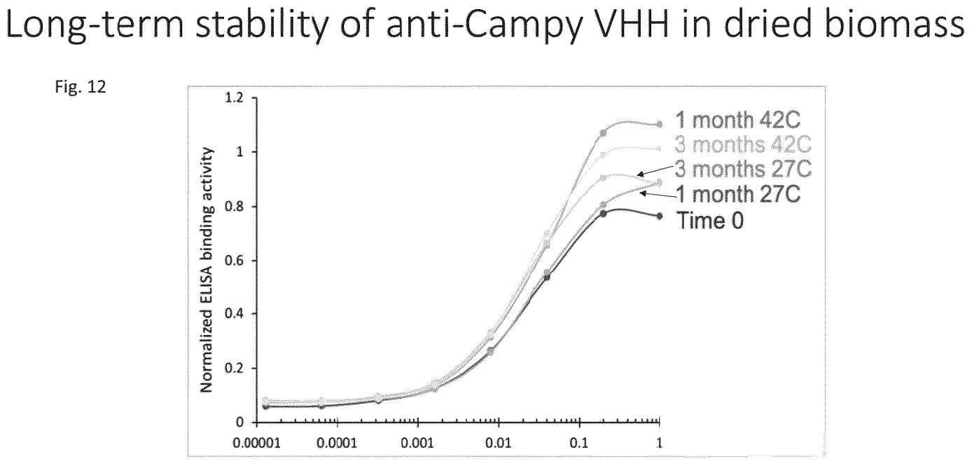

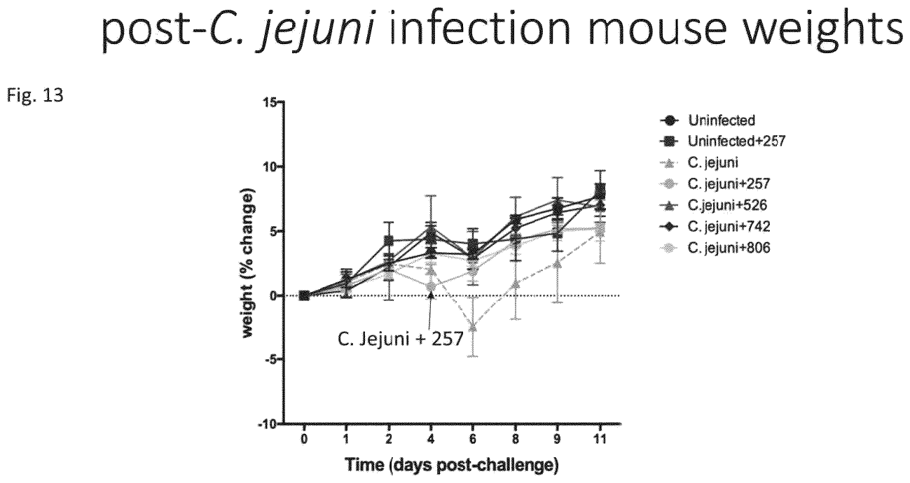

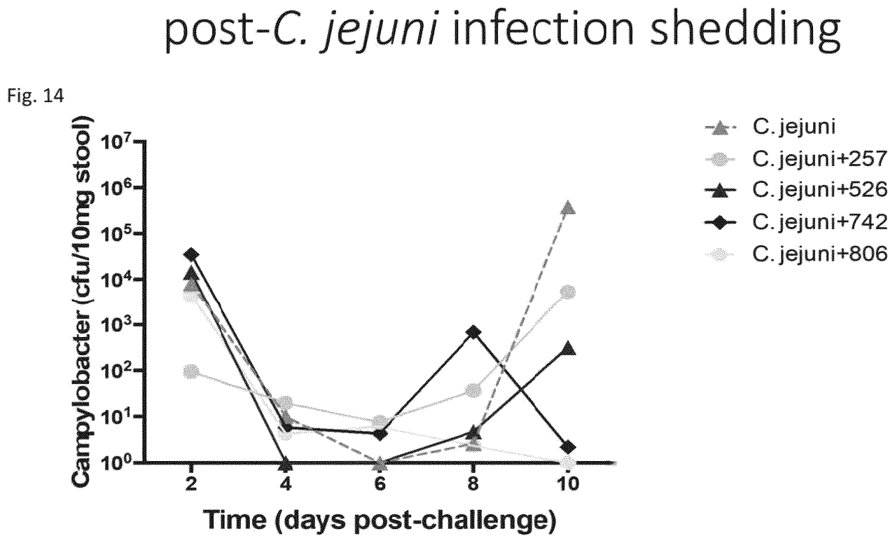

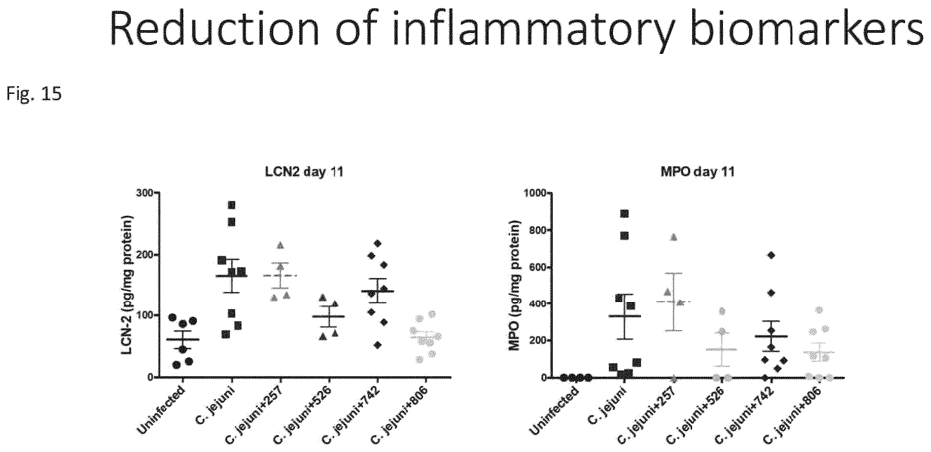

To test the effect of long-term storage of dried biomass on polypeptide stability, Spirulina expressing monomeric anti-campylobacter VHH were spray dried and stored: 1) 1 month at 27° C.; 2) 3 months at 27° C.; 3) 1 month at 42° C.; or 4) 3 months at 42° C. At the various time points, the VHH was purified from the Spirulina and tested for binding activity. As shown in FIG. 12, no decrease in anti-campylobacter VHH bioactivity was observed with prolonged incubation at elevated temperatures.

### Example 7: Preclinical Efficacy of Multiple Doses in Mice Challenged with Campylobacter

### Test Articles:

Spirulina strain SP651 (expressed anti-C. jejuni VHH FlagV6)
Spirulina strain SP806 (expressed anti-C. jejuni VHH FlagV6-F23)
Spirulina strain SP257 (expressed irrelevant VHH)
Spirulina strain SP526 (expressed anti-C. jejuni VHH FlagV6)

Using a mouse model of C. jejuni infection developed at the Institute Research in Biomedicine in Switzerland, the efficacy of prophylactic treatment with anti-C. jejuni VHH expressing spirulina was assessed. Several spirulina strains expressing either the anti-C. jejuni VHH FlagV6, a protease-resistant form of FlagV6 (FlagV6-F23) (Hussack et al. 2014; Riazi et al. 2013), or an irrelevant VHH were tested. Biomass was prepared by spray drying a 3% spirulina biomass resuspension in a solution containing 2% trehalose. To prepare for C. jejuni infection, 21-day old C57BL/6 mice were treated with vancomycin from 48-12 hours prior to treatment. On day 0, mice were given an inoculum of 10⁸ C. jejuni, strain 81-176.

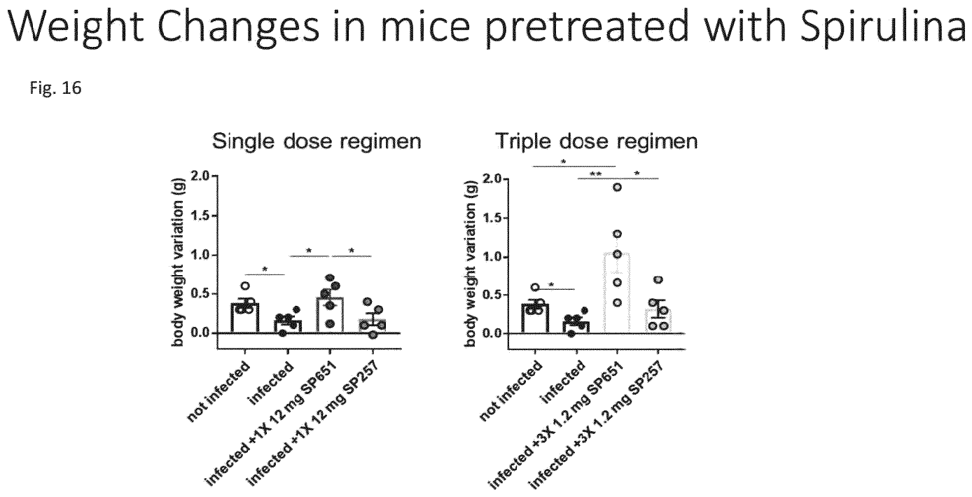

To determine how well the mice would tolerate being given spirulina by gavage, two dosing regimens were tested: 1) a single 400 μL dose of 5% spirulina powder w/v resuspended in phosphate buffer saline (PBS), gavaged 1.5 h before inoculation (equivalent to 12 mg spirulina per dose), 2) three 400 μL doses of 0.5% spirulina powder w/v resuspended in PBS, gavaged at 1.5 h before and 24 and 48 h after inoculation (equivalent to 1.2 mg spirulina per dose). Under both regimens, infected mice treated with spirulina (either SP257 or SP651) showed weight gain similar to the uninfected control group (FIG. 16). Spirulina was considered well tolerated because no adverse effects were observed in mice treated with the highest dose of 12 mg spirulina.

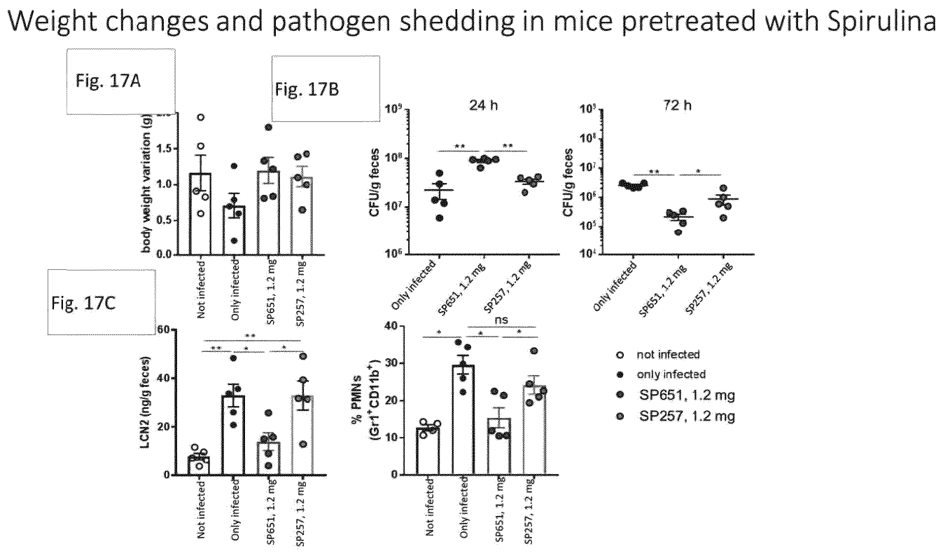

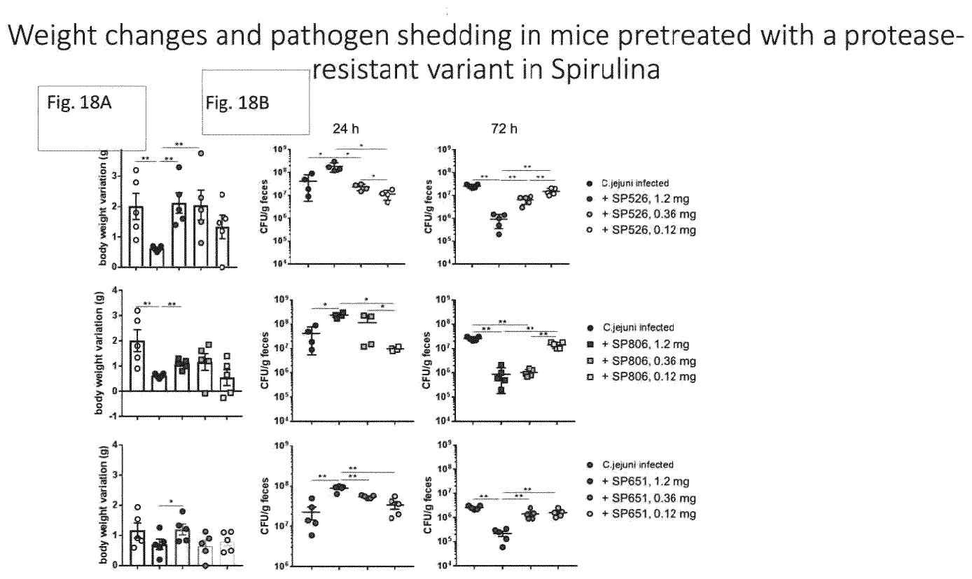

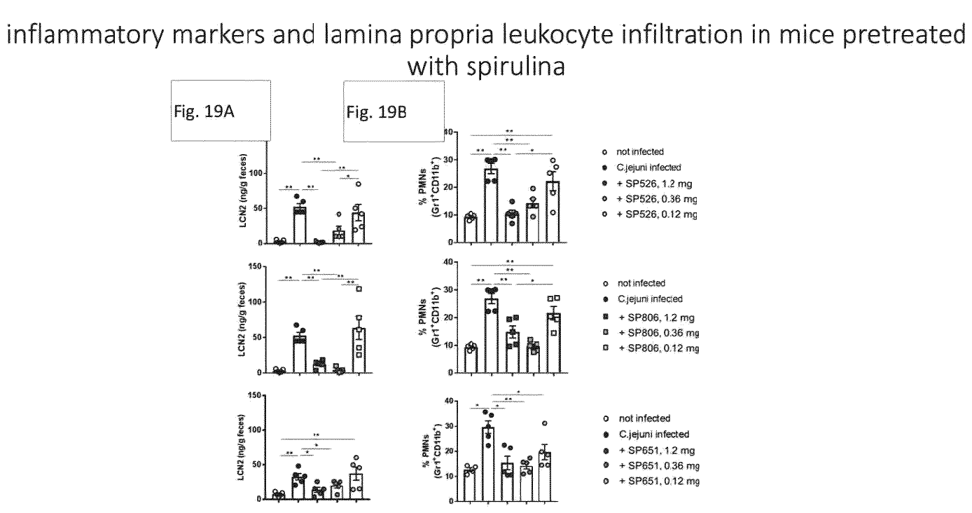

A single 1.2 mg dose of spirulina was used to determine the efficacy of an anti-C. jejuni VHH strain (SP651) compared to spirulina expressing an irrelevant VHH (SP257). 1.5 h before infection with C. jejuni, mice were given a single 400 μL dose containing 1.2 mg of spirulina in PBS. Compared to untreated, infected mice, mice that received anti-C. jejuni spirulina demonstrated good weight gain, an increase in shedding at 24 h followed by a decrease at 72 h, and reduced levels of biomarkers of inflammation (FIG. 17A-C). Spirulina containing an irrelevant VHH had little to no effect on shedding and inflammatory biomarker reduction.

Increasingly dilute single doses of spirulina were tested to determine the limiting amount of spirulina required to observe a positive result. Three spirulina strains expressing the anti-C. jejuni FlagV6 VHH in different forms were compared for potency. Notably, SP526 was selected for its high expression level of the anti-C. jejuni FlagV6 VHH, and SP806 was identical to SP651, with the exception that SP806 contained two mutations in the VHH reported to confer resistance to chymotrypsin (Hussack et al. 2014). 1.5 h prior to infection with C. jejuni, mice were given a single 400 μL dose containing 1.2 mg, 0.36 mg, or 0.12 mg of spirulina in PBS. All three strains showed good efficacy at the 1.2 mg dose and varying degrees of reduced effectiveness at the 0.36 mg and 0.12 mg doses. Mice treated with SP526 exhibited the best weight gain, while SP806 reduced shedding at 72 h at the intermediate dose concentration (FIG. 18A-C). All three strains significantly reduced levels of biomarkers of inflammation at a 0.36 mg dose (FIG. 19A-B), but the protease resistant strain (SP806) conferred the greatest reduction in both lipocalin-2 levels and lamina propria infiltrating myeloid cells. At the 0.12 mg dose of spirulina, all strains behaved similarly to the C. jejuni-only control, suggesting that this amount was below the effective therapeutic dose.

In summary, all spirulina strains expressing the anti-C. jejuni VHH FlagV6 gave positive results for treating C. jejuni infected mice with a single dose of 1.2 mg spirulina-VHH. Compared to mice receiving no treatment, these mice had better weight gain and reduced levels of inflammatory markers.

No adverse events were observed in these experiments, up to the highest biomass dose administered. The drug material was well tolerated and no signs of toxicity were observed.

### Example 8: Effect of Spirulina-VHH in Chickens Challenged with C. jejuni Strain 81-176

### Test Article:

Spirulina strain SP257 (irrelevant VHH)
Spirulina strain SP526 (anti-C. jejuni VHH FlagV6)
Spirulina strain SP651 (anti-C. jejuni VHH FlagV6)

The efficacy of orally delivered spirulina-VHH in blocking the colonization of the chicken intestinal tract was investigated. A chicken model of C. jejuni enteric colonization was used to assess the prophylactic efficacy of an anti-C. jejuni VHH expressed in spirulina. Spirulina strains expressing either a monomeric anti-campylobacter VHH (SP526), a homotrimeric multimer of the VHH (SP651), or an irrelevant VHH (SP257) were tested. Strains were cultivated and spray dried at a concentration of 3% biomass in 2% trehalose. This experiment was designed to assess the therapeutic efficacy of different spirulina strains on their ability to block gastrointestinal tract colonization with C. jejuni, a very common occurrence in commercial flocks and a major source of human food-borne illness.

Study animals were 14-day old SPF leghorn mixed-sex chicks. A 13.3-mg spirulina-VHH dose (150 mg/kg) was administered in 200 μL PBS by oral gavage 1 hour prior to a challenge inoculum of 10⁸ C. jejuni, strain 81-176. Chicks were randomly assigned into negative control, positive control, and treatment groups, housed in isolator units and provided standard feed and water ad libitum. Two days following isolation, chicks were treated with one dose by gavage with PBS or with Spirulina suspended in PBS. One hour later birds were inoculated with 108 CFU of C. jejuni 81-176, or sham inoculated with PBS, by gavage. Body weights were measured at 24, 48 and 72 hours post-inoculation. At 72 hours, birds were euthanized and cecal contents were aseptically collected for quantitative assessment of C. jejuni colonization.

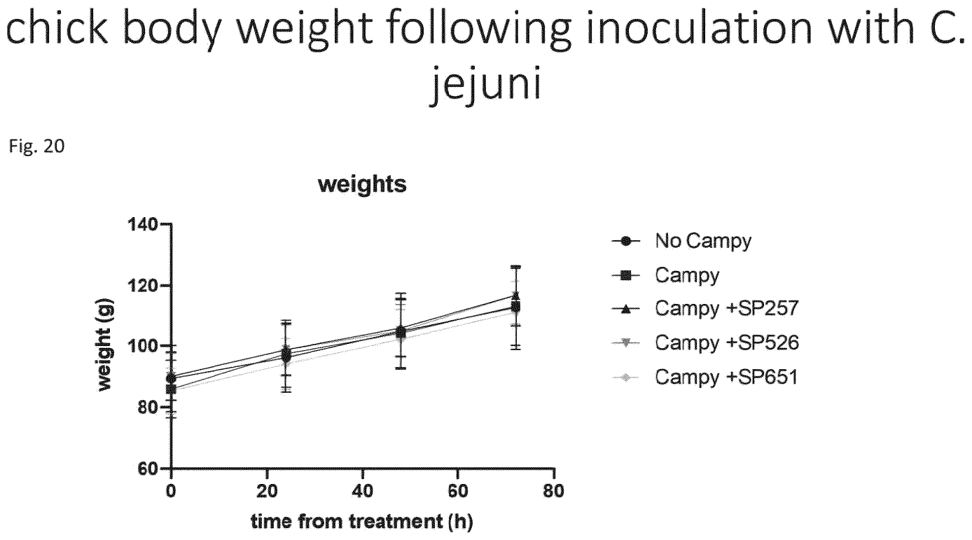

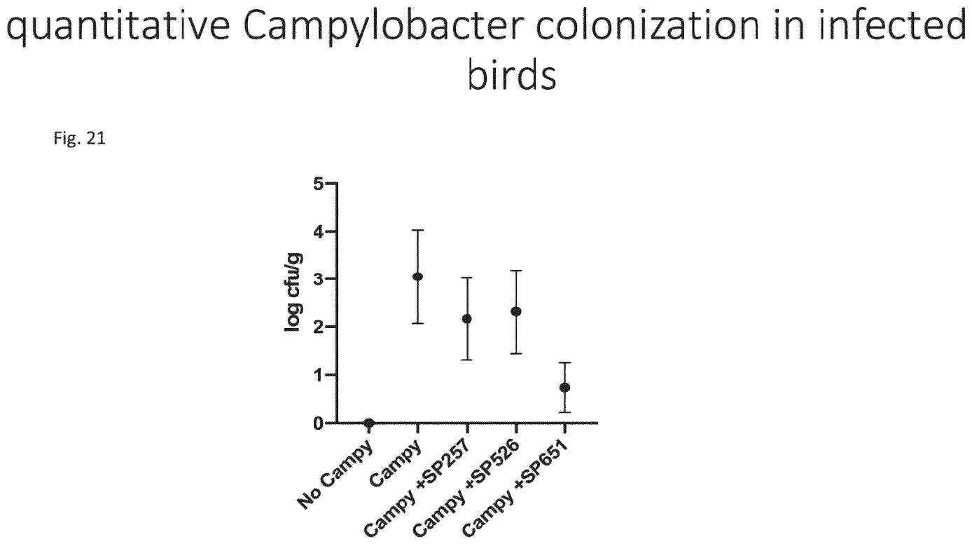

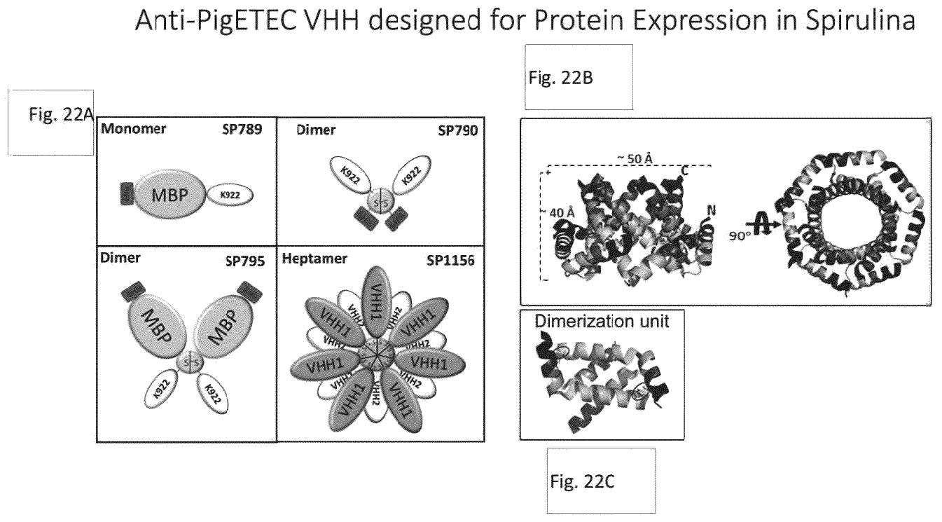

Birds were observed to gain weight normally, without deficit independent of Campylobacter inoculation or prophylactic therapy (FIG. 20). Cecal colony counts were used to assess bacterial burden. Cecal colonization with Campylobacter significantly reduced following pretreatment with Spirulina SP651, a strain expressing anti-Campylobacter FlagV6 in homotrimeric configuration. Treatment with SP257, expressing an irrelevant VHH, and SP526, expressing monomeric VHH FlagV6, resulted in reduced Campylobacter colonization, though to an insignificant degree compared to no Spirulina treatment (FIG. 21).

### Example 9: ETEC Therapeutics: Spirulina Expressed Anti Adhesion VHHs

Enterotoxigenic Escherichia coli (ETEC) is one of the causative agents of diarrhea in children in developing countries and traveler's diarrhea in persons who travel to areas where ETEC is endemic. According to the WHO, the pathogen is responsible for over 200 million illnesses and around 0.5 million deaths worldwide annually. ETEC caused diarrhea has a long-lasting effect on young patients, including stunted growth, decreased intellectual aptitude, and associated long term economic disadvantages. The adverse impact of ETEC pathogenesis necessitates effective preventive post-infection treatment or preventive therapeutics like passive immunization. The two main virulence factors in ETEC infection targeted by vaccine development or prophylactic therapy are the enterotoxins and colonization factors (CFs) or pili. Enterotoxins are directly responsible for causing diarrhea following bacterial colonization of gut intestinal epithelial cells. In the other hand, ETEC CFs allow the organisms to readily colonize the small intestine and subsequently result in the expression of enterotoxins close to mucosal cells causing diarrhea.

The Inventors have developed single-domain camelid antibody (VHH)-based therapeutics that target ETEC fimbriae tip domain and inhibit bacterial attachment to host intestinal epithelial cells and hence block bacterial colonization. The VHHs are derived from either Llama Immunization with the fimbriae tip adhesion protein CfaE or screened against the same antigen from a yeast-based synthetic library. VHHs that exhibit higher antigen binding and bacterial inhibition in hemagglutination or cell-based assay were designed for spirulina expression as monomers, dimers, trimers, tetramers, pentamers, heptamers and displayed on nanoparticles. Chaperone proteins like Maltose Binding Protein (MBP), Thioredoxin A (TxnA) and Neutrophil Gelatinase-Associated Lipocalin (LCN) were used to increase heterologous protein solubility which can result in higher protein expression levels of therapeutic VHH in Spirulina.

Spirulina strains expressing the anti-CfaE VHHs show good binding activity to the Adhesion domain of CFA/I fimbriae tip. An increased multimeric state of VHHs correspond to increased binding activity ELISA.

### Example 10: Pig ETEC Therapeutics: Spirulina Expressed Anti Adhesion VHHs

Porcine Enterotoxigenic Escherichia coli (ETEC) is the number one cause of piglet diarrhea. The infection of ETEC in nursery pigs may induce diarrhea during the first 1 or 2 weeks of postweaning periods usually resulting in dehydration, reduced weight gain, and death. The economic challenge on the porcine industry makes Post-weaning diarrhea and the causative agent ETEC, an economically significant disease in the pig farming industry. The main virulence factor in ETEC strains is the adhesins expressed as part of the fimbriae (pili) structures where the most common in porcine ETEC are adhesins K88 (also called F4), K99 (F5), 987P (F6), F41, and F18 of which K88 and F18 are the most prevalent in the swine industry.

The Inventors have developed a system to cost-effectively produce a multivalent camelid single domain antibodies (VHHs) targeting the virulence factors in K88 and F18 in the Spirulina platform, which enable oral delivery of protein therapeutics to farm animals to protect the gastrointestinal tract through passive immunization without the need for purification or expensive preservatives and delivery methods. The therapeutics can be incorporated as part of animal feed.

The Inventors have designed VHHs that target the ETEC virulence factor important in the attachment to host cells for spirulina expression as monomers, dimers, and, heptamers. To achieve higher protein expression levels of therapeutic VHH in spirulina, chaperone proteins like Maltose Binding Protein (MBP), or Thioredoxin A (TxnA) are used to increase heterologous protein solubility. Expression constructs are designed with affinity tags to facilitate downstream protein expression, purification, and ELISA assays.

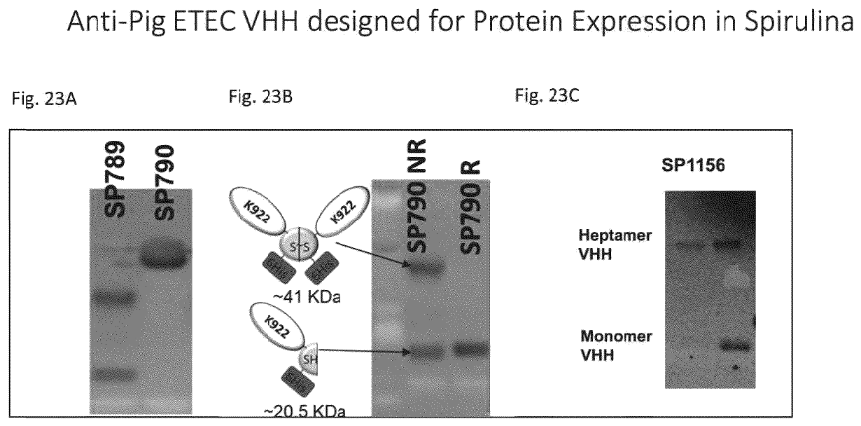

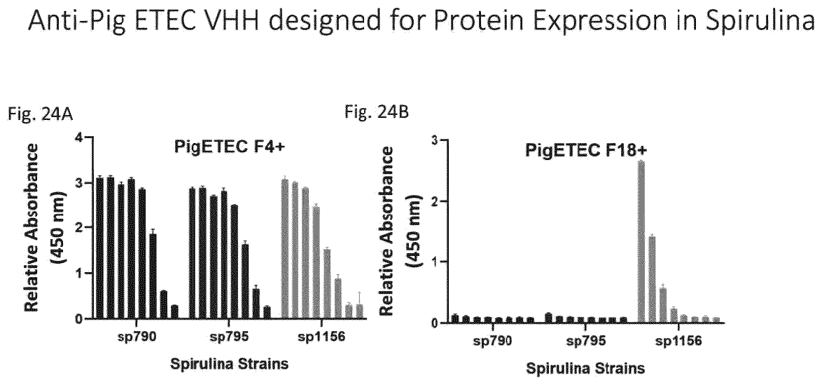

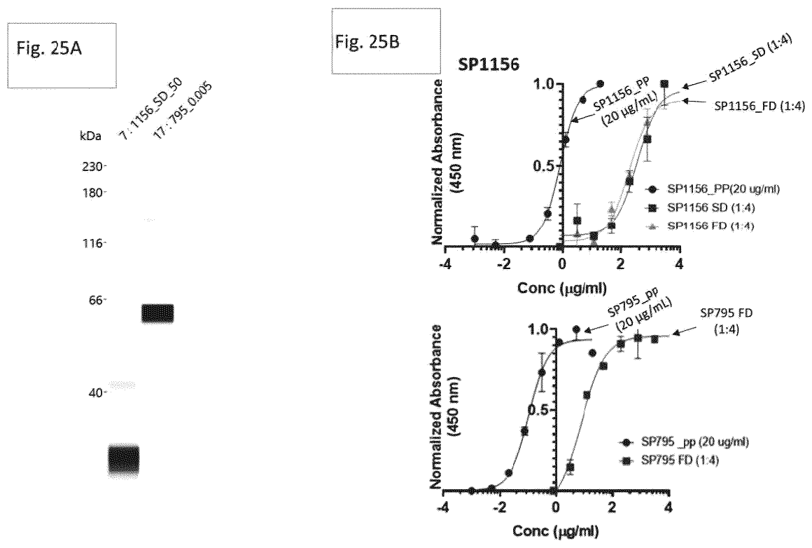

The expression level of protein of interest is determined by Western Blotting using anti-tag or anti-VHH primary and appropriate secondary antibody combinations. Binding activity of protein expressed in Spirulina strains are assessed using ELISA where the antigen is coated onto high binding plates, and antibody-expressing Spirulina strain crude cell lysate titrated in dilutions. We have expressed monomeric, dimeric and hetero-heptameric anti-adhesin VHHs in Spirulina. (FIG. 22A-C). VHH binding activity against antigen by ELISA shows that the VHHs are active as spirulina crude lysates. The hetero-pentameric construct the express VHHs targeting the F4+ and F18+ adhesin bind both the F4+ adhesin domain FaeG and the F18+ adhesin domain FedF. (FIG. 23A-C).

### Example 11: VHHs that Target the ETEC Fimbrial Domain Inhibit Bacterial Attachment in the Gnobiotic Piglet Model

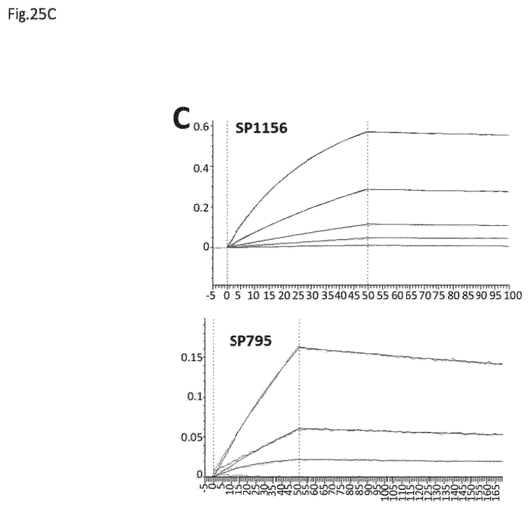

VHHs were designed that target the fimbrial domain of the ETEC strain K88ac+, an ETEC strain that causes post-weaning diarrhea in piglets. This VHH was expressed in Spirulina as a homodimer (SP795) and heteroheptamer (SP1156). (FIG. 22A). The spirulina biomass was dried, and protein expression confirmed. (FIG. 25A). The VHH in spirulina slurry from spray-dried and freeze-dried powder show comparable ELISA based binding. (FIG. 25B). The antigen binding efficiency of spirulina expressed VHH was further assessed using BLI based kinetics measurement. (FIG. 25C).

Table 2 shows the total VHH expression per mass of dried spirulina biomass assessed using Western Blot. Binding strength was assessed using ELISA EC50, and KD as measured from BLI based kinetic measurements. The level of active VHH was determined by comparing observed activity from spirulina biomass to binding activity by purified protein.

TABLE 2

| | | Active | | Total |
|---|---|---|---|---|
| | EC50 | VHH | KD | VHH |
| Strain (protein) | (μg/ml) | % | (nM) | % |
| SP1156 (purified | 0.14 | 100 | | |
| Protein) | | | | |
| SP1156 (Spray | 28.01 | 0.5 | | |
| dried biomass (SD)) | | | | |
| SP1156 (Freeze | 18.65 | 0.7 | ~8.9 | ~1.3 |
| dried biomass (FD)) | | | | |
| SP795 (purified | 0.11 | 100 | | |
| protein) | | | | |
| SP795 (Freeze dried | 7.78 | 1.4 | ~1.4 | ~3.5 |
| Biomass (FD)) | | | | |

The level of active protein in SP1156 was determined to be 0.5%, while the level of activity in SP795 is determined from 1.4%.

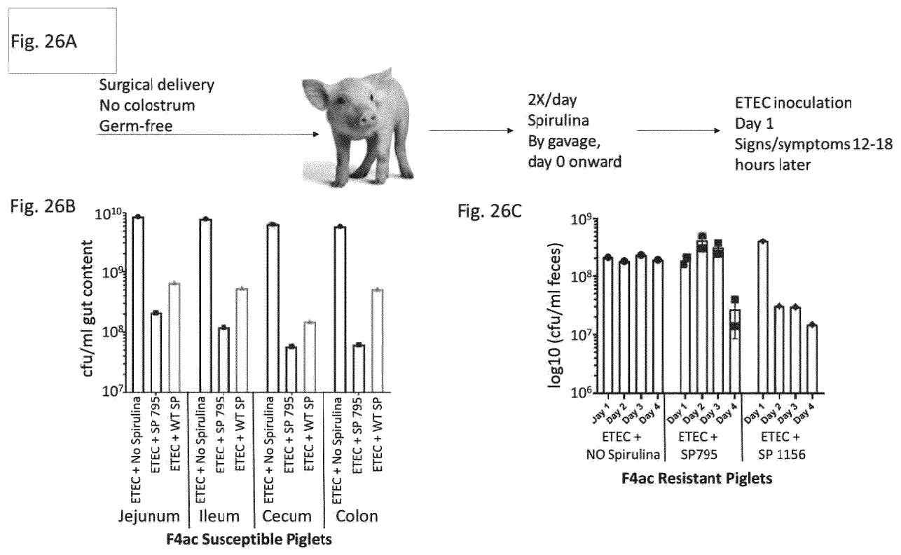

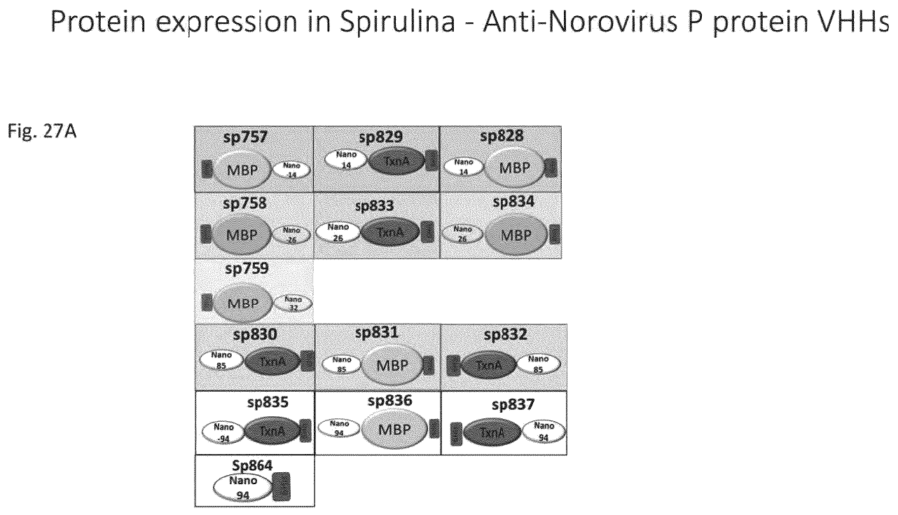

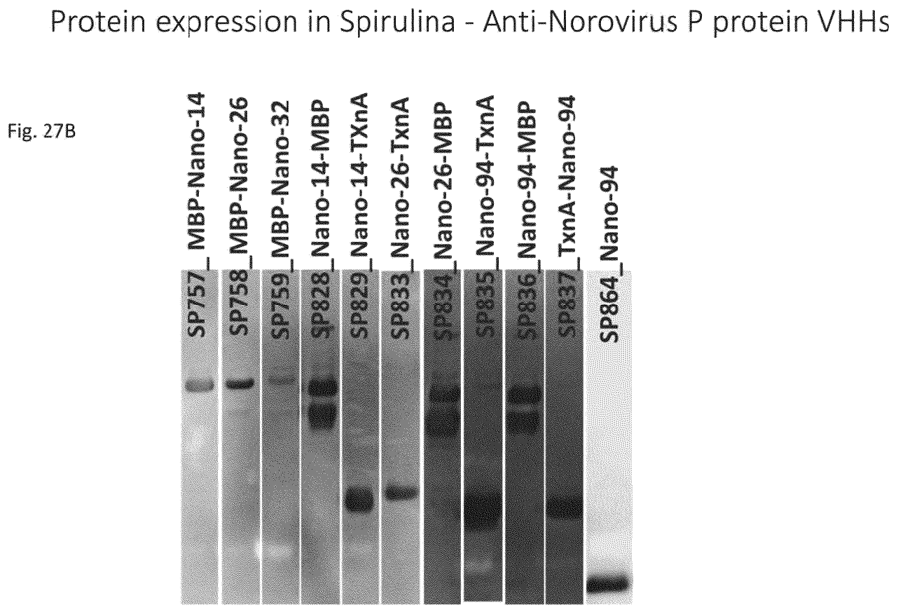

Further, VHHs that target the fimbrial domain of the ETEC strain K88ac+ (F4+ac), an ETEC strain that causes post-weaning diarrhea in piglets, affect bacterial load in gnotobiotic piglets. Surgically delivered gnotibiotic piglets were treated with wild type or therapeutic VHH expressing Spirulina powder slurry by oral gavage twice a day from day 0 onward. The piglets were then challenged with 10¹⁰ ETEC one day later. (FIG. 26A). K88 (F4ac)-susceptible piglets were administered the 0.5 g Spirulina biomass in 10 ml of aqueous diluent VH795 Spirulina, SP1156 Spirulina, or wild type Spirulina. K88 (F4ac)-resistant piglets were administered Spirulina containing either an SP795 or the SP1156 VHH by oral gavage twice a day, from day 0 onward.

On Day 1, the piglets, both K88 susceptible and K88 resistant piglets, showed signs of infection and symptoms at 12-18 hours post-infection. The bacterial dose used was too high, and susceptible piglets had to be euthanized at Day 2. The K88 susceptible piglets were necropsied due to severe symptoms, and intestinal samples were assayed for bacterial load. Piglets treated with therapeutic Spirulina powder containing the SP1156 VHH showed decreased bacterial load in all tissues assayed. (FIG. 26B).

The high bacterial dose lead even the resistant piglets to exhibit symptoms. K88-resistant piglets were maintained for four days, and bacterial shedding was assessed by taking fecal swabs. Piglets treated with the Spirulina strain SP1156 showed decreased bacterial load after challenge. (FIG. 26C). These piglets, while showing symptoms, were still healthy enough to stop the treatment after the challenge to divert them to a different study.

### Example 12: Norovirus Therapeutics: Spirulina Expressed Anti Norovirus Capsid Protrusion Domain VHHs

Human Norovirus (HuNoV) is one of the most important causative agents of gastroenteritis with about one-fifth of all acute infections attributed to this virus. HuNoV is the primary causative agent of acute gastroenteritis. According to a study that looked at the burden of diarrheal diseases in the US, HuNoV infections result in approximately 2 million outpatient visits, 800 deaths, 70,000 hospitalizations, and nearly 400,000 emergency room visits per year in the US. According to the CDC, HuNoV is the leading cause of food-borne illnesses. HuNoV is a single strand RNA virus where its genome has genes that encode for the viral capsid protein (VP1). Based on the sequence diversity in the gene that encodes for the capsid (VP1), Noroviruses are classified into various genogroups (GI-GVII). The genogroups are further divided into genotypes. The most prominent genogroups isolated from recent incidents of human infection are Genogroup GI, GII, and GIV. of which over 25 genotypes have been identified. The most prevalent genotypes in recent HuNoV outbreaks are GI.1, GII.4, and GII.10.

Orally delivered single domain antibody (VHH) based prophylactic therapeutics will be developed. The approach combines the favorable VHH properties that make these class of antibody suitable for oral delivery (properties like high solubility, increased pH stability, and resistance to enzymatic degradation) and Spirulina-based oral delivery of therapeutics. VHHs that target the viral Capsid protein where in some disassemble viral particles upon binding and neutralize infective virus are designed for expression in Spirulina.

Multivalent camelid single domain antibodies (VHHs) targeting the viral capsid protein will be developed to enable oral delivery of protein therapeutics against HuNoV to protect the gastrointestinal tract through passive immunization without the need for purification of the therapeutic agent or expensive preservatives and delivery methods. VHHs that are designed for spirulina expression as monomers with or without chaperone proteins like Maltose Binding Protein (MBP), or Thioredoxin A (TxnA) to increase heterologous protein solubility. Expression constructs are engineered with affinity tags.

The expression level of protein of interest is determined by Western Blotting using anti-tag or anti-VHH primary and appropriate secondary antibody combinations. Binding activity of protein expressed in Spirulina strains are assessed using ELISA where the antigen is coated onto high binding plates, and antibody-expressing Spirulina strain crude cell lysate is titrated in dilutions.

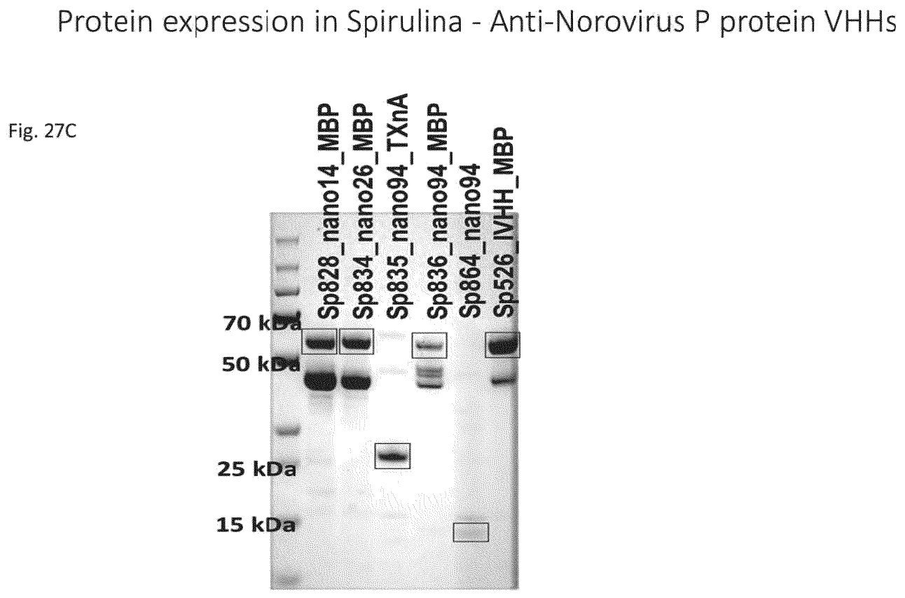

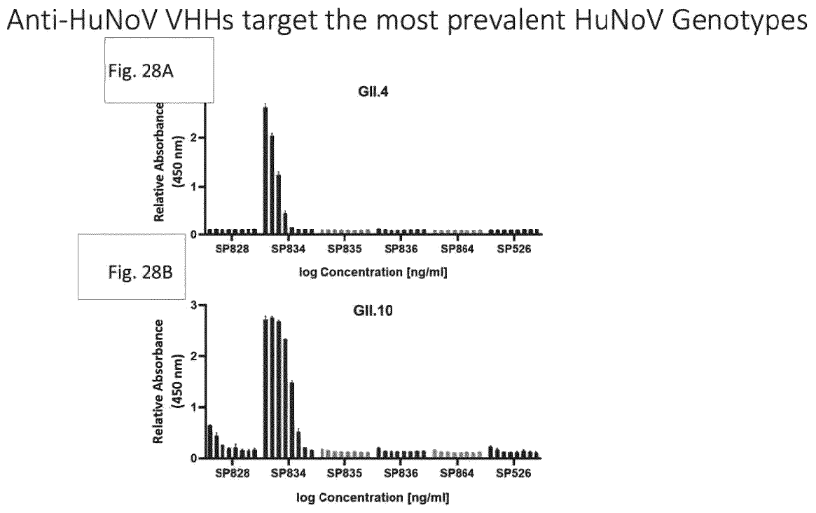

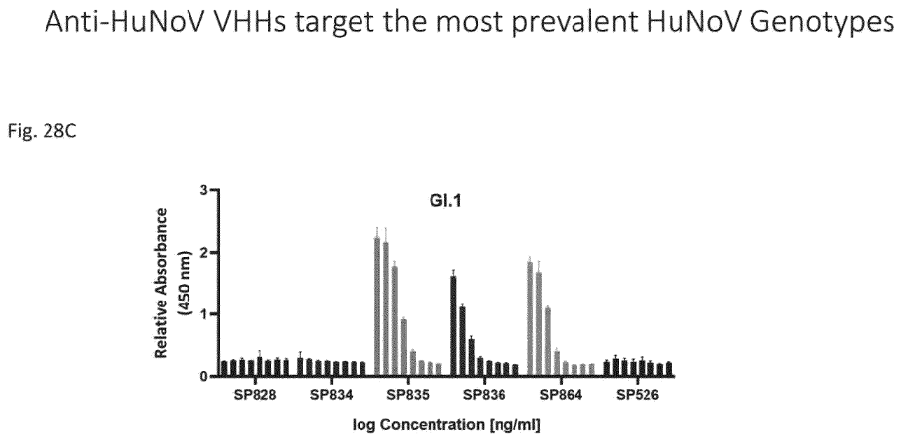

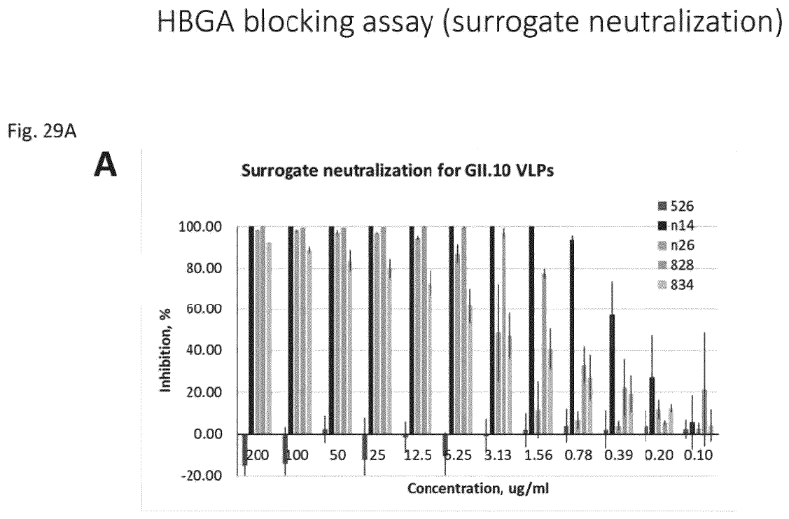

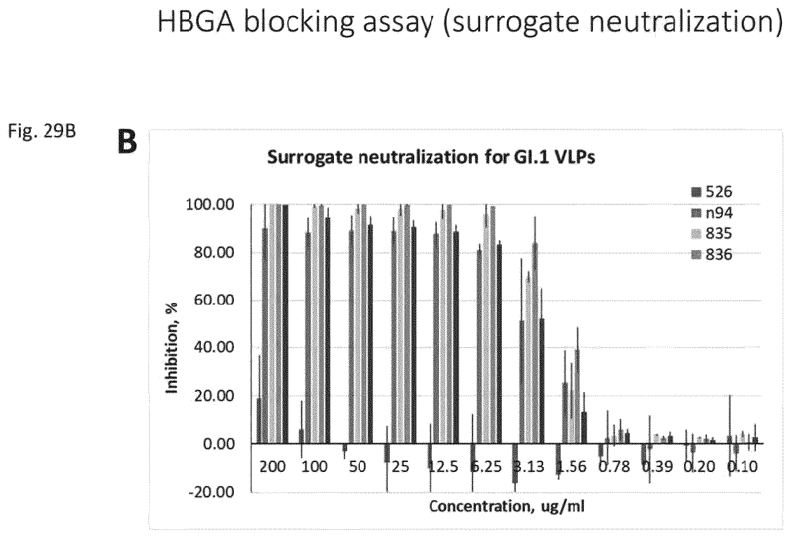

We have expressed monomeric anti HuNoV capsid protrusion protein VHHs with and without chaperone fusion partners in Spirulina. (FIG. 27A-C) ELISA based binding assay show that Spirulina expressed VHHs are active as spirulina crude lysates. (FIG. 28A-C). Furthermore, VHHs purified from Spirulina crude lysate exhibit the expected virus Capsid disassembly and block virus attachment to tissue biopsy that mimic intestinal environment. (FIG. 29A-B).

### Example 13: Development of VHHs to Treat Norovirus Infection

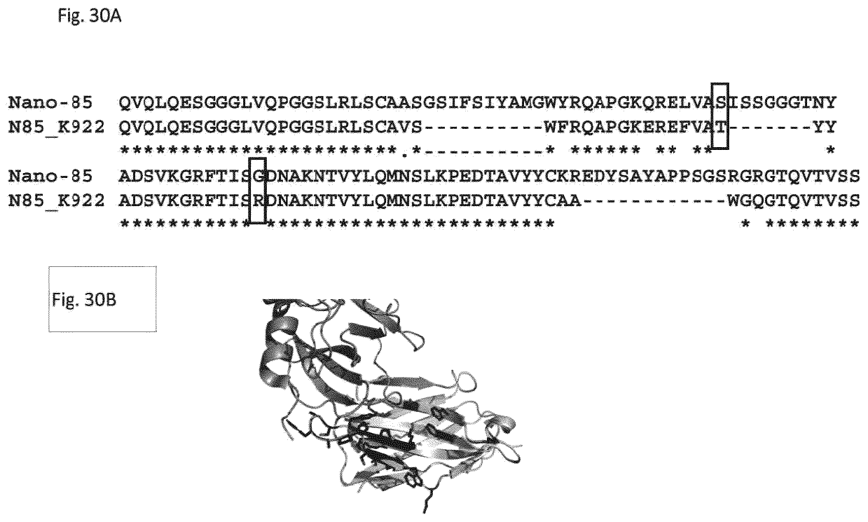

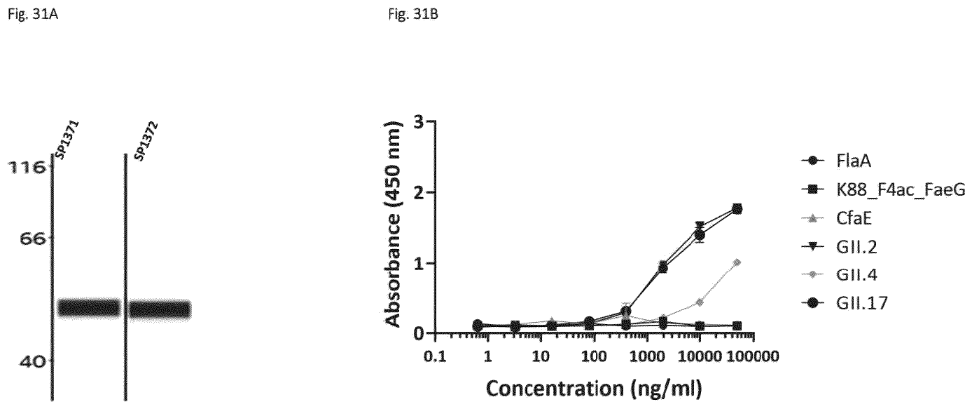

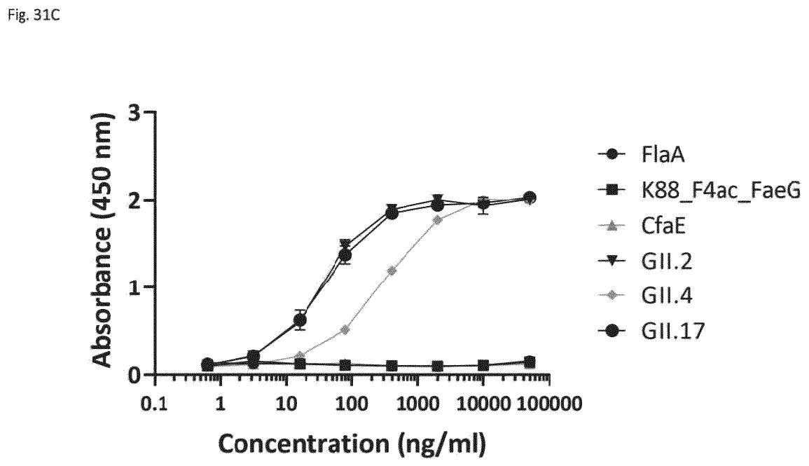

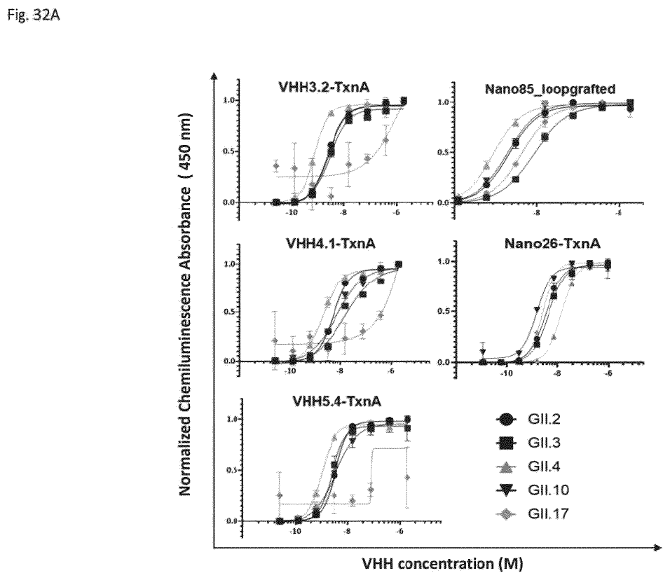

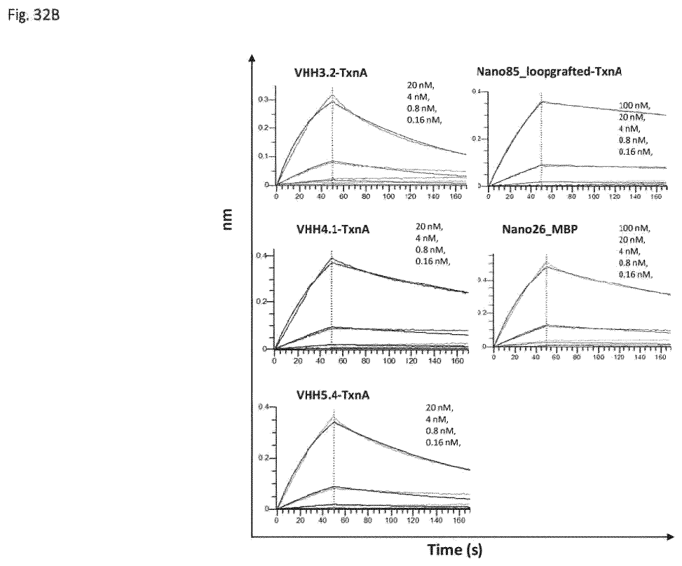

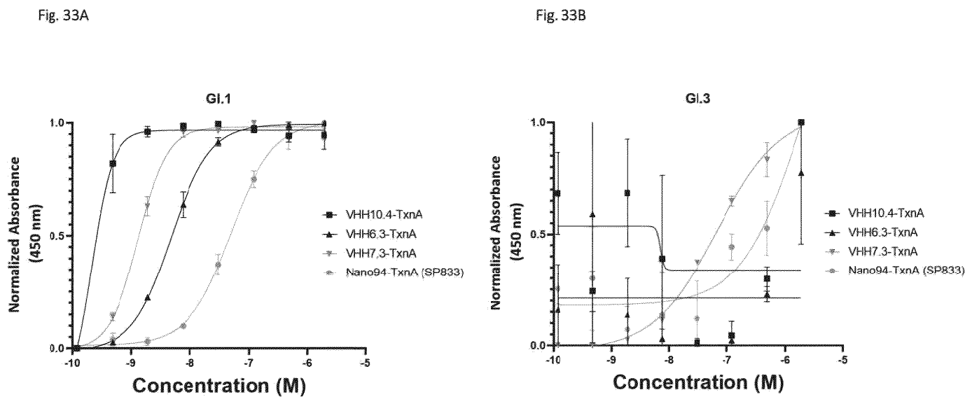

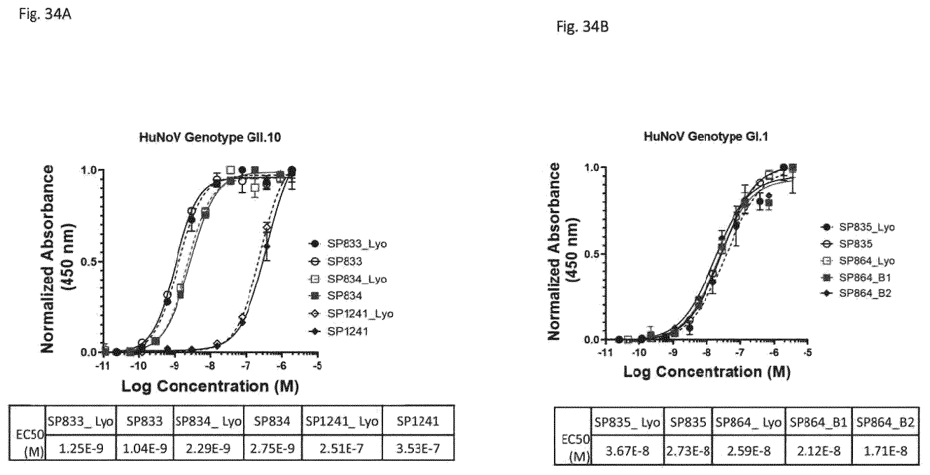

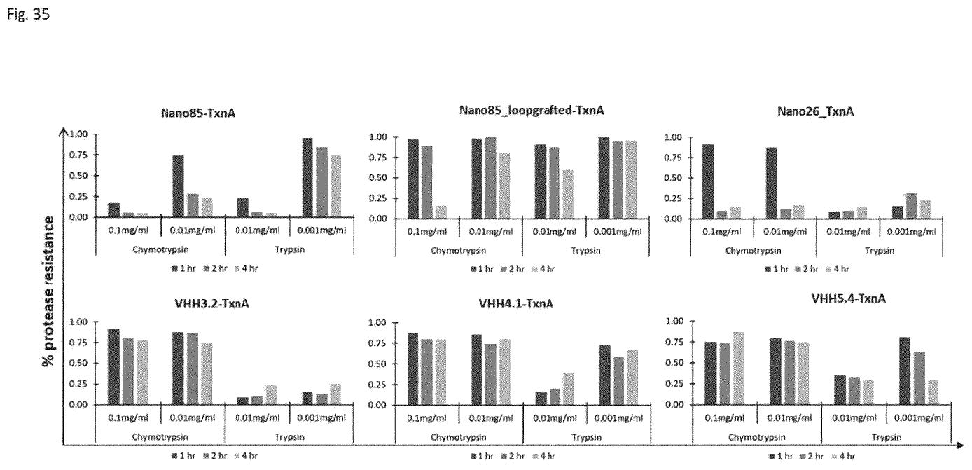

To create novel VHHs that target norovirus, Nano85, an anti-human Norovirus (HuNoV) protrusion (P) domain antibody was modified by grafing the binding regions of Nano85 onto the framework of the K922 antibody (SEQ ID NO: 18) which is known to be resistant to gut proteases and allow increased expression in Spirulina. (FIG. 30). Constructs comprising the unmodified Nano85 having a C-terminal maltose binding protein (MPB) (SP1371) and the modified Nano85 having a C-terminal MPB (SP1372) were expressed in Spirulina. (FIG. 31A). Further, SP1371 and SP1372 bind to various recombinant P domains derived from different human norovirus Gii strains (GII.2, GII.4, and GII.17). (FIGS. 31B and 31C). The purified proteins also show measurable binding to irrelevant antigens, including the campylobacter flagellin protein FlaA, Swine ETEC adhesin protein FaeG, and Human ETEC fimbrial adhesion domain CfaE.

Moreover, the binding kinetics and cross-reactivity of various recombinant anti-human P domain targeting VHH sequences was studied. Nano26 (SEQ ID NO: 73) and Nano85 (SEQ ID NO: 71) show broad cross-reactivity, while VHH3.2, VHH4.1, and VHH5.4 show no binding against the GII.17 P domain. (FIG. 32A-B)

TABLE 3
ELISA based binding against HuNoV GII.2, GII.3,
GII.4, GII > 4, GII.10 and GII.17 P domains.

| | | | | Nano85_loop- | |
|---|---|---|---|---|---|
| EC50 | VHH3.2 - | VHH4.1 - | VHH5.4 | grafted- | Nano26- |
| (nM) | TxnA | TxnA | TxnA | TxnA | TxnA |
| GII.2 | 2.45 | 5.31 | 3.43 | 2.14 | 3.38 |
| GII.3 | 2.93 | 15.62 | 2.52 | 8.67 | 4.46 |
| GII.4 | 0.77 | 2.05 | 1.07 | 0.91 | 15.45 |
| GII.10 | 2.58 | 7.34 | 3.46 | 1.99 | 1.46 |
| GII.17 | ND | ND | ND | 4.32 | 2.52 |

TABLE 4
BLI based binding kinetics to HuNoV GII.2 P domain

| | KD | ka | kdis |
|---|---|---|---|
| | (nM) | (1/Ms) | (1/s) |
| VHH3.2 -TxnA | 7.12 | 1.18E6 | 8.41E−3 |
| VHH4.1 -TxnA | 5.51 | 6.70E5 | 3.70E−3 |
| VHH5.4 TxnA | 8.73 | 7.73E5 | 6.75E−3 |
| Nano85_loopgrafted- | 10.1 | 1.36E5 | 1.38E−3 |
| MBP | | | |
| Nano26-MBP | 21.9 | 1.72E5 | 3.78E−3 |

Additionally, the binding and cross-reactivity of anti-Human Norovirus (HuNoV) P domain targeting VHHs Nano94 (SEQ ID NO: 75), VHH10, VHH6.3, and VHH7.3 were assessed. The VHHs tested exhibit binding EC50 ranging from 0.21 nM to 50.07 nM, where spirulina expressed recombinant nano94-TxnA shows the weakest binding. (FIG. 33A) The VHH7.3 was cross-reactive binding against the GI.3 P domain. (FIG. 33B)

TABLE 5
EC50 values from ELISA based binding
against HuNoV GI.1 and GI.3

| EC50 | VHH10.4 - | VHH6.3 - | VHH7.3 | Nano94- |
|---|---|---|---|---|
| (nM) | TxnA | TxnA | TxnA | TxnA |
| GI.1 | 0.21 | 4.75 | 1.34 | 50.07 |
| GI.3 | ND | ND | 66.28 | ND |

To create effective Spirulina expressing anti-human norovirus VHHs, the stability of the recombinant Spirulina when lyophilized was determined. The constructs from the SP833, SP834, SP835, SP864, and SP1241 were lyophilized and tested for stability. (FIG. 33A-B). Comparison of the stability of the lyophilized proteins with that of purified protein stored at 4° C. show no loss in binding activity.

The protease sensitivity of various anti-human norovirus P domain VHH constructs was assessed by incubating 1 μg bacterial expressed recombinant VHHs with 20 μL of chymotrypsin (0.1 mg/mL or 0.01 mg/mL) or Trypsin (0.01 mg/mL or 0.001 mg/mL) in digestion buffer (1 mM Tris pH 8.0, 20 mM CaCl₂)) for one hour, two hours, or 4 hours. Protease sensitivity was measured using ELISA-based binding as shown in Figure BB6. The loop-grafterd Nano85 exhibits the best protease resistance compared to recombinant Nano85 and the other tested. VHH3.2, VHH4.1, and VHH5.4 show resistance against chymotrypsin while exhibiting varying sensitivity to Trypsin.

### Example 14: Inflammatory Bowel Disease Therapeutics: Spirulina Expressed Anti TNF Alpha VHHs

Inflammatory bowel diseases (IBD) are chronic disorders of the gastrointestinal tracts. IBD, which include Chron's disease and ulcerative colitis, are relapsing diseases with a tendency of being progressive. IBD treatments include anti-inflammatory drugs, immunosuppressive drugs, and anti-TNF α biologics. Tumor Necrosis Factor alpha (TNF-α) is a cytokine involved in inflammation. In chronic IBD, TNF α accumulated in the lamina propria of the gut mucosa. Increased accumulation of TNF α is responsible for chronic inflammation and subsequent damage to the intestinal epithelial cells. Current anti-TNF α biological therapeutics under investigation include infliximab, adalimumab, golimumab, and certolizumab. Given the chronic nature of IBD, oral delivery of biologics is ideal for patient comfort, ease of treatment, willingness to adhere to prescription regimen and cost. However, biologics that are developed for IBD are currently delivered intravenously or subcutaneously due to physiological barriers that render biologics not effective for oral delivery. These challenges include instability of protein-based therapeutics in the GI tract, extreme pH environments, and high enzymatic activity in the GI tract.

Singe domain Llama antibodies (VHHs) possess properties that make them amenable for oral delivery. VHHs retain antigen binding specificity and potency comparable to traditional IgG antibodies. The small size of VHHs and rigid structural nature, solubility, ease of expression and stability under the GI environment makes VHHs suitable for oral-based therapeutics. Given these properties, VH Squared had developed VHH (V565) that can bind TNF α and can be used for the management of IBD through oral delivery.

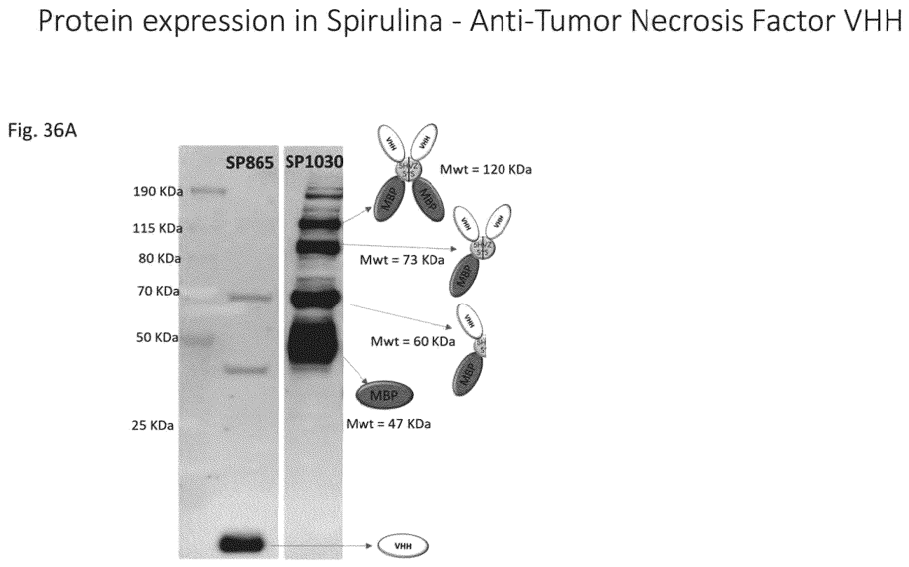

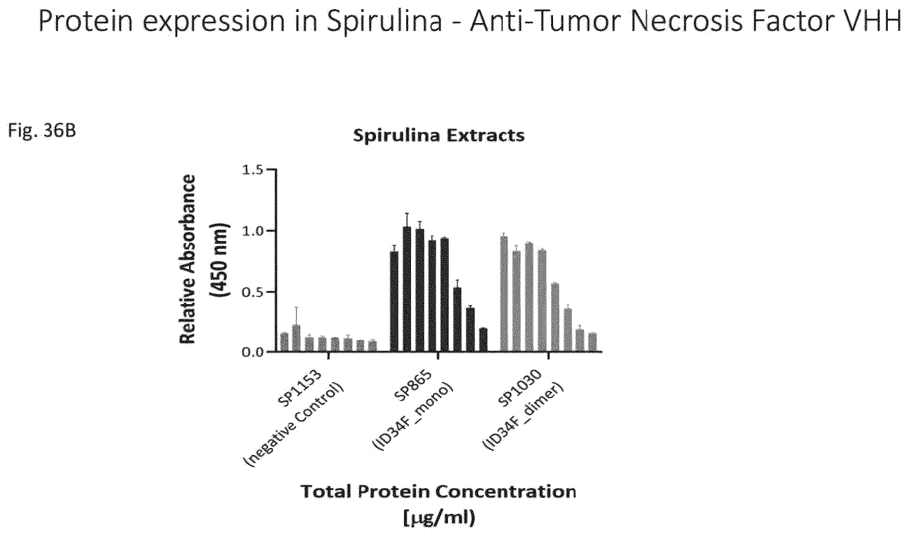

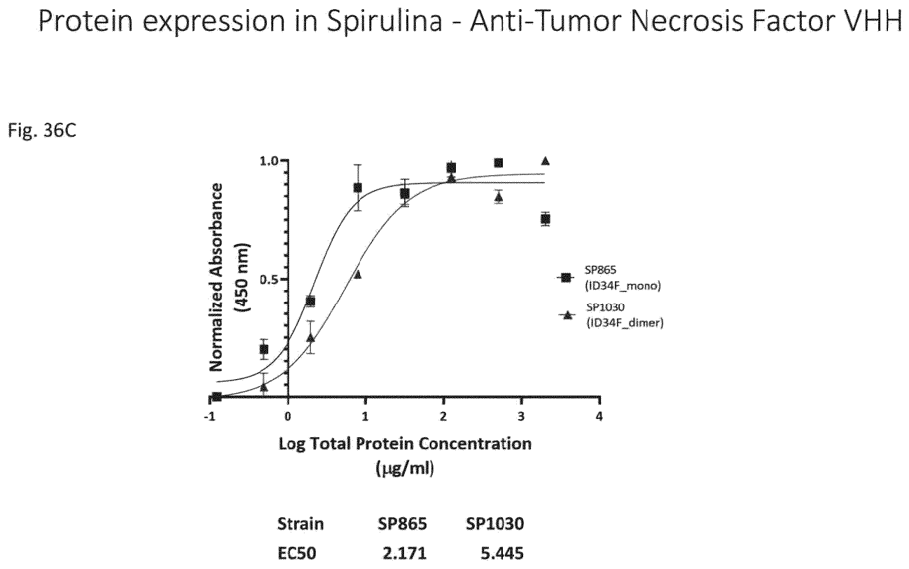

The anti-TNF-α VHH from VH squared as monomer and dimer has been expressed. (FIG. 36A-C) The expression level of anti-TNF-α VHH is determined by Western Blotting using anti-tag or anti-VHH primary and appropriate secondary antibody combinations. Binding activity of protein expressed in Spirulina strains are assessed using ELISA where the antigen is coated onto high binding plates, and antibody-expressing Spirulina strain crude cell lysate is titrated in dilutions. Both monomeric and dimeric forms of the VHH show good binding to recombinant human TNF-α.

### Example 15: Clostridium Difficle Toxin B (tcdB) Specific VHHs in Spirulina

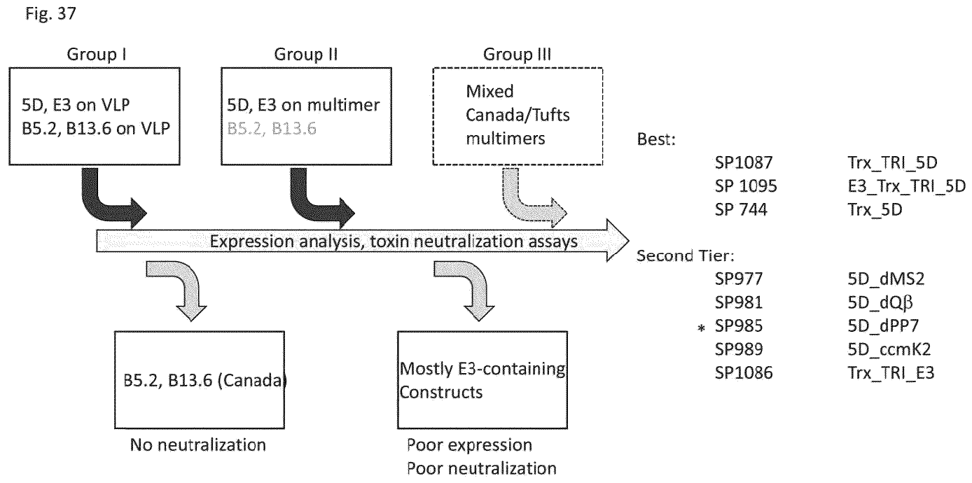

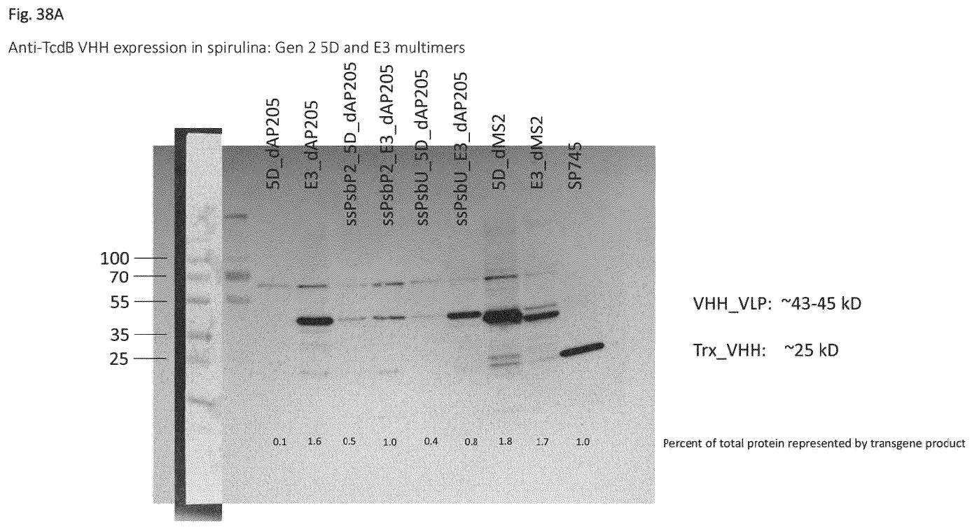

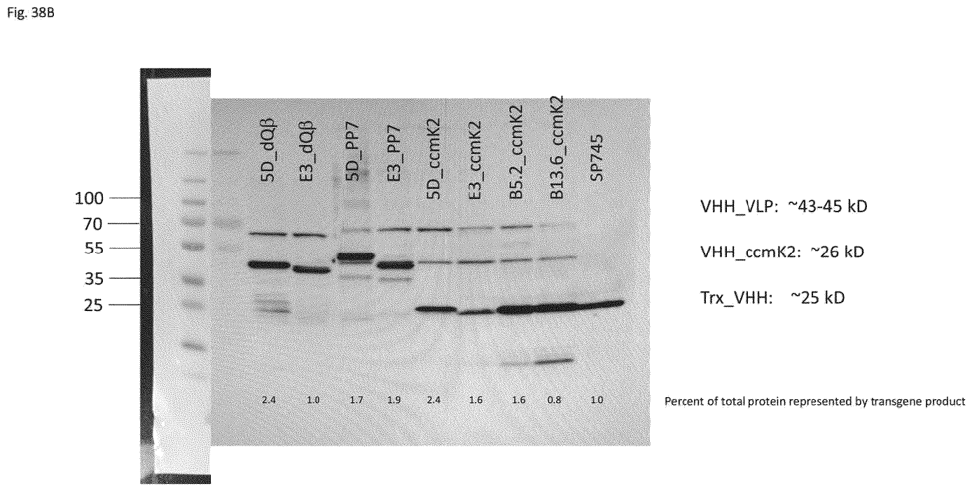

Anti-tcdB VHHs 5D (SEQ ID NO: 5) and E3 (SEQ ID NO: 6) were constructed into various scaffolds and expressed in Spirulina. (FIG. 37) Scaffolds include E. coli-derived thioredoxin (Trx), virus-like particles derived from a number of RNA phages (MS2, Q_{β}, PP7 and AP205), and computationally-designed trimers and pentamers.

For the trimers and pentamers, thioredoxin was always used in the scaffold structure; some are designed as homomultimers (eg. Trx-Trimer-VHH), some as homo-multivalent structures (eg E3.VHH-Trx-TRIMER-E3.VHH) and some as hetero-multivalent structures (eg. E3. VHH-Trx-TRIMER-5D.VHH).

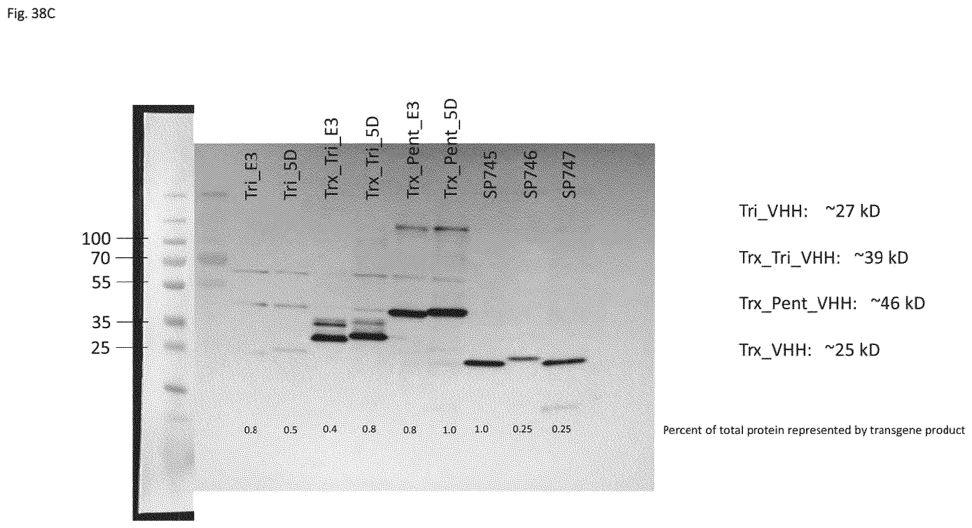

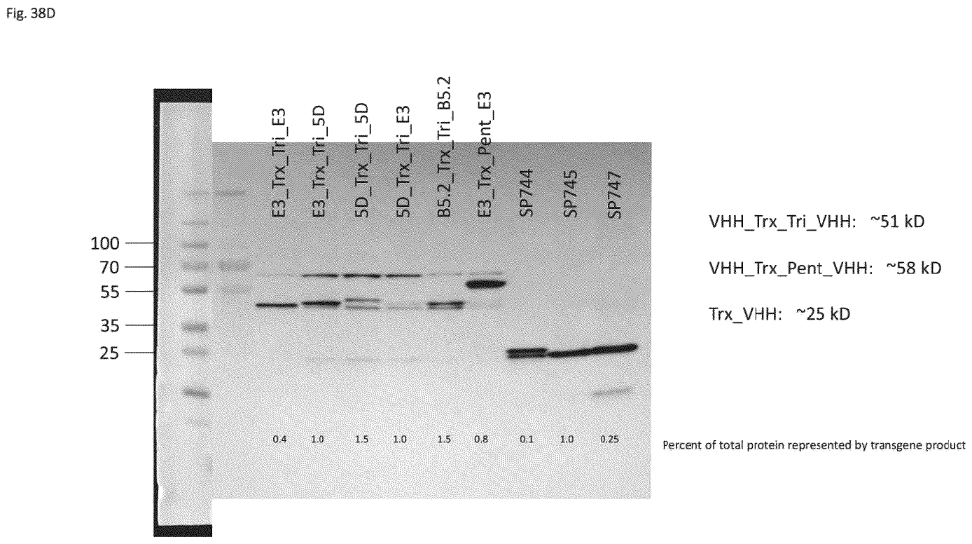

Constructs containing VHH.5D express at higher levels than those with VHH.E3. Certain hetero-multivalent structures express at higher level if E3 is at the N-terminus as opposed to 5D. (FIG. 38A-C)

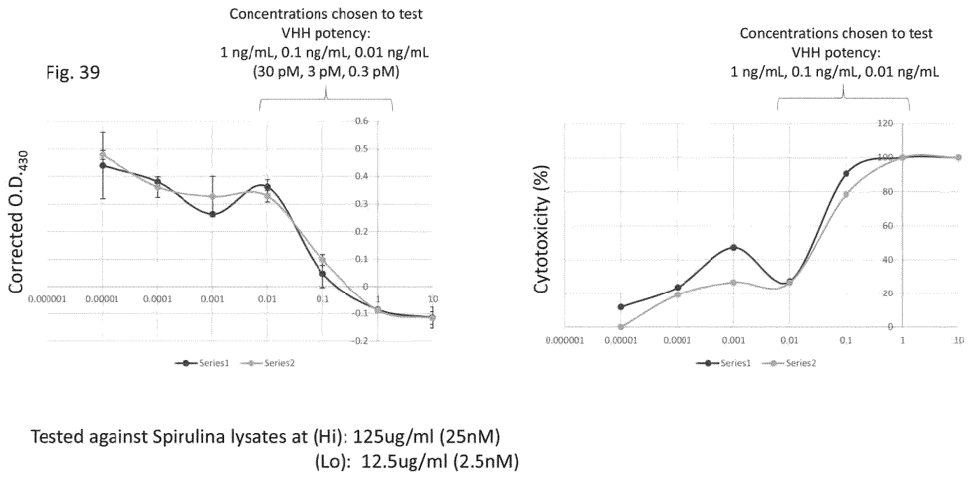

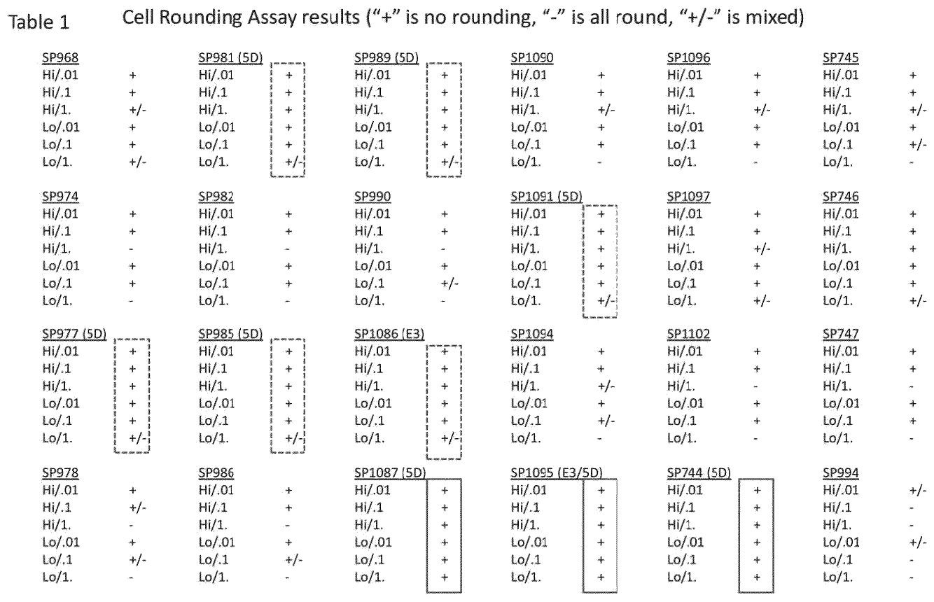

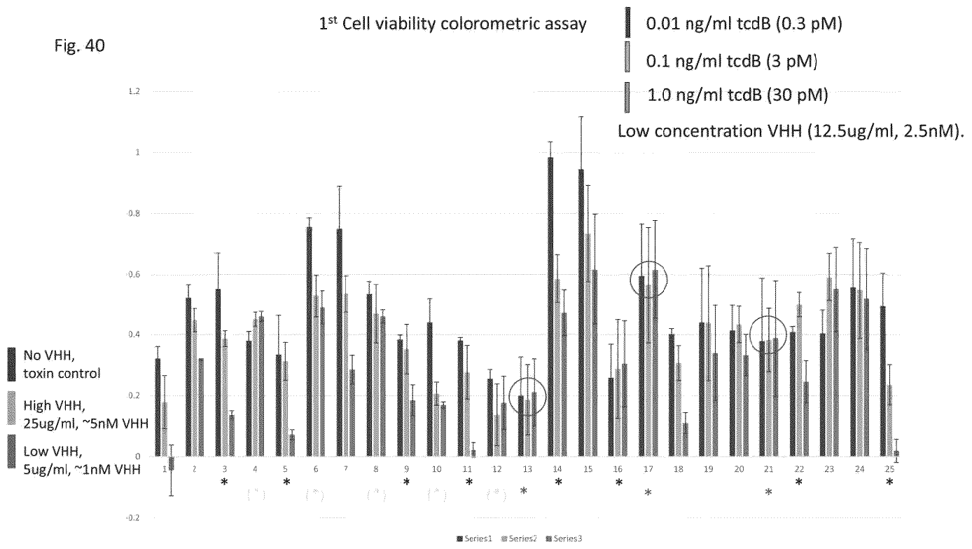

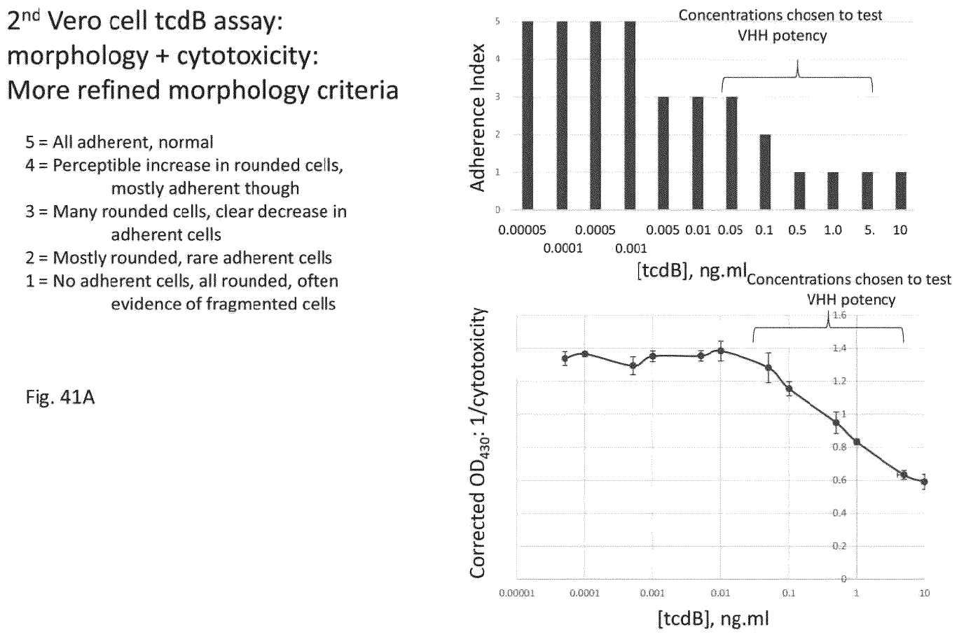

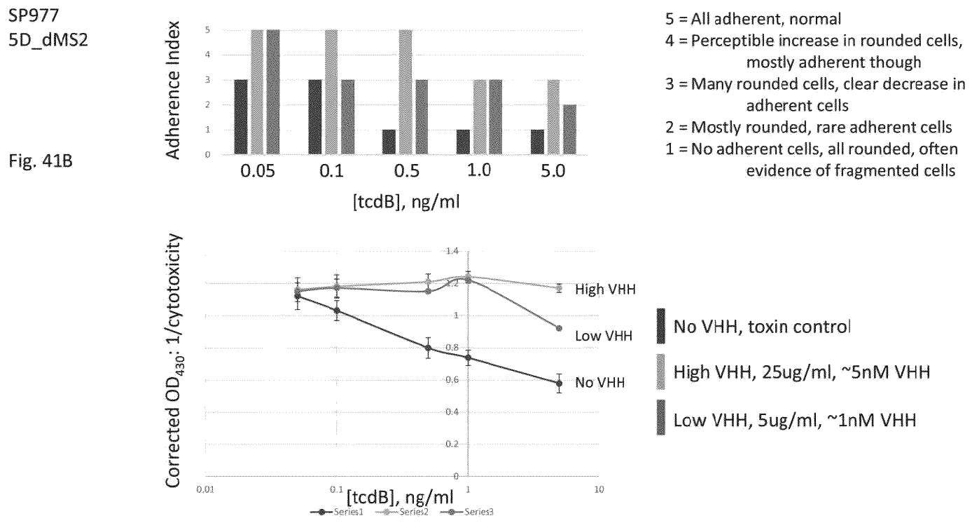

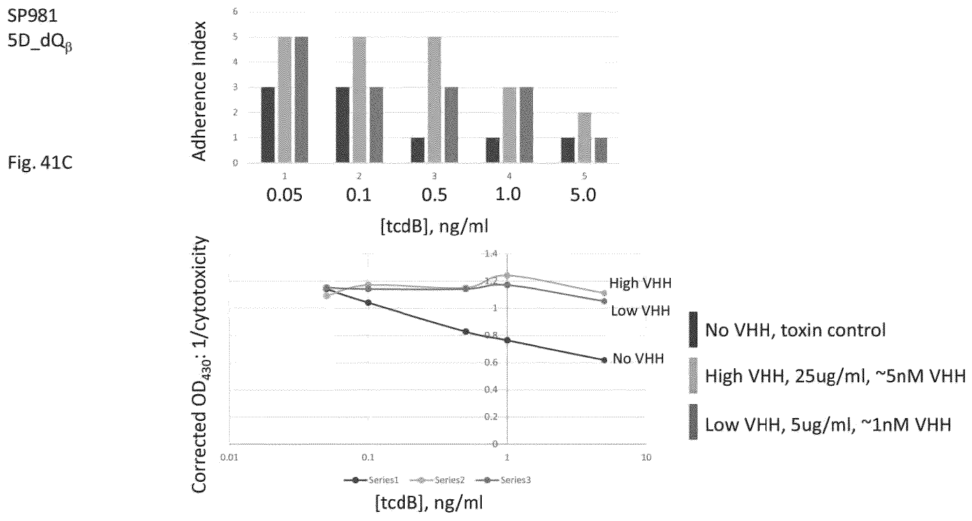

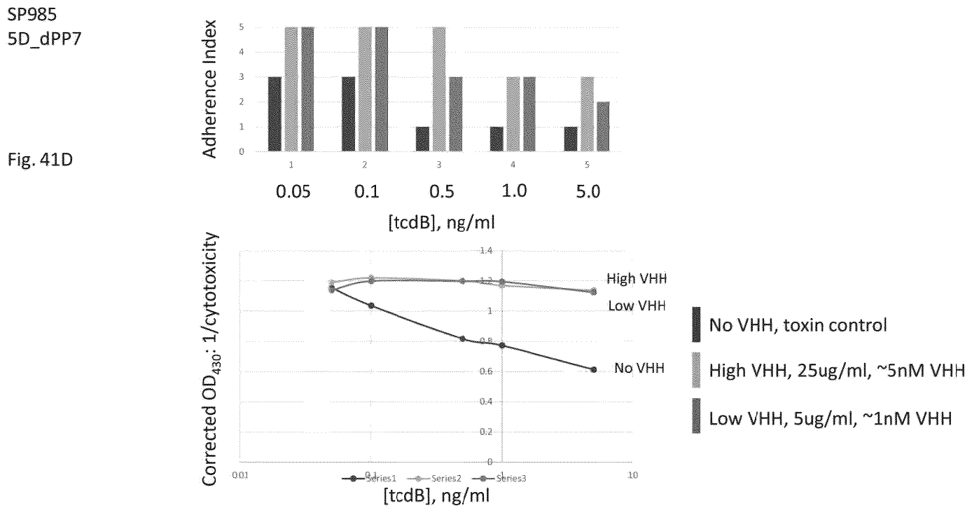

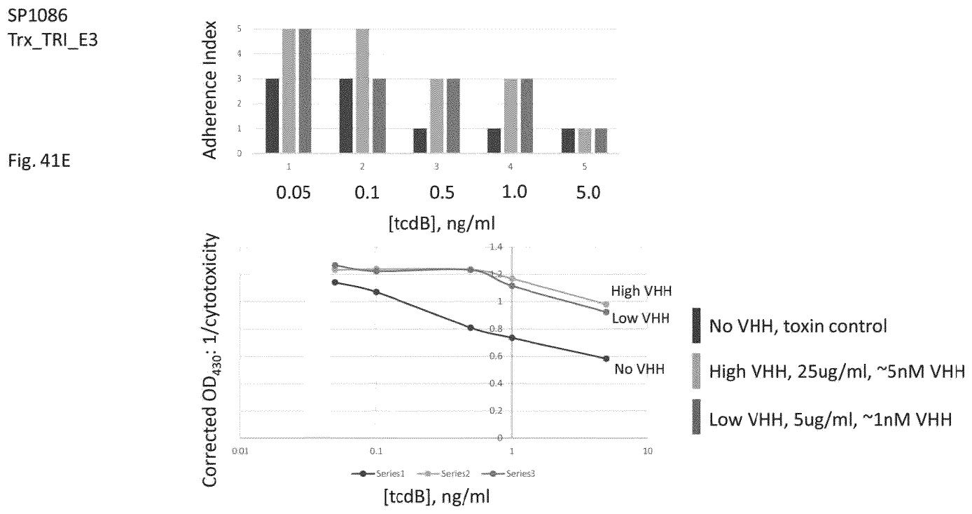

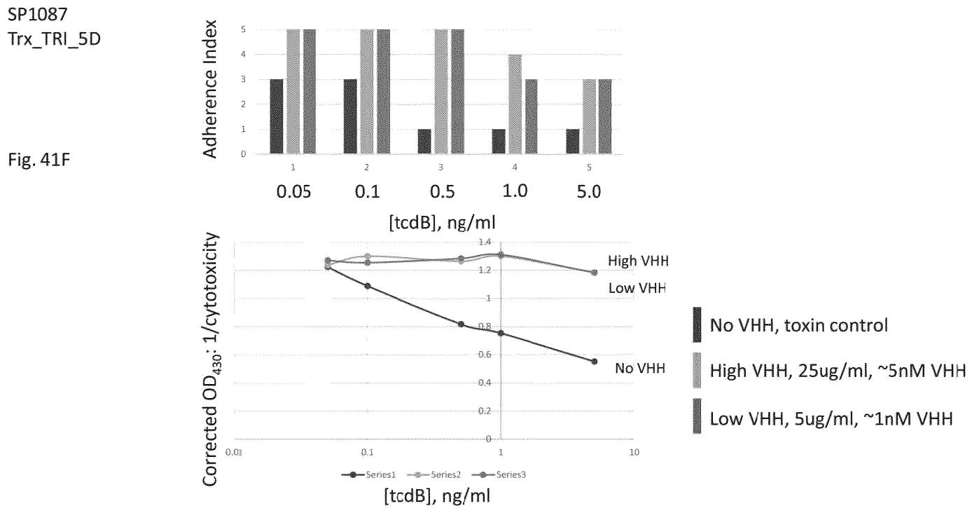

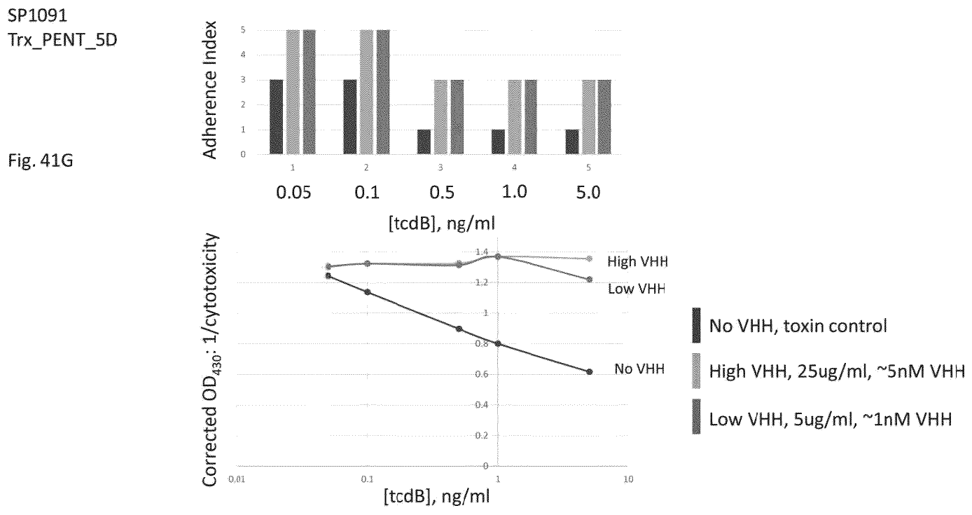

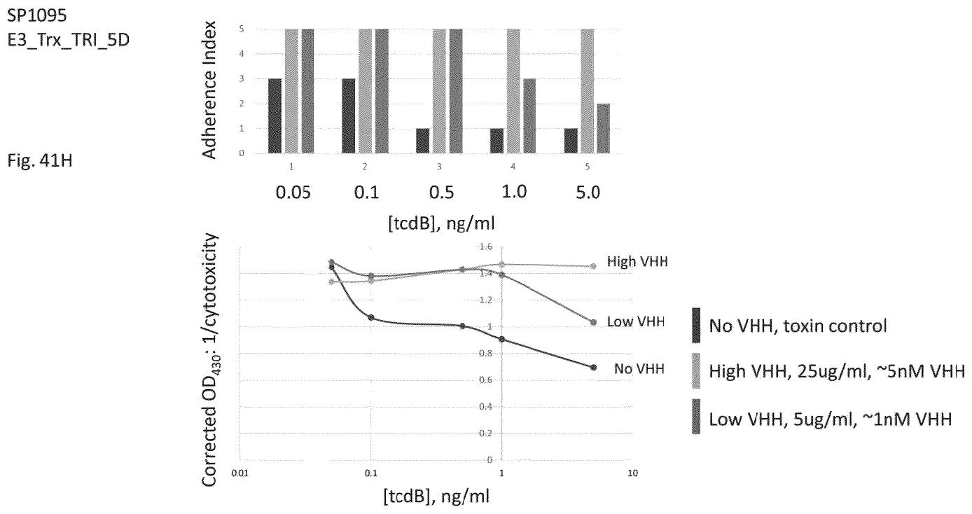

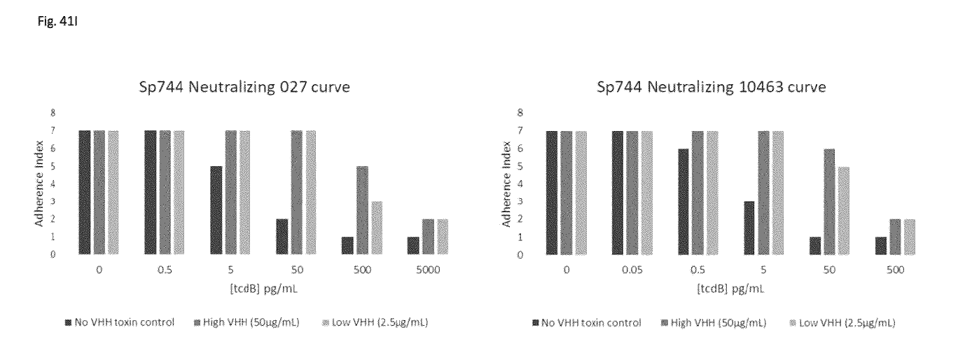

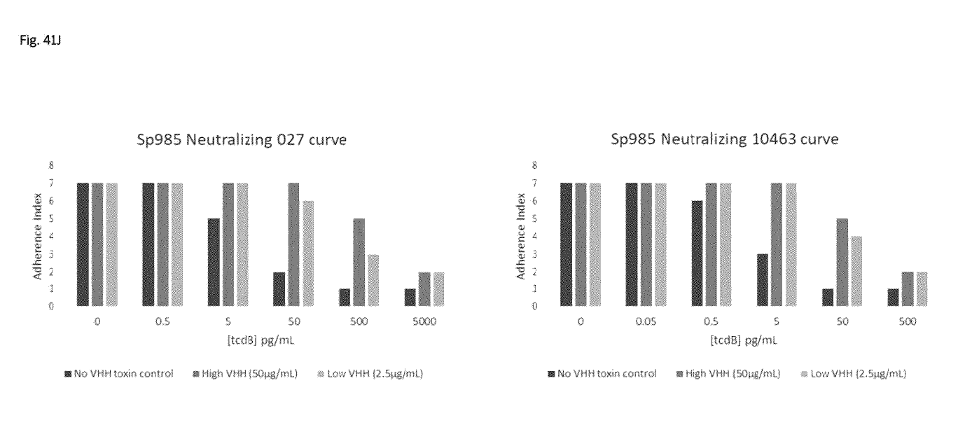

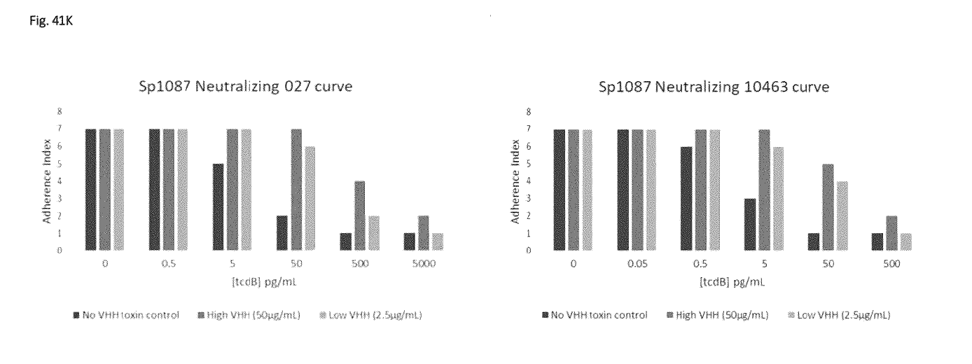

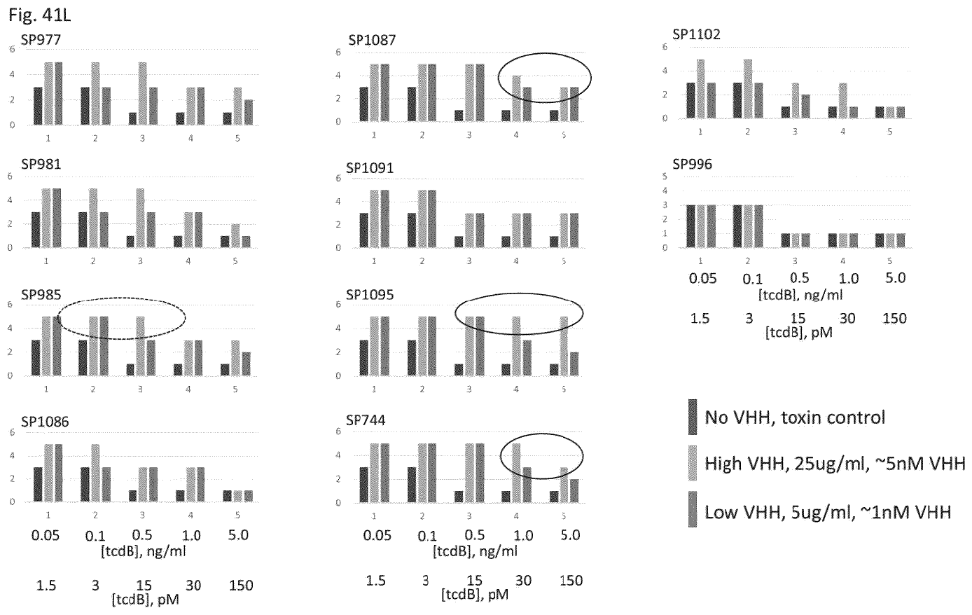

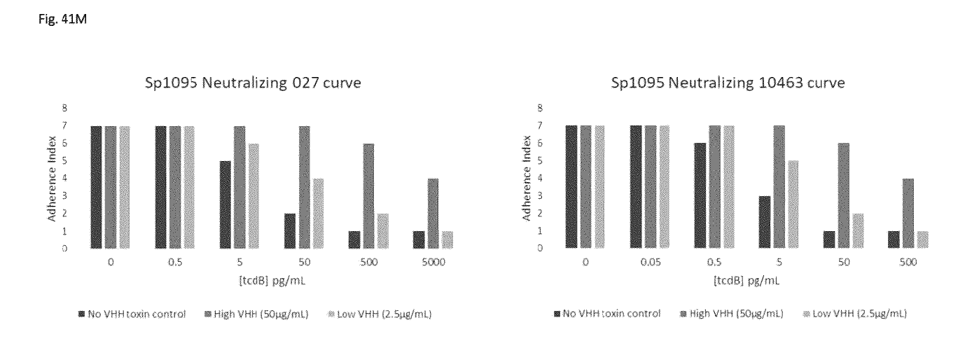

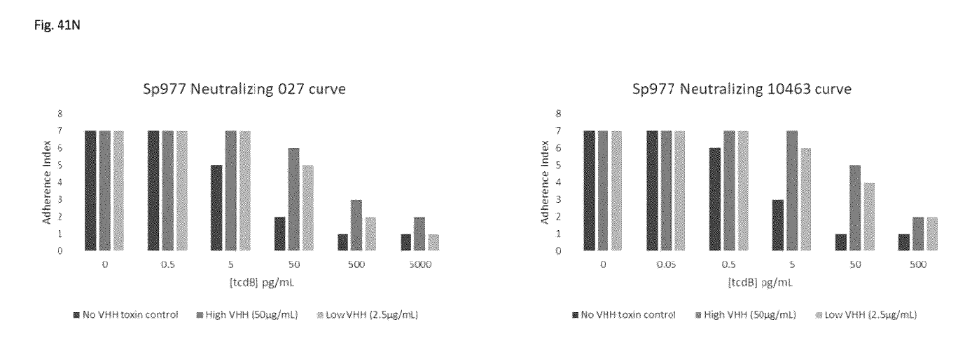

Constructs were evaluated for their neutralizing activity against tcdB in vitro. (FIG. 39). Vero cells (African green monkey epithelial cells) were exposed to dose ranges of tcdB with or without the addition of Spirulina extracts containing VHHs. The biologic effect was measured in two ways: first, a colormetric reagent that linearly reacts with heathy, metabolizing cells was used as a quantitative measure (FIG. 40) second, visual microscopy was used to assess the degree of “rounding”, that is, the degree to which the normally adherent and angulated Vero cells detach from the plastic substrate and appear round. (FIGS. 41A-O). These methods generally agree, though the visual rounding assay was consistently more sensitive.

### Results

i. B5.2, B13.6 VHHs (Canada) do not neutralize when expressed on VLPs.
ii. Tufts VHHs E3 and 5D both demonstrate neutralizing activity
iii. Generally, 5D-containing constructs express more abundantly, and demonstrate
more potent neutralizing activity.
iv. The best in vitro activity was shown by the following strains:
   SP1095, a heterobifunctional trimer construct, E3_Trx_TRI_5D
   SP747, monomeric Trx_5D
   SP1087, trimer construct Trx_TRI_5D
   Slightly less potent in vitro were:
   SP985, RNA phage VLP PP7 hybridized to VHH 5D
   SP1091, pentamer construct Trx_PENT_5D.

VHH-5E (SEQ ID NO: 7) constructs were also constructed. VHH.5E-containing constructs performed more potently than those bearing VHH.E3, though the most potent, on a per-mole basis was a trimer containing both VHH.E3 and VHH.5D. Potency generally followed expression level, though the most effective/potent structure was VHH.E3-Trx-Trimer-VHH.5D, which expressed at only ˜0.1% total protein, and was more potent than Trx-VHH.5D, which expressed at ˜2% total protein and was the next most potent extract. Spirulina extracts with no VHH displayed no inherent neutralizing activity.

The three or four best performing strains will be expanded for bioreactor and spray drying. Further, next generation constructs will be designed and new strains will be built (e.g. markerless versions of present strains, native Arthrospira thioredoxin, hetero-multimers with 5D and new Tufts VHHs directed at RBD). Also, animal studies will be initiated using the present hit strains: 1) Mouse model I: Lyras/Australia; 2) Mouse model II: Guerrant/Virginia; 3) Pig model: Tzipori/Tufts.

### Example 16—Combinations of VHHs Exhibit a Synergistic Increase in Binding to C. difficile Toxin

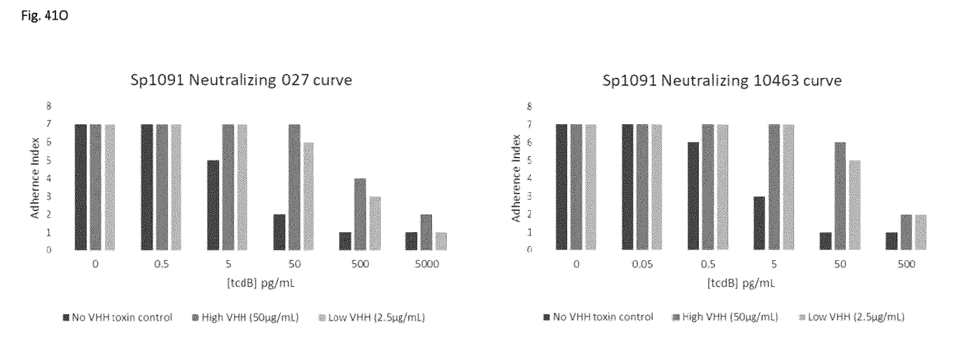

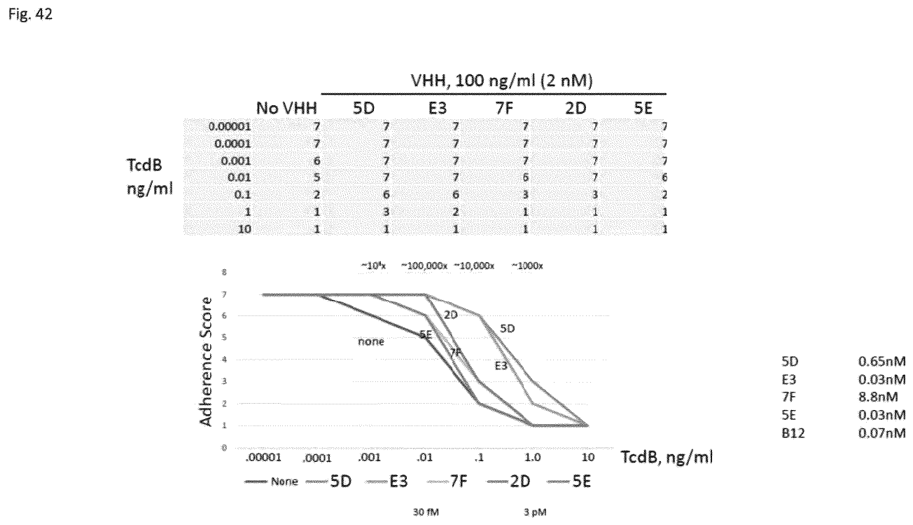

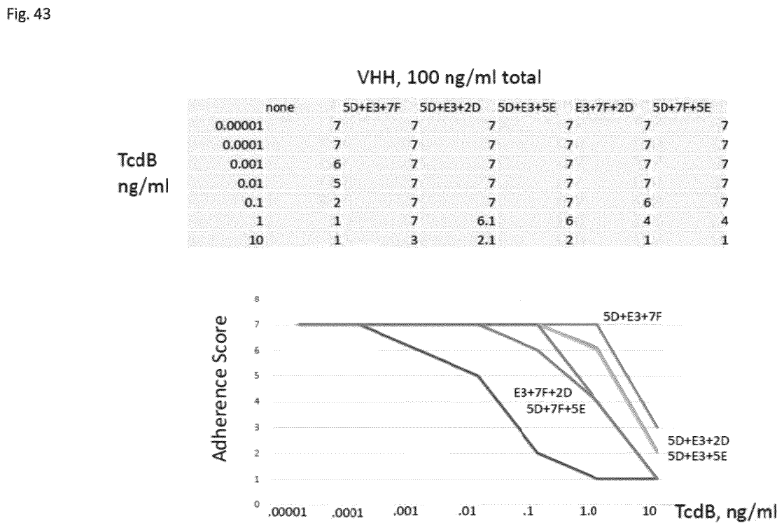

The binding strength of various VHHs alone and in combination to C. difficile TcdB toxin was tested. The VHHs were produced in E. coli and tested in vitro. FIG. 42 shows the binding strength of the VHHs 5D (SEQ ID NO: 5), E3 (SEQ ID NO: 6), 7F (SEQ ID NO: 13), 2D (SEQ ID NO: 65), and 5E (SEQ ID NO: 7) alone to TedB at various concentrations. The 5D VHH shows the greatest binding, with 2D showing the least binding. FIG. 43 shows the binding strength of different combinations of the VHHs 5D, E3, 7F, 2D, and 5E. FIG. 44 shows the binding strength of the VHHs 5D, E3, and 7F alone and in combination. FIGS. 45A-B show the binding strength of the VHHs 5D, E3, and 7F alone and in combination at different concentrations. An increase of the concentration of solitary VHHs had little increase in efficacy. In contrast, higher concentrations of the combination VHH (i.e. a VHH cocktail) showed a surprising increase in efficacy with increased concentration.

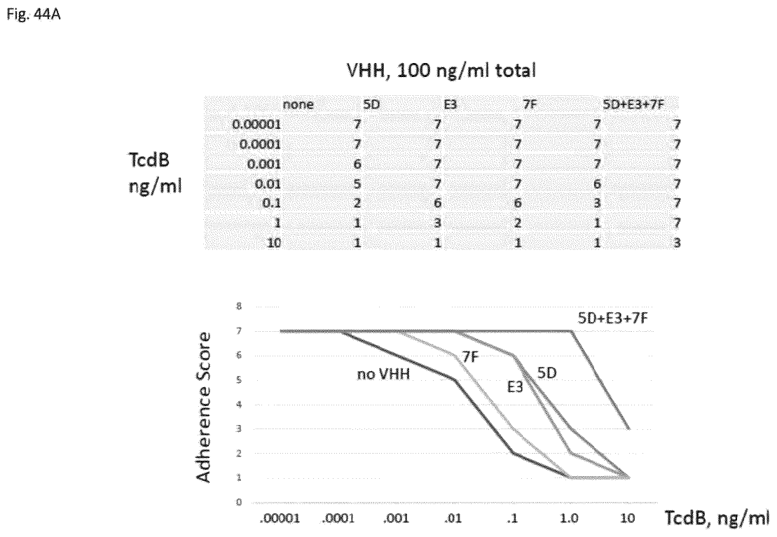

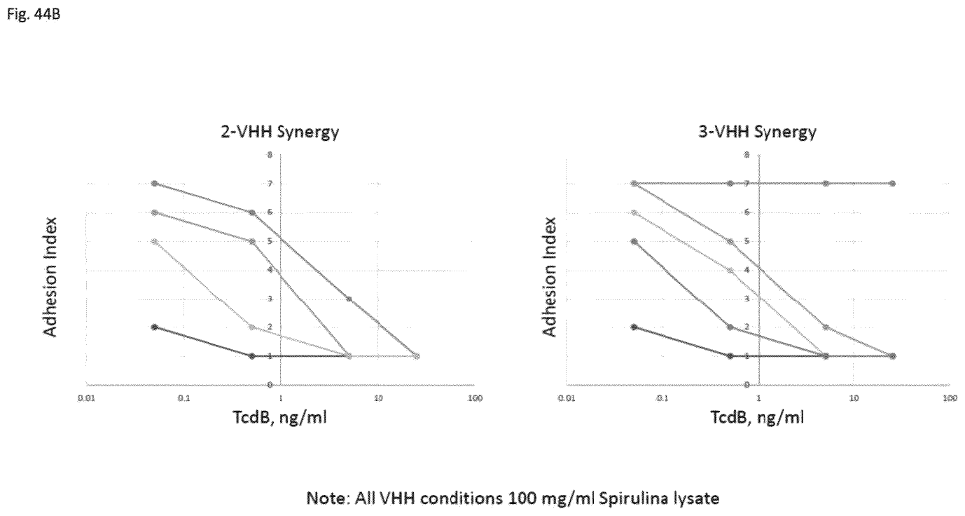

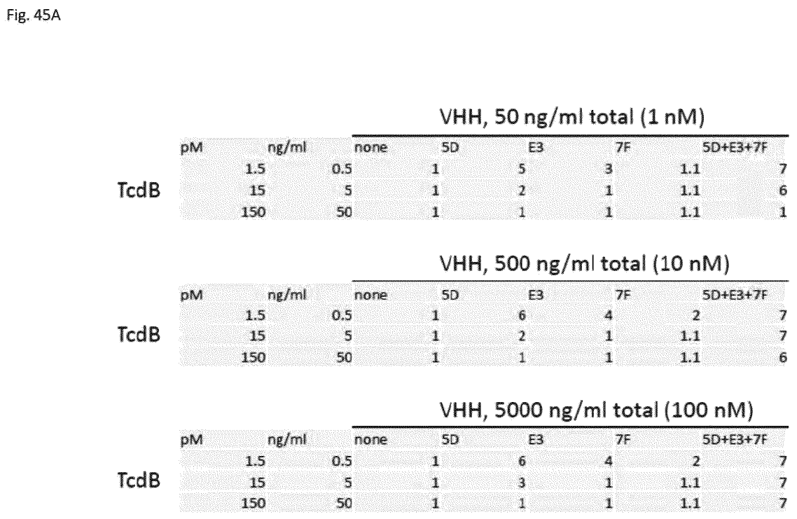

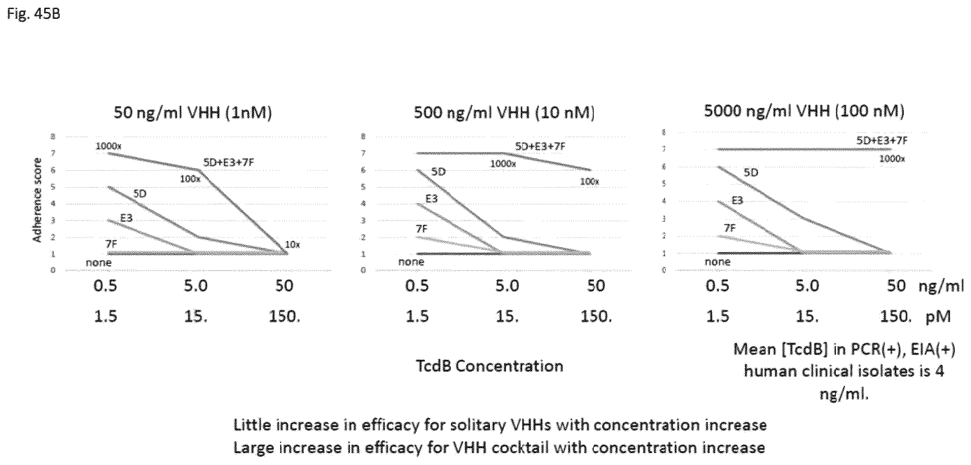

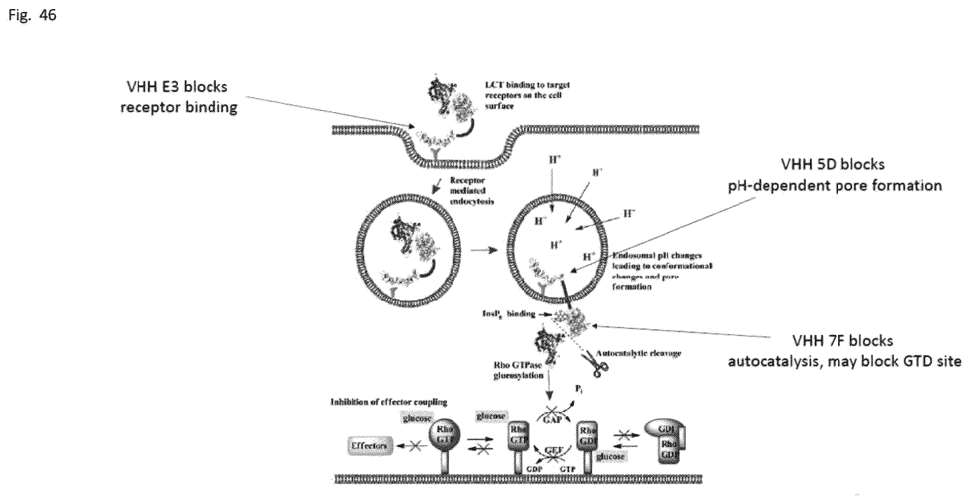

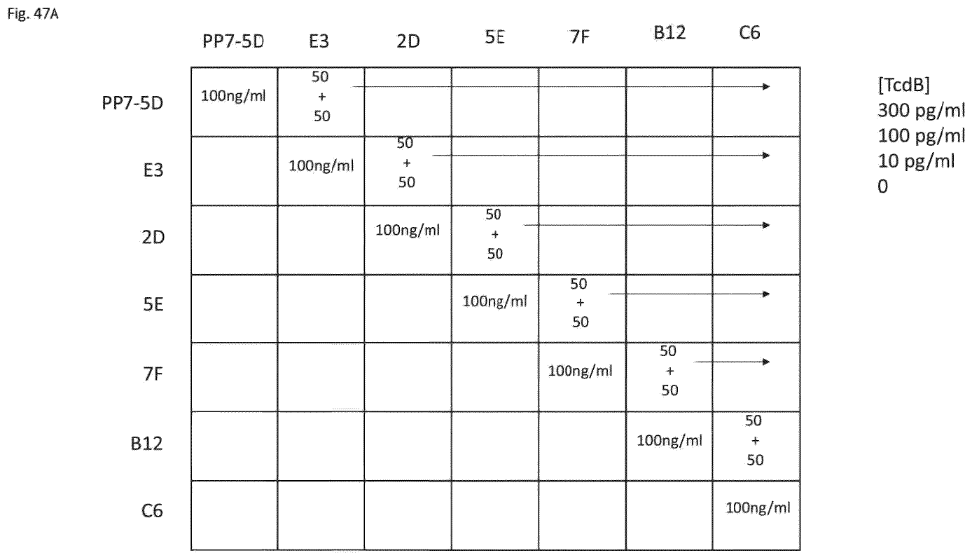

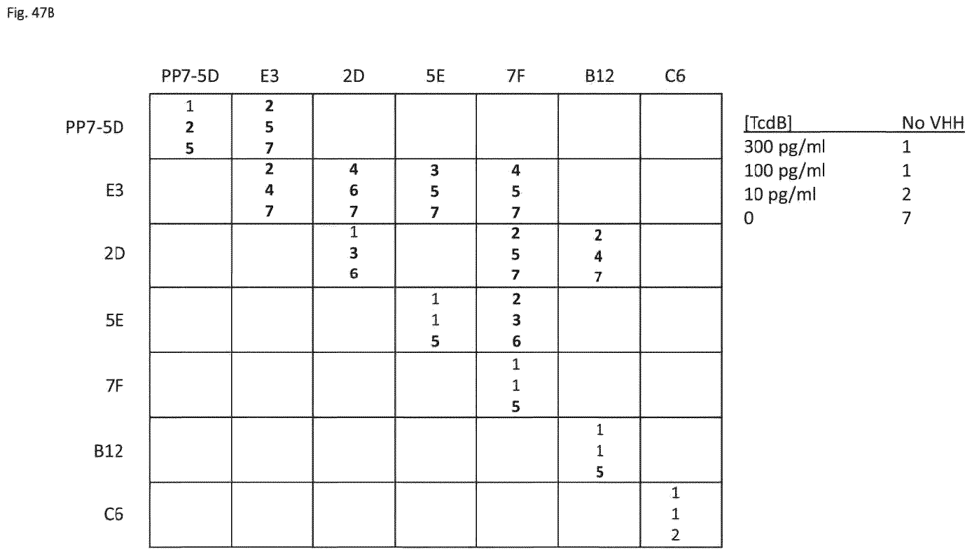

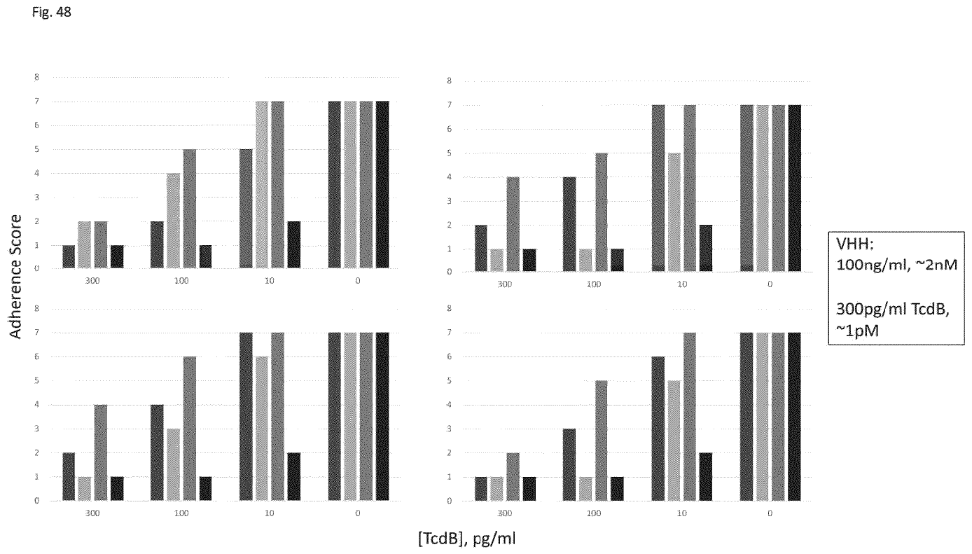

The increased efficacy of the combination of VHHs may be explained by the different targets of the different VHHs. For example, as shown in FIG. 46, the VHHs may act at different points of the process of the TedB singalling pathway. The VHH E3 blocks the receptor binding, the VHH 5D blocks the pH-dependent pore formation, and the VHH 7F blocks autocatalysis, and potentially the GTD site. This could explain the synergistic effect of the VHH cocktail over the effect of a single VHH.

TABLE 6
anti-TcdB VHHs

| | | | Target | Target |
|---|---|---|---|---|
| | | | Domain | Domain |
| Name | KD | | (Crystal) | (Reported) |
| 5D | 0.65 | mM | PFD | GTD |
| E3 | 0.03 | nM | GTD/FHD | GTD |
| 2D | Nd | | | GTD |
| 2Ds | Nd | | | |
| 5E | 0.03 | nM | | PFD |
| 7F | 8.8 | nM | GTD | GTD |
| A1 | Nd | | | RBD/CHOP |
| A11 | Nd | | | RBD/CHOP |
| AB8 | Nd | | | |
| B12 | 0.07 | nM | | RBD/CHOP |
| C6 | 0.89 | nM | | GTD |

Bacterial lysates of VHHs constructed in fusion with maltose binding protein (MBP) in a MBP-VHH orientation (with the exception of 5D, which was used as Spirulina lysate expressing a PP7 particle decorated with VHH 5d), were used at the concentrations indicated in Figures CC1 and CC2. Individual VHHs were used at 100 ng/ml, and 2-way combinations were used at 50 ng/ml each, for a total VHH concentration of 100 ng/ml. VHHs were tested against 3 concentrations of TcdB 027-type, as indicated.

### Example 17—Anti-TedB (Clostridium difficile Toxin B) VHHs Produced in Spirulina

Multimerizing single-domain antibodies in a single polypeptide chain increases avidity, and often biologic activity. Multi-VHH single polypeptides have been produced in E. coli, though have proven very challenging to express in Spirulina. Recently, the crystal structure of the TedB protein in its entirety (˜300 kDa) was solved with three VHHs bound (VHHs 5D, E3, and 7F). See FIG. 52 which shows TcdB bound to E3. Each VHH bound to a distinct domain spatially distant from one another. Two of the three domains have had essential biologic activities identified in the intoxication process, and the bound VHHs were shown to disrupt structural changes necessary for these functions. The third bound a domain that in homologous toxins has been linked to localization to the target cell's membrane. Each VHH had previously been shown to have some degree of toxin neutralizing activity on its own.

A single polypeptide containing the three VHHs will be sterically disfavored to either bind all three epitopes on one toxin, or to bind distinct epitopes on multiple toxin molecules. Given their demonstrated individual neutralizing activities, a simple mixture of the three VHHs will have neutralizing activity in excess of simply additive effects. Using bacterially-expressed protein, mixtures of two VHHs from a panel of 10 were tested, and it was identified independently that VHHs E3, 5D and 7F were particularly active when mixed in 2-member mixtures with each other, or with a number of other less active VHHs. Following on with 3-fold, 4-fold and 5-fold mixtures of the 10 VHHs, maximal neutralizing activity was found to coincide with any combination containing E3, 5D and 7F, the simplest being those three together.

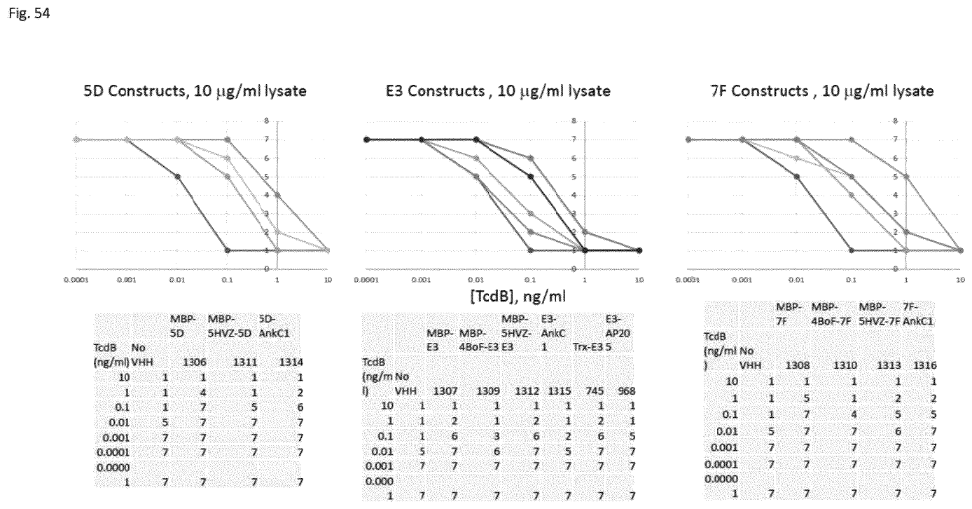

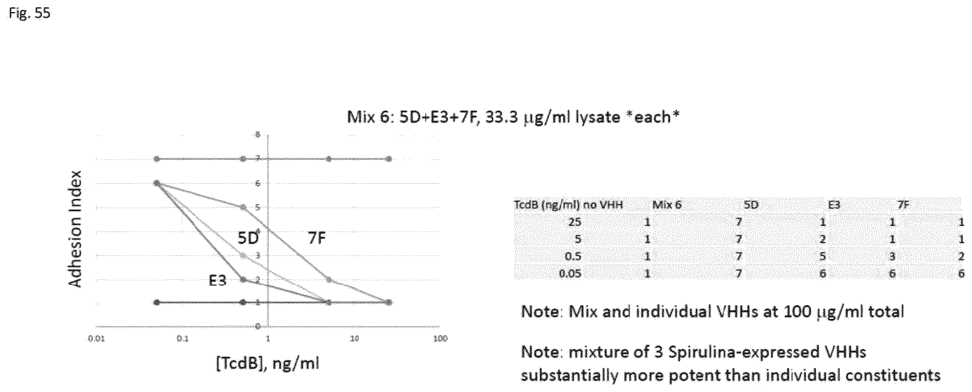

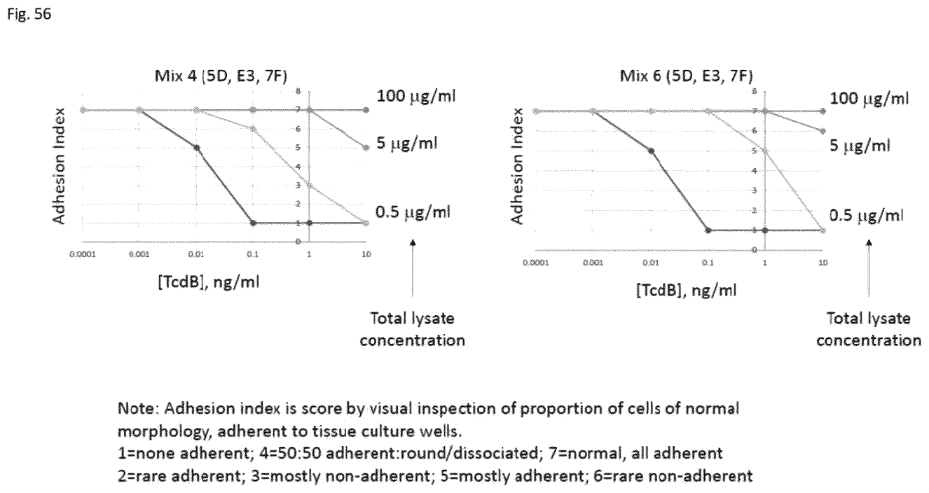

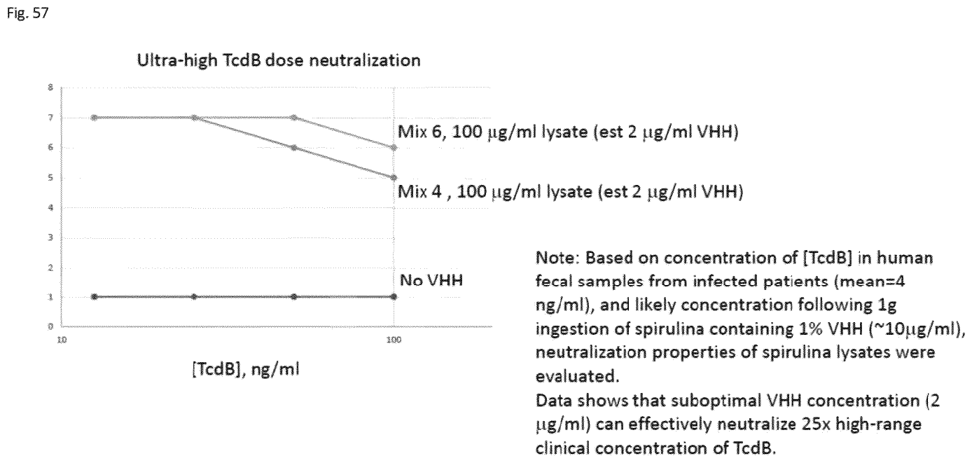

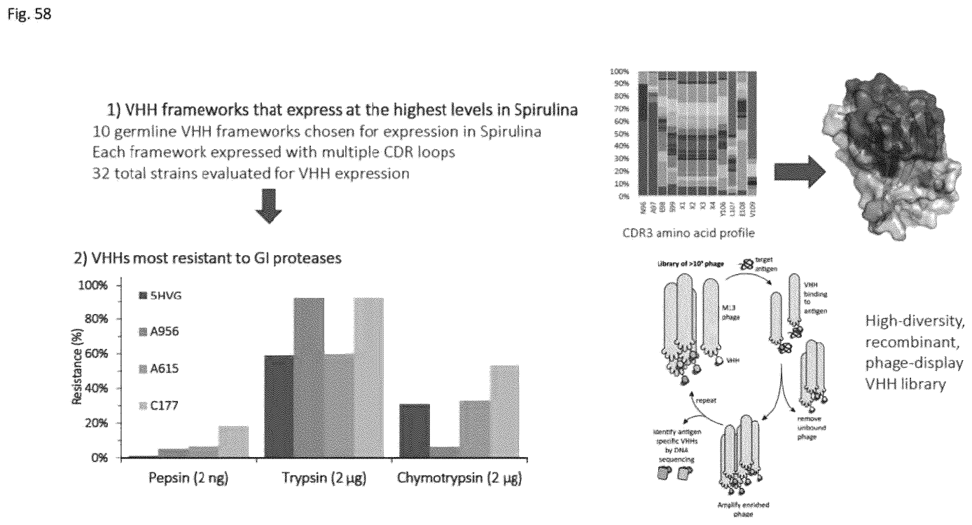

Each of the three VHHs were engineered into hybrid structures with known solubility- or folding-optimizing partners (chaperones), to maximize accumulation of biologically active VHHs in Spirulina. Spirulina lysates containing individual constructs containing E3, 5D or 7F were assayed for TcdB neutralizing activity in isolation (FIG. 54), and in various combinations containing all three VHHs (FIGS. 55 and 56). Surprisingly, lysate combinations containing all three VHHs appeared to have >1000-fold greater neutralizing activity than any single VHH lysate. Complete neutralization of TcdB was seen at toxin concentrations far in excess of that seen in human clinical isolates, by concentrations of VHHs well below that predicted to be available following human administration (FIG. 57).

### Example 18—Administration of VHHs with Other Therapeutic Molecules

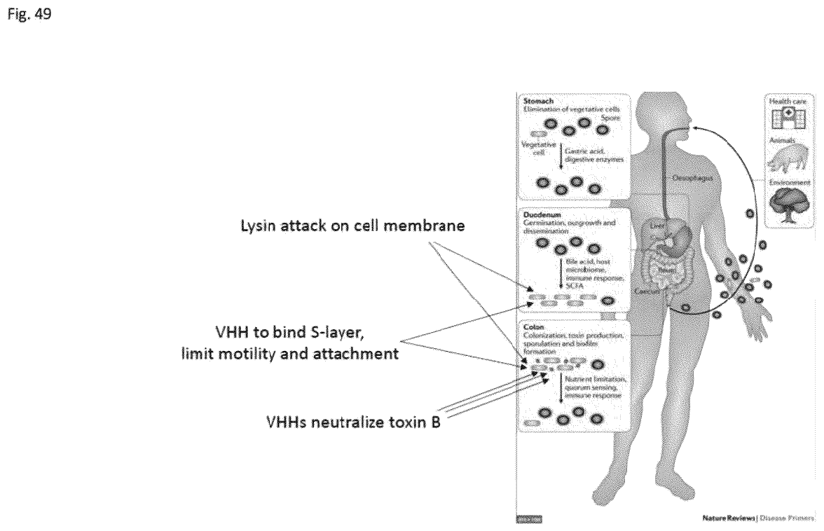

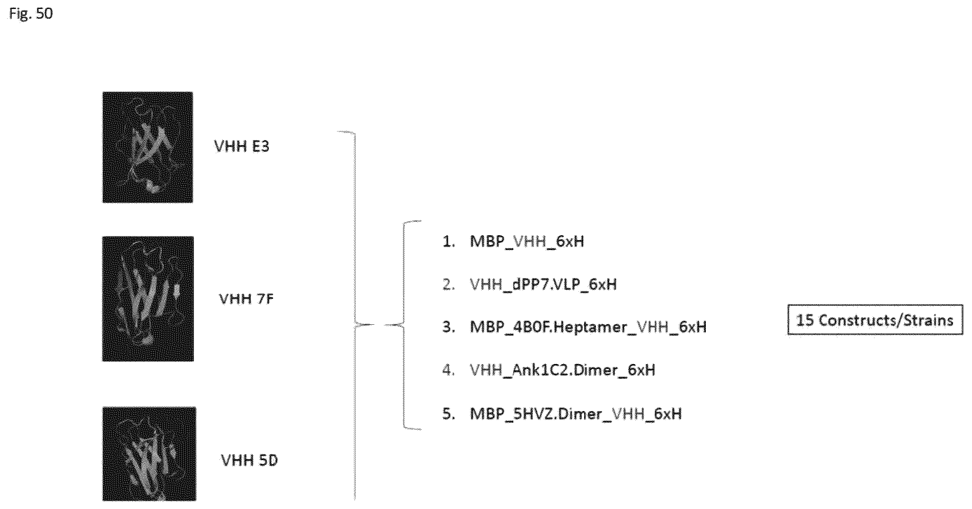

In addition to different VHHs, other therapeutics may be present in the recombinant Spirulina to further increase the efficacy of the orally-delivered therapeutic. The effect of multi-drug cocktails has been demonstrated for numerous organisms, including M. tuberculosis, where therapeutics that target cell wall synthesis, replication and transcription, energy metabolism, and translation may be combined to target different parts of the pathogen life-cycle (see FIG. 49). In the same way, targeting different aspects of the C. difficile receptor activation and the cell membrane may increase efficacy of an orally-delivered therapeutic. To demonstrate this, a recombinant Spirulina is produced that expresses one or more VHHs that bind to the S-layer of the C. difficile, one or more VHHs that neutralize toxin B, and a polypeptide such as a lysin to attack the cell membrane (see FIG. 50).

### Example 19: Lysin Expressed from Spirulina is Active

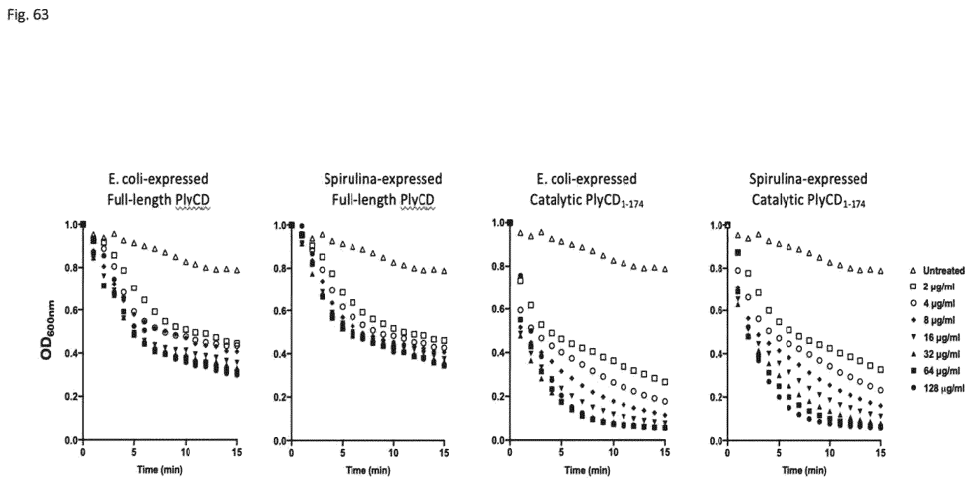

PlyCD and the catalytic domain fragment PlyCD1-174 have previously been expressed in E. coli and shown to be bacteriocidal in vitro and in vivo. To determine whether phage-derived anti-Clostridium cell wall digesting lysin PlyCD expressed from Spirulina was active, the genes for PlyCD and PlyCD1-174 were inserted into spirulina under the control of the cpc600 promoter and expression was confirmed by Western blot. Various concentrations were tested in a standard cell-lysis assay. FIG. 63 shows cell lysis assay results for both E. coli-expressed and Spirulina-expressed proteins. The Spirulina-expressed lysins are catalytically active.

### Example 20: Effect of Linkers on Neutralizing Ability of Anti-TcdB VHH Sequences

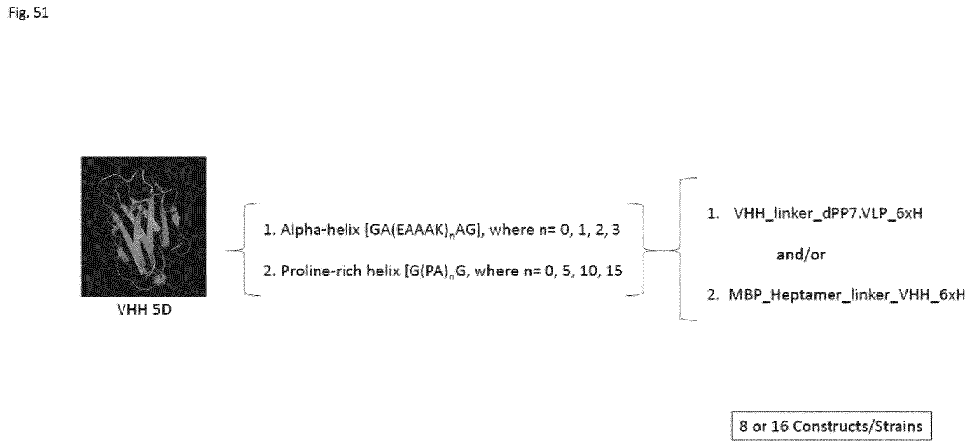

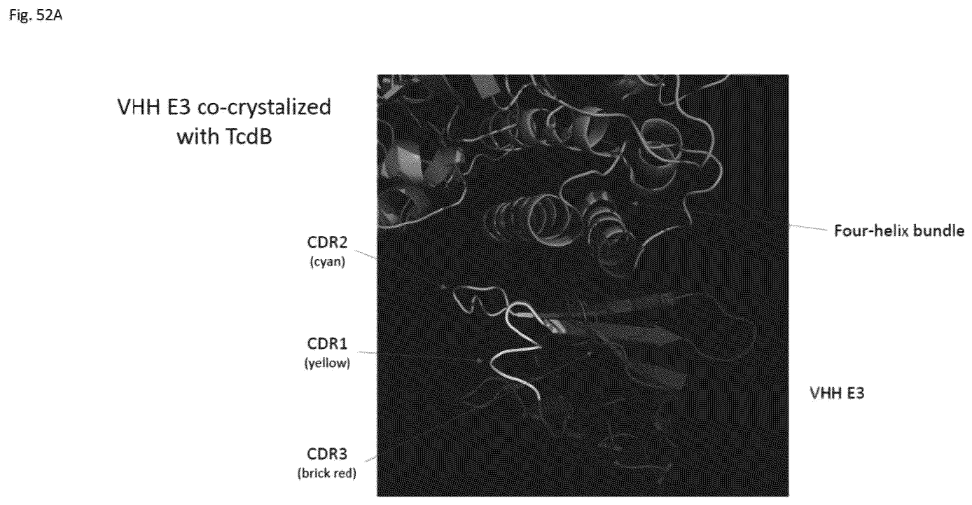

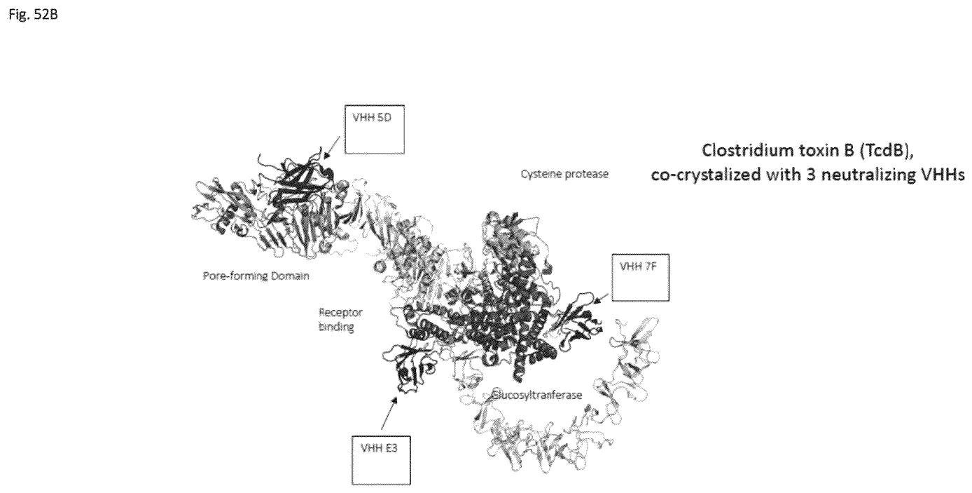

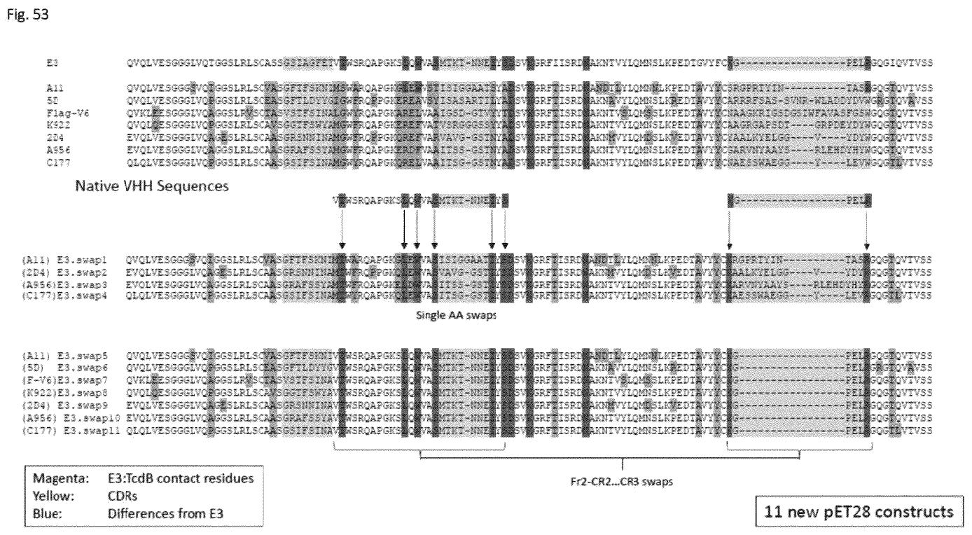

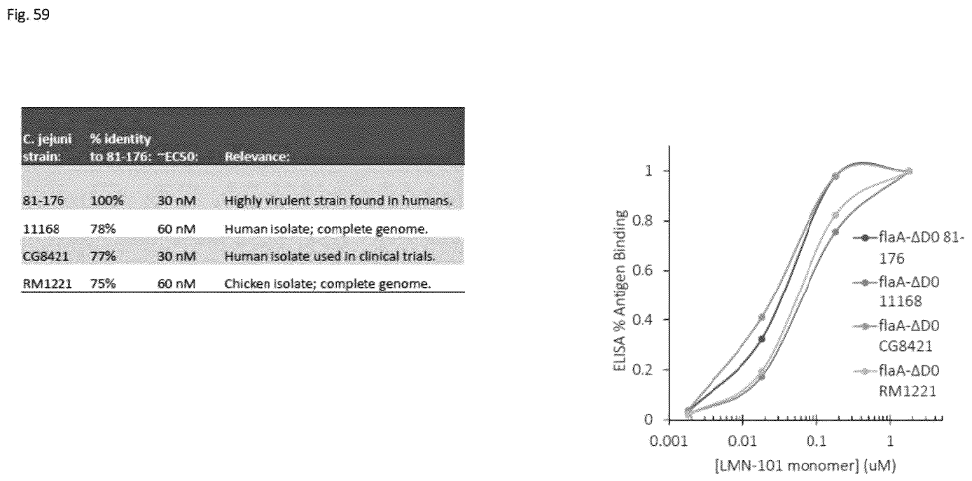

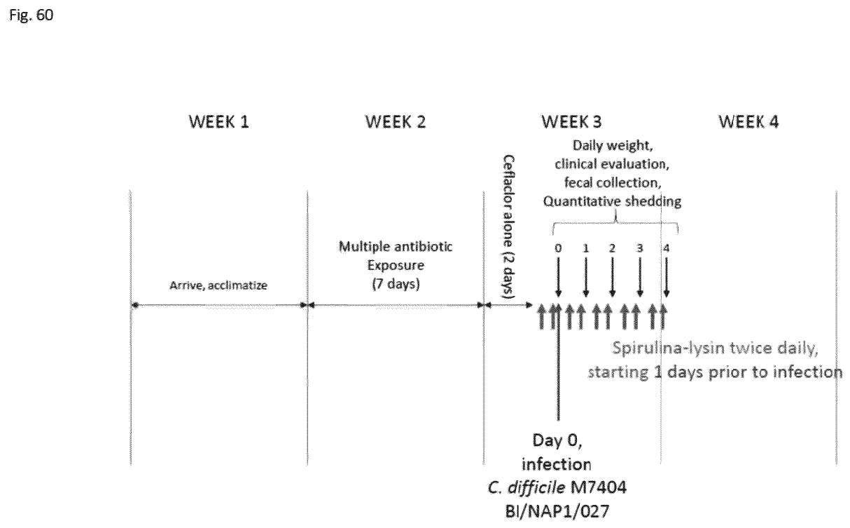

Various constructs were made containing different rigid linkers joining a series of transgenes encoding the anti-TcdB VHH 5D to chaperone partners. (FIG. 51) The particular constructs tested in this experiment are listed in FIG. 59.

The control strain uses a flexible (GGS)x linker between 5D and a computationally designed dimer.

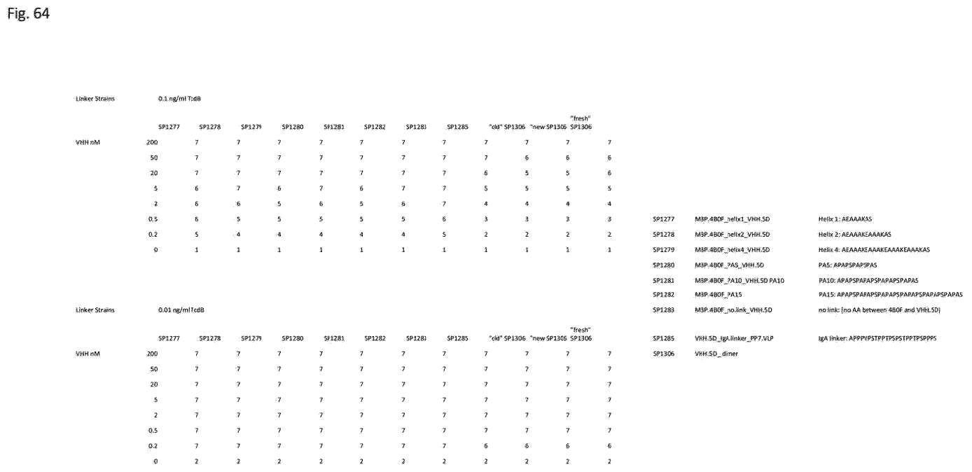

FIG. 64 demonstrates neutralization data for the strains expressing the array of linkers joining 5D to MBP, as well as a single strains with an IgA-derived linker joining 5D and the PP7 VLP.

### Example 21: Stability of Spirulina Constructs in Water and Potable Liquids

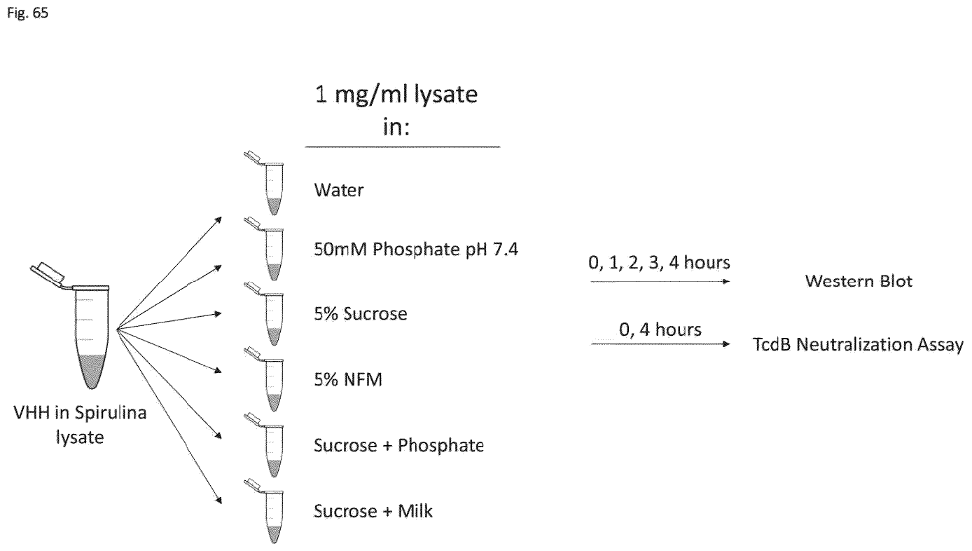

The recombinant Spirulina may be administered orally, and addition of VHHs to drinking water would greatly increase the dose of VHH deliverable to animals. To test the stability and activity of VHHs held at room temperature in various buffers palatable to mice, rats, or pigs, 1 mg/mL Spirulina lysate was mixed into water, 50 mM phosphate pH 7.4, 5% sucrose, 5% Non-fat milk (NFM), sucrose+phosphate, or sucrose+milk. (FIG. 65) Western Blots were performed at 0, 1, 2, 3, and 4 hours. TcdB neutralization assays were performed at 0 and 4 hours.

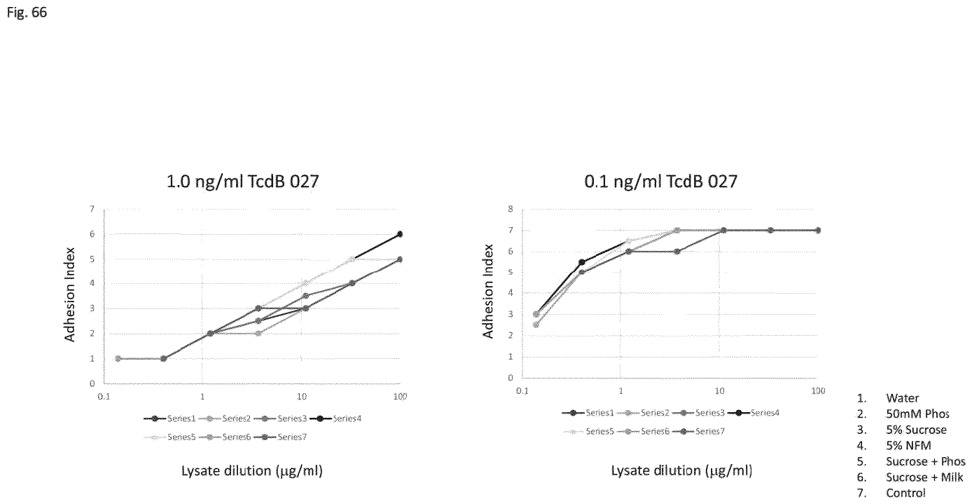

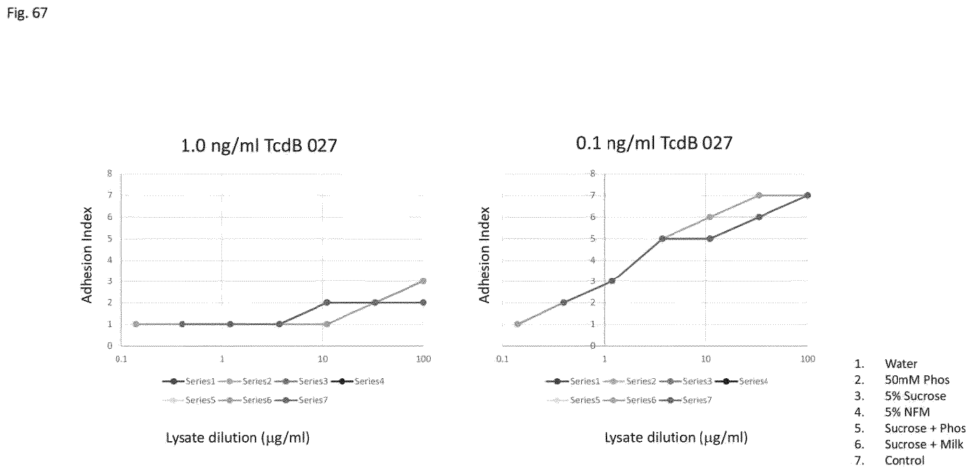

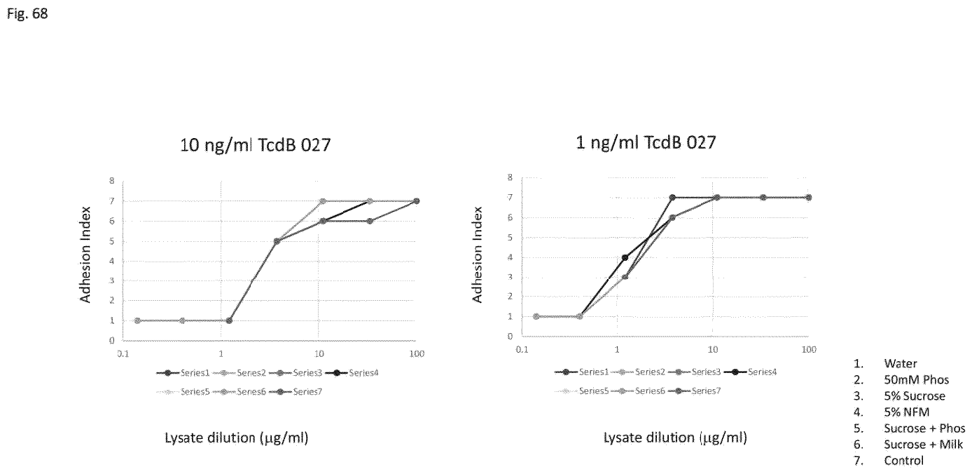

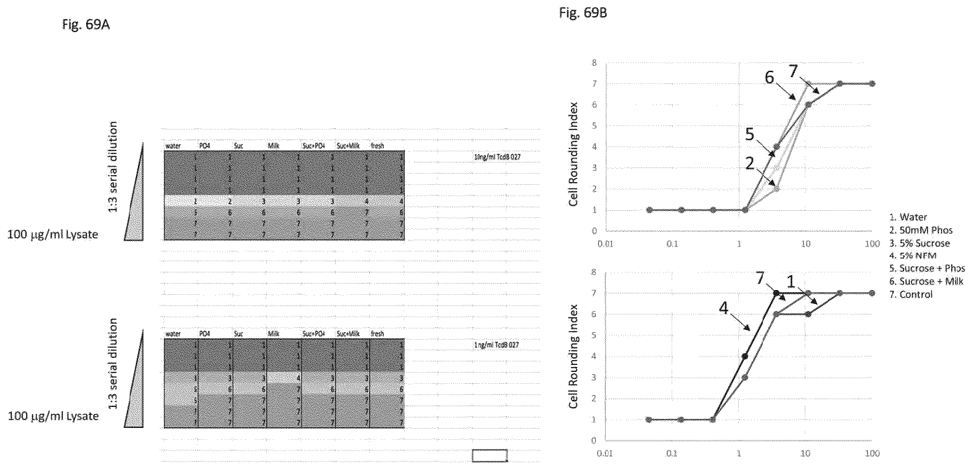

Western blotting showed no decrease in his-tagged protein abundance over the timecourse. No decrease was observed in TcdB-neutralizing potency in any aqueous medium at either the 4 or 12 hour timepoint. (FIG. 66 and FIG. 67) Similar results were obtained for the individual VHHs 5D and E3, and for the 3-way synergistic combination of 5D, E3, and 7F. (FIG. 68).

### Example 22: Study of C. difficile Protection in Gnotobiotic Pig Model

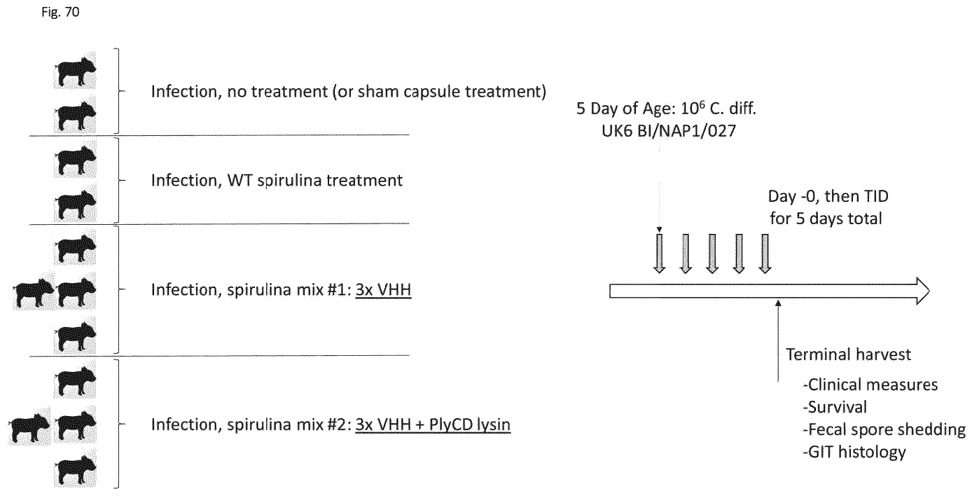

To study the effect of Spirulina expressing anti-TcdB VHHs on protection from C. difficile challenge, the gnotobiotic pig model was used. (FIG. 70) In this study, pigs were divided into 4 groups as indicated below:
Group 1 (two pigs)—infection, no treatment (or sham capsule treatment)
Group 2 (two pigs)—infection, wild-type spirulina treatment
Group 3 (four pigs)—infection, spirulina mix #1: 3×VHH
Group 4 (four pigs)—infection, spirulina mix #2: 3×VHH+PlyCD lysin.

At five days of age, the animals were infected with 10⁶ C. diff. UK6 BI/NAP1/027. After infection, the animals were treated three times a day for five days starting at Day −0. After treatment, animals were measured for clinical measures, survival, fecal spore shedding, and GIT histology.

FIG. 71A-B shows that after day 4, the animals in both Groups 3 and 4 demonstrated reduced incidence of diarrhea compared to infected animals treated with wild type spirulina or PBS.

### Example 23: Study of the Effect of Prophylactic Administration of Anti-TedB VHHs on C. difficile Infection in the Monash Mouse CDI Model

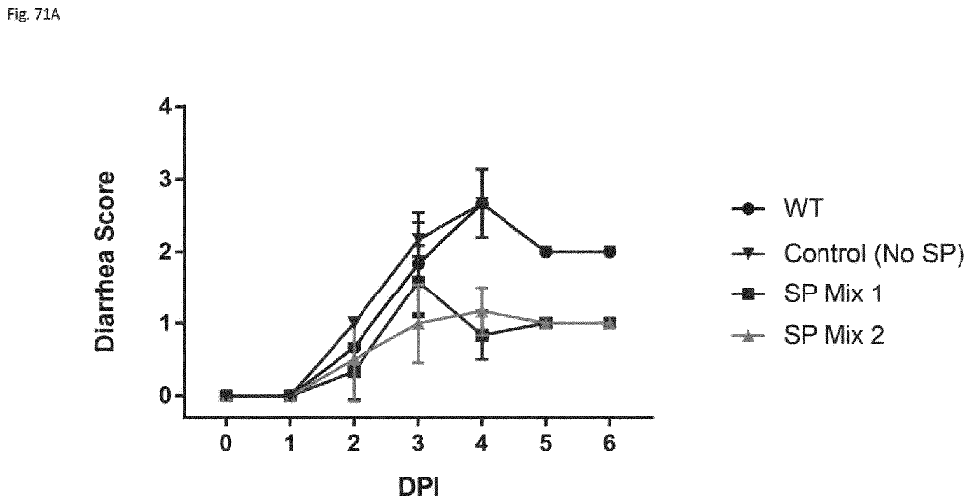

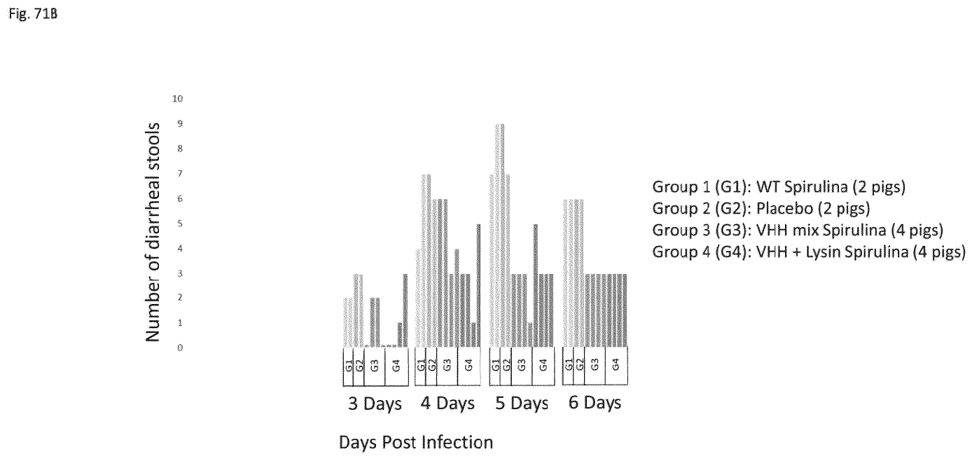

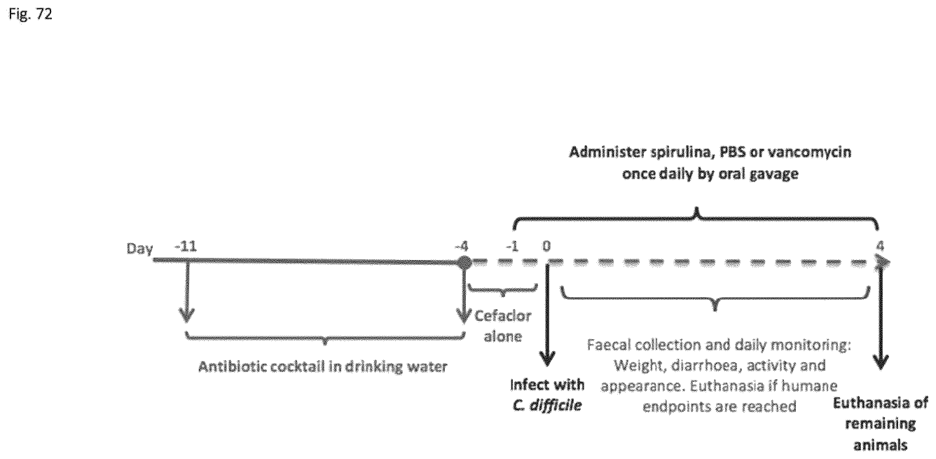

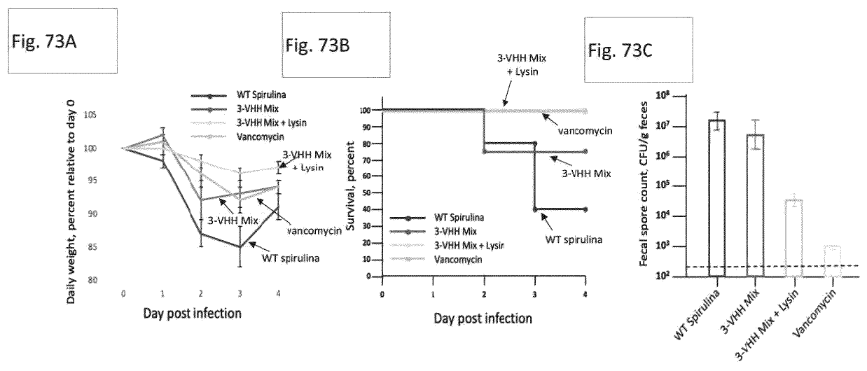

Mice were administered an antibiotic cocktail in the drinking water from Day −11 to Day −4. From Day −4 to Day 0, the mice were administered cefaclor alone, and on Day 0 infected with C. difficile. From day −1 to day 4, mice were administered Spirulina (3×VHH mix or 3×VHH mix+lysin), PBS, or vancomycin once daily by oral gavage. During this period, the mice were monitored daily for weight diarrhea, activity, and appearance, and feces collected. (FIG. 72). Administration of an anti-TcdB VHH mix reduced weight loss associated with C. difficile infection. (FIG. 73A). Mice treated with the VHHs alone had improved survival over those treated with wild type spirulina, and those treated with the 3×VHH mix+PlyCD lysin achieved 100% survival comparable to vancomycin. (FIG. 73B). Finally, administration of the 3×VHH mix+lysin reduced fecal C. difficile spore shedding by >2 logs. (FIG. 73C).

### Example 24: Effect of pH on Release of VHH from LMN-101

Therapeutic VHHs encapsulated in spirulina biomass are not released to gastric-fluid-simulating buffers. Bioencapsulation also prevents enzymatic degradation of VHHs under simulated gastric-digest conditions To analyze the effect of low pH on the release of VHH from spirulina biomass, dried spirulina-VHH biomass was resuspended in different pH buffers. Spray-dried spirulina-VHH biomass used in LMN-101 (strain SP1182) was resuspended in citrate phosphate buffers ranging from pH 3 to pH 7, at 50 mg/mL, and incubated with gentle agitation at room temperature for 60 minutes. Resuspended biomass was clarified by centrifugation at 14,000 RPM for 1 min in a refrigerated microcentrifuge. The clarified extracts were used in an ELISA-based binding assay with recombinant C. jejuni flagellin to determine the amount of aa682 present. High-binding ELISA plates were coated with antigen, and SP1182 extracts were assayed as 4-fold serial dilutions in PBS supplemented with 0.05% Tween-20, and 5% non-fat dried milk. Bound aa682 was detected using a mouse anti-His-tag primary antibody and a goat anti-mouse-HRP secondary antibody.

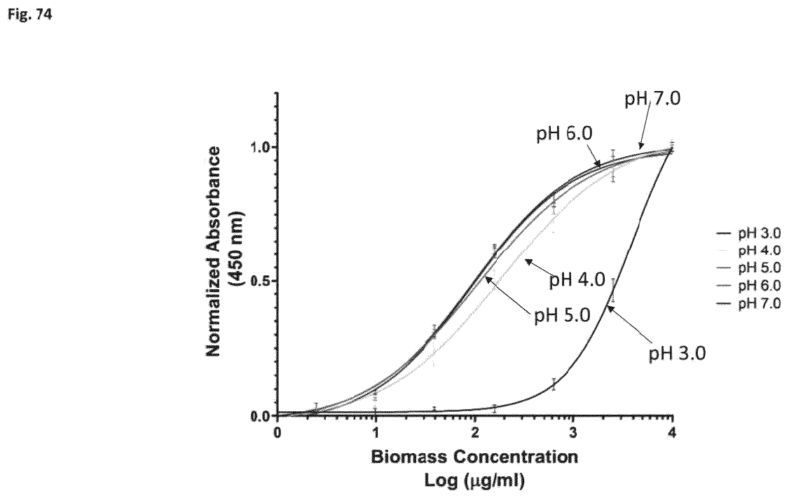

In this ELISA, the relative binding activity of extracts corresponds to the amount of aa682 extracted at each pH. Calculated EC50 values indicated a comparable amount of aa682 binding activity when spirulina biomass was resuspended in pH 5, pH 6, and pH 7 buffer solutions (FIG. 74 and Table 7). The amount of binding activity decreased by 50% when spirulina biomass was extracted in pH 4 buffer. In contrast, the extract prepared in pH 3 buffer demonstrated a relatively small amount of binding activity. The EC50 of extract from biomass resuspended in pH 3 suggested that 40-fold less aa682 was released relative to release in pH 7 buffer. To assess the effect of pH on VHH stability and activity, purified aa682 was incubated in pH 3 buffer and VHH integrity was assessed by an ELISA-based binding assay as above. No measurable loss of binding was observed due to exposure to low pH buffer (data not shown).

TABLE 7
EC50s for SP1182 biomass resuspended in various pH buffers

| Buffer pH: | pH 3 | pH 4 | pH 5 | pH 6 | pH 7 |
|---|---|---|---|---|---|
| EC50 (μg | 3921 | 190.7 | 107.7 | 92.96 | 97.25 |
| biomass/mL) | | | | | |

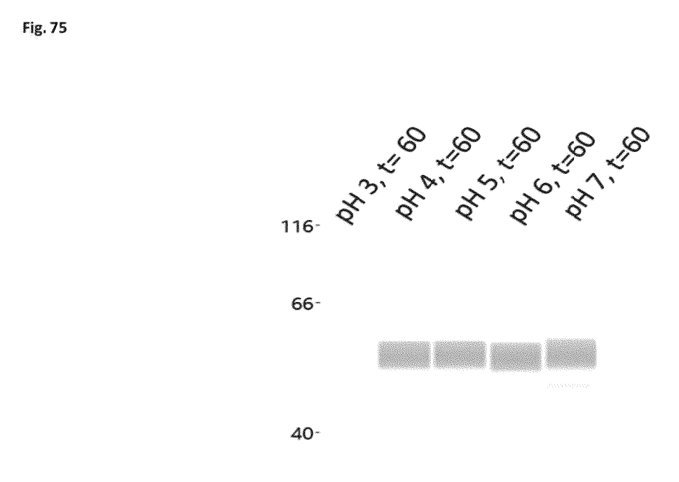

To further demonstrate that the difference in binding activities was the result of a difference in VHH concentration, clarified spirulina extracts were also assayed using a capillary electrophoresis immunoassay. Clarified extracts were prepared as above. VHHs were detected using mouse anti-His-tag primary antibody (Genscript), and HRP-conjugated anti-mouse secondary antibody (ProteinSimple). The amount of VHH protein released from spirulina biomass increased with an increase in pH, with minimal VHH observed at pH 3 (FIG. 75).

These results suggest that at low pH, gastric-like conditions, the VHH may remain encapsulated in the spirulina biomass and protected from the harsh environment of the stomach until transiting to the higher pH conditions of the small intestine.

### Example 25—Phase 1 Clinical Trial of LMN-101

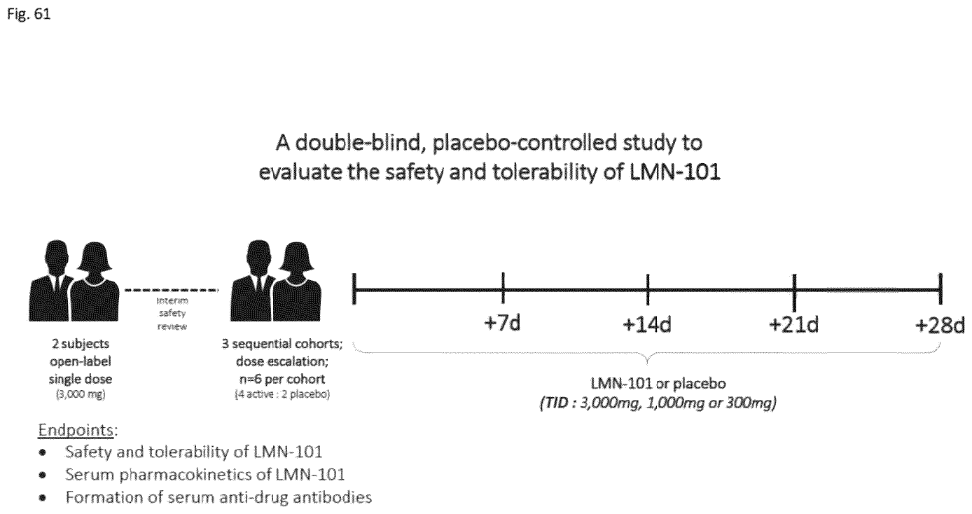

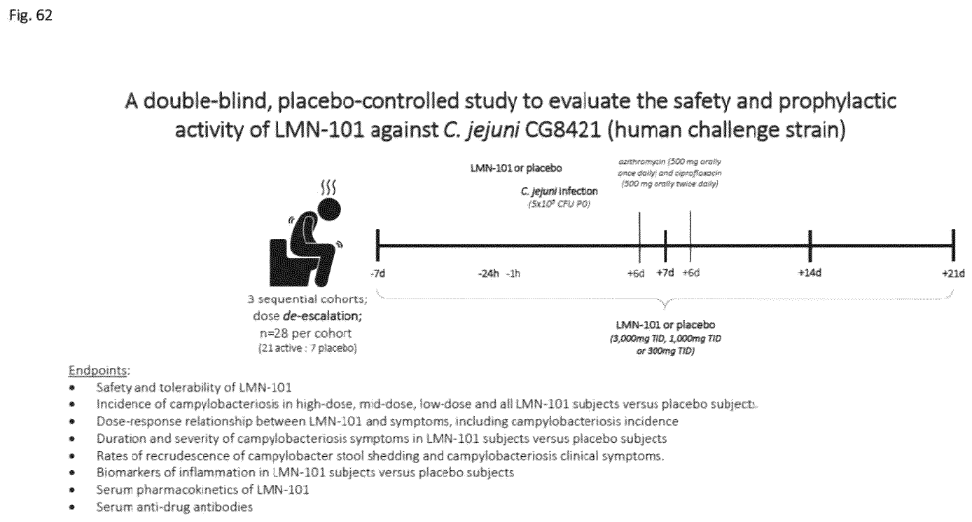

A Phase 1 safety and tolerability study was conducted in healthy volunteers with LMN-101 (SP1182), a single spirulina strain that has been engineered to express a binding protein that inhibits C. jejuni (CG8421) infection. Part A of the study was an open-label oral administration of a single 3000-mg dose of LMN-101. Part B was a randomized, double-blind, placebo-controlled, dose-escalation study of 3 dose levels of LMN-101: 300 mg, 1000 mg, or 3000 mg. (FIG. 61 & FIG. 62) Wild type Spirulina was used as a control. In Part B, healthy volunteers took LMN-101 or placebo orally at one of these three dose levels three times daily for 28 days. No significant adverse events were reported. In addition, pharmacokinetic data showed that there was no significant systemic absorption, indicating that the Spirulina was able to pass through the stomach and deliver the VHH to the gastrointestinal tract. Orally delivered LMN-101 was safe and well tolerated at doses up to 3000 mg TID for 28 days, and no significant adverse events due to LMN-101 were observed.

### Example 26: In Vitro Stability of Spirulina-VHH Biomass in Simulated Intestinal Fluids

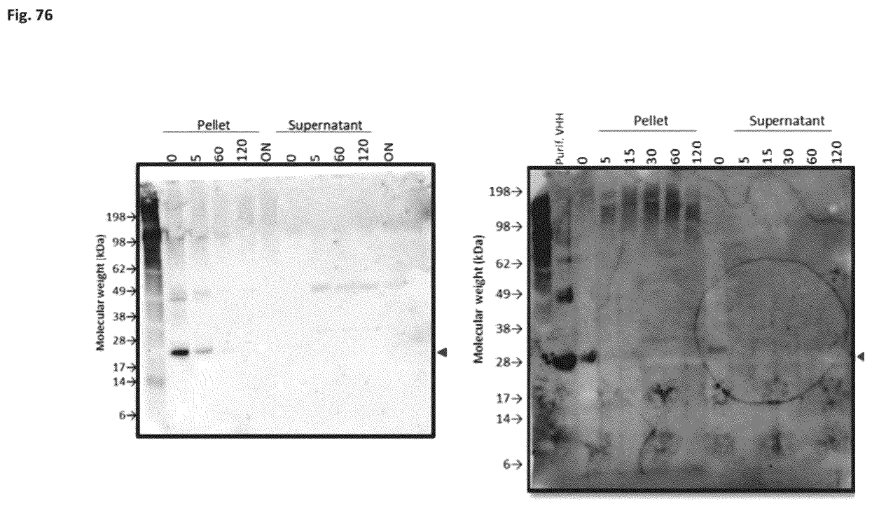

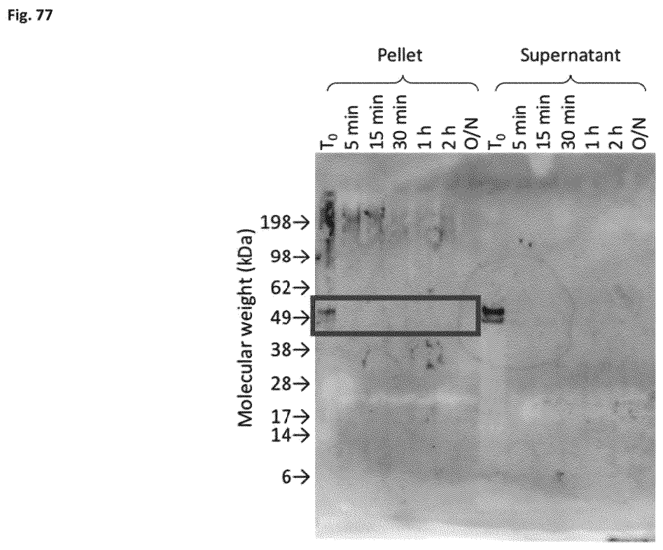

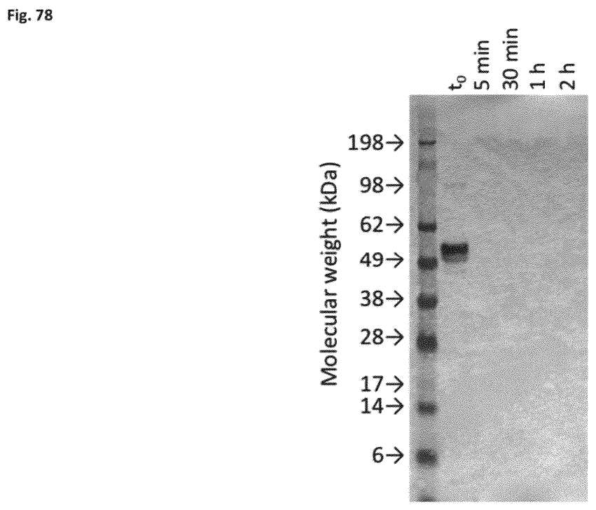

To model the intestinal phase of delivery, dried spirulina-VHH biomass was incubated in simulated intestinal fluid (SIF): 50 mM citrate-phosphate buffer, pH 7.0, 164 mM NaCl, 85 mM NaHCO₃, 3 mM CaCl₂, and 1 mg/L Pancreatin with 10 mM Porcine Bile Extract, incubated at 37° C. Integrity of the intact anti-campylobacter-binding protein was determined by western blot. In two independent experiments using a dried biomass of anti-campylobacter spirulina-VHH expressing a trimeric VHH (strain SP806), it was observed that more than 80% of the binding protein was released from the biomass within 5 minutes and more than 95% within 30 minutes (FIG. 76). In a similar experiment using the spirulina-VHH present in LMN-101 (strain SP1182), more than 95% of the binding protein was released in 5 minutes (FIG. 77). A fully intact released binding protein did not accumulate to measurable levels in the simulated intestinal fluid in either case, indicating that its rate of proteolytic cleavage was faster than its release rate. The limit of detection in this experiment was approximately 20% recovery of released, intact anti-campylobacter-binding protein. Consistent with this interpretation, purified anti-campylobacter-binding protein added directly to the simulated intestinal fluid had a half-life for proteolytic cleavage of less than 5 minutes (FIG. 78).

The rapid release in the simulated intestinal environment suggests that aa682 is released in the proximal small intestine and be accessible to bind campylobacter in that milieu. The rapid degradation of aa682 suggests that detectable levels does remain in fecal contents.

### Example 27: In Vitro Stability of Spirulina-VHH Biomass in Simulated Gastric Fluids

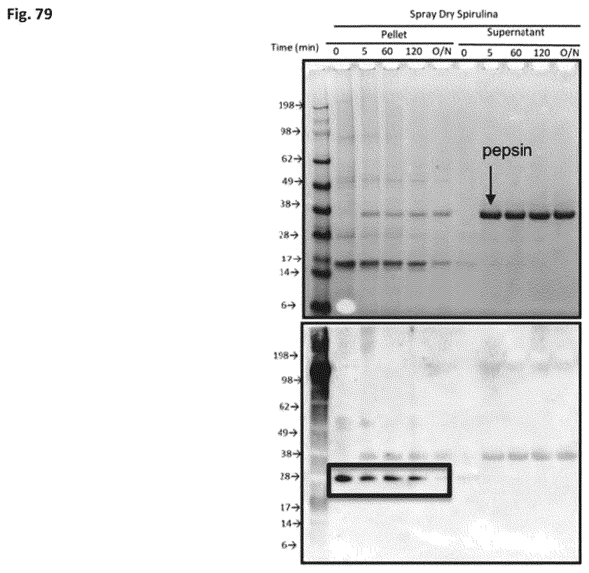

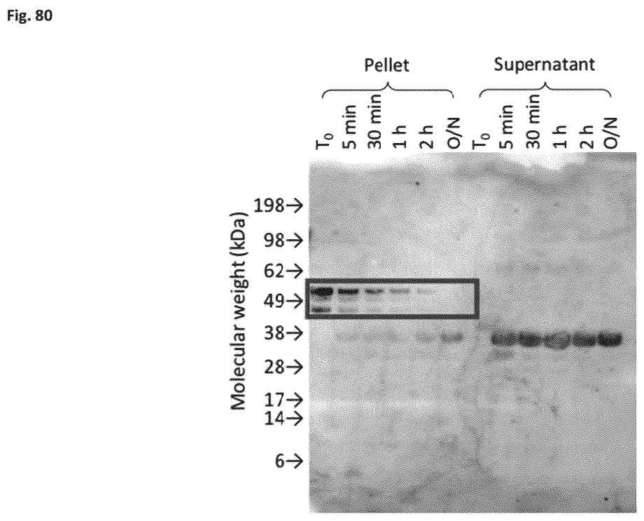

Spirulina biomass protects the campylobacter-binding protein while in transit through the harsh environment of the stomach. Dried biomass of an anti-campylobacter spirulina-VHH was incubated in a simulated gastric fluid (SGF): 10 mM citrate-phosphate buffer, pH 3.5, 94 mM NaCl, 13 mM KCl, and 2,000 units/mL pepsin, incubated at 37° C. Western blotting of digested spirulina-VHH biomass demonstrated that the campylobacter-binding protein expressed within this biomass was 50% intact for 120 minutes (FIG. 79). The analysis was repeated with the spirulina-VHH present in LMN-101 (strain SP1182) but otherwise under identical conditions. Western blotting demonstrated that the campylobacter-binding protein expressed within this biomass was 20% intact after 120 minutes (FIG. 80).

### Example 28: Intranasal Administration of SP648 Elicits Production of Antibodies in Murine Model

Mice were tested to determine whether intranasal administration of a Spirulina expressing a malarial antigen, NANP, or Spirulina extract containing the malarial antigen, NANP, demonstrate an IgG response to NANP. The mice were further analyzed for survival of a malaria infection.

Mice were immunized with PfCSP-VLP (SP648—a malaria vaccine based on the NANP repeat region of P. falciparum CSP fused within a virus-like particle or empty VLP (SP79). Mice were assigned to 6 groups (5 mice/group) and treated as indicated in Table 8.

TABLE 8

| | | DAY | | | | | | | | | | |
|---|---|---|---|---|---|---|---|---|---|---|---|---|
| | | Day −1 | D 0 | D 14 | Day 27 | D 28 | Day 41 | D 42 | Day 56 | D 57 | Day 69 | D 70 |
| Grp | Oral PfCSP-VLP Spirulina −> 3x Oral | Draw | Prime | Draw | Draw | Boost 1 | Draw | Boost 2 | Draw | Boost 3 | Draw | Challenge |
| 1 | PfCSP-VLP Spirulina | | | | | | | | | | | |
| Grp | Intranasal PfCSP-VLP Spirulina −> 3x | Draw | Prime | Draw | Draw | Boost 1 | Draw | Boost 2 | Draw | Boost 3 | Draw | Challenge |
| 2 | Oral PfCSP-VLP Spirulina | | | | | | | | | | | |
| Grp | Intranasal PfCSP-VLP Extract −> 3x | Draw | Prime | Draw | Draw | Boost 1 | Draw | Boost 2 | Draw | Boost 3 | Draw | Challenge |
| 3 | Oral PfCSP-VLP Spirulina | | | | | | | | | | | |
| Grp | Oral Control-VLP Spirulina −> 3x | Draw | Prime | Draw | Draw | Boost 1 | Draw | Boost 2 | Draw | Boost 3 | Draw | Challenge |
| 4 | Oral Control-VLP Spirulina | | | | | | | | | | | |
| Grp | Intranasal Control-VLP Spirulina −> IN | Draw | Prime | Draw | Draw | NO Tx | Draw | RE- | Draw | Boost 1 | Draw | Challenge |
| 5 | PfCSP Extract −> Oral PfCSP | | | | | | | Prime | | | | |
| | Spirulina | | | | | | | IN | | | | |
| | | | | | | | | sp648 | | | | |
| Grp | Intranasal Control Extract −> IN | Draw | Prime | Draw | Draw | RE- | Draw | Boost 1 | Draw | Boost 2 | Draw | Challenge |
| 6 | PfCSP extract −> 2x Oral PfCSP | | | | | Prime | | | | | | |
| | Spirulina | | | | | IN | | | | | | |
| | | | | | | sp648 | | | | | | |

PfCSP-VLP whole biomass resuspended in PBS-oral administration (PO)
PfCSP-VLP whole biomass resuspended in PBS-intranasal administration (IN)
PfCSP-VLP extract-intranasal administration (IN)
Empty VLP whole biomass resuspended in PBS-oral administration (PO)
Empty VLP whole biomass resuspended in PBS-intranasal administration (IN)
Empty VLP extract-intranasal administration (IN)

Groups 5 and 6 both had a period of re-priming on a day Groups 1-4 may have received a boost. At the time Groups 1-4 received a first boost, Group 5 was not treated. When Groups 1-4 received their second boost, Group 5 was given a “Re-priming” with intranasal administration of PfCSP-VLP extract which was followed by one boost with orally administered PfCSP Spirulina biomass. At the time Groups 1-4 received their first boost, Group 6 was given a “Re-priming” with intranasal administration of PfCSP-VLP extract which was followed by two boosts with orally administered PfCSP Spirulina biomass. This re-priming was done to determine if the number of boosts administered to the mice influenced IgG production. Group 3 received three oral boosts; Group 6 received two; and Group 5 received only one.

### IgG Measurement

Sera was collected at days 14, 27, 41, 56, and 69 as indicated in Table 8. The amount of IgG produced in the different groups was measured by indirect ELISA. The antigen coated on the plate, NANP, is covered by mouse sera containing varying amounts of antibodies specific for the NANP antigen followed by a secondary antibody that is conjugated to horseradish peroxidase (HRP). The substrate was added in the presence of hydrogen peroxide as an indirect way to measure how much antibody specific for NANP is present in each serum sample. ELISAs were performed on serial dilutions of the sera of each animal to detect the lowest amount of sera that can still generate a positive response to the antigen.

### Results

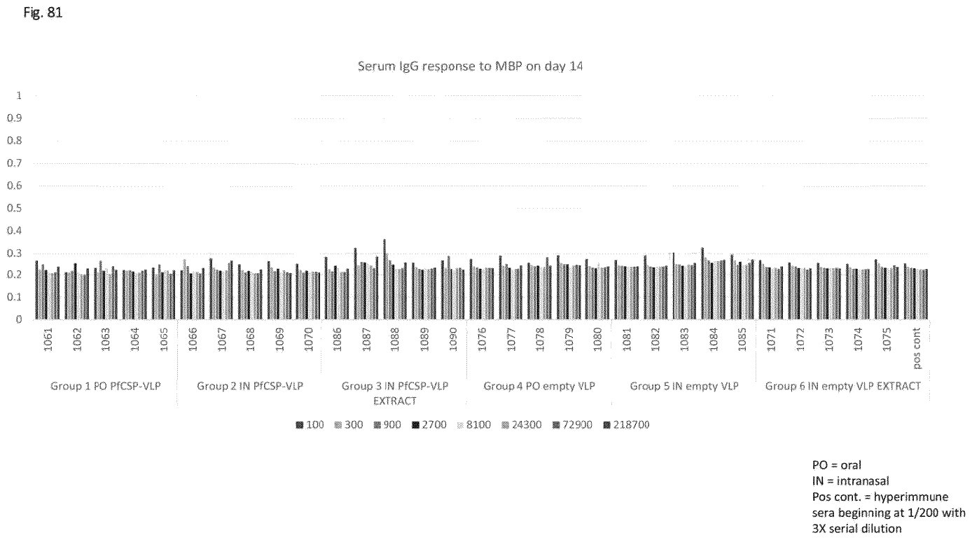

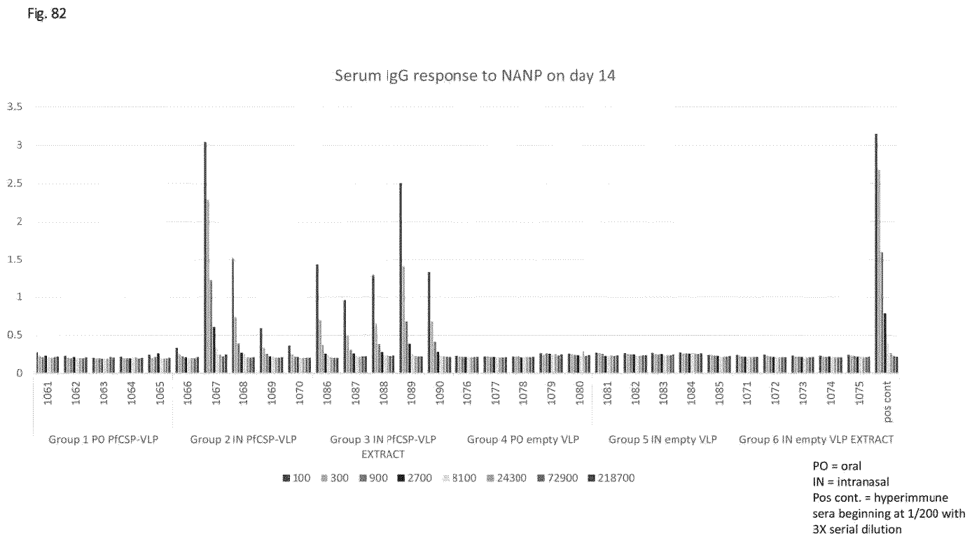

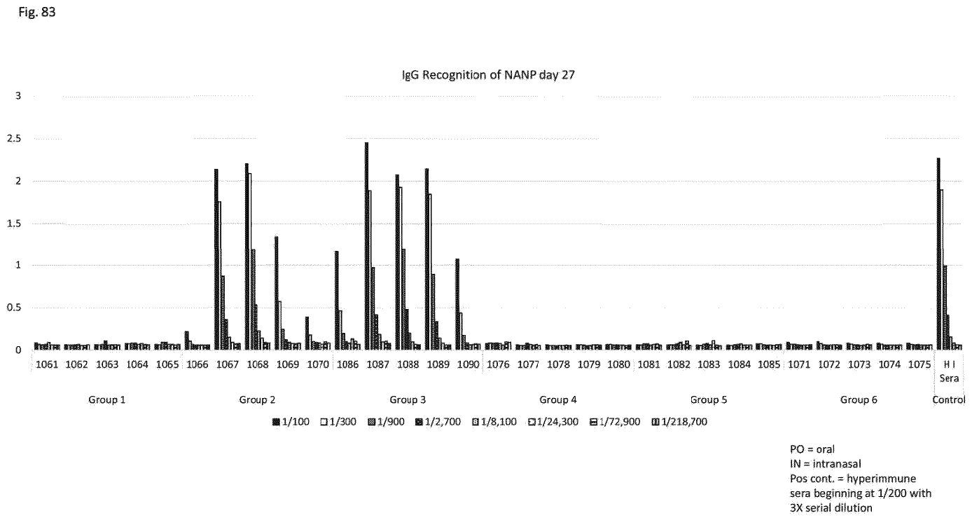

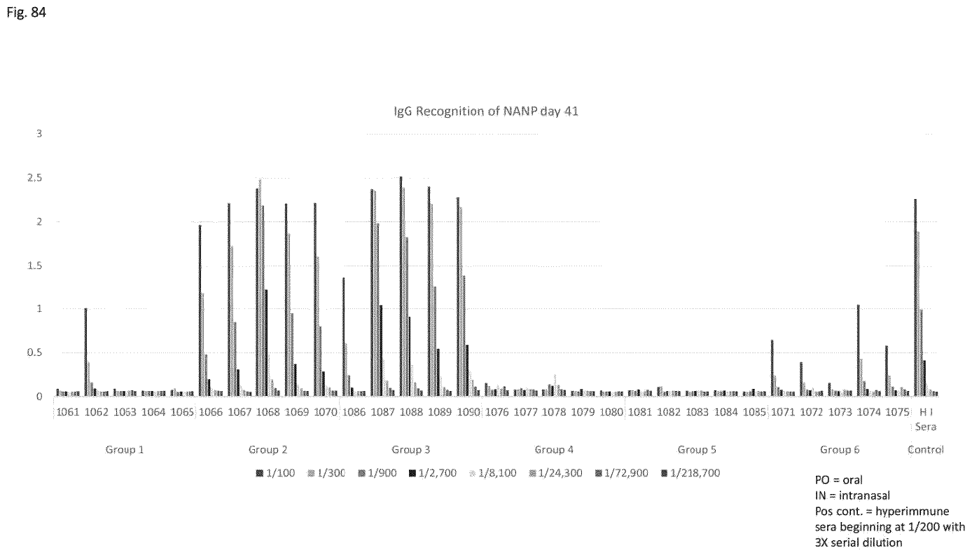

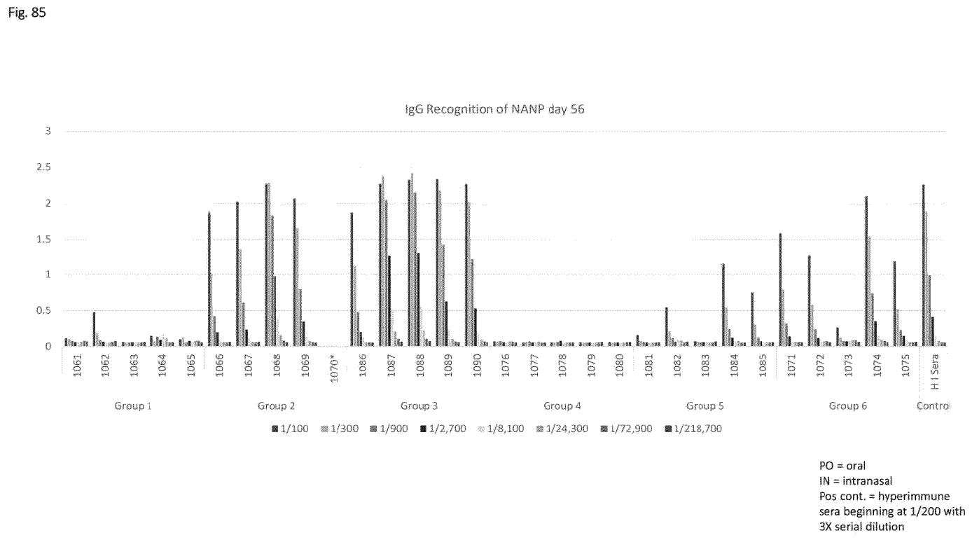

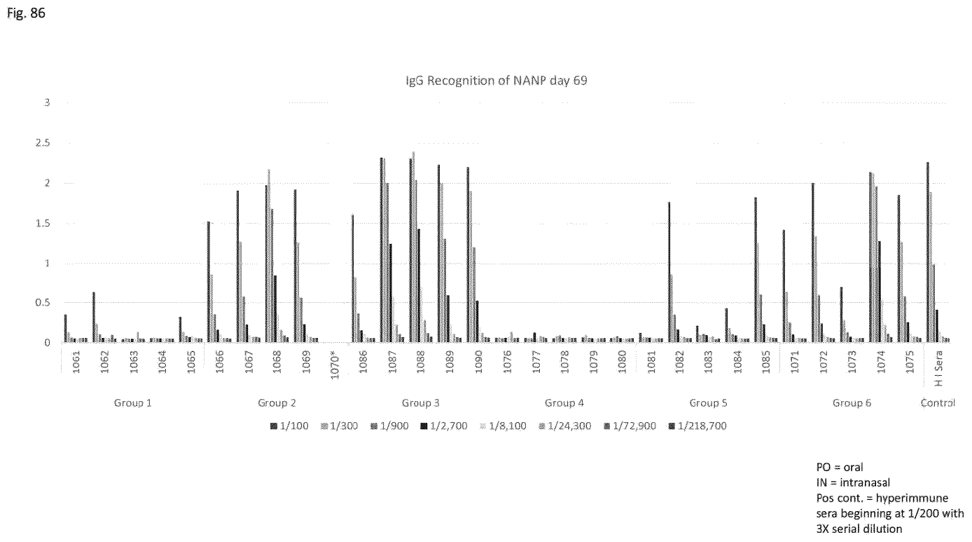

The results shown in FIGS. 81 to 86 show the serum IgG response NANP at various timepoints after administration of the malaria vaccines or controls as outlined above. Measuring IgG response to Maltose Binding Protein (MBP) acted as the control. The Y axis on each is the absorbance value measure from the plate reader. Positive responses are those that approach the amount of IgG found in the positive control, hyperimmune sera. The dilutions for the hyperimmune sera are different from those for the experimental groups since the hyperimmune sera is so potent, and greater amounts are not required to detect IgG.

The data shown in FIG. 76 (day 14) measures serum IgG response against a different substrate (MBP) as a control. Since the NANP protein is fused to MBP, it is important that the sera not be reacting to MBP. FIG. 76 shows no IgG response to MBP at Day 14 after vaccination with the malaria vaccines tested here. Similar results were obtained for the other days tested (data not shown).

Previous reports demonstrated that after oral administration, mice showed production of IgG in response to NANP by 28 days, but as seen here, not by day 14. In contrast, intranasal administration offers a fairly robust serum IgG response against NANP even by day 14.

As shown in FIG. 81-FIG. 86, the serum IgG production in the mice of Group 3 was more uniform than that of Group 2 which may reflect the difference between administration of an extract and administration of a resuspended biomass. The extract is a homogenous solution, whereas the resuspended biomass may not be, leading mice within a given group to inadvertently receive different amounts of Spirulina.

Further, mice inoculated with the extract are readily exposed to the vaccine antigen, whereas those administered the Spirulina biomass might not be exposed to the vaccine antigen as efficiently or evenly. The encapsulation of the vaccine antigen in the Spirulina may not be an important component of vaccines administered nasally as opposed to orally where protection of the vaccine is important for crossing the stomach.

Importantly, nasal administration of extract yields a more robust and uniform response than administration of Spirulina biomass, whether given orally or intranasaly.

Challenge with Malaria

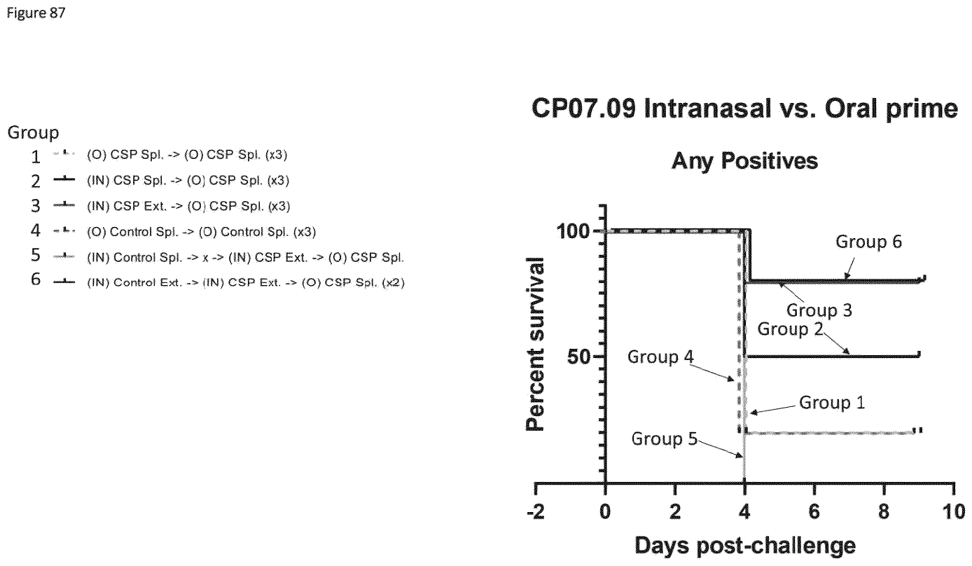

FIG. 87 shows the survival rate of the various groups after challenge with P. falciuparum.

Some mice appear to be protected from challenge even though they have a lower detectable serum IgG response indicating other elements play a role in the immune response, including other types of antibody response. The data shown here looks only at serum IgG—the mice also produce serum IgA and IgM. Further, an analysis of fecal samples will yield information regarding mucosal IgA which is an indicator that there has been a good mucosal response. Generally, however, a high serum IgG titer indicates protection from challenge, and indeed the 50% protection observed in Group 2 is quite good as it is hard to protect a mouse from malaria. Thus, the demonstrated protection of up to 80% is surprising.

### Examples of Non-Limiting Embodiments of the Disclosure

Embodiments, of the present subject matter disclosed herein may be beneficial alone or in combination, with one or more other embodiments. Without limiting the foregoing description, certain non-limiting embodiments of the disclosure, are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

Embodiment 1. A non-parenterally delivered composition comprising a recombinant Spirulina, wherein the recombinant Spirulina comprises at least one therapeutic or prophylactic molecule.

Embodiment 2. The non-parenterallydelivered composition of embodiment 1, wherein the therapeutic or prophylactic molecule is delivered to the gastrointestinal tract.

Embodiment 3. The non-parenterally delivered composition of embodiment 1, wherein the therapeutic or prophylactic molecule is delivered systemically.

Embodiment 4. The non-parenterally delivered composition of any of embodiments 1-3, wherein the therapeutic or prophylactic molecule is an endogenous Spirulina molecule.

Embodiment 5. The non-parenterally delivered composition of embodiment 4, wherein the endogenous Spirulina molecule is found in higher concentrations than found in naturally-occurring Spirulina.

Embodiment 6. The non-parenterally delivered composition of any of embodiments 1-3 wherein the therapeutic or prophylactic molecule is exogenous to Spriulina.

Embodiment 7. The non-parenterally delivered composition of embodiment 6, wherein the exogenous molecule is produced by a different bacteria or plant.

Embodiment 8. The non-parenterally delivered composition of embodiment 7, wherein the exogenous therapeutic is a malacidin.

Embodiment 9. The non-parenterally delivered composition of embodiment 6, wherein the exogenous molecule is a polypeptide or a fragment thereof.

Embodiment 10. The non-parenterallydelivered composition of embodiment 9, wherein the exogenous polypeptide is an antibody or fragment thereof.

Embodiment 11. The non-parenterally delivered 1 composition of embodiment 10, wherein the antibody or fragment thereof is selected from the group consisting: of full length antibody, a monospecific antibody, a bispecific antibody, a trispecific antibody, an antigen-binding region, heavy chain, light chain, VHH, VH, VL, a CDR, a variable domain, scFv, Fc, Fv, Fab, F(ab)₂, reduced IgG (rIgG), monospecific Fab₂, bispecific Fab₂, trispecific Fab₃, diabody, bispecific diabody, trispecific triabody, minibody, IgNAR, V-NAR, HcIgG, or a combination thereof.

Embodiment 12. The non-parenterally delivered composition of embodiment 9, wherein the exogenous polypeptide is selected from the group consisting of: insulin, C-peptide, amylin, interferon, a hormone, a receptor, a receptor agonist, a receptor antagonist, an incretin, GLP-1, glucose-dependent insulinotropic peptide (GIP), an immunomodulatory, an immunosuppressor, a peptide chemotherapeutic, an anti-microbial peptide, magainin, NRc-3, NRC-7, buforin IIb, BR2, p16, Tat, TNFalpha, and chlorotoxin.

Embodiment 13. The delivered composition of embodiment 9, wherein the exogenous polypeptide is an antigen or epitope.

Embodiment 14. The non-parenterally delivered composition of embodiment 13, wherein the antigen or epitope is derived from an infectious microorganism, a tumor antigen or a self-antigen associated with an autoimmune disease

Embodiment 15. The non-parenterally delivered composition of any of embodiments 1-14, wherein administration of the recombinant Spirulina to a subject prevents, treats or ameliorates a disease or disorder.

Embodiment 16. The non-parenterally delivered composition of embodiment 15, wherein the disease or disorder is selected from the group consisting of: Type 1 diabetes, Type 2 diabetes, cancer, an inflammatory disorder, a gastrointestinal disease, an autoimmune disease or disorder, an endocrine disorder, gastroesophageal reflux disease (GERD), ulcers, high cholesterol, inflammatory bowel disorder, irritable bowel syndrome, crohn's disease, ulcerative colitis, constipation, vitamin deficiency, iron deficiency, and diarrhea.

Embodiment 17. The non-parenterally delivered composition of embodiment 15, wherein administration of the recombinant Spirulina to a subject treats, prevents, or ameliorates an infection.

Embodiment 18. The non-parenterally delivered composition of embodiment 17, wherein the infection is bacterial, viral, fungal, or parasitical.

Embodiment 19. The non-parenterallydelivered composition of embodiment 18, wherein the bacteria causing the infection is selected from the group consisting of: E. coli, Enterotoxigenic E. coli (ETEC), Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella, heliobacteter, anthrax, ETEC, EHEC, EAEC, and Legionella.

Embodiment 20. The non-parenterally delivered composition of embodiment 18, wherein the virus causing the infection is selected from the group consisting of: bacteriophage, RNA bacteriophage (e.g. MS2, AP205, PP7 and Qβ), Helicobacter pylori, Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, White Spot Syndrome Virus, a coronavirus, MERS, SARS, and SARS-CoV-2.

Embodiment 21. The non-parenterally delivered composition of embodiment 18, wherein the fungus causing the infection is selected from the group consisting of: Aspergillus, Candida, Blastomyces, Coccidioides, Cryptococcus, and Histoplasma.

Embodiment 22. The non-parenterally delivered composition of embodiment 18, wherein the parasite causing the infection is selected from the group consisting of: Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. and Wuchereria spp., Taenia spp., Echinococcus spp., and Diphyllobothrium spp., Fasciola spp., and Schistosoma spp..

Embodiment 23. The non-parenterallydelivered composition of any of embodiments 9-22, wherein the exogenous polypeptide or a fragment thereof is in a fusion protein.

Embodiment 24. The non-parenterally delivered composition of any of embodiments 9-22, wherein the recombinant Spirulina comprises a nucleic acid encoding the exogenous polypeptide or fragment thereof.

Embodiment 25. The non-parenterally delivered composition of embodiment 24, wherein at least 2, at least 3, at least 4, or at least 5 copies of a nucleic acid sequence encoding the at least one exogenous polypeptide or fragment thereof are present in the recombinant Spirulina.

Embodiment 26. The non-parenterally delivered composition of any of embodiments 24-25, wherein 2, 3, 4, 5, 6, 8, 10, 15, 20, 25, 30, 40, or 50 copies of a nucleic acid sequence encoding the at least one exogenous polypeptide or fragment thereof are present in the recombinant Spirulina.

Embodiment 27. The non-parenterally delivered composition of embodiment 25, wherein at least 2, at least 3, at least 4, or at least 5 copies of the at least one exogenous polypeptide or fragment thereof are present in a single molecule of the exogenous polypeptide expressed in the recombinant Spirulina.

Embodiment 28. The non-parenterally delivered composition of embodiment 25 or 27, wherein 2, 3, 4, 5, 6, 8, 10, 15, 20, 25, 30, 40, or 50 copies of the at least one exogenous polypeptide or fragment thereof are present in a single molecule of the exogenous polypeptide expressed in the recombinant Spirulina.

Embodiment 29. The non-parenterally delivered composition of any of embodiments 25 or 27-28, wherein, within the molecule of the exogenous polypeptide, the copies of the exogenous polypeptide are linked in tandem.

Embodiment 30. The non-parenterally delivered composition of any of embodiments 25 or 27-28, wherein, within the molecule of exogenous polypeptide or fragment thereof, the copies of the exogenous polypeptide or fragment thereof are separated by a spacer sequence.

Embodiment 31. The non-parenterally delivered composition of any of embodiments 25-30, wherein, within the molecule of exogenous polypeptide or fragment thereof, some of the copies of the exogenous polypeptide or fragment thereof are linked in tandem and the remaining copies of the exogenous polypeptide or fragment thereof are separated by a spacer sequence.

Embodiment 32. The non-parenterally delivered composition of embodiment 30 or 31, wherein the spacer sequence is between about 1 and 50 amino acids long.

Embodiment 33. The non-parenterally delivered composition of any of embodiments 30-32, wherein more than one spacer sequence is present within the molecule of the exogenous polypeptide or fragment thereof.

Embodiment 34. The non-parenterally delivered composition of any one of embodiments 9-34, wherein the recombinant Spirulina comprises at least 2, at least 3, at least 4, or at least 5 different exogenous polypeptides or fragments thereof.

Embodiment 35. The non-parenterallydelivered composition of any one of embodiments 23-34, wherein the fusion protein comprises a carrier protein.

Embodiment 36. The non-parenterally delivered composition of embodiment 35, wherein the carrier protein is selected from the group consisting of: maltose binding protein, hedgehog hepatitis virus-like particle, thioredoxin, and phycocyanin.

Embodiment 37. The non-parenterally delivered composition of any one of embodiments 23-36, wherein the fusion protein comprises a scaffold protein.

Embodiment 38. The non-parenterally delivered composition of embodiment 37, wherein the at least one exogenous polypeptide is linked to a scaffold protein at the N-terminus or the C-terminus, or in the body of the scaffold protein.

Embodiment 39. The non-parenterally delivered composition of embodiment 37 or 38, wherein the scaffold protein is selected from the oligomerization domain of C4b-binding protein (C4BP), cholera toxin b subunit, or oligomerization domains of extracellular matrix proteins.

Embodiment 40. The non-parenterally delivered composition of any of embodiments 37-39, wherein the at least one exogenous polypeptide and the scaffold protein are separated by about 1 to about 50 amino acids.

Embodiment 41. The non-parenterally delivered composition of any of embodiments 37-40, wherein the fusion protein comprises multiple copies of the at least one exogenous polypeptide or fragment thereof, wherein the at least one exogenous polypeptide or fragment thereof and the scaffold protein are arranged in any one of the following patterns: (E)n-(SP), (SP)-(E)n, (SP)-(E)n-(SP), (E)n1-(SP)-(E)n2, (SP)-(E)n1-(SP)-(E)n2, and (SP)-(E)n1-(SP)-(E)n2-(SP), wherein E is the at least one exogenous polypeptide or fragment thereof, SP is the scaffold protein, n, n1, and n2 represent the number of copies of the at least one exogenous polypeptide or fragment thereof.

Embodiment 42. The non-parenterally delivered composition of any of embodiments 9-42, wherein the recombinant Spirulina comprises an anti-Campylobacter VHH.

Embodiment 43. The non-parenterally delivered composition of embodiment 42, wherein the campylobacter is a C. jejuni.

Embodiment 44. The non-parenterally delivered composition of any of embodiments 42-43, wherein the VHH binds to a campylobacter component.

Embodiment 45. The non-parenterally delivered composition of embodiment 44, wherein the VHH binds flagellin.

Embodiment 46. The non-parenterally delivered composition of any of embodiments 42-45, wherein administration increases Campylobacter shedding.

Embodiment 47. The non-parenterally delivered composition of any of embodiments 42-46, wherein administration reduces the levels of biomarkers.

Embodiment 48. The non-parenterally delivered composition of embodiment 47, wherein the biomarker is an inflammation biomarker.

Embodiment 49. The non-parenterally delivered composition of any of embodiments 9-42 wherein the recombinant Spirulina comprises a VHH that binds to an anti-Clostridium toxin.

Embodiment 50. The non-parenterally delivered composition of embodiment 49, wherein Clostridium is C. difficile.

Embodiment 51. The non-parenterally delivered composition of any one of embodiments 48-49, wherein the VHH binds to a Clostridium component, toxin A, or toxin B.

Embodiment 52. The non-parenterallydelivered composition of any of embodiments 49-51, wherein the VHH comprises the amino acid sequence of any of SEQ ID NO:s 5-10.

Embodiment 53. The non-parenterally delivered composition of any of embodiments 1-52, wherein the therapeutic or prophylactic molecule is monomeric.

Embodiment 54. The non-parenterally delivered composition of any of embodiments 1-52, wherein the therapeutic or prophylactic molecule is multimeric.

Embodiment 55. The non-parenterally delivered composition of embodiment 54, wherein the therapeutic or prophylactic molecule is trimeric.

Embodiment 56. The non-parenterally delivered composition of any of embodiments 54-55, wherein the multimer is heteromeric.

Embodiment 57. The non-parenterally delivered composition of any of embodiments 54-55, wherein the multimer is homomeric.

Embodiment 58. The non-parenterally delivered composition of any of embodiments 54-57, wherein the multimer is arranged in a nanoparticle.

Embodiment 59. The non-parenterally delivered composition of any of embodiments 54-57, wherein the multimer binds to a target or target molecule at a high affinity.

Embodiment 60. The non-parenterally delivered composition of embodiment 59, wherein the multimer binding affinity is greater than that of a monomer or a dimer.

Embodiment 61. The non-parenterally delivered composition of embodiment 60, wherein the multimer has an EC₅₀ of over 5 μg/mL.

Embodiment 62. The non-parenterally delivered composition of embodiment 61, wherein the multimer has an EC₅₀ of over 10 μg/mL.

Embodiment 63. The orally delivered composition of embodiment 61, wherein the multimer has an EC₅₀ of about 5 μg/mL to about 40 μg/mL.

Embodiment 64. The non-parenterally delivered composition of any of embodiments 59-63, wherein the multimer binding affinity is greater than that of a multimer comprising fewer copies of the exogenous therapeutic or fewer copies of combinations of exogenous therapeutics.

Embodiment 65. The non-parenterally delivered composition of any of embodiments 59-64, wherein administration of Spirulina comprising multimeric exogenous therapeutics results in a smaller dose of Spirulina for efficacy than administration of a Spirulina comprising a monomer of the same exogenous therapeutic.

Embodiment 66. The non-parenterally delivered composition of any of embodiments 1-65, wherein the recombinant Spirulina is selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor

Embodiment 67. The non-parenterally delivered composition of any one of embodiments 1-66, wherein the recombinant Spirulina is non-living.

Embodiment 68. The non-parenterally delivered composition of any one of embodiments 1-67, wherein the recombinant Spirulina is dried, spray dried, freeze-dried, or lyophilized.

Embodiment 69. The non-parenterally delivered composition of any one of embodiments 1-68, wherein the oral composition comprises a pharmaceutically acceptable excipient.

Embodiment 70. The non-parenterally delivered composition of any of embodiments 1-69, wherein the composition survives in the gastrointestinal tract or a simulated stomach environment.

Embodiment 71. The non-parenterally delivered composition of embodiment 70, wherein the composition survives in the gastrointestinal tract or a simulated stomach environment for at least 5 minutes.

Embodiment 72. The non-parenterally delivered composition of embodiment 71, wherein the composition survives in the gastrointestinal tract or a simulated stomach environment overnight.

Embodiment 73. A method of treating or preventing a disease or disorder in a subject in need thereof, comprising administering to the subject the non-parenterally delivered composition of any one of embodiments 1-72 or 87-92.

Embodiment 74. The method of embodiment 73, wherein the disease or disorder is an infection.

Embodiment 75. The method of embodiment 74, wherein the infection is bacterial, viral, fungal, or parasitical.

Embodiment 76. The method of embodiment 75, wherein the bacteria causing the infection is selected from the group consisting of: E. coli, Enterotoxigenic E. coli (ETEC), Shigella, Mycobacterium, Streptococcus, Staphylococcus, Shigella, Campylobacter, Salmonella, Clostridium, Corynebacterium, Pseudomonas, Neisseria, Listeria, Vibrio, Bordetella, and Legionella.

Embodiment 77. The method of embodiment 75, wherein the virus causing the infection is selected from the group consisting of: bacteriophage, RNA bacteriophage (e.g. MS2, AP205, PP7 and Qβ), Infectious Haematopoietic Necrosis Virus, Parvovirus, Herpes Simplex Virus, Hepatitis A virus, Hepatitis B virus, Hepatitis C virus, Measles virus, Mumps virus, Rubella virus, HIV, Influenza virus, Rhinovirus, Rotavirus A, Rotavirus B, Rotavirus C, Respiratory Syncytial Virus (RSV), Varicella zoster, Poliovirus, Norovirus, Zika Virus, Denge Virus, Rabies Virus, Newcastle Disease Virus, White Spot Syndrome Virus, a coronavirus, MERS, SARS, and SARS-CoV-2.

Embodiment 78. The method of embodiment 75, wherein the fungus causing the infection is selected from the group consisting of: Aspergillus, Candida, Blastomyces, Coccidioides, Cryptococcus, and Histoplasma.

Embodiment 79. The method of embodiment 75, wherein the parasite causing the infection is selected from the group consisting of: Plasmodium, P. falciparum, P. malariae, P. ovale, P. vivax, Trypanosoma, Toxoplasma, Giardia, Leishmania Cryptosporidium, helminthic parasites: Trichuris spp., Enterobius spp., Ascaris spp., Ancylostoma spp. and Necatro spp., Strongyloides spp., Dracunculus spp., Onchocerca spp. and Wuchereria spp., Taenia spp., Echinococcus spp., and Diphyllobothrium spp., Fasciola spp., and Schistosoma spp..

Embodiment 80. The method of embodiment 73, wherein the disease or disorder is selected from the list consisting of: Celiac disease, Type 1 diabetes, Type 2 diabetes, cancer, an inflammatory disorder, a gastrointestinal disease, an autoimmune disease or disorder, an endocrine disorder, gastroesophageal reflux disease (GERD), ulcers, high cholesterol, inflammatory bowel disorder, irritable bowel syndrome, crohn's disease, ulcerative colitis, constipation, and diarrhea

Embodiment 81. A method of treating or preventing a Campylobacter infection comprising administering to a subject the non-parenterally delivered composition of any of embodiments 1-72.

Embodiment 82. The method of embodiment 81, wherein administration of the non-parenterally delivered composition decreases or prevents development of campylobacter symptoms.

Embodiment 83. The method of any of embodiments 81-82, wherein administration of the non-parenterally delivered composition decreases or prevents the development of inflammation in the subject.

Embodiment 83. A method of treating or preventing a C. difficile infection comprising administering to a subject the non-parenterally delivered composition of any of embodiments 1-72.

Embodiment 84. The method of embodiment 83, wherein administration of the non-parenterally delivered composition decreases or prevents development of C. difficile symptoms.

Embodiment 85. The method of any one of embodiments 81-84, wherein administration of the non-parenterally delivered composition decreases or prevents the development of diarrhea in the subject.

Embodiment 86. The non-parenterally delivered composition or method of any one of embodiments 1-85 wherein the therapeutic or prophylactic molecule is not an antigen or epitope.

Embodiment 88. The non-parenterally delivered composition or method of any of the preceding embodiments, wherein the administration of two or more different recombinant Spirulina comprising different exogenous polypeptides or antigens or fragments thereof exert a synergistic effect.

Embodiment 89. The non-parenterally delivered composition or method of embodiment 88, wherein the different recombinant Spirulina administered each comprise a different VHH.

Embodiment 90. The non-parenterally delivered composition or method of any of the preceding embodiments, wherein the administration of a recombinant Spirulina comprising different exogenous polypeptides or antigens or fragments thereof exerts a synergistic effect.

Embodiment 91. The non-parenterally delivered composition or method of embodiment 90, wherein the recombinant Spirulina comprises two or more different VHH sequences.

Embodiment 92. The non-parenterally delivered composition or method of any of embodiments 88-92, wherein the recombinant Spirulina comprises a lysin.

Embodiment 93. The non-parenterally delivered composition or method of any of embodiments 88-92, wherein the recombinant Spirulina comprises a lysin and a exogenous polypeptide.

Embodiment 94. The non-parenterally delivered composition or method of embodiment 94, wherein the recombinant Spirulina comprises a lysin and a VHH.

Embodiment 95. The non-parenterally delivered composition or method of any of the preceding embodiments, wherein the composition is administered orally.

Embodiment 96. The non-parenterally delivered composition or method of any of the preceding embodiments, wherein the composition is delivered to the respiratory tract.

Embodiment 97. The non-parenterally delivered composition or method of embodiment 88, wherein the composition is delivered via inhalation or intranasally.

Embodiment 98. The non-parenterally delivered composition or method of any of the preceding embodiments, wherein the composition is a Spirulina biomass.

Embodiment 99. The non-parenterally delivered composition or method of any of the preceding embodiments, wherein the composition is delivered as an extract of a Spirulina biomass.

Embodiment 100. The non-parenterally delivered composition or method of any of the preceding embodiments, wherein the composition is delivered as a purified composition obtained from a Spirulina biomass.

### REFERENCES

Giallourou et al. A novel mouse model of Campylobacter jejuni enteropathy and diarrhea. PLoS Pathog. 2018 March; 14 (3): e1007083.
Riazi et al. Pentavalent Single-Domain Antibodies Reduce Campylobacter jejuni Motility and Colonization in Chickens. PLoS One. 2013; 8 (12): e83928.

### INCORPORATION BY REFERENCE

This patent application incorporates by reference in their entireties for all purposes the following patent publications and applications: U.S. Pat. No. 10,131,870, U.S. 62/672,891 filed May 17, 2018, and PCT/US2019/032998 filed May 17, 2019.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

TABLE 1
Cell Rounding Assay results (“+” is no rounding, “−” is all round, “+/−” is mixed)

| | | SP981 | | SP989 | | | | | | | |
|---|---|---|---|---|---|---|---|---|---|---|---|
| SP968 | | (5D) | | (5D) | | SP1090 | | SP1096 | | SP745 | |
| Hi/.01 | + | Hi/.01 | + | Hi/.01 | + | Hi/.01 | + | Hi/.01 | + | Hi/.01 | + |
| Hi/.1 | + | Hi/.1 | + | Hi/.1 | + | Hi/.1 | + | Hi/.1 | + | Hi/.1 | + |
| Hi/1. | +/− | Hi/1. | + | Hi/1. | + | Hi/1. | +/− | Hi/1. | +/− | Hi/1. | +/− |
| Lo/.01 | + | Lo/.01 | + | Lo/.01 | + | Lo/.01 | + | Lo/.01 | + | Lo/.01 | + |
| Lo/.1 | + | Lo/.1 | + | Lo/.1 | + | Lo/.1 | + | Lo/.1 | + | Lo/.1 | +/− |
| Lo/1. | +/− | Lo/1. | +/− | Lo/1. | +/− | Lo/1. | − | Lo/1. | − | Lo/1. | − |
| | | | | | | SP1091 | | | | | |
| SP974 | | SP982 | | SP990 | | (5D) | | SP1097 | | SP746 | |
| Hi/.01 | + | Hi/.01 | + | Hi/.01 | + | Hi/.01 | + | Hi/.01 | + | Hi/.01 | + |
| Hi/.1 | + | Hi/.1 | + | Hi/.1 | + | Hi/.1 | + | Hi/.1 | + | Hi/.1 | + |
| Hi/1. | − | Hi/1. | − | Hi/1. | − | Hi/1. | + | Hi/1. | +/− | Hi/1. | + |
| Lo/.01 | + | Lo/.01 | + | Lo/.01 | + | Lo/.01 | + | Lo/.01 | + | Lo/.01 | + |
| Lo/.1 | + | Lo/.1 | + | Lo/.1 | +/− | Lo/.1 | + | Lo/.1 | + | Lo/.1 | + |
| Lo/1. | − | Lo/1. | − | Lo/1. | − | Lo/1. | +/− | Lo/1. | +/− | Lo/1. | +/− |
| SP977 | | SP985 | | SP1086 | | | | | | | |
| (5D) | | (5D) | | (E3) | | SP1094 | | SP1102 | | SP747 | |
| Hi/.01 | + | Hi/.01 | + | Hi/.01 | + | Hi/.01 | + | Hi/.01 | + | Hi/.01 | + |
| Hi/.1 | + | Hi/.1 | + | Hi/.1 | + | Hi/.1 | + | Hi/.1 | + | Hi/.1 | + |
| Hi/1. | + | Hi/1. | + | Hi/1. | + | Hi/1. | +/− | Hi/1. | − | Hi/1. | − |
| Lo/.01 | + | Lo/.01 | + | Lo/.01 | + | Lo/.01 | + | Lo/.01 | + | Lo/.01 | + |
| Lo/.1 | + | Lo/.1 | + | Lo/.1 | + | Lo/.1 | +/− | Lo/.1 | + | Lo/.1 | + |
| Lo/1. | +/− | Lo/1. | +/− | Lo/1. | +/− | Lo/1. | − | Lo/1. | − | Lo/1. | − |

| | | | | SP1087 | | SP1095 | | SP744 | | | |
|---|---|---|---|---|---|---|---|---|---|---|---|
| SP978 | | SP986 | | (5D) | | (E3/5D) | | (5D) | | SP994 | |
| Hi/.01 | + | Hi/.01 | + | Hi/.01 | + | Hi/.01 | + | Hi/.01 | + | Hi/.01 | +/− |
| Hi/.1 | +/− | Hi/.1 | + | Hi/.1 | + | Hi/.1 | + | Hi/.1 | + | Hi/.1 | − |
| Hi/1. | − | Hi/1. | − | Hi/1. | + | Hi/1. | + | Hi/1. | + | Hi/1. | − |
| Lo/.01 | + | Lo/.01 | + | Lo/.01 | + | Lo/.01 | + | Lo/.01 | + | Lo/.01 | +/− |
| Lo/.1 | +/− | Lo/.1 | +/− | Lo/.1 | + | Lo/.1 | + | Lo/.1 | + | Lo/.1 | − |
| Lo/1. | − | Lo/1. | − | Lo/1. | + | Lo/1. | + | Lo/1. | + | Lo/1. | − |

1. A composition comprising recombinant Spirulina encapsulating a plurality of VHH molecules or binding fragments thereof that bind to a C. difficile toxin, and wherein the plurality of VHH molecules or binding fragments thereof comprise:
a) amino acid residues 26-32 (CDR1), 52-57 (CDR2), and 98-116 (CDR3) of SEQ ID NO: 5;
b) amino acid residues 26-32 (CDR1), 52-56 (CDR2), and 98-100 (CDR3) of SEQ ID NO: 6; and
c) amino acid residues 26-32 (CDR1), 52-56 (CDR2), and 98-105 (CDR3) of SEQ ID NO: 13.

2. The composition of claim 1, wherein the plurality of VHH molecules or binding fragments thereof comprise:
a) an amino acid sequence consisting of amino acid residues 2-126 of SEQ ID NO: 5;
b) an amino acid sequence consisting of amino acid residues 2-110 of SEQ ID NO: 6; and
c) an amino acid sequence consisting of amino acid residues 2-115 of SEQ ID NO: 13.

3. The composition of claim 1, further comprising a lysin.

4. The composition of claim 1, wherein at least one of the plurality of VHH molecules or binding fragments thereof is fused to a maltose binding protein (MBP).

5. The composition of claim 1, further comprising a lysin, and wherein at least one of the VHH molecules or binding fragments thereof is fused to a maltose binding protein (MBP).

6. The composition of claim 3, wherein the lysin is PlyCD.

7. The composition of claim 1, further comprising a PlyCD₁₋₁₇₄ lysin.

8. The composition of claim 1, comprising three recombinant Spirulina, wherein each of the three recombinant Spirulina comprises a different VHH molecule or binding fragment thereof.

9. The composition of claim 1, wherein the composition comprises a pharmaceutically acceptable excipient.

10. The composition of claim 1, wherein the composition retains its efficacy in binding to a C. difficile toxin after passage through the stomach.

11. The composition of claim 1, wherein the recombinant Spirulina are dried, spray dried, freeze-dried, or lyophilized.

12. The composition of claim 11, wherein the recombinant Spirulina are spray dried.

13. The composition of claim 1, wherein the recombinant Spirulina are non-living.

14. A composition comprising recombinant Spirulina encapsulating:
i) a plurality of VHH molecules or binding fragments thereof that bind to a C. difficile toxin, wherein the plurality of VHH molecules or binding fragments thereof comprise:
a) amino acid residues 26-32 (CDR1), 52-57 (CDR2), and 98-116 (CDR3) of SEQ ID NO: 5;
b) amino acid residues 26-32 (CDR1), 52-56 (CDR2), and 98-100 (CDR3) of SEQ ID NO: 6; and
c) amino acid residues 26-32 (CDR1), 52-56 (CDR2), and 98-105 (CDR3) of SEQ ID NO: 13; and
ii) a lysin.

15. The composition of claim 14, wherein the plurality of VHH molecules or binding fragments thereof further comprise:
a) an amino acid sequence consisting of amino acid residues 2-126 of SEQ ID NO: 5;
b) an amino acid sequence consisting of amino acid residues 2-110 of SEQ ID NO: 6; and
c) an amino acid sequence consisting of amino acid residues 2-115 of SEQ ID NO: 13.

16. The composition of claim 14, comprising three recombinant Spirulina, wherein each of the three recombinant Spirulina comprises a different VHH molecule or binding fragment thereof.

17. A method of treating an infection in a subject, comprising administering to the subject the composition of claim 1, orally.

18. The method of claim 17, wherein the infection is a C. difficile infection.

19. The method of claim 18, wherein administration of the composition decreases or prevents development of C. difficile symptoms.

20. A method of treating an infection in a subject, comprising administering to the subject the composition of claim 14, orally.

21. The method of claim 20, wherein the infection is a C. difficile infection.

22. The method of claim 21, wherein administration of the oral composition decreases or prevents development of C. difficile symptoms.

23. The composition of claim 14, wherein the lysin is PlyCD₁₋₁₇₄.
